SEQUENCE LISTING (65 sequences)

SEQ ID NO: 1 - DNA, 3822 bp, Artificial Sequence
  SARS-CoV-2 S Protein
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 2 - protein, 1273 aa, Artificial Sequence
  SARS-CoV-2 S Protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 3822 bp, Artificial Sequence
  SARS-CoV-2 D614G S Protein
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 4 - protein, 1273 aa, Artificial Sequence
  SARS-CoV-2 D614G S Protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - DNA, 672 bp, Artificial Sequence
  SARS-CoV-2 RBD S Protein
cgggtccaacccaccgaaagcattgtgcggttcccaaatatcaccaatctgtgtccctttggcgaagtgttcaatgctacaaggtttgcttctgtgtacgcatggaataggaaacgcatctccaattgtgtcgctgattactccgtgctgtacaattccgcctctttctcaaccttcaagtgttatggcgtttcacctaccaaacttaacgacctgtgcttcactaatgtgtatgccgactcttttgtgatacgaggcgatgaagtgagacagattgcaccagggcagaccggcaaaattgccgactacaactacaagcttccagatgactttaccggatgtgttattgcatggaactcaaacaatctggattccaaggtgggtggcaactataactacctgtatagactgttcaggaaatccaacctgaaaccattcgagcgagatataagcacagaaatctaccaggctggaagtacgccctgcaacggcgtggaagggttcaactgctacttcccattgcagagttacggattccagcctacaaacggggtgggttaccaaccctatcgtgtcgtagtcctgagttttgagctcctccatgccccagccacagtctgtggccccaagaaaagcaccaatctggtgaagaacaaatgcgtgaacttttag

SEQ ID NO: 6 - protein, 224 aa, Artificial Sequence
  SARS-CoV-2 RBD S Protein
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFG

SEQ ID NO: 7 - DNA, 668 bp, Artificial Sequence
  SARS-CoV-2 M Protein
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgcttcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 8 - protein, 222 aa, Artificial Sequence
  SARS-CoV-2 M Protein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 9 - DNA, 669 bp, Artificial Sequence
  WA9-UW6 Variant M Protein
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctatttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 10 - protein, 222 aa, Artificial Sequence
  WA9-UW6 Variant M Protein
MADSNGTITVEELKKLLEQWNLVIGFLFLTWICLLQFAYANRNRFLYIIKLIFLWLLWPVTLACFVLAAIYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 11 - DNA, 669 bp, Artificial Sequence
  NIHE Variant M Protein
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctggtatggccagtaactttagcttgttttgtgcttgctgccgtttacagaataaattggatcaccggtggaattgctatcgcaatggcttgtcttgtacgcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 12 - protein, 222 aa, Artificial Sequence
  NIHE Variant M Protein
MADSNGTITVEELKKLLEQWNLVIGFLFLTWICLLQFAYANRNRFLYIIKLIFLWLVWPVTLACFVLAAVYRINWITGGIAIAMACLVRLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 13 - DNA, 669 bp, Artificial Sequence
  Wuhan_IME_BJO7 Variant M Protein
atgtcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 14 - protein, 222 aa, Artificial Sequence
  Wuhan_IME_BJO7 Variant M Protein
MSDSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 15 - DNA, 669 bp, Artificial Sequence
  WA-UW-1753 Variant M Protein
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatgtcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 16 - protein, 222 aa, Artificial Sequence
  WA-UW-1753 Variant M Protein
MADSNGTITVEELKKLLEQWNLVIGFLFLTWICLLQFAYANRNRFLYIIKLIFLWLLWPVTLACFVLAAVYRINWITGGIAIAMSCLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 17 - DNA, 669 bp, Artificial Sequence
  WA UW-1755 Variant M Protein
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaatgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 18 - protein, 222 aa, Artificial Sequence
  WA UW-1755 Variant M Protein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRMLSYYKLGASQRVAGDSGFAAYSRYRIGNYKLNTDHSSSSDNIALLVQ

SEQ ID NO: 19 - DNA, 669 bp, Artificial Sequence
  USA_SC_3572 Variant M Protein
atggcaggttccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 20 - protein, 222 aa, Artificial Sequence
  USA_SC_3572 Variant M Protein
MAGSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 21 - DNA, 228 bp, Artificial Sequence
  SARS-CoV-2 E Protein
atgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 22 - protein, 75 aa, Artificial Sequence
  SARS-CoV-2 E Protein
MYSFVSEETGTLIVNSVLLFLAFVVFLLVTLAILTALRLCAYCCNIVNVSLVKPSFYVYSRVKNLNSSRVPDLLV

SEQ ID NO: 23 - DNA, 228 bp, Artificial Sequence
  USA_WA-UW-1588 Variant E Protein
atgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcttgatcttctggtctaa

SEQ ID NO: 24 - protein, 75 aa, Artificial Sequence
  USA_WA-UW-1588 Variant E Protein
MYSFVSEETGTLIVNSVLLFLAFVVFLLVTLAILTALRLCAYCCNIVNVSLVKPSFYVYSRVKNLNSSRVLDLLV

SEQ ID NO: 25 - DNA, 228 bp, Artificial Sequence
  SNU01 Variant E Protein
atgtactcattcgtttcggaagagacaggtacgttaatagttaatagcgtacttctttttcttgctttcgtggtattcttgctagttacactagccatccttactgcgcatcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 26 - protein, 75 aa, Artificial Sequence
  SNU01 Variant E Protein
MYSFVSEETGTLIVNSVLLFLAFVVFLLVTLAILTAHRLCAYCCNIVNVSLVKPSFYVYSRVKNLNSSRVPDLLV

SEQ ID NO: 27 - DNA, 1260 bp, Artificial Sequence
  SARS-CoV-2 N Protein
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 28 - protein, 419 aa, Artificial Sequence
  SARS-CoV-2 N Protein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 29 - DNA, 234 bp, Artificial Sequence
  SV40 p(A) tail
ctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatgg

SEQ ID NO: 30 - DNA, 225 bp, Artificial Sequence
  bGH p(A) tail
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 31 - DNA, 742 bp, Artificial Sequence
  Human CMV promoter
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatc

SEQ ID NO: 32 - DNA, 418 bp, Artificial Sequence
  SV40 promoter
gcgcagcaccatggcctgaaataacctctaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 33 - DNA, 508 bp, Artificial Sequence
  CAG promoter
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 34 - protein, 14 aa, Artificial Sequence
  Chikungunya virus antigen
PPFGAGRPGQFGDI

SEQ ID NO: 35 - protein, 9 aa, Artificial Sequence
  Chikungunya virus antigen
TAECKDKNL

SEQ ID NO: 36 - protein, 9 aa, Artificial Sequence
  Chikungunya virus antigen
VRYKCNCGG

SEQ ID NO: 37 - DNA, 251 bp, Artificial Sequence
  SV40 Enhancer
ggacctgcagggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagt

SEQ ID NO: 38 - DNA, 601 bp, Artificial Sequence
  hEF1-HTLV Promoter
gctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgcagttacagatccaagctgtgaccggcgcctacctgagatcaccggtcaccatggagatcaaggtgctgtttgccctcatctgtattgctgttgctgaggca

SEQ ID NO: 39 - DNA, 2010 bp, Artificial Sequence
  SARS-CoV-2 D614G Spike Truncated S1
gttaatctcaccacccgaactcaactcccacccgcatatacaaattccttcaccagaggagtgtactatcctgacaaagtgtttcggtcaagtgtcctccactctactcaggacctctttctgcctttcttttctaacgttacatggtttcatgcaatccatgtgtctgggacaaacggcaccaaacgcttcgacaaccctgtattgccattcaatgatggggtgtactttgcctccacagagaaatccaacatcattcgaggatggattttcgggactactctggactcaaagacacagagcctgctgatcgttaacaacgccacaaacgttgtcatcaaagtgtgcgaattccagttttgcaatgatcccttcctgggagtgtactatcacaagaataacaagtcctggatggagagcgaatttcgggtctacagcagcgcaaacaactgcaccttcgagtacgtgagtcaaccctttctgatggacctggaagggaaacagggaaacttcaagaacctgagagagtttgtctttaagaacatcgacggctattttaagatctatagtaagcatacgcctatcaacctggtaagggatcttccccagggcttttcagccctggaacctttggttgacttgcctattggtatcaatatcaccagatttcagacccttctggcattgcatcggtcttatcttactccaggtgattcctcctccgggtggactgccggcgccgctgcctactatgtcggctatctgcaaccaagaacgttcctgctcaagtacaacgaaaacggcactattacggatgctgttgattgtgccctggaccctctgtctgagactaaatgcaccctcaagagctttaccgttgagaaggggatttaccaaaccagtaatttccgggtccaacccaccgaaagcattgtgcggttcccaaatatcaccaatctgtgtccctttggcgaagtgttcaatgctacaaggtttgcttctgtgtacgcatggaataggaaacgcatctccaattgtgtcgctgattactccgtgctgtacaattccgcctctttctcaaccttcaagtgttatggcgtttcacctaccaaacttaacgacctgtgcttcactaatgtgtatgccgactcttttgtgatacgaggcgatgaagtgagacagattgcaccagggcagaccggcaaaattgccgactacaactacaagcttccagatgactttaccggatgtgttattgcatggaactcaaacaatctggattccaaggtgggtggcaactataactacctgtatagactgttcaggaaatccaacctgaaaccattcgagcgagatataagcacagaaatctaccaggctggaagtacgccctgcaacggcgtggaagggttcaactgctacttcccattgcagagttacggattccagcctacaaacggggtgggttaccaaccctatcgtgtcgtagtcctgagttttgagctcctccatgccccagccacagtctgtggccccaagaaaagcaccaatctggtgaagaacaaatgcgtgaactttaactttaacggactcacaggaaccggcgtattgacggagagtaacaagaagttcctgccattccagcagttcggtcgcgatattgccgacactaccgacgctgtccgagatccccagacattggagattcttgatatcacaccctgtagtttcggcggagtgagcgtgattacgcccggaaccaataccagcaatcaggttgccgtcctgtatcaggacgtgaattgcaccgaggtacctgtcgccatccacgctgaccaacttacacccacatggcgagtatattccaccggctccaacgtctttcagacacgtgctggatgtctgatcggtgcagaacacgttaataatagctacgagtgtgatatccccatcggtgctggaatatgcgcctcttatcaaactcaaaccaactctcctaggcgggcacgt

SEQ ID NO: 40 - protein, 670 aa, Artificial Sequence
  SARS-CoV-2 D614G Spike Truncated S1
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 41 - DNA, 568 bp, Artificial Sequence
  IRES Bridge
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacga

SEQ ID NO: 42 - DNA, 648 bp, Artificial Sequence
  Mouse IL-12 p35
atgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctga

SEQ ID NO: 43 - protein, 215 aa, Artificial Sequence
  Mouse IL-12 p35
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 44 - DNA, 1008 bp, Artificial Sequence
  Mouse IL-12 p40
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatagacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctag

SEQ ID NO: 45 - protein, 335 aa, Artificial Sequence
  Mouse IL-12 p40
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNIDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 46 - DNA, 660 bp, Artificial Sequence
  Human IL-12 p35
atgggtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 47 - protein, 219 aa, Artificial Sequence
  Human IL-12 p35
MGPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 48 - DNA, 989 bp, Artificial Sequence
  Human IL-12 p40
atgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttagac

SEQ ID NO: 49 - protein, 328 aa, Artificial Sequence
  Human IL-12 p40
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 50 - skipped

SEQ ID NO: 51 - DNA, 59 bp, Artificial Sequence
  AgeIMhis-F
accggcgcctacctgagatcaccggtaacgtagataagcgcttgccacaacccgggatc

SEQ ID NO: 52 - DNA, 72 bp, Artificial Sequence
  pAcelsion-R
tatttgaatgtatttagaaaaataaacaaatagggaaatgaagagcccataccacatttgtagaggttttac

SEQ ID NO: 53 - DNA, 57 bp, Artificial Sequence
  CelsionAgeIsp1-F
accggcgcctacctgagatcaccggtgccgccaccatggagatcaaggtgctgtttg

SEQ ID NO: 54 - DNA, 80 bp, Artificial Sequence
  IRESSalI-R
tgagggaagcggccgcccgggtcgactctagaggatcccgggttgtggcaagcttatcatcgtgtttttcaaaggaaaac

SEQ ID NO: 55 - skipped

SEQ ID NO: 56 - DNA, 41 bp, Artificial Sequence
  PspXI-SV40pA-F
cggctaagcggccgcagcctcgagcagctagctggccagac

SEQ ID NO: 57 - DNA, 34 bp, Artificial Sequence
  Cls-SapI-R
tatttgaatgtatttagaaaaataaacaaatagg

SEQ ID NO: 58 - DNA, 48 bp, Artificial Sequence
  Cls-BglII-R-2
attggacatgagccaatataaatgtacgaagatctgtcgagccatgtg

SEQ ID NO: 59 - DNA, 25 bp, Artificial Sequence
  Cls-XmnI-R
agaactttaaaagtgctcatcattg

SEQ ID NO: 60 - DNA, 23 bp, Artificial Sequence
  CLS-PspXI-IRES-F
atacgtaagcggccgcagcctcg

SEQ ID NO: 61 - DNA, 24 bp, Artificial Sequence
  IREScelsion-R
tcgtgtttttcaaaggaaaaccac

SEQ ID NO: 62 - DNA, 47 bp, Artificial Sequence
  Mhiscelsion-F
acgtggttttcctttgaaaaacacgatgataagcttgccacaacccg

SEQ ID NO: 63 - DNA, 20 bp, Artificial Sequence
  CLS-M-R
atcatgtctggccagctagc

SEQ ID NO: 64 - DNA, 49 bp, Artificial Sequence
  CLS-XbaI-M-F
ctagactcgagacagtccaccacaatctagagccaccatggctgactct

SEQ ID NO: 65 - DNA, 47 bp, Artificial Sequence
  CLS-MluI-M-R
actcatcaatgtatcttatcatgtctgctcgaacgcgtttattgtac

(19) United States
(12) Patent Application Publication
(10) Pub. No.: US 2025/0213679 A1
(43) Pub. Date: Jul. 3, 2025

(54) POLYNUCLEOTIDE VACCINES AND METHODS OF USING THE SAME

(71) Applicant: CELSION CORPORATION, Lawrenceville, NJ (US)

(72) Inventors: Khursheed ANWER, Madison, AL (US); Jason Glenn FEWELL, Madison, AL (US); Majed Mohd MATAR, Madison, AL (US); Anthony Joseph RECUPERO, Doylestown, PA (US); Brian Jeffrey SPARKS, Huntsville, AL (US); Daishui SU, Princeton, NJ (US); Michael Henry TARDUGNO, Princeton, NJ (US); Carlo IAVARONE, Lawrenceville, NJ (US); Subeena SOOD, Lawrenceville, NJ (US); John HENDERSON, Lawrenceville, NJ (US); Jessica KIM, Lawrenceville, NJ (US)

(21) Appl. No.: 18/247,588

(22) PCT Filed: Oct. 2, 2021
(86) PCT No.: PCT/US2021/053275

§ 371 Date: Mar. 31, 2023

Related U.S. Application Data
(60) Provisional application No. 63/087,118, filed on Oct. 2, 2020.

(51) Int. Cl. A61K 39/295; A61K 39/00; A61P 31/14

(52) U.S. Cl. CPC A61K 39/295; A61P 31/14; A61K 2039/53; A61K 2039/55522; A61K 2039/55527; A61K 2039/55533; A61K 2039/55555; A61K 2039/605; A61K 2039/6075; C12N 2770/20034

(57) ABSTRACT

Disclosed herein are polynucleotides comprising a first nucleic acid encoding a first pathogen antigen and, optionally, a second nucleic acid encoding a second pathogen antigen, and, optionally, a nucleic acid encoding an immune modifier. In some aspects, the first pathogen antigen is a SARS-CoV-2 spike protein or antigenic fragment thereof. In some aspects, the second pathogen antigen is a SARS-CoV-2 protein or an antigenic fragment thereof. In some aspects, polynucletide includes two or more different immune modifiers. Also disclosed herein are vectors, compositions, pharmaceutical compositions, vaccines, lyophilized compositions, and cells comprising such polynucleotides. Methods of production and therapeutic use are also disclosed herein.

### CROSS-REFERENCE TO RELATED APPLICATIONS

This application claims the benefit of U.S. Provisional Application No. 63/087,118, filed Oct. 2, 2020, which is incorporated by reference in its entirety.

### REFERENCE TO SEQUENCE LISTING SUBMITTED ELECTRONICALLY

The content of the electronically submitted sequence listing in ASCII text file (Name: 2437_049PC01_Seglisting_ST25.txt; Size: 98,606 bytes; and Date of Creation: Oct. 1, 2021 filed with the application is incorporated herein by reference in its entirety.

### FIELD

The present disclosure relates generally to immunology, vaccines, and gene therapy. In certain aspects, the disclosure relates to compositions and methods of generating an immune response to one or more viral antigens (e.g., SARS-CoV-2 antigens), bacterial antigens, or parasite antigens for treating, reducing the likelihood of, or preventing infection and disease in mammals.

### BACKGROUND OF THE DISCLOSURE

Severe acute respiratory syndrome (SARS) is a contagious viral respiratory illness caused by a coronavirus called SARS-associated coronavirus (SARS-CoV). Severe acute respiratory syndrome coronavirus 2 (SARS-CoV-2) is a strain of coronavirus that causes coronavirus disease 2019 (COVID-19) and is responsible for the COVID-19 pandemic. A wide range of symptoms have been reported for patients suffering from COVID-19-ranging from mild symptoms to severe illness, which can lead to death. Common symptoms include: fever, respiratory symptoms, fatigue, aches, loss of taste and/or smell, and intestinal distress (e.g., nausea, vomiting, and/or diarrhea).

The development of a safe and effective vaccine for SARS-CoV-2 is urgently needed. Several potential vaccine approaches including inactivated virus, antigen subunits, and nucleic acid (DNA and RNA) vaccines are being investigated in clinics across the globe. Recently, plasmid vectors encoding SARS-CoV-2 “S” protein were administered intramuscularly with the aid of an electric pulse in rodents or without a delivery system or adjuvant in rhesus monkeys, these DNA vaccines resulted in the production of neutralizing antibodies and antigen-specific T-cell responses. See Smith, T. R. F., et al., Nature Communications, 11(2601):1-13 (2020) (hereafter “Smith 2020”) and Hirao, L. A., et al., Science, 369(6505):806-11 (2020) hereafter “Hirao 2020”).

DNA vaccines can have certain advantages over conventional inactivated or protein subunit vaccines due to their potential to generate humoral and cellular immunity and low risk of virulence and folding problems associated with inactivated viruses and subunit vaccines, respectively. Furthermore, in comparison to RNA vaccines, DNA vaccines can have the potential for better stability, durability, lower cost, and longer development history. Despite their attractiveness, issues such as suboptimal immunogenicity and effective delivery have been concerns with DNA vaccines, which must be overcome for DNA vaccines to become a viable option.

Thus, a need remains for improved DNA vaccine compositions that are effective in prophylactic and therapeutic settings.

### BRIEF SUMMARY OF THE DISCLOSURE

Certain aspects of the disclosure relate to compositions comprising (i) a vector (e.g., a multicistronic DNA plasmid vector or a multicistronic messenger RNA (mRNA) vector) comprising a nucleic acid sequence encoding one or more viral antigens (e.g., a SARS CoV-2 antigen) and (ii) a delivery component (e.g., a cationic polymer, a poly-inosinic-polycytidylic acid, or a poloxamer). In some aspects, the vector further comprises a nucleic acid sequence encoding one or more immune modifiers. In some aspects, the vector comprises a nucleic acid sequence encoding a SARS CoV-2 antigen and, optionally, a second viral antigen. Some aspects relate to methods of eliciting humoral and/or cellular immune response against a pathogen (e.g., SARS-CoV-2) challenge or infection following in vivo administration of a vector or composition of the disclosure.

Certain aspects of the disclosure relate to compositions including (i) a multicistronic vector (e.g., a multicistronic DNA plasmid vector or a multicistronic messenger RNA (mRNA) vector) comprising a nucleic acid sequence encoding one or more antigens and a nucleic acid sequence encoding one or more immune modifiers and (ii) a delivery component, such as a synthetic non-viral carrier/adjuvant. Some aspects relate to methods of eliciting humoral and/or cellular immune response against a pathogen (e.g., SARS-CoV-2) challenge or infection following in vivo administration of a multicistronic DNA plasmid, a multicistronic mRNA vector, or a composition comprising the same of the disclosure.

Certain aspects of the disclosure relate to compositions including (i) a multicistronic DNA plasmid vector comprising a DNA sequence of one or more antigens and a DNA sequence of one or more immune modifiers and (ii) a delivery component, such as a synthetic non-viral DNA carrier/adjuvant. Certain aspects of the disclosure relate to compositions including (i) a multicistronic mRNA vector comprising an RNA sequence of one or more antigens and an RNA sequence of one or more immune modifiers and (ii) a delivery component, such as a synthetic non-viral RNA carrier/adjuvant. Some aspects relate to methods of eliciting a humoral and/or cellular immune response against a pathogen (e.g., SARS-CoV-2) challenge or infection following in vivo administration of a multicistronic DNA plasmid vector, multicistronic mRNA vector, or a composition comprising the same of the disclosure.

Certain aspects of the disclosure relate to compositions including (i) a vector (e.g., a DNA plasmid vector or a messenger RNA (mRNA) vector) comprising a nucleic acid sequence encoding one or more antigens and (ii) a delivery component, such as a synthetic non-viral carrier/adjuvant (e.g., a cationic polymer, a poly-inosinic-polycytidylic acid, or a poloxamer). Some aspects relate to methods of eliciting humoral and/or cellular immune response against a pathogen (e.g., SARS-CoV-2) challenge or infection following in vivo administration of a DNA plasmid vector, a mRNA vector, or a composition comprising the same of the disclosure.

Certain aspects of the disclosure relate to compositions including (i) a DNA plasmid vector comprising a DNA sequence of one or more antigens and (ii) a delivery component, such as a synthetic non-viral DNA carrier/adjuvant (e.g., a cationic polymer, a poly-inosinic-polycytidylic acid, or a poloxamer). Certain aspects of the disclosure relate to compositions including (i) a mRNA vector comprising an RNA sequence of one or more antigens and (ii) a delivery component, such as a synthetic non-viral RNA carrier/adjuvant (e.g., a cationic polymer, a poly-inosinic-polycytidylic acid, or a poloxamer). Some aspects relate to methods of eliciting a humoral and/or cellular immune response against a pathogen (e.g., SARS-CoV-2) challenge or infection following in vivo administration of a DNA plasmid vector, a mRNA vector, or a composition comprising the same of the disclosure. In some aspects, the delivery component further comprises benzalkonium chloride.

The delivery component of the compositions disclosed herein may comprise any combination of delivery components disclosed herein.

In some aspects, the pathogen is a virus, a bacterium or a parasite. In some aspects, the one or more antigens comprise one or more viral antigens, one or more bacterial antigens, or one or more parasite antigens. In some aspects the one or more viral antigens, one or more bacterial antigens, or one or more parasite antigens comprise two or more variants of the same antigen (e.g., two or more variants of an antigen from different strains of the virus, bacterium, or parasite).

In some aspects, the bacterial antigen is selected from the group consisting of a Yersinia pestis antigen, a Mycobacterium tuberculosis antigen, any antigenic fragments thereof, or any combination thereof. In some aspects, the Yersinia pestis antigen is a Yersinia pestis capsular antigen. In some aspects, the Yersinia pestis capsular antigen is F1-Ag or virulence antigen (V-Ag). In some aspects, the Mycobacterium tuberculosis antigen is an Apa antigen, an HP65 antigen, a rAg85A antigen, any antigenic fragments thereof, and any combinations thereof.

In some aspects, the viral antigen is selected from the group consisting of: an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, any antigenic fragments thereof, or any combination thereof. In some aspects, the enterovirus antigen is an enterovirus 71 (E71) antigen, a coxsackievirus (Cox) protein antigen, any antigenic fragments thereof, or any combination thereof. In some aspects, the E71 antigen is a E71-VP1 antigen, a glutathione S-transferase (GST)-tagged E71-VP1 antigen, any antigenic fragments thereof, or any combination thereof. In some aspects, the Cox protein antigen is GST-tagged Cox protein antigen. In some aspects, the HSV antigen is an HSV-1 envelope antigen, an HSV-2 envelope antigen, an HSV-2 surface glycoprotein antigen, any antigenic fragments thereof, or any combination thereof. In some aspects, the HSV-2 surface glycoprotein antigen is a gB2 antigen, a gC2 antigen, a gD2 antigen, a gE2 antigen, any antigenic fragments thereof, or any combination thereof. In some aspects, the HIV antigen is an Env antigen, a Gag antigen, a Nef antigen, a Pol antigen, any antigenic fragments thereof, or any combination thereof. In some aspects, the HPV antigen is a minor capsid protein L2 antigen. In some aspects, the minor capsid protein L2 antigen comprises one or more epitope domains (amino acids 10-36 and/or amino acids 65-89) of minor capsid protein L2. In some aspects, the HCV antigen is a nonstructural 3 (NS3) antigen. In some aspects, the RSV antigen is an F antigen, a G antigen, any antigenic fragments thereof, or a combination thereof. In some aspects, the Dengue virus antigen is an E protein antigen, an E protein domain III (EDIII) antigen, a non-structural protein 1 (NS1) antigen, a DEN-80E antigen, any antigenic fragments thereof, or any combination thereof. In some aspects, the Ebola virus antigen is a spike glycoprotein (GB) antigen, a VP24 antigen, a VP40 antigen, a nucleoprotein (NP) antigen, a VP30 antigen, a VP35 antigen, any antigenic fragments thereof, or any combination thereof. In some aspects the Zika virus antigen is an envelope domain III antigen, a CKD antigen, any antigenic fragments thereof, or any combination thereof. In some aspects, the Chikungunya virus antigen is an E1 glycoprotein subunit antigen, the MHC class I epitope PPFGAGRPGQFGDI (SEQ ID NO: 34), the MHC class I epitope TAECKDKNL (SEQ ID NO: 35), the MHC class II epitope VRYKCNCGG (SEQ ID NO: 36), any antigenic fragments thereof, or any combination thereof. In some aspects, the measles virus antigen is a hemagglutinin protein MV-H antigen, a fusion protein MV-F antigen, any antigenic fragments thereof, or any combination thereof. In some aspects, the MERS-CoV antigen is a spike (S) protein antigen, an antigen from the receptor-binding domain of the S protein, an antigen from the membrane fusion domain of the S protein, any antigenic fragments thereof, or any combination thereof. In some aspects, the SARS-CoV antigen is a spike (S) protein antigen, an antigen from the receptor binding domain of the S protein, an antigen from the membrane fusion domain of the S protein, an envelope (E) protein antigen, an M protein antigen, any antigenic fragments thereof, or any combinations thereof.

In some aspects, the one or more viral antigens comprise one or more influenza virus antigens from any influenza virus type or subtype. In some aspects, the one or more influenza virus antigens are selected from the group consisting of: an influenza virus hemagglutinin (HA) antigen, an influenza virus neuraminidase (NA) antigen, an influenza virus matrix-2 (M2) protein antigen, antigenic fragments thereof, and any combination thereof. In some aspects, the one or more influenza virus antigens are derived from influenza virus type A, type B, type C, type D, or any combination thereof. In some spects, the one or more influenza virus antigens are derived from influenza virus type A. In some aspects, the one or more influenza virus antigens derived from influenza virus type A have (a) a HA subtype selected from H1 through H18 or any combination thereof and (b) a NA subtype selected from N1 through N11 or any combination thereof. In some aspects, the one or more influenza virus antigens derived from influenza virus type A, subtype H1N1; influenza virus type A, subtype H2N2; influenza virus type A, subtype H3N2; influenza virus type A, subtype H5N1; influenza virus type A, subtype H7N7; influenza virus type A, subtype H7N9; influenza virus type A, subtype H9N2; or any combination thereof. In some aspects, the one or more influenza virus antigens are derived from influenza virus type A, subtype H1N1; influenza virus type A, subtype H3N2; or the combination thereof. In some spects, the one or more influenza virus antigens are derived from influenza virus type B. In some aspects, the one or more viral antigens comprise one or more SARS-CoV-2 antigens or antigenic fragments thereof disclosed herein and one or more influenza virus antigens or antigenic fragments thereof disclosed herein. In some aspects, the parasite antigen is a protozoan antigen. In some aspects, the parasite antigen is selected from the group consisting of a Toxoplasma gondii antigen, a Plasmodium falciparum antigen, any antigenic fragments thereof, or any combination thereof. In some aspects, the Toxoplasma gondii antigen is antigen MIC8. In some aspects, the Plasmodium falciparum antigen is a SERA5 polypeptide antigen, a circumsporozite protein antigen, any antigenic fragments thereof, or any combinations thereof. In some aspects, the parasite antigen is a parasitic or pathogenic fungus antigen. In some aspects, the parasitic or pathogenic fungus antigen is selected from the group consisting of a Candida spp. antigen (e.g., a Candida albicans antigen, a Candida glabrata antigen, a Candida parapsilosis antigen, a Candida tropicalis antigen, a Candida lusitaniae antigen, a Candida krusei antigen), a Pneumocystis spp. antigen, a Malassezia spp. antigen (e.g., a Malassezia furfur antigen), an Aspergillus fumigatus antigen, a Cryptococcus spp. antigen (e.g., a Cryptococcus neoformans antigen, a Cryptococcus gattii antigen), a Histoplasma capsulatum antigen, a Blastomyces dermatitidis antigen, a Paracoccidioides spp. antigen (e.g., a Paracoccidioides brasiliensis antigen, a Paracoccidioides lutzii antigen), a Coccidioides spp. antigen (e.g., a Coccidioides immitis antigen, a Coccidioides posadasii antigen), a Penicillium marneffei antigen, a Sporothrix schenckii antigen, a Trichosporon asahii antigen, a Fusarium spp. antigen (e.g., a Fusarium solanum antigen, a Fusarium oxysporum antigen), a Nectria spp. antigen, a Pseudoallescheria boydii antigen, a Cladophialphora bantianum antigen, a Ramichloridium spp. antigen, a Dactylaria gallopava antigen, an Exophiala spp. antigen (e.g., an Exophiala jeanselmei antigen, an Exophiala dermatitidis antigen), a Curvularia spp. antigen, a Bipolaris spp. antigen, an Alternaria spp. antigen, a Lacazia loboi antigen, a Conidiobolus spp. antigen (e.g., a Conidiobolus coronatus antigen, a Conidiobolus incongruus antigen), and any combination thereof.

In some aspects, the one or more antigens are viral antigens. In some aspects, the one or more viral antigens comprises a SARS-CoV-2 antigen or an antigenic fragment thereof. In some aspects, the one or more viral antigens comprise one or more viral antigens (e.g., a S protein, a S1 subunit of a S protein, a RBD of a S protein, a membrane fusion domain of a S protein, a M protein, an E protein, or an antigenic fragment thereof) from one or more SARS-CoV-2 strains selected from the group consisting of: an Alpha SARS-CoV-2 strain (e.g., strains B.1.1.7 and Q.1-Q.8); a Beta SARS-CoV-2 strain (e.g., strains B.1.351, B.1.351.2, and B.1.351.3); a Delta SARS-CoV-2 strain (e.g., strain B.1.617.2 and AY.1 sublineages); a SARS-CoV-2 strain Gamma strain (e.g., strains P.1, P.1.1, and P.1.2); an Epsilon SARS-CoV-2 strain (e.g., strains B.1.427 and B.1.429); an Eta SARS-CoV-2 strain (e.g., strain B.1.525); an Iota SARS-CoV-2 strain (e.g., strain B.1.526); a Kappa SARS-CoV-2 strain (e.g., strain B.1.617.1); a Lambda SARS-CoV-2 strain; a B.1.617.3 SARS-CoV-2 strain; a Mu SARS-CoV-2 strain (e.g., strains B.1.621 and B.1.621.1); a Zeta strain (e.g., strain P.2); and any combination thereof. In some aspects, the one or more viral antigens are one or more SARS-CoV-2 S proteins or antigenic fragments thereof from one or more SARS-CoV-2 strains selected from the group consisting of: an Alpha SARS-CoV-2 strain (e.g., strains B.1.1.7 and Q.1-Q.8); a Beta SARS-CoV-2 strain (e.g., strains B.1.351, B.1.351.2, and B.1.351.3); a Delta SARS-CoV-2 strain (e.g., strain B.1.617.2 and AY.1 sublineages); a SARS-CoV-2 strain Gamma strain (e.g., strains P.1, P.1.1, and P.1.2); an Epsilon SARS-CoV-2 strain (e.g., strains B.1.427 and B.1.429); an Eta SARS-CoV-2 strain (e.g., strain B.1.525); an Iota SARS-CoV-2 strain (e.g., strain B.1.526); a Kappa SARS-CoV-2 strain (e.g., strain B.1.617.1); a Lambda SARS-CoV-2 strain; a B.1.617.3 SARS-CoV-2 strain; a Mu SARS-CoV-2 strain (e.g., strains B.1.621 and B.1.621.1); a Zeta strain (e.g., strain P.2); and any combination thereof. In some aspects, the one or more viral antigens comprise one or more viral antigens (e.g., a S protein, a S1 subunit of a S protein, a RBD of a S protein, a M protein, or an antigenic fragment thereof) from one or more SARS-CoV-2 strains selected from the group consisting of: B.1.1.7, Q.1, Q.2, Q.3, Q.4, Q.5, Q.6, Q.7, Q.8, B.1.351, B.1.351.2, B.1.351.3, B.1.617.2, AY.1 sublineages, P.1, P.1.1, P.1.2, B.1.427, B.1.429, B.1.525, B.1.526, B.1.617.1, B.1.617.3, B.1.621, B.1.621.1, P.2, and any combination thereof. In some aspects, the one or more viral antigens are one or more SARS-CoV-2 S proteins or antigenic fragments thereof from one or more SARS-CoV-2 strains selected from the group consisting of: B.1.1.7, Q.1, Q.2, Q.3, Q.4, Q.5, Q.6, Q.7, Q.8, B.1.351, B.1.351.2, B.1.351.3, B.1.617.2, AY.1 sublineages, P.1, P.1.1, P.1.2, B.1.427, B.1.429, B.1.525, B.1.526, B.1.617.1, B.1.617.3, B.1.621, B.1.621.1, P.2, and any combination thereof.

In some aspects, the one or more viral antigens comprise a SARS-CoV-2 S protein or antigenic fragment thereof from an Alpha SARS-CoV-2 strain, wherein the SARS-CoV-2 S protein or antigenic fragment thereof comprises the amino acid sequence of SEQ ID NO: 2 or SEQ ID NO: 4 with one or mutations selected from ΔH69-V70, Δ144, E484K, N501Y, A570D, D614G, P681H, T716I, S982A, and D1118H wherein the amino acid locations correspond to SEQ ID NO: 2 or SEQ ID NO: 4. In some aspects, the one or more viral antigens comprise a SARS-CoV-2 S protein or antigenic fragment thereof from a Beta SARS-CoV-2 strain, wherein the SARS-CoV-2 S protein or antigenic fragment thereof comprises the amino acid sequence of SEQ ID NO: 2 or SEQ ID NO: 4 with one or mutations selected from L18F, D80A, D215G, AL241-S243, K417N, E484K, N501Y, D614G, and A701V, wherein the amino acid locations correspond to SEQ ID NO: 2 or SEQ ID NO: 4. In some aspects, the one or more viral antigens comprise a SARS-CoV-2 S protein or antigenic fragment thereof from a Gamma SARS-CoV-2 strain, wherein the SARS-CoV-2 S protein or antigenic fragment thereof comprises the amino acid sequence of SEQ ID NO: 2 or SEQ ID NO: 4 with one or mutations selected from L18F, T20N, P26S, D138Y, R190S, K417T, E484K, N501Y, D614G, H655Y, T1027I, and V1176F, wherein the amino acid locations correspond to SEQ ID NO: 2 or SEQ ID NO: 4. In some aspects, the one or more viral antigens comprise a SARS-CoV-2 S protein or antigenic fragment thereof from a Delta SARS-CoV-2 strain, wherein the SARS-CoV-2 S protein or antigenic fragment thereof comprises the amino acid sequence of SEQ ID NO: 2 or SEQ ID NO: 4 with one or mutations selected from T19R, AD 119-F120, AE156-F157, R158G, L452R, T478K, D614G, P681R, and D950N, wherein the amino acid locations correspond to SEQ ID NO: 2 or SEQ ID NO: 4. In some aspects, the one or more viral antigens comprise a SARS-CoV-2 S protein or antigenic fragment thereof from a Kappa SARS-CoV-2 strain, wherein the SARS-CoV-2 S protein or antigenic fragment thereof comprises the amino acid sequence of SEQ ID NO: 2 or SEQ ID NO: 4 with one or mutations selected from E154K, L452R, E484Q, D614G, P681R, and Q1071H, wherein the amino acid locations correspond to SEQ ID NO: 2 or SEQ ID NO: 4. In some aspects, the one or more viral antigens comprise a SARS-CoV-2 S protein or antigenic fragment thereof from an Eta SARS-CoV-2 strain, wherein the SARS-CoV-2 S protein or antigenic fragment thereof comprises the amino acid sequence of SEQ ID NO: 2 or SEQ ID NO: 4 with one or mutations selected from Q52R, A67V, ΔH69-V70, AY144, E484K, D614G, Q677H, and F888L, wherein the amino acid locations correspond to SEQ ID NO: 2 or SEQ ID NO: 4. In some aspects, the one or more viral antigens comprise a SARS-CoV-2 S protein or antigenic fragment thereof from an Iota SARS-CoV-2 strain, wherein the SARS-CoV-2 S protein or antigenic fragment thereof comprises the amino acid sequence of SEQ ID NO: 2 or SEQ ID NO: 4 with one or mutations selected from L5F, T95I, D253G, E484K, D614G, and A701V, wherein the amino acid locations correspond to SEQ ID NO: 2 or SEQ ID NO: 4. In some aspects, the one or more viral antigens comprise a SARS-CoV-2 S protein or antigenic fragment thereof from a Lambda SARS-CoV-2 strain, wherein the SARS-CoV-2 S protein or antigenic fragment thereof comprises the amino acid sequence of SEQ ID NO: 2 or SEQ ID NO: 4 with one or mutations selected from G75V, T76I, AR246-G252, D253N, L452Q, F490S, D614G, and T859N, wherein the amino acid locations correspond to SEQ ID NO: 2 or SEQ ID NO: 4. In some aspects, the one or more viral antigens comprise a SARS-CoV-2 S protein or antigenic fragment thereof from a Mu SARS-CoV-2 strain, wherein the SARS-CoV-2 S protein or antigenic fragment thereof comprises the amino acid sequence of SEQ ID NO: 2 or SEQ ID NO: 4 with one or mutations selected from T95I, Y144S, Y145N, R346K, E484K, N501Y, D614G, P681H, and D950N, wherein the amino acid locations correspond to SEQ ID NO: 2 or SEQ ID NO: 4. In some aspects, the one or more viral antigens comprise a SARS-CoV-2 S protein or antigenic fragment thereof from an Epsilon SARS-CoV-2 strain, wherein the SARS-CoV-2 S protein or antigenic fragment thereof comprises the amino acid sequence of SEQ ID NO: 2 or SEQ ID NO: 4 with one or mutations selected from S131, W152C, L452R, and D614G, wherein the amino acid locations correspond to SEQ ID NO: 2 or SEQ ID NO: 4.

In some aspects, the one or more viral antigens comprise at least two viral antigens (e.g., two or more antigens or antigenic fragments from a S protein, a S1 subunit of a S protein, a RBD of a S protein, a membrane fusion domain of a S protein, a M protein, an E protein, or any combination thereof) from two or more different strains of SARS-CoV-2. In some aspects, the one or more viral antigens comprise at least two SARS-CoV-2 S proteins or antigenic fragments thereof from two or more different strains of SARS-CoV-2. In some aspects, the two or more different strains of SARS-CoV-2 are selected from the group consisting of: an Alpha SARS-CoV-2 strain (e.g., strains B.1.1.7 and Q.1-Q.8); a Beta SARS-CoV-2 strain (e.g., strains B.1.351, B.1.351.2, and B.1.351.3); a Delta SARS-CoV-2 strain (e.g., strain B.1.617.2 and AY.1 sublineages); a SARS-CoV-2 strain Gamma strain (e.g., strains P.1, P.1.1, and P.1.2); an Epsilon SARS-CoV-2 strain (e.g., strains B.1.427 and B.1.429); an Eta SARS-CoV-2 strain (e.g., strain B.1.525); an Iota SARS-CoV-2 strain (e.g., strain B.1.526); a Kappa SARS-CoV-2 strain (e.g., strain B.1.617.1); a Lambda SARS-CoV-2 strain; a B.1.617.3 SARS-CoV-2 strain; a Mu SARS-CoV-2 strain (e.g., strains B.1.621 and B.1.621.1); a Zeta strain (e.g., strain P.2); and any combination thereof. In some aspects, the two or more different strains of SARS-CoV-2 are selected from the group consisting of: B.1.1.7, Q.1, Q.2, Q.3, Q.4, Q.5, Q.6, Q.7, Q.8, B.1.351, B.1.351.2, B.1.351.3, B.1.617.2, AY.1 sublineages, P.1, P.1.1, P.1.2, B.1.427, B.1.429, B.1.525, B.1.526, B.1.617.1, B.1.617.3, B.1.621, B.1.621.1, P.2, and any combination thereof. In some aspects, the at least two SARS-CoV-2 S proteins or antigenic fragments thereof from two or more different strains of SARS-CoV-2 comprise one or more mutations previously reported in Li, T. et al., Emerg Microbes Infect. 9(1):2076-90 (2020); Lee, P. et al., Immune Netw. 21(1):e4 (2021); Yu, J. et al., Science 369(6505):806-11 (2020); Cattin-Ortola, J. et al., Nat Commun. 12(1):5333 (2021); Corbett, K. et al., Nature 586(7830):567-71 (2020); Hsieh, C. et al., Science 369(6510):1501-5 (2020); and Harvey, W. et al., Nat Rev Microbiol. 19(7):409-24 (2021), each of which is incorporated by reference herein in its entirety.

Mutations and viral sequence data for the SARS-CoV-2 variants are publically available at the CoVariants website (https://covariants.org/) and the National Center for Biotechnology Information (NCBI) website (https://www.ncbi.nlm.nih.gov/labs/virus/vssi/#/sars-cov-2), each of which is incorporated by reference in its entirety. In some aspects, the SARS-CoV-2 S proteins or antigenic fragments thereof disclosed herein include a SARS-CoV-2 S protein or antigenic fragment thereof from an Alpha SARS-CoV-2 strain, wherein the SARS-CoV-2 S protein or antigenic fragment thereof comprises the amino acid sequence of SEQ ID NO: 2 or SEQ ID NO: 4 with one or mutations selected from ΔH69-V70, Δ144, E484K, N501Y, A570D, D614G, P681H, T716I, S982A, and D1118H wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from a Beta SARS-CoV-2 strain, wherein the SARS-CoV-2 S protein or antigenic fragment thereof comprises the amino acid sequence of SEQ ID NO: 2 or SEQ ID NO: 4 with one or mutations selected from L18F, D80A, D215G, AL241-S243, K417N, E484K, N501Y, D614G, and A701V, wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from a Gamma SARS-CoV-2 strain, wherein the SARS-CoV-2 S protein or antigenic fragment thereof comprises the amino acid sequence of SEQ ID NO: 2 or SEQ ID NO: 4 with one or mutations selected from L18F, T20N, P26S, D138Y, R190S, K417T, E484K, N501Y, D614G, H655Y, T1027I, and V1176F, wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from a Delta SARS-CoV-2 strain, wherein the SARS-CoV-2 S protein or antigenic fragment thereof comprises the amino acid sequence of SEQ ID NO: 2 or SEQ ID NO: 4 with one or mutations selected from T19R, AD119-F120, AE156-F157, R158G, L452R, T478K, D614G, P681R, and D950N, wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from a Kappa SARS-CoV-2 strain, wherein the SARS-CoV-2 S protein or antigenic fragment thereof comprises the amino acid sequence of SEQ ID NO: 2 or SEQ ID NO: 4 with one or mutations selected from E154K, L452R, E484Q, D614G, P681R, and Q1071H, wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from an Eta SARS-CoV-2 strain, wherein the SARS-CoV-2 S protein or antigenic fragment thereof comprises the amino acid sequence of SEQ ID NO: 2 or SEQ ID NO: 4 with one or mutations selected from Q52R, A67V, ΔH69-V70, AY144, E484K, D614G, Q677H, and F888L, wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from an Iota SARS-CoV-2 strain, wherein the SARS-CoV-2 S protein or antigenic fragment thereof comprises the amino acid sequence of SEQ ID NO: 2 or SEQ ID NO: 4 with one or mutations selected from L5F, T95I, D253G, E484K, D614G, and A701V, wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from a Lambda SARS-CoV-2 strain, wherein the SARS-CoV-2 S protein or antigenic fragment thereof comprises the amino acid sequence of SEQ ID NO: 2 or SEQ ID NO: 4 with one or mutations selected from G75V, T76I, AR246-G252, D253N, L452Q, F490S, D614G, and T859N, wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from a Mu SARS-CoV-2 strain, wherein the SARS-CoV-2 S protein or antigenic fragment thereof comprises the amino acid sequence of SEQ ID NO: 2 or SEQ ID NO: 4 with one or mutations selected from T95I, Y144S, Y145N, R346K, E484K, N501Y, D614G, P681H, and D950N, wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from an Epsilon SARS-CoV-2 strain, wherein the SARS-CoV-2 S protein or antigenic fragment thereof comprises the amino acid sequence of SEQ ID NO: 2 or SEQ ID NO: 4 with one or mutations selected from S131, W152C, L452R, and D614G, wherein the amino acid locations correspond to SEQ ID NO: 2 or SEQ ID NO: 4.

Certain aspects of the disclosure relate to compositions including (i) a multicistronic DNA plasmid vector comprising a DNA sequence of one or more SARS-CoV-2 viral antigens and a DNA sequence of one or more immune modifiers and (ii) a delivery component, such as a synthetic non-viral DNA carrier/adjuvant (e.g., a cationic polymer, a poly-inosinic-polycytidylic acid, or a poloxamer). In some aspects, the compositions further comprise one or more immunopotentiators that are capable of activating the innate immunity system. In some aspects, the immunopotentiators are selected from a non-coding DNA (e.g., concatamers of non-coding 5′-C-phosphate-G-3′ (CpG) dinucleotides), a non-coding RNA, a small molecule, or any combination thereof. Certain aspects of the disclosure relate to compositions including (i) a multicistronic RNA vector comprising an RNA sequence of one or more SARS-CoV-2 viral antigens and an RNA sequence of one or more immune modifiers and (ii) a delivery component, such as a synthetic non-viral RNA carrier/adjuvant (e.g., a cationic polymer, a poly-inosinic-polycytidylic acid, or a poloxamer). Some aspects relate to methods of eliciting humoral and/or cellular immune response against a SARS-CoV-2 challenge or infection following in vivo administration of a multicistronic DNA plasmid vector, a multicistronic mRNA vector, or a composition comprising the same of the disclosure.

Certain aspects of the disclosure relate to compositions including (i) a DNA plasmid vector comprising a DNA sequence of one or more SARS-CoV-2 viral antigens and (ii) a delivery component, such as a synthetic non-viral DNA carrier/adjuvant (e.g., a cationic polymer, a poly-inosinic-polycytidylic acid, or a poloxamer). Certain aspects of the disclosure relate to compositions including (i) a RNA vector comprising an RNA sequence of one or more SARS-CoV-2 viral antigens and (ii) a delivery component, such as a synthetic non-viral RNA carrier/adjuvant (e.g., a cationic polymer, a poly-inosinic-polycytidylic acid, or a poloxamer). Some aspects relate to methods of eliciting humoral and/or cellular immune response against a SARS-CoV-2 challenge or infection following in vivo administration of a DNA plasmid vector, a mRNA vector, or a composition comprising the same of the disclosure.

Certain aspects of the disclosure relate to polynucleotide comprising: (a) a first antigen nucleic acid which encodes a first pathogen protein or an antigenic fragment thereof; and (b) a nucleic acid encoding an immune modifier. In some aspects, the polynucleotide is multicistronic (e.g., a multicistronic DNA plasmid or a multicistronic messenger RNA (mRNA)). In some aspects, the polynucleotide is combined with a delivery component, such as a synthetic non-viral carrier/adjuvant.

The delivery component combined with the polynucleotides disclosed herein may comprise any combination of delivery components disclosed herein.

Certain aspects of the disclosure relate to a polynucleotide comprising: (a) a first antigen nucleic acid which encodes a first pathogen protein or an antigenic fragment thereof, wherein the first antigen nucleic acid is operably linked to a first promoter; and (b) a nucleic acid encoding an immune modifier. In some aspects, the first pathogen protein is a viral antigen, a bacterial antigen, or a parasite antigen. In some aspects, the pathogen protein is a viral antigen, e.g., a SARS-CoV-2 spike (S) protein or an antigenic fragment thereof. In some aspects, the pathogen is a viral antigen, e.g., a SARS-CoV-2 spike (S) protein or an antigenic fragment thereof, from a SARS-CoV-2 strain selected from the group consisting of: an Alpha SARS-CoV-2 strain (e.g., strains B.1.1.7 and Q.1-Q.8); a Beta SARS-CoV-2 strain (e.g., strains B.1.351, B.1.351.2, and B.1.351.3); a Delta SARS-CoV-2 strain (e.g., strain B.1.617.2 and AY.1 sublineages); a SARS-CoV-2 strain Gamma strain (e.g., strains P.1, P.1.1, and P.1.2); an Epsilon SARS-CoV-2 strain (e.g., strains B.1.427 and B.1.429); an Eta SARS-CoV-2 strain (e.g., strain B.1.525); an Iota SARS-CoV-2 strain (e.g., strain B.1.526); a Kappa SARS-CoV-2 strain (e.g., strain B.1.617.1); a Lambda SARS-CoV-2 strain; a B.1.617.3 SARS-CoV-2 strain; a Mu SARS-CoV-2 strain (e.g., strains B.1.621 and B.1.621.1); and a Zeta strain (e.g., strain P.2). In some aspects, the pathogen protein is a bacterial antigen. In some aspects, the pathogen protein is a parasite antigen. In some aspects, the polynucleotide is a multicistronic DNA vector. In some aspects, the polynucleotide is a multicistronic mRNA vector.

In some aspects, provided herein is a polynucleotide comprising: (a) a first antigen nucleic acid which encodes a SARS-CoV-2 spike (S) protein or an antigenic fragment thereof, wherein the first antigen nucleic acid is operably linked to a first promoter; and (b) a nucleic acid encoding an immune modifier. In some aspects, the polynucleotide is a multicistronic DNA vector. In some aspects, the SARS-CoV-2 S protein or antigenic fragment thereof is a SARS-CoV-2 S protein or antigenic fragment thereof from a SARS-CoV-2 strain selected from the group consisting of: an Alpha SARS-CoV-2 strain (e.g., strains B.1.1.7 and Q.1-Q.8); a Beta SARS-CoV-2 strain (e.g., strains B.1.351, B.1.351.2, and B.1.351.3); a Delta SARS-CoV-2 strain (e.g., strain B.1.617.2 and AY.1 sublineages); a SARS-CoV-2 strain Gamma strain (e.g., strains P.1, P.1.1, and P.1.2); an Epsilon SARS-CoV-2 strain (e.g., strains B.1.427 and B.1.429); an Eta SARS-CoV-2 strain (e.g., strain B.1.525); an Iota SARS-CoV-2 strain (e.g., strain B.1.526); a Kappa SARS-CoV-2 strain (e.g., strain B.1.617.1); a Lambda SARS-CoV-2 strain; a B.1.617.3 SARS-CoV-2 strain; a Mu SARS-CoV-2 strain (e.g., strains B.1.621 and B.1.621.1); and a Zeta strain (e.g., strain P.2).

In some aspects, the SARS-CoV-2 S protein or antigenic fragment thereof is from an Alpha SARS-CoV-2 strain, wherein the SARS-CoV-2 S protein or antigenic fragment thereof comprises the amino acid sequence of SEQ ID NO: 2 or SEQ ID NO: 4 with one or mutations selected from ΔH69-V70, Δ144, E484K, N501Y, A570D, D614G, P681H, T716I, S982A, and D1118H wherein the amino acid locations correspond to SEQ ID NO: 2 or SEQ ID NO: 4. In some aspects, the SARS-CoV-2 S protein or antigenic fragment thereof is from a Beta SARS-CoV-2 strain, wherein the SARS-CoV-2 S protein or antigenic fragment thereof comprises the amino acid sequence of SEQ ID NO: 2 or SEQ ID NO: 4 with one or mutations selected from L18F, D80A, D215G, AL241-S243, K417N, E484K, N501Y, D614G, and A701V, wherein the amino acid locations correspond to SEQ ID NO: 2 or SEQ ID NO: 4. In some aspects, the SARS-CoV-2 S protein or antigenic fragment thereof is from a Gamma SARS-CoV-2 strain, wherein the SARS-CoV-2 S protein or antigenic fragment thereof comprises the amino acid sequence of SEQ ID NO: 2 or SEQ ID NO: 4 with one or mutations selected from L18F, T20N, P26S, D138Y, R190S, K417T, E484K, N501Y, D614G, H655Y, T1027I, and V1176F, wherein the amino acid locations correspond to SEQ ID NO: 2 or SEQ ID NO: 4. In some aspects, the SARS-CoV-2 S protein or antigenic fragment thereof is from a Delta SARS-CoV-2 strain, wherein the SARS-CoV-2 S protein or antigenic fragment thereof comprises the amino acid sequence of SEQ ID NO: 2 or SEQ ID NO: 4 with one or mutations selected from T19R, AD119-F120, AE156-F157, R158G, L452R, T478K, D614G, P681R, and D950N, wherein the amino acid locations correspond to SEQ ID NO: 2 or SEQ ID NO: 4. In some aspects, the SARS-CoV-2 S protein or antigenic fragment thereof is from a Kappa SARS-CoV-2 strain, wherein the SARS-CoV-2 S protein or antigenic fragment thereof comprises the amino acid sequence of SEQ ID NO: 2 or SEQ ID NO: 4 with one or mutations selected from E154K, L452R, E484Q, D614G, P681R, and Q1071H, wherein the amino acid locations correspond to SEQ ID NO: 2 or SEQ ID NO: 4. In some aspects, the SARS-CoV-2 S protein or antigenic fragment thereof is from an Eta SARS-CoV-2 strain, wherein the SARS-CoV-2 S protein or antigenic fragment thereof comprises the amino acid sequence of SEQ ID NO: 2 or SEQ ID NO: 4 with one or mutations selected from Q52R, A67V, ΔH69-V70, AY144, E484K, D614G, Q677H, and F888L, wherein the amino acid locations correspond to SEQ ID NO: 2 or SEQ ID NO: 4. In some aspects, the SARS-CoV-2 S protein or antigenic fragment thereof is from an Iota SARS-CoV-2 strain, wherein the SARS-CoV-2 S protein or antigenic fragment thereof comprises the amino acid sequence of SEQ ID NO: 2 or SEQ ID NO: 4 with one or mutations selected from L5F, T95I, D253G, E484K, D614G, and A701V, wherein the amino acid locations correspond to SEQ ID NO: 2 or SEQ ID NO: 4. In some aspects, the SARS-CoV-2 S protein or antigenic fragment thereof is from a Lambda SARS-CoV-2 strain, wherein the SARS-CoV-2 S protein or antigenic fragment thereof comprises the amino acid sequence of SEQ ID NO: 2 or SEQ ID NO: 4 with one or mutations selected from G75V, T76I, AR246-G252, D253N, L452Q, F490S, D614G, and T859N, wherein the amino acid locations correspond to SEQ ID NO: 2 or SEQ ID NO: 4. In some aspects, the SARS-CoV-2 S protein or antigenic fragment thereof is from a Mu SARS-CoV-2 strain, wherein the SARS-CoV-2 S protein or antigenic fragment thereof comprises the amino acid sequence of SEQ ID NO: 2 or SEQ ID NO: 4 with one or mutations selected from T95I, Y144S, Y145N, R346K, E484K, N501Y, D614G, P681H, and D950N, wherein the amino acid locations correspond to SEQ ID NO: 2 or SEQ ID NO: 4. In some aspects, the SARS-CoV-2 S protein or antigenic fragment thereof is from an Epsilon SARS-CoV-2 strain, wherein the SARS-CoV-2 S protein or antigenic fragment thereof comprises the amino acid sequence of SEQ ID NO: 2 or SEQ ID NO: 4 with one or mutations selected from S13I, W152C, L452R, and D614G, wherein the amino acid locations correspond to SEQ ID NO: 2 or SEQ ID NO: 4.

In some aspects, the polynucleotide comprises two or more nucleic acids encoding an immune modifier. In some aspects, each of the nucleic acids encoding an immune modifier encodes a different immune modifier. In some aspects, the two or more of the nucleic acids encoding an immune modifier encode IL-12 p35 and IL-12 p40.

In some aspects, the polynucleotide further comprises: (c) a second antigen nucleic acid which encodes a second pathogen protein or an antigenic fragment thereof. In some aspects, the second pathogen protein or antigenic fragment thereof is selected from the group consisting of: a second viral antigen protein, a second bacterial antigen protein or a second parasite antigen protein.

In some aspects, the polynucleotide further comprises: (c) a second antigen nucleic acid which encodes a SARS-CoV-2 protein or an antigenic fragment thereof. In some aspects, the SARS-CoV-2 protein or antigenic fragment thereof is selected from the group consisting of: a SARS-CoV-2 membrane (M) protein or an antigenic fragment thereof, a SARS-CoV-2 envelope (E) protein or an antigenic fragment thereof, a SARS-CoV-2 nucleocapsid (N) protein or an antigenic fragment thereof, and any combination thereof. In some aspects, the first antigen nucleic acid and the second antigen nucleic acid encode SARS-CoV-2 proteins or antigenic fragments thereof from different strains of SARS-CoV-2. In some aspects, the first antigen nucleic acid and the second antigen nucleic acid encode different variants of the same SARS-CoV-2 protein or antigenic fragment thereof, wherein the different variants of the same SARS-CoV2 protein or antigenic fragment thereof are derived from different strains of SARS-CoV-2. In some aspects, the first antigen nucleic acid and the second antigen nucleic acid encode SARS-CoV-2 S proteins or antigenic fragments thereof from different strains of SARS-CoV-2. In some aspects, the SARS-CoV-2 S proteins or antigenic fragments thereof from different strains of SARS-CoV-2 comprise one or more mutations previously reported in Li, T. et al., Emerg Microbes Infect. 9(1):2076-90 (2020); Lee, P. et al., Immune Netw. 21(1):e4 (2021); Yu, J. et al., Science 369(6505):806-11 (2020); Cattin-Ortola, J. et al., Nat Commun. 12(1):5333 (2021); Corbett, K. et al., Nature 586(7830):567-71 (2020); Hsieh, C. et al., Science 369(6510):1501-5 (2020); and Harvey, W. et al., Nat Rev Microbiol. 19(7):409-24 (2021), each of which is incorporated by reference herein in its entirety. In some aspects, the different strains of SARS-CoV-2 are selected from the group consisting of: an Alpha SARS-CoV-2 strain (e.g., strains B.1.1.7 and Q.1-Q.8); a Beta SARS-CoV-2 strain (e.g., strains B.1.351, B.1.351.2, and B.1.351.3); a Delta SARS-CoV-2 strain (e.g., strain B.1.617.2 and AY.1 sublineages); a SARS-CoV-2 strain Gamma strain (e.g., strains P.1, P.1.1, and P.1.2); an Epsilon SARS-CoV-2 strain (e.g., strains B.1.427 and B.1.429); an Eta SARS-CoV-2 strain (e.g., strain B.1.525); an Iota SARS-CoV-2 strain (e.g., strain B.1.526); a Kappa SARS-CoV-2 strain (e.g., strain B.1.617.1); a Lambda SARS-CoV-2 strain; a B.1.617.3 SARS-CoV-2 strain; a Mu SARS-CoV-2 strain (e.g., strains B.1.621 and B.1.621.1); a Zeta strain (e.g., strain P.2); and any combination thereof. In some aspects, the different strains of SARS-CoV-2 are selected from the group consisting of: B.1.1.7, Q.1, Q.2, Q.3, Q.4, Q.5, Q.6, Q.7, Q.8, B.1.351, B.1.351.2, B.1.351.3, B.1.617.2, AY.1 sublineages, P.1, P.1.1, P.1.2, B.1.427, B.1.429, B.1.525, B.1.526, B.1.617.1, B.1.617.3, B.1.621, B.1.621.1, P.2, and any combination thereof.

In some aspects, the second antigen nucleic acid is operably linked to the first promoter through an internal ribosome entry site (IRES) sequence. In some aspects, the IRES sequence comprises a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 41.

In some aspects, the polynucleotide comprises one or more second promoters. In some aspects, the second antigen nucleic acid is operably linked to the one or more second promoters. In some aspects, one or more nucleic acids encoding an immune modifier is operably linked to the one or more second promoters. In some aspects, one or more of the nucleic acids encoding an immune modifier is operably linked to the first promoter or the one or more second promoters through an internal ribosome entry site (IRES) sequence. In some aspects, the IRES sequence comprises a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 41.

In some aspects, the first promoter or the one or more second promoters is selected from the group consisting of: a cytomegalovirus (CMV) promoter (SEQ ID NO: 31), a Rouse sarcoma virus (RSV) promoter, a Moloney murine leukemia virus (Mo-MuLV) long terminal repeat (LTR) promoter, a mammalian elongation factor 1 (EF1) promoter, a cytokeratin 18 (CK18) promoter, a cytokeratin 19 (CK19) promoter, a simian virus 40 (SV40) promoter (SEQ ID NO: 32), a murine U6 promoter, a skeletal α-actin promoter, a β-actin promoter, a murine phosphoglycerate kinase 1 (PGK1) promoter, a human PGK1 promoter, a CBA promoter, a CAG promoter (SEQ ID NO: 33), and any combination thereof. In some aspects, the mammalian EF1 promoter is a hEF1-HTLV promoter (SEQ ID NO: 38). In some aspects, the one or more second promoters is the CMV promoter.

In some aspects, each of the nucleic acids which encodes an immune modifier is under the control of a promoter selected from the group consisting of a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CBA promoter, a CAG promoter, and any combination thereof. In some aspects, the mammalian EF1 promoter is a hEF1-HTLV promoter.

In some aspects, each of the second antigen nucleic acids is under the control of a promoter selected from the group consisting of a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CBA promoter, a CAG promoter, and any combination thereof. In some aspects, the mammalian EF1 promoter is a hEF1-HTLV promoter.

In some aspects, the immune modifier is selected from the group consisting of: interleukin (IL) 2 (IL-2), IL-12 p35, IL-12 p40, IL-12 p70, IL-15, IL-18, tumor necrosis factor alpha (TNFα), granulocyte-macrophage colony-stimulating factor (GM-CSF), interferon (IFN) α (IFN-α), IFN-β, a chemokine, major histocompatibility complex (MHC) class I (MHC I), MHC class II (MHC II), human leukocyte antigen (HLA)-DR isotype (HLA-DR), CD80, CD86, and any combination thereof. In some aspects, the chemokine is selected from the group consisting of: C-C motif chemokine ligand (CCL) 3 (CCL3), CCL4, CCL5, CCL21, CCL28, C-X-C motif chemokine ligand (CXCL) 10 (CXCL10), and any combination thereof.

In some aspects, the IL-12 p35 immune modifier comprises an amino acid sequence having at least 70%, at least 75%, at least 80%, at least 85%, at least 90%, at least 95%, at least 99%, or 100% sequence identity to SEQ ID NO: 43 (mouse IL-12 p35) or SEQ ID NO: 47 (human IL-12 p35). In some aspects, the IL-12 p40 immune modifier comprises an amino acid sequence having at least 70%, at least 75%, at least 80%, at least 85%, at least 90%, at least 95%, at least 99%, or 100% sequence identity to SEQ ID NO: 45 (mouse IL-12 p40) or SEQ ID NO: 49 (human IL-12 p40).

In some aspects, the nucleic acid encoding IL-12 p35 has at least 70%, at least 75%, at least 80%, at least 85%, at least 90%, at least 95%, at least 99%, or 100% sequence identity to SEQ ID NO: 42 (nucleic acid sequence encoding mouse IL-12 p35) or SEQ ID NO: 46 (nucleic acid sequence encoding human IL-12 p35). In some aspects, the nucleic acid encoding IL-12 p40 has at least 70%, at least 75%, at least 80%, at least 85%, at least 90%, at least 95%, at least 99%, or 100% sequence identity to SEQ ID NO: 44 (nucleic acid sequence encoding mouse IL-12 p40) or SEQ ID NO: 48 (nucleic acid sequence encoding human IL-12 p40).

In some aspects, the immune modifier comprises a viral protein (e.g., SARS-CoV-2 non-structural protein 1 (Nsp1), SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b) that attenuates a local inflammatory response and/or interferon response. In some aspects, the viral protein is from the same virus as a viral antigen encoded by an antigen nucleic acid. In some aspects, In some aspects, the viral protein is from a different virus than a viral antigen encoded by an antigen nucleic acid. In some aspects, the viral protein attenuates a local inflammatory response and/or interferon response elicited by a pathogen antigen disclosed herein. In some aspects, the immune modifier comprises SARS-CoV-2 Nsp1, SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b, or any combination thereof.

In some aspects, the immune modifier comprises one or more concatamers of non-coding 5′-C-phosphate-G-3′ (CpG) dinucleotides. In some aspects, the one or more concatamers of non-coding CpG dinucleotides activate the Toll-like receptor 9 (TLR9) signaling pathway. In some aspects, the one or more concatamers of non-coding CpG dinucleotides comprise one or more concatamers of non-coding CpG dinucleotides previously reported in Bauer, A. et al., Nucleic Acids Research 38(12):3891-908 (2010); Cornelie, S. et al., Journal of Biological Chemistry 279(15):15124-9 (2004); Klinman, D. et al., J Immunol. 158(8):3635-9 (1997); Klinman, D. et al., Immunological Reviews 199(1):201-16 (2004); Luo, Z. et al., Mol Med Rep. 6(6):1309-14 (2012); Bode, C. et al., Expert Rev Vaccines 10(4):499-511 (2011); and Kuo, T. et al., Scientific Reports 10:20085 (2020), each of which is incorporated by reference herein in its entirety.

In some aspects, the nucleic acid encoding an immune modifier comprises a combination (i) a nucleic acid encoding an interleukin, and (ii) a nucleic acid encoding a major histocompatibility complex and/or a chemokine.

In some aspects, the nucleic acid encoding an immune modifier comprises a nucleic acid encoding IL-12 p35, a nucleic acid encoding IL-12 p40, or the combination thereof. In some aspects, the nucleic acid encoding IL-12 p35 has at least 70%, at least 75%, at least 80%, at least 85%, at least 90%, at least 95%, at least 99%, or 100% sequence identity to SEQ ID NO: 42 (nucleic acid sequence encoding mouse IL-12 p35) or SEQ ID NO: 46 (nucleic acid sequence encoding human IL-12 p35). In some aspects, the nucleic acid encoding IL-12 p40 has at least 70%, at least 75%, at least 80%, at least 85%, at least 90%, at least 95%, at least 99%, or 100% sequence identity to SEQ ID NO: 44 (nucleic acid sequence encoding mouse IL-12 p40) or SEQ ID NO: 48 (nucleic acid sequence encoding human IL-12 p40). In some aspects, the nucleic acid encoding an immune modifier further comprises a nucleic acid encoding MHC I, a nucleic acid encoding MHC II, or the combination thereof.

In some aspects, the nucleic acid encoding an immune modifier comprises a nucleic acid encoding IL-12, a nucleic acid encoding IL-15, or the combination thereof.

In some aspects, the nucleic acid encoding an immune modifier comprises a combination of a nucleic acid encoding IL-12 and a nucleic acid encoding IL-15.

In some aspects, the nucleic acid encoding an immune modifier comprises a nucleic acid encoding IL-2, a nucleic acid encoding IL-15, a nucleic acid encoding MHC I, a nucleic acid encoding MHC II, a nucleic acid encoding CCL3, a nucleic acid encoding CCL4, or any combination thereof.

In some aspects, the nucleic acid encoding an immune modifier comprises a nucleic acid encoding MHC I, a nucleic acid encoding MHC II, a nucleic acid encoding CCL3, a nucleic acid encoding CCL4, or any combination thereof.

In some aspects, the nucleic acid encoding an immune modifier comprises a nucleic acid encoding CCL3, a nucleic acid encoding CCL4, or the combination thereof.

In some aspects, the polynucleotides or nucleic acids can comprise DNA or mRNA sequences.

In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 (amino acid sequence for SARS-CoV-2 full-length S protein) or SEQ ID NO: 4 (amino acid sequence for SARS-CoV-2 full length S protein+D614G). In some aspects, the first antigen nucleic acid encodes a polypeptide having at least 70%, at least 75%, at least 80%, at least 85%, at least 90%, at least 95%, or at least 99% sequence identity to the amino acid sequence of SEQ ID NO: 2 or SEQ ID NO: 4. In some aspects, the first antigen nucleic acid encodes a polypeptide comprising the amino acid sequence of SEQ ID NO: 2 or SEQ ID NO: 4. In some aspects, the first antigen nucleic acid comprises a nucleic acid sequence having at least 70%, at least 75%, at least 80%, at least 85%, at least 90%, at least 95%, at least 99%, or 100% sequence identity to SEQ ID NO: 1 (nucleic acid sequence encoding SARS-CoV-2 full-length S protein) or SEQ ID NO: 3 (nucleic acid sequence encoding SARS-CoV-2 full-length S protein+D614G).

In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 (amino acid sequence for SARS-CoV-2 full-length S protein) or SEQ ID NO: 4 (amino acid sequence for SARS-CoV-2 full length S protein+D614G). In some aspects, the second antigen nucleic acid encodes a polypeptide having at least 70%, at least 75%, at least 80%, at least 85%, at least 90%, at least 95%, or at least 99% sequence identity to the amino acid sequence of SEQ ID NO: 2 or SEQ ID NO: 4. In some aspects, the second antigen nucleic acid encodes a polypeptide comprising the amino acid sequence of SEQ ID NO: 2 or SEQ ID NO: 4. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 1 (nucleic acid sequence encoding SARS-CoV-2 full-length S protein) or SEQ ID NO: 3 (nucleic acid sequence encoding SARS-CoV-2 full-length S protein+D614G).

In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4, wherein the contiguous amino acids of SEQ ID NO: 2 or SEQ ID NO: 4 comprise one or more mutations (i.e., one or more substitutions, deletions, insertions, or any combination thereof). In some aspects, the first antigen nucleic acid encodes a polypeptide comprising the amino acid sequence of SEQ ID NO: 2 or SEQ ID NO: 4, wherein the polypeptide comprises one or more mutations (i.e., one or more substitutions, deletions, insertions, or any combination thereof).

In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4, wherein the contiguous amino acids of SEQ ID NO: 2 or SEQ ID NO: 4 comprise one or more mutations (i.e., one or more substitutions, deletions, insertions, or any combination thereof). In some aspects, the second antigen nucleic acid encodes a polypeptide comprising the amino acid sequence of SEQ ID NO: 2 or SEQ ID NO: 4, wherein the polypeptide comprises one or more mutations (i.e., one or more substitutions, deletions, insertions, or any combination thereof).

In some aspects, the first antigen nucleic acid encodes the receptor binding domain (RBD) of the SARS-Cov-2 S protein or an antigenic fragment thereof. In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6 (amino acid sequence for RBD of SARS-CoV-2 S protein). In some aspects, the first antigen nucleic acid encodes a polypeptide having at least 70%, at least 75%, at least 80%, at least 85%, at least 90%, at least 95%, or at least 99% sequence identity to the amino acid sequence of SEQ ID NO: 6. In some aspects, the first antigen nucleic acid encodes a polypeptide comprising the amino acid sequence of SEQ ID NO: 6. In some aspects, the first antigen nucleic acid comprises a nucleic acid sequence having at least 70%, at least 75%, at least 80%, at least 85%, at least 90%, at least 95%, at least 99%, or 100% sequence identity to SEQ ID NO: 5 (nucleic acid sequence encoding RBD of SARS-CoV-2 S protein).

In some aspects, the second antigen nucleic acid encodes the receptor binding domain (RBD) of the SARS-Cov-2 S protein or an antigenic fragment thereof. In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6 (amino acid sequence for RBD of SARS-CoV-2 S protein). In some aspects, the second antigen nucleic acid encodes a polypeptide having at least 70%, at least 75%, at least 80%, at least 85%, at least 90%, at least 95%, or at least 99% sequence identity to the amino acid sequence of SEQ ID NO: 6. In some aspects, the second antigen nucleic acid encodes a polypeptide comprising the amino acid sequence of SEQ ID NO: 6. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 5 (nucleic acid sequence encoding RBD of SARS-CoV-2 S protein).

In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6, wherein the contiguous amino acids of SEQ ID NO: 6 comprise one or more mutations (i.e., one or more substitutions, deletions, insertions, or any combination thereof). In some aspects, the first antigen nucleic acid encodes a polypeptide comprising the amino acid sequence of SEQ ID NO: 6, wherein the polypeptide comprises one or more mutations (i.e., one or more substitutions, deletions, insertions, or any combination thereof).

In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6, wherein the contiguous amino acids of SEQ ID NO: 6 comprise one or more mutations (i.e., one or more substitutions, deletions, insertions, or any combination thereof). In some aspects, the second antigen nucleic acid encodes a polypeptide comprising the amino acid sequence of SEQ ID NO: 6, wherein the polypeptide comprises one or more mutations (i.e., one or more substitutions, deletions, insertions, or any combination thereof).

In some aspects, the first antigen nucleic acid encodes the S1 subunit of the SARS-CoV-2 S protein or an antigenic fragment thereof. In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40 (amino acid sequence for the S1 subunit of the SARS-CoV-2 D614G S protein). In some aspects, the first antigen nucleic acid encodes a polypeptide having at least 70%, at least 75%, at least 80%, at least 85%, at least 90%, at least 95%, or at least 99% sequence identity to the amino acid sequence of SEQ ID NO: 40. In some aspects, the first antigen nucleic acid encodes a polypeptide comprising the amino acid sequence of SEQ ID NO: 40. In some aspects, the first antigen nucleic acid comprises a nucleic acid sequence having at least 70%, at least 75%, at least 80%, at least 85%, at least 90%, at least 95%, at least 99%, or 100% sequence identity to SEQ ID NO: 39 (nucleic acid sequence encoding the S1 subunit of the SARS-CoV-2 D614G S protein).

In some aspects, the second antigen nucleic acid encodes the S1 subunit of the SARS-CoV-2 S protein or an antigenic fragment thereof. In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40 (amino acid sequence for the S1 subunit of the SARS-CoV-2 D614G S protein). In some aspects, the second antigen nucleic acid encodes a polypeptide having at least 70%, at least 75%, at least 80%, at least 85%, at least 90%, at least 95%, or at least 99% sequence identity to the amino acid sequence of SEQ ID NO: 40. In some aspects, the second antigen nucleic acid encodes a polypeptide comprising the amino acid sequence of SEQ ID NO: 40. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 39 (nucleic acid sequence encoding the S1 subunit of the SARS-CoV-2 D614G S protein).

In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40, wherein the contiguous amino acids of SEQ ID NO: 40 comprise one or more mutations (i.e., one or more substitutions, deletions, insertions, or any combination thereof). In some aspects, the first antigen nucleic acid encodes a polypeptide comprising the amino acid sequence of SEQ ID NO: 40, wherein the polypeptide comprises one or more mutations (i.e., one or more substitutions, deletions, insertions, or any combination thereof).

In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40, wherein the contiguous amino acids of SEQ ID NO: 40 comprise one or more mutations (i.e., one or more substitutions, deletions, insertions, or any combination thereof). In some aspects, the second antigen nucleic acid encodes a polypeptide comprising the amino acid sequence of SEQ ID NO: 40, wherein the polypeptide comprises one or more mutations (i.e., one or more substitutions, deletions, insertions, or any combination thereof).

In some aspects, the one or more mutations in the SARS-CoV-2 full-length S protein, the RBD of the SARS-Cov-2 S protein, the S1 subunit of the SARS-CoV-2 S protein, or antigenic fragments thereof comprise one or more mutations previously reported in Li, T. et al., Emerg Microbes Infect. 9(1):2076-90 (2020); Lee, P. et al., Immune Netw. 21(1):e4 (2021); Yu, J. et al., Science 369(6505):806-11 (2020); Cattin-Ortola, J. et al., Nat Commun. 12(1):5333 (2021); Corbett, K. et al., Nature 586(7830):567-71 (2020); Hsieh, C. et al., Science 369(6510):1501-5 (2020); and Harvey, W. et al., Nat Rev Microbiol. 19(7):409-24 (2021), each of which is incorporated by reference herein in its entirety.

In some aspects, the one or more mutations in the SARS-CoV-2 full-length S protein, the RBD of the SARS-Cov-2 S protein, the S1 subunit of the SARS-CoV-2 S protein, or antigenic fragments thereof are selected from: ΔM1-S13, S12P, S131, L5F, L18F, T19R, T20N, P26S, Q52R, A67V, ΔH69-V70, G75V, T76I, D80A, T95I, R102I, ΔD119-F120, C136Y, D138Y, ΔF140, ΔL141-Y144, AY144, Y144S, Y145N, ΔH146, N148S, K150R, K150E, K150T, K150Q, S151P, W152C, E154K, AE156-F157, F157L, F157A, R158G, R190S, ΔI210, D215G, A222V, ΔL241-S243, ΔL242-L244, ΔA243-L244, AR246-G252, R246I, 11-amino acid residue insertion between Y248 and L249, D253G, D253N, R346K, V367F, E406W, K417E, K417V, K417N, K417T, N439K, K444A, K444R, K444N, K444Q, V445A, V445E, G447A, N450D, L452R, L452Q, Y453F, L455F, N460I, S477G, S477N, S477R, T478I, T478K, V483I, E484K, E484Q, G485R, F486A, F486V, F486L, N487A, F490S, Q493E, Q493K, S494P, N501Y, A570D, Q613H, D614G, H655Y, Q677H, A678-679, Δ681-682, Δ681-684, Δ682-685, P681H, P681R, R682S, R682A, R682Q, R683S, R683G, R683Q, R685G, R685Q, I692V, A701V, T716I, F817P, T859N, F888L, A892P, A899P, A942P, D950N, S982A, K986P, V987P, T1027I, Q1071H, D1118H, V1176F, M1229I, ΔC1253-T1273, ΔC1254-T1273, ΔK1255-T1273, D1257A, E1258A, D1259A, D1260A, E1262A, K1269A, H1271K, T1273A, or any combination thereof, wherein the amino acid locations correspond to SEQ ID NO: 2 or SEQ ID NO: 4.

In some aspects, the one or more mutations comprise one or more mutations in the N-terminal signal peptide, which corresponds to amino acids 1-13 of SEQ ID NO: 2 or SEQ ID NO: 4. In some aspects, the one or more mutations in the N-terminal signal peptide is ΔM1-S13, wherein the amino acid locations correspond to SEQ ID NO: 2 or SEQ ID NO: 4.

In some aspects, the one or mutations comprise one or more mutations in the C-terminus of the full-length SARS-CoV-2 S protein. In some aspects, the one or more mutations in the C-terminus of the full-length SARS-CoV-2 S protein comprise one or more mutations in the C-terminal endoplasmic reticulum (ER) retention peptide, which corresponds to amino acids 1254-1273 of SEQ ID NO: 2 or SEQ ID NO: 4. In some aspects, the one or more mutations in the C-terminal ER retention peptide are selected from D1257A, E1258A, D1259A, D1260A, E1262A, K1269A, H1271K, T1273A, or any combination thereof, wherein the amino acid locations correspond to SEQ ID NO: 2 or SEQ ID NO: 4. In some aspects, the one or more mutations in the C-terminal ER retention peptide comprise D1257A+E1258A+D1259A+D1260A+E1262A (i.e., a D/E to A mutant), wherein the amino acid locations correspond to SEQ ID NO: 2 or SEQ ID NO: 4. In some aspects, the one or mutations in the C-terminal ER retention peptide is ΔC1253-T1273, ΔC1254-T1273, or ΔK1255-T1273.

In some aspects, the one or more mutations comprise K986P+V987P (i.e., a S-2P mutation), wherein the amino acid locations correspond to SEQ ID NO: 2 or SEQ ID NO: 4. In some aspects, the one or more mutations comprise F817P+A892P+A899P+A942P (i.e., a hexa-proline S mutation), wherein the amino acid locations correspond to SEQ ID NO: 2 or SEQ ID NO: 4. In some aspects, the one or more mutations comprise one or more mutations in the 681-PRRAR/SVA-688 S1/S2 furin cleavage site, wherein the amino acid locations correspond to SEQ ID NO: 2 or SEQ ID NO: 4. In some aspects, the one or more mutations in the 681-PRRAR/SVA-688 S1/S2 furin cleavage site are: (a) R682S+R683S (i.e., a SSAR mutation), (b) Δ681-684 (i.e., a ΔPRRA mutation), (c) Δ678-679+Δ681-682, (d) R682A+R683G+R685G (i.e., a 682-AGAG-685 mutation), (e) R682Q+R683Q+R685Q, (f) R682S+R685G, or (g) Δ682-685 (i.e., a ΔRRAR mutation), wherein the amino acid locations correspond to SEQ ID NO: 2 or SEQ ID NO: 4.

In some aspects, the one or more mutations comprise: (a) F817P+A892P+A899P+A942P (i.e., a hexa-proline S mutation) and (b) K986P+V987P (i.e., a S-2P mutation), wherein the amino acid locations correspond to SEQ ID NO: 2 or SEQ ID NO: 4. In some aspects, the one or more mutations comprise: (a) R682A+R683G+R685G (i.e., a 682-AGAG-685 mutation) and (b) K986P+V987P (i.e., a S-2P mutation), wherein the amino acid locations correspond to SEQ ID NO: 2 or SEQ ID NO: 4. In some aspects, the one or more mutations comprise: (a) R682A+R683G+R685G (i.e., a 682-AGAG-685 mutation), (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations comprise: (a) R682Q+R683Q+R685Q and (b) K986P+V987P (i.e., a S-2P mutation). In some aspects, the one or more mutations comprise:: (a) R682Q+R683Q+R685Q, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations comprise: (a) R682S+R685G and (b) K986P+V987P (i.e., a S-2P mutation), wherein the amino acid locations correspond to SEQ ID NO: 2 or SEQ ID NO: 4. In some aspects, the one or more mutations comprise: (a) R682S+R685G, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4.

In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8 (amino acid sequence for SARS-CoV-2 M protein), SEQ ID NO: 10 (amino acid sequence for SARS-CoV-2 WA9-UW6 Variant M protein), SEQ ID NO: 12 (amino acid sequence for SARS-CoV-2 NIHE Variant M protein), SEQ ID NO: 14 (amino acid sequence for SARS-CoV-2 Wuhan_IME_BJO7 Variant M protein), SEQ ID NO: 16 (amino acid sequence for SARS-CoV-2 WA-UW-1753 Variant M protein), SEQ ID NO: 18 (amino acid sequence for SARS-CoV-2 WA UW-1755 Variant M protein), or SEQ ID NO: 20 (amino acid sequence for SARS-CoV-2 USA_SC_3572 Variant M protein). In some aspects, the second antigen nucleic acid encodes a polypeptide having at least 70%, at least 75%, at least 80%, at least 85%, at least 90%, at least 95%, or at least 99% sequence identity to the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the second antigen nucleic acid encodes a polypeptide comprising the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20.

In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20, wherein the contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20 comprise one or more mutations selected from A2S, F28L, I48V, V70L, I82T, M84T, or any combination thereof, wherein the amino acid locations correspond to SEQ ID NO: 8. In some aspects, the second antigen nucleic acid encodes a polypeptide comprising the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20, wherein the polypeptide comprises one or more mutations selected from A2S, F28L, I48V, V70L, I82T, M84T, or any combination thereof, wherein the amino acid locations correspond to SEQ ID NO: 8.

In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 7 (nucleic acid sequence encoding SARS-CoV-2 M protein), SEQ ID NO: 9 (nucleic acid sequence encoding SARS-CoV-2 WA9-UW6 Variant M protein), SEQ ID NO: 11 (nucleic acid sequence encoding SARS-CoV-2 NIHE Variant M protein), SEQ ID NO: 13 (nucleic acid sequence encoding SARS-CoV-2 Wuhan_IME_BJO7 Variant M protein), SEQ ID NO: 15 (nucleic acid sequence encoding SARS-CoV-2 WA-UW-1753 Variant M protein), SEQ ID NO: 17 (nucleic acid sequence encoding SARS-CoV-2 WA UW-1755 Variant M protein), or SEQ ID NO: 19 (nucleic acid sequence encoding SARS-CoV-2 USA_SC_3572 Variant M protein).

In some aspects, the second antigen nucleic acid encodes at least 8, at least 10, at least 15, at least 20, at least 25, at least 30, at least 35, at least 40, at least 45, at least 50, at least 55, at least 60, at least 65, at least 70, or at least 75 contiguous amino acids of SEQ ID NO: 22, SEQ ID NO: 24, or SEQ ID NO: 26. In some aspects, the second antigen nucleic acid encodes a polypeptide having at least 70%, at least 75%, at least 80%, at least 85%, at least 90%, at least 95%, or at least 99% sequence identity to the amino acid sequence of SEQ ID NO: 21, SEQ ID NO: 23, or SEQ ID NO: 25. In some aspects, the second antigen nucleic acid encodes a polypeptide comprising the amino acid sequence of SEQ ID NO: 22, SEQ ID NO: 24, or SEQ ID NO: 26. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 21, SEQ ID NO: 23, or SEQ ID NO: 25.

In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, or at least 400 contiguous amino acids of SEQ ID NO: 28. In some aspects, the second antigen nucleic acid encodes a polypeptide having at least 70%, at least 75%, at least 80%, at least 85%, at least 90%, at least 95%, or at least 99% sequence identity to the amino acid sequence of SEQ ID NO: 28. In some aspects, the second antigen nucleic acid encodes a polypeptide comprising the amino acid sequence of SEQ ID NO: 28. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 27.

In some aspects, the polynucleotide comprises: (a) a first antigen nucleic acid encoding the S1 subunit of the SARS-Cov-2 S protein or an antigenic fragment thereof and (b) two or more nucleic acids encoding an immune modifier, wherein the two or more nucleic acids encoding an immune modifier comprise a nucleic acid encoding IL-12 p35 and a nucleic acid encoding IL-12 p40, wherein the first antigen nucleic acid is operably linked to a mammalian elongation factor 1 (EF1) promoter, wherein the nucleic acid encoding IL-12 p35 is operably linked to a first CMV promoter, and wherein the nucleic acid encoding IL-12 p40 is operably linked to a second CMV promoter. In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the first antigen nucleic acid encodes a polypeptide having at least 70%, at least 75%, at least 80%, at least 85%, at least 90%, at least 95%, or at least 99% sequence identity to the amino acid sequence of SEQ ID NO: 40. In some aspects, the first antigen nucleic acid encodes a polypeptide comprising the amino acid sequence of SEQ ID NO: 40. In some aspects, the first antigen nucleic acid comprises a nucleic acid sequence having at least 70%, at least 75%, at least 80%, at least 85%, at least 90%, at least 95%, at least 99%, or 100% sequence identity to SEQ ID NO: 39. In some aspects, the nucleic acid encoding IL-12 p35 comprises a nucleic acid sequence having at least 70%, at least 75%, at least 80%, at least 85%, at least 90%, at least 95%, at least 99%, or 100% sequence identity to SEQ ID NO: 46. In some aspects, the IL-12 p35 comprises the amino acid sequence of SEQ ID NO: 47. In some aspects, the nucleic acid encoding IL-12 p40 comprises a nucleic acid sequence having at least 70%, at least 75%, at least 80%, at least 85%, at least 90%, at least 95%, at least 99%, or 100% sequence identity to SEQ ID NO: 48. In some aspects, the IL-12 p40 comprises the amino acid sequence of SEQ ID NO: 49.

In some aspects, the polynucleotide comprises: (a) a first antigen nucleic acid encoding the S1 subunit of the SARS-Cov-2 S protein or an antigenic fragment thereof, (b) a second antigen nucleic acid encoding a SARS-CoV-2 membrane (M) protein or an antigenic fragment thereof and (c) two or more nucleic acids encoding an immune modifier, wherein the two or more nucleic acids encoding an immune modifier comprise a nucleic acid encoding IL-12 p35 and a nucleic acid encoding IL-12 p40, wherein the first antigen nucleic acid is operably linked to a mammalian elongation factor 1 (EF1) promoter, wherein the second antigen nucleic acid is operably linked to the EF1 promoter through an internal ribosome entry site (IRES) sequence, wherein the nucleic acid encoding IL-12 p35 is operably linked to a first CMV promoter, and wherein the nucleic acid encoding IL-12 p40 is operably linked to a second CMV promoter. In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the first antigen nucleic acid encodes a polypeptide having at least 70%, at least 75%, at least 80%, at least 85%, at least 90%, at least 95%, or at least 99% sequence identity to the amino acid sequence of SEQ ID NO: 40. In some aspects, the first antigen nucleic acid encodes a polypeptide comprising the amino acid sequence of SEQ ID NO: 40. In some aspects, the first antigen nucleic acid comprises a nucleic acid sequence having at least 70%, at least 75%, at least 80%, at least 85%, at least 90%, at least 95%, at least 99%, or 100% sequence identity to SEQ ID NO: 39. In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the first antigen nucleic acid encodes a polypeptide having at least 70%, at least 75%, at least 80%, at least 85%, at least 90%, at least 95%, or at least 99% sequence identity to the amino acid sequence of SEQ ID NO: 8, SEQ ID NO: 10 SEQ ID NO: 12, SEQ ID NO: 14, SEQ ID NO: 16, SEQ ID NO: 18, or SEQ ID NO: 20. In some aspects, the second antigen nucleic acid encodes a polypeptide comprising the amino acid sequence of SEQ ID NO: 8, SEQ ID NO: 10 SEQ ID NO: 12, SEQ ID NO: 14, SEQ ID NO: 16, SEQ ID NO: 18, or SEQ ID NO: 20. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 7, SEQ ID NO: 9, SEQ ID NO: 11, SEQ ID NO: 13, SEQ ID NO: 15, SEQ ID NO: 17, or SEQ ID NO: 19. In some aspects, the nucleic acid encoding IL-12 p35 comprises a nucleic acid sequence having at least 70%, at least 75%, at least 80%, at least 85%, at least 90%, at least 95%, at least 99%, or 100% sequence identity to SEQ ID NO: 46. In some aspects, the IL-12 p35 comprises the amino acid sequence of SEQ ID NO: 47. In some aspects, the nucleic acid encoding IL-12 p40 comprises a nucleic acid sequence having at least 70%, at least 75%, at least 80%, at least 85%, at least 90%, at least 95%, at least 99%, or 100% sequence identity to SEQ ID NO: 48. In some aspects, the IL-12 p40 comprises the amino acid sequence of SEQ ID NO: 49.

In some aspects, the polynucleotide comprises: (a) a first antigen nucleic acid encoding a full-length SARS-Cov-2 S protein or an antigenic fragment thereof and (b) two or more nucleic acids encoding an immune modifier, wherein the two or more nucleic acids encoding an immune modifier comprise a nucleic acid encoding IL-12 p35 and a nucleic acid encoding IL-12 p40, wherein the first antigen nucleic acid is operably linked to a mammalian elongation factor 1 (EF1) promoter, wherein the nucleic acid encoding IL-12 p35 is operably linked to a first CMV promoter, and wherein the nucleic acid encoding IL-12 p40 is operably linked to a second CMV promoter. In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the first antigen nucleic acid encodes a polypeptide having at least 70%, at least 75%, at least 80%, at least 85%, at least 90%, at least 95%, or at least 99% sequence identity to the amino acid sequence of SEQ ID NO: 2 or SEQ ID NO: 4. In some aspects, the first antigen nucleic acid encodes a polypeptide comprising the amino acid sequence of SEQ ID NO: 2 or SEQ ID NO: 4. In some aspects, the first antigen nucleic acid comprises a nucleic acid sequence having at least 70%, at least 75%, at least 80%, at least 85%, at least 90%, at least 95%, at least 99%, or 100% sequence identity to SEQ ID NO: 1 or SEQ ID NO: 3. In some aspects, the nucleic acid encoding IL-12 p35 comprises a nucleic acid sequence having at least 70%, at least 75%, at least 80%, at least 85%, at least 90%, at least 95%, at least 99%, or 100% sequence identity to SEQ ID NO: 46. In some aspects, the IL-12 p35 comprises the amino acid sequence of SEQ ID NO: 47. In some aspects, the nucleic acid encoding IL-12 p40 comprises a nucleic acid sequence having at least 70%, at least 75%, at least 80%, at least 85%, at least 90%, at least 95%, at least 99%, or 100% sequence identity to SEQ ID NO: 48. In some aspects, the IL-12 p40 comprises the amino acid sequence of SEQ ID NO: 49.

In some aspects, the polynucleotide comprises: (a) a first antigen nucleic acid encoding a full-length SARS-Cov-2 S protein or an antigenic fragment thereof, (b) a second antigen nucleic acid encoding a SARS-CoV-2 membrane (M) protein or an antigenic fragment thereof, and (c) two or more nucleic acids encoding an immune modifier, wherein the first antigen nucleic acid is operably linked to a mammalian elongation factor 1 (EF1) promoter, wherein the second antigen nucleic acid is operably linked to the EF1 promoter through an internal ribosome entry site (IRES) sequence, wherein the nucleic acid encoding IL-12 p35 is operably linked to a first CMV promoter, and wherein the nucleic acid encoding IL-12 p40 is operably linked to a second CMV promoter. In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the first antigen nucleic acid encodes a polypeptide having at least 70%, at least 75%, at least 80%, at least 85%, at least 90%, at least 95%, or at least 99% sequence identity to the amino acid sequence of SEQ ID NO: 2 or SEQ ID NO: 4. In some aspects, the first antigen nucleic acid encodes a polypeptide comprising the amino acid sequence of SEQ ID NO: 2 or SEQ ID NO: 4. In some aspects, the first antigen nucleic acid comprises a nucleic acid sequence having at least 70%, at least 75%, at least 80%, at least 85%, at least 90%, at least 95%, at least 99%, or 100% sequence identity to SEQ ID NO: 1 or SEQ ID NO: 3. In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the second antigen nucleic acid encodes a polypeptide having at least 70%, at least 75%, at least 80%, at least 85%, at least 90%, at least 95%, or at least 99% sequence identity to the amino acid sequence of SEQ ID NO: 8, SEQ ID NO: 10 SEQ ID NO: 12, SEQ ID NO: 14, SEQ ID NO: 16, SEQ ID NO: 18, or SEQ ID NO: 20. In some aspects, the second antigen nucleic acid encodes a polypeptide comprising the amino acid sequence of SEQ ID NO: 8, SEQ ID NO: 10 SEQ ID NO: 12, SEQ ID NO: 14, SEQ ID NO: 16, SEQ ID NO: 18, or SEQ ID NO: 20. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 7, SEQ ID NO: 9, SEQ ID NO: 11, SEQ ID NO: 13, SEQ ID NO: 15, SEQ ID NO: 17, or SEQ ID NO: 19. In some aspects, the nucleic acid encoding IL-12 p35 comprises a nucleic acid sequence having at least 70%, at least 75%, at least 80%, at least 85%, at least 90%, at least 95%, at least 99%, or 100% sequence identity to SEQ ID NO: 46. In some aspects, the IL-12 p35 comprises the amino acid sequence of SEQ ID NO: 47. In some aspects, the nucleic acid encoding IL-12 p40 comprises a nucleic acid sequence having at least 70%, at least 75%, at least 80%, at least 85%, at least 90%, at least 95%, at least 99%, or 100% sequence identity to SEQ ID NO: 48. In some aspects, the IL-12 p40 comprises the amino acid sequence of SEQ ID NO: 49.

In some aspects, the polynucleotide further comprises one or more post-transcriptional regulatory elements. In some aspects, the post-transcriptional regulatory element is a wood chuck hepatitis virus post-transcriptional regulatory element (WPRE).

In some aspects, the polynucleotide further comprises at least one 3′ UTR poly(a) tail sequence operably linked to the first antigen nucleic acid, the second antigen nucleic acid, the nucleic acid encoding an immune modifier, or any combination thereof. In some aspects, the 3′ UTR poly(a) tail sequence is a 3′ UTR SV40 poly(a) tail sequence (SEQ ID NO: 29), a 3′ UTR bovine growth hormone (bGH) poly(A) sequence (SEQ ID NO: 30), a 3′ UTR actin poly(A) tail sequence, a 3′ UTR hemoglobin poly(A) sequence, or any combinations thereof.

In some aspects, the polynucleotide further comprises at least one enhancer sequence. In some aspects, the enhancer sequence is a human actin enhancer sequence, a human myosin enhancer sequence, a human hemoglobin enhancer sequence, a human muscle creatine enhancer sequence, a viral enhancer sequence, or a polynucleotide function enhancer sequence. In some aspects, the enhancer sequence is a CMV intronic sequence or a β-actin intronic sequence. In aspects, the enhancer sequence is a SV40 enhancer sequence (SEQ ID NO: 37).

In some aspects, the polynucleotide further comprises an inverted terminal repeat (ITR). In some aspects, the polynucleotide comprises a first ITR and a second ITR. In some aspects, the first ITR and the second ITR are derived from an adeno-associated virus (AAV).

In some aspects, the polynucleotide is a multicistronic mRNA comprising a 5′ cap and a 3′ UTR poly(A) tail sequence. In some aspects, the 3′ UTR poly(a) tail sequence is a 3′ UTR SV40 poly(a) tail sequence (SEQ ID NO: 29), a 3′ UTR bovine growth hormone (bGH) poly(A) sequence (SEQ ID NO: 30), a 3′ UTR actin poly(A) tail sequence, a 3′ UTR hemoglobin poly(A) sequence, or any combinations thereof. In some aspects, the multicistronic mRNA comprises a 5′ UTR and/or a 3′ UTR.

Also provided herein is a vector comprising any polynucleotide described or exemplified herein, wherein the vector is a DNA plasmid, a multicistronic mRNA, a viral vector, a bacterial vector, a cosmid, or an artificial chromosome. In some aspects, the vector is selected from an AAV vector, adenoviral vector, retroviral vector, poxvirus vector, baculovirus vector, herpes viral vector, or combinations thereof.

Also provided herein is a composition, pharmaceutical composition, or vaccine comprising any polynucleotide or vector described or exemplified herein. In some aspects, the composition, pharmaceutical composition, or vaccine further comprises a pharmaceutically acceptable carrier. In some aspects, the composition, pharmaceutical composition, or vaccine further comprises a second polynucleotide encoding at least one immune modifier selected from the group consisting of: IL-2, IL-12 p35, IL-12 p40, IL-12 p70, IL-15, IL-18, TNFα, GM-CSF, IFN-α, IFN-β, a chemokine, MHC I, MHC II, HLA-DR, CD80, and CD86, wherein the polynucleotide encoding the at least one immune modifier is operably linked to a promoter. In some aspects, the composition, pharmaceutical composition, or vaccine further comprises a delivery component.

The delivery component of the compositions, pharmaceutical compositions, or vaccines may comprise any combination of delivery components disclosed herein.

In some aspects, the IL-12 p35 immune modifier comprises an amino acid sequence having at least 70%, at least 75%, at least 80%, at least 85%, at least 90%, at least 95%, at least 99%, or 100% sequence identity to SEQ ID NO: 43 (mouse IL-12 p35) or SEQ ID NO: 47 (human IL-12 p35). In some aspects, the IL-12 p40 immune modifier comprises an amino acid sequence having at least 70%, at least 75%, at least 80%, at least 85%, at least 90%, at least 95%, at least 99%, or 100% sequence identity to SEQ ID NO: 45 (mouse IL-12 p40) or SEQ ID NO: 49 (human IL-12 p40).

In some aspects, the second polynucleotide encodes IL-12 p35, IL-12 p40, or the combination thereof. In some aspects, the IL-12 p35 immune modifier is encoded by a nucleic acid sequence having at least 70%, at least 75%, at least 80%, at least 85%, at least 90%, at least 95%, at least 99%, or 100% sequence identity to SEQ ID NO: 42 (nucleic acid sequence encoding mouse IL-12 p35) or SEQ ID NO: 46 (nucleic acid sequence encoding human IL-12 p35). In some aspects, the IL-12 p40 immune modifier is encoded by a nucleic acid sequence having at least 70%, at least 75%, at least 80%, at least 85%, at least 90%, at least 95%, at least 99%, or 100% sequence identity to SEQ ID NO: 44 (nucleic acid sequence encoding mouse IL-12 p40) or SEQ ID NO: 48 (nucleic acid sequence encoding human IL-12 p40).

In some aspects, the at least one immune modifier encoded by the second polynucleotide comprises a viral protein (e.g., SARS-CoV-2 non-structural protein 1 (Nsp1), SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b) that attenuates a local inflammatory response and/or interferon response. In some aspects, the viral protein is from the same virus as a viral antigen encoded by an antigen nucleic acid. In some aspects, In some aspects, the viral protein is from a different virus than a viral antigen encoded by an antigen nucleic acid. In some aspects, the viral protein attenuates a local inflammatory response and/or interferon response elicited by a pathogen antigen disclosed herein. In some aspects, the at least one immune modifier encoded by the second polynucleotide comprises SARS-CoV-2 Nsp1, SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b, or any combination thereof. In some aspects, the at least one immune modifier comprises one or more concatamers of non-coding 5′-C-phosphate-G-3′ (CpG) dinucleotides. In some aspects, the one or more concatamers of non-coding CpG dinucleotides activate the Toll-like receptor 9 (TLR9) signaling pathway. In some aspects, the one or more concatamers of non-coding CpG dinucleotides comprise one or more concatamers of non-coding CpG dinucleotides previously reported in Bauer, A. et al., Nucleic Acids Research 38(12):3891-908 (2010); Cornelie, S. et al., Journal of Biological Chemistry 279(15):15124-9 (2004); Klinman, D. et al., J Immunol. 158(8):3635-9 (1997); Klinman, D. et al., Immunological Reviews 199(1):201-16 (2004); Luo, Z. et al., Mol Med Rep. 6(6):1309-14 (2012); Bode, C. et al., Expert Rev Vaccines 10(4):499-511 (2011); and Kuo, T. et al., Scientific Reports 10:20085 (2020), each of which is incorporated by reference herein in its entirety.

In some aspects, the composition, pharmaceutical composition, or vaccine further comprises a third polynucleotide encoding at least one immune modifier selected from the group consisting of: IL-2, IL-12 p35, IL-12 p40, IL-12 p70, IL-15, IL-18, TNFα, GM-CSF, IFN-α, IFN-β, a chemokine, MHC I, MHC II, HLA-DR, CD80, and CD86, wherein the polynucleotide encoding the at least one immune modifier is operably linked to a promoter. In some aspects, the second polynucleotide encoding at least one immune modifier encodes the IL-12 p35 immune modifier, and the third polynucleotide encoding at least one immune modifier encodes the IL-12 p40 immune modifier. In some aspects, the second polynucleotide encoding the IL-12 p35 modifier comprises a nucleic acid sequence having at least 70%, at least 75%, at least 80%, at least 85%, at least 90%, at least 95%, at least 99%, or 100% sequence identity to SEQ ID NO: 42 (nucleic acid sequence encoding mouse IL-12 p35) or SEQ ID NO: 46 (nucleic acid sequence encoding human IL-12 p35). In some aspects, the third polynucleotide encoding the IL-12 p40 immune modifier comprises a nucleic acid sequence having at least 70%, at least 75%, at least 80%, at least 85%, at least 90%, at least 95%, at least 99%, or 100% sequence identity to SEQ ID NO: 44 (nucleic acid sequence encoding mouse IL-12 p40) or SEQ ID NO: 48 (nucleic acid sequence encoding human IL-12 p40).

In some aspects, the at least one immune modifier encoded by the third polynucleotide comprises a viral protein (e.g., SARS-CoV-2 non-structural protein 1 (Nsp1), SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b) that attenuates a local inflammatory response and/or interferon response. In some aspects, the viral protein is from the same virus as a viral antigen encoded by an antigen nucleic acid. In some aspects, In some aspects, the viral protein is from a different virus than a viral antigen encoded by an antigen nucleic acid. In some aspects, the viral protein attenuates a local inflammatory response and/or interferon response elicited by a pathogen antigen disclosed herein. In some aspects, the at least one immune modifier encoded by the third polynucleotide comprises SARS-CoV-2 Nsp1, SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b, or any combination thereof. In some aspects, the at least one immune modifier comprises one or more concatamers of non-coding 5′-C-phosphate-G-3′ (CpG) dinucleotides. In some aspects, the one or more concatamers of non-coding CpG dinucleotides activate the Toll-like receptor 9 (TLR9) signaling pathway. In some aspects, the one or more concatamers of non-coding CpG dinucleotides comprise one or more concatamers of non-coding CpG dinucleotides previously reported in Bauer, A. et al., Nucleic Acids Research 38(12):3891-908 (2010); Cornelie, S. et al., Journal of Biological Chemistry 279(15):15124-9 (2004); Klinman, D. et al., J Immunol. 158(8):3635-9 (1997); Klinman, D. et al., Immunological Reviews 199(1):201-16 (2004); Luo, Z. et al., Mol Med Rep. 6(6):1309-14 (2012); Bode, C. et al., Expert Rev Vaccines 10(4):499-511 (2011); and Kuo, T. et al., Scientific Reports 10:20085 (2020), each of which is incorporated by reference herein in its entirety. In some aspects, the composition, pharmaceutical composition, or vaccine further comprises an adjuvant. In some aspects, the adjuvant comprises one or more concatamers of non-coding 5′-C-phosphate-G-3′ (CpG) dinucleotides. In some aspects, the one or more concatamers of non-coding CpG dinucleotides activate the Toll-like receptor 9 (TLR9) signaling pathway. In some aspects, the one or more concatamers of non-coding CpG dinucleotides comprise one or more concatamers of non-coding CpG dinucleotides previously reported in Bauer, A. et al., Nucleic Acids Research 38(12):3891-908 (2010); Cornelie, S. et al., Journal of Biological Chemistry 279(15):15124-9 (2004); Klinman, D. et al., J Immunol. 158(8):3635-9 (1997); Klinman, D. et al., Immunological Reviews 199(1):201-16 (2004); Luo, Z. et al., Mol Med Rep. 6(6):1309-14 (2012); Bode, C. et al., Expert Rev Vaccines 10(4):499-511 (2011); and Kuo, T. et al., Scientific Reports 10:20085 (2020), each of which is incorporated by reference herein in its entirety.

Also provided herein is a vector (e.g., a plasmid vector) comprising any polynucleotide (e.g., DNA or mRNA) described or exemplified herein. In some aspects, the plasmid vector is a DNA plasmid vector.

Also provided herein is a composition, pharmaceutical composition, or vaccine comprising any polynucleotide, vector, multicistronic mRNA vector, or DNA plasmid vector described or exemplified herein. In some aspects, the composition, pharmaceutical composition, or vaccine comprises a second polynucleotide encoding at least one immune modifier selected from the group consisting of: IL-2, IL-12 p35, IL-12 p40, IL-12 p70, IL-15, IL-18, TNFα, GM-CSF, IFN-α, IFN-β, a chemokine, MHC I, MHC II, HLA-DR, CD80, and CD86, wherein the second polynucleotide encoding the at least one immune modifier is operably linked to a promoter. In some aspects, the composition, pharmaceutical composition, or vaccine further comprises a third polynucleotide encoding at least one immune modifier selected from the group consisting of: IL-2, IL-12 p35, IL-12 p40, IL-12 p70, IL-15, IL-18, TNFα, GM-CSF, IFN-α, IFN-β, a chemokine, MHC I, MHC II, HLA-DR, CD80, and CD86, wherein the third polynucleotide encoding the at least one immune modifier is operably linked to a promoter. In some aspects, the second polynucleotide encoding at least one immune modifier encodes IL-12 p35, and the third polynucleotide encoding at least one immune modifier encodies IL-12 p40.

In some aspects, the at least one immune modifier encoded by the second polynucleotide comprises a viral protein (e.g., SARS-CoV-2 non-structural protein 1 (Nsp1), SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b) that attenuates a local inflammatory response and/or interferon response. In some aspects, the viral protein is from the same virus as a viral antigen encoded by an antigen nucleic acid. In some aspects, In some aspects, the viral protein is from a different virus than a viral antigen encoded by an antigen nucleic acid. In some aspects, the viral protein attenuates a local inflammatory response and/or interferon response elicited by a pathogen antigen disclosed herein. In some aspects, the at least one immune modifier encoded by the second polynucleotide comprises SARS-CoV-2 Nsp1, SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b, or any combination thereof. In some aspects, the at least one immune modifier comprises one or more concatamers of non-coding 5′-C-phosphate-G-3′ (CpG) dinucleotides. In some aspects, the one or more concatamers of non-coding CpG dinucleotides activate the Toll-like receptor 9 (TLR9) signaling pathway. In some aspects, the one or more concatamers of non-coding CpG dinucleotides comprise one or more concatamers of non-coding CpG dinucleotides previously reported in Bauer, A. et al., Nucleic Acids Research 38(12):3891-908 (2010); Cornelie, S. et al., Journal of Biological Chemistry 279(15):15124-9 (2004); Klinman, D. et al., J Immunol. 158(8):3635-9 (1997); Klinman, D. et al., Immunological Reviews 199(1):201-16 (2004); Luo, Z. et al., Mol Med Rep. 6(6):1309-14 (2012); Bode, C. et al., Expert Rev Vaccines 10(4):499-511 (2011); and Kuo, T. et al., Scientific Reports 10:20085 (2020), each of which is incorporated by reference herein in its entirety.

In some aspects, the at least one immune modifier encoded by the third polynucleotide comprises a viral protein (e.g., SARS-CoV-2 non-structural protein 1 (Nsp1), SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b) that attenuates a local inflammatory response and/or interferon response. In some aspects, the viral protein is from the same virus as a viral antigen encoded by an antigen nucleic acid. In some aspects, In some aspects, the viral protein is from a different virus than a viral antigen encoded by an antigen nucleic acid. In some aspects, the viral protein attenuates a local inflammatory response and/or interferon response elicited by a pathogen antigen disclosed herein. In some aspects, the at least one immune modifier encoded by the third polynucleotide comprises SARS-CoV-2 Nsp1, SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b, or any combination thereof. In some aspects, the at least one immune modifier comprises one or more concatamers of non-coding 5′-C-phosphate-G-3′ (CpG) dinucleotides. In some aspects, the one or more concatamers of non-coding CpG dinucleotides activate the Toll-like receptor 9 (TLR9) signaling pathway. In some aspects, the one or more concatamers of non-coding CpG dinucleotides comprise one or more concatamers of non-coding CpG dinucleotides previously reported in Bauer, A. et al., Nucleic Acids Research 38(12):3891-908 (2010); Cornelie, S. et al., Journal of Biological Chemistry 279(15):15124-9 (2004); Klinman, D. et al., J Immunol. 158(8):3635-9 (1997); Klinman, D. et al., Immunological Reviews 199(1):201-16 (2004); Luo, Z. et al., Mol Med Rep. 6(6):1309-14 (2012); Bode, C. et al., Expert Rev Vaccines 10(4):499-511 (2011); and Kuo, T. et al., Scientific Reports 10:20085 (2020), each of which is incorporated by reference herein in its entirety.

Also provided herein is a composition, pharmaceutical composition, or vaccine comprising: (a) a polynucleotide or a vector comprising the polynucleotide, and (b) a delivery component, wherein the polynucleotide comprises a first antigen nucleic acid which encodes a first pathogen protein or an antigenic fragment thereof, wherein the first antigen nucleic acid is operably linked to a first promoter, optionally wherein the delivery component is a cationic polymer, a poly-inosinic-polycytidylic acid, or a poloxamer or derivative thereof. In some aspects, the delivery component is a cationic polymer, a poly-inosinic-polycytidylic acid, or a poloxamer or derivative thereof. In some aspects, the first antigen nucleic acid which encodes a first pathogen protein is selected from the group consisting of a viral protein, a bacterial protein, a parasite protein, and any antigenic fragment thereof. In some aspects, the delivery component further comprises benzalkonium chloride.

The delivery component of the compositions, pharmaceutical compositions, or vaccines may comprise any combination of delivery components disclosed herein.

In some aspects, the polynucleotide further comprises a second antigen nucleic acid which encodes a second pathogen protein or an antigenic fragment thereof. In some aspects, the second antigen nucleic acid which encodes a second pathogen protein is selected from the group consisting of a viral protein, a bacterial protein, a parasite protein, and any antigenic fragment thereof.

In some aspects, the first pathogen protein and/or the second pathogen protein is/are selected from the group consisting of a Yersiniapestis antigen, a Mycobacterium tuberculosis antigen, an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, a Toxoplasma gondii antigen, a Plasmodium falciparum: antigen, an influenza virus antigen, antigenic fragments thereof, and any combinations thereof.

In some aspects, the first pathogen protein and/or the second pathogen protein is/are selected from the group consisting of: a Yersiniapestis F1-Ag, a Yersiniapestis V-Ag, a Mycobacterium tuberculosis Apa antigen, a Mycobacterium tuberculosis H1P65 antigen, a Mycobacterium tuberculosis rAg85A antigen, an E71 VP1 antigen, a GST-tagged E71-VP1 antigen, a Cox protein antigen, a GST-tagged Cox protein antigen, an HSV-1 envelope antigen, an HSV-2 envelope antigen, an HSV-2 gB2 antigen, an HSV-2 gC2 antigen, an HSV-2 gD2 antigen, an HSV-2 gE2 antigen, an HIV Env antigen, an HIV Gag antigen, an HIV Nef antigen, an HIV Pol antigen, an HPV minor capsid protein L2 antigen, an HCV NS3 antigen, a RSV F antigen, a RSV G antigen, a Dengue virus E protein antigen, a Dengue virus EDIII antigen, a Dengue virus NS1 antigen, a Dengue virus DEN-80E antigen, an Ebola virus GB antigen, an Ebola virus VP24 antigen, an Ebola virus VP40 antigen, an Ebola virus NP antigen, an Ebola virus VP30 antigen, an Ebola virus VP35 antigen, a Zika virus envelope domain III antigen, a Zika virus CKD antigen, a Chikungunya virus E1 glycoprotein subunit antigen, the MHC class I epitope PPFGAGRPGQFGDI (SEQ ID NO: 34), the MHC class I epitope TAECKDKNL (SEQ ID NO: 35), the MHC class II epitope VRYKCNCGG (SEQ ID NO: 36), a measles virus hemagglutinin protein MV-H antigen, a measles virus fusion protein MV-F antigen, a MERS-CoV S protein antigen, an antigen from the receptor-binding domain of the MERS-CoV S protein, an antigen from the membrane fusion domain of the MERS-CoV S protein, a SARS-CoV S protein antigen, an antigen from the receptor binding domain of the SARS-CoV S protein, an antigen from the membrane fusion domain of the SARS-CoV S protein, a SARS-CoV E protein antigen, a SARS-CoV M protein antigen, a Toxoplasma gondii MIC8 antigen, a Plasmodium falciparum: SERA5 polypeptide antigen, a Plasmodium falciparum: circumsporozite protein antigen, an influenza virus hemagglutinin (HA) antigen, an influenza virus neuraminidase (NA) antigen, an influenza virus matrix-2 (M2) protein antigen, antigenic fragments thereof, and any combination thereof.

In some aspects, the first antigen nucleic acid encodes a SARS CoV-2 spike (S) protein or an antigenic fragment thereof. In some aspects, the second pathogen protein or antigenic fragment thereof is selected from the group consisting of: a SARS-CoV-2 membrane (M) protein or an antigenic fragment thereof, a SARS-CoV-2 envelope (E) protein or an antigenic fragment thereof, a SARS-CoV-2 nucleocapsid (N) protein or an antigenic fragment thereof, and any combination thereof. In some aspects, the first antigen nucleic acid and the second antigen nucleic acid encode SARS-CoV-2 proteins or antigenic fragments thereof from different strains of SARS-CoV-2. In some aspects, the first antigen nucleic acid and the second antigen nucleic acid encode different variants of the same SARS-CoV-2 protein or antigenic fragment thereof, wherein the different variants of the same SARS-CoV2 protein or antigenic fragment thereof are derived from different strains of SARS-CoV-2. In some aspects, the first antigen nucleic acid and the second antigen nucleic acid encode SARS-CoV-2 S proteins or antigenic fragments thereof from different strains of SARS-CoV-2. In some aspects, the SARS-CoV-2 S proteins or antigenic fragments thereof from different strains of SARS-CoV-2 comprise one or more mutations previously reported in Li, T. et al., Emerg Microbes Infect. 9(1):2076-90 (2020); Lee, P. et al., Immune Netw. 21(1):e4 (2021); Yu, J. et al., Science 369(6505):806-11 (2020); Cattin-Ortola, J. et al., Nat Commun. 12(1):5333 (2021); Corbett, K. et al., Nature 586(7830):567-71 (2020); Hsieh, C. et al., Science 369(6510):1501-5 (2020); and Harvey, W. et al., Nat Rev Microbiol. 19(7):409-24 (2021), each of which is incorporated by reference herein in its entirety. In some aspects, the different strains of SARS-CoV-2 are selected from the group consisting of: an Alpha SARS-CoV-2 strain (e.g., strains B.1.1.7 and Q.1-Q.8); a Beta SARS-CoV-2 strain (e.g., strains B.1.351, B.1.351.2, and B.1.351.3); a Delta SARS-CoV-2 strain (e.g., strain B.1.617.2 and AY.1 sublineages); a SARS-CoV-2 strain Gamma strain (e.g., strains P.1, P.1.1, and P.1.2); an Epsilon SARS-CoV-2 strain (e.g., strains B.1.427 and B.1.429); an Eta SARS-CoV-2 strain (e.g., strain B.1.525); an Iota SARS-CoV-2 strain (e.g., strain B.1.526); a Kappa SARS-CoV-2 strain (e.g., strain B.1.617.1); a Lambda SARS-CoV-2 strain; a B.1.617.3 SARS-CoV-2 strain; a Mu SARS-CoV-2 strain (e.g., strains B.1.621 and B.1.621.1); a Zeta strain (e.g., strain P.2); and any combination thereof. In some aspects, the different strains of SARS-CoV-2 are selected from the group consisting of: B.1.1.7, Q.1, Q.2, Q.3, Q.4, Q.5, Q.6, Q.7, Q.8, B.1.351, B.1.351.2, B.1.351.3, B.1.617.2, AY.1 sublineages, P.1, P.1.1, P.1.2, B.1.427, B.1.429, B.1.525, B.1.526, B.1.617.1, B.1.617.3, B.1.621, B.1.621.1, P.2, and any combination thereof.

In some aspects, the first antigen nucleic acid and/or the second antigen nucleic acid encodes a SARS CoV-2 S protein or an antigenic fragment thereof from an Alpha SARS-CoV-2 strain, wherein the SARS-CoV-2 S protein or antigenic fragment thereof comprises the amino acid sequence of SEQ ID NO: 2 or SEQ ID NO: 4 with one or mutations selected from ΔH69-V70, Δ144, E484K, N501Y, A570D, D614G, P681H, T716I, S982A, and D1118H wherein the amino acid locations correspond to SEQ ID NO: 2 or SEQ ID NO: 4. In some aspects, the first antigen nucleic acid and/or the second antigen nucleic acid encodes a SARS CoV-2 S protein or an antigenic fragment thereof from a Beta SARS-CoV-2 strain, wherein the SARS-CoV-2 S protein or antigenic fragment thereof comprises the amino acid sequence of SEQ ID NO: 2 or SEQ ID NO: 4 with one or mutations selected from L18F, D80A, D215G, ΔL241-S243, K417N, E484K, N501Y, D614G, and A701V, wherein the amino acid locations correspond to SEQ ID NO: 2 or SEQ ID NO: 4. In some aspects, the first antigen nucleic acid and/or the second antigen nucleic acid encodes a SARS CoV-2 S protein or an antigenic fragment thereof from a Gamma SARS-CoV-2 strain, wherein the SARS-CoV-2 S protein or antigenic fragment thereof comprises the amino acid sequence of SEQ ID NO: 2 or SEQ ID NO: 4 with one or mutations selected from L18F, T20N, P26S, D138Y, R190S, K417T, E484K, N501Y, D614G, H655Y, T1027I, and V1176F, wherein the amino acid locations correspond to SEQ ID NO: 2 or SEQ ID NO: 4. In some aspects, the first antigen nucleic acid and/or the second antigen nucleic acid encodes a SARS CoV-2 S protein or an antigenic fragment thereof from a Delta SARS-CoV-2 strain, wherein the SARS-CoV-2 S protein or antigenic fragment thereof comprises the amino acid sequence of SEQ ID NO: 2 or SEQ ID NO: 4 with one or mutations selected from T19R, ΔD119-F120, AE156-F157, R158G, L452R, T478K, D614G, P681R, and D950N, wherein the amino acid locations correspond to SEQ ID NO: 2 or SEQ ID NO: 4. In some aspects, the first antigen nucleic acid and/or the second antigen nucleic acid encodes a SARS CoV-2 S protein or an antigenic fragment thereof from a Kappa SARS-CoV-2 strain, wherein the SARS-CoV-2 S protein or antigenic fragment thereof comprises the amino acid sequence of SEQ ID NO: 2 or SEQ ID NO: 4 with one or mutations selected from E154K, L452R, E484Q, D614G, P681R, and Q1071H, wherein the amino acid locations correspond to SEQ ID NO: 2 or SEQ ID NO: 4. In some aspects, the first antigen nucleic acid and/or the second antigen nucleic acid encodes a SARS CoV-2 S protein or an antigenic fragment thereof from an Eta SARS-CoV-2 strain, wherein the SARS-CoV-2 S protein or antigenic fragment thereof comprises the amino acid sequence of SEQ ID NO: 2 or SEQ ID NO: 4 with one or mutations selected from Q52R, A67V, ΔH69-V70, AY144, E484K, D614G, Q677H, and F888L, wherein the amino acid locations correspond to SEQ ID NO: 2 or SEQ ID NO: 4. In some aspects, the first antigen nucleic acid and/or the second antigen nucleic acid encodes a SARS CoV-2 S protein or an antigenic fragment thereof from an Iota SARS-CoV-2 strain, wherein the SARS-CoV-2 S protein or antigenic fragment thereof comprises the amino acid sequence of SEQ ID NO: 2 or SEQ ID NO: 4 with one or mutations selected from L5F, T95I, D253G, E484K, D614G, and A701V, wherein the amino acid locations correspond to SEQ ID NO: 2 or SEQ ID NO: 4. In some aspects, the first antigen nucleic acid and/or the second antigen nucleic acid encodes a SARS CoV-2 S protein or an antigenic fragment thereof from a Lambda SARS-CoV-2 strain, wherein the SARS-CoV-2 S protein or antigenic fragment thereof comprises the amino acid sequence of SEQ ID NO: 2 or SEQ ID NO: 4 with one or mutations selected from G75V, T76I, AR246-G252, D253N, L452Q, F490S, D614G, and T859N, wherein the amino acid locations correspond to SEQ ID NO: 2 or SEQ ID NO: 4. In some aspects, the first antigen nucleic acid and/or the second antigen nucleic acid encodes a SARS CoV-2 S protein or an antigenic fragment thereof from a Mu SARS-CoV-2 strain, wherein the SARS-CoV-2 S protein or antigenic fragment thereof comprises the amino acid sequence of SEQ ID NO: 2 or SEQ ID NO: 4 with one or mutations selected from T95I, Y144S, Y145N, R346K, E484K, N501Y, D614G, P681H, and D950N, wherein the amino acid locations correspond to SEQ ID NO: 2 or SEQ ID NO: 4. In some aspects, the first antigen nucleic acid and/or the second antigen nucleic acid encodes a SARS CoV-2 S protein or an antigenic fragment thereof from an Epsilon SARS-CoV-2 strain, wherein the SARS-CoV-2 S protein or antigenic fragment thereof comprises the amino acid sequence of SEQ ID NO: 2 or SEQ ID NO: 4 with one or mutations selected from S131, W152C, L452R, and D614G, wherein the amino acid locations correspond to SEQ ID NO: 2 or SEQ ID NO: 4.

In some aspects, the second antigen nucleic acid is operably linked to the first promoter through an internal ribosome entry site (IRES) sequence. In some aspects, the IRES sequence comprises a nucleic acid sequence having at least 70%, at least 75%, at least 80%, at least 85%, at least 90%, at least 95%, at least 99%, or 100% sequence identity to SEQ ID NO: 41.

In some aspects, the polynucleotide comprises one or more second promoters. In some aspects, the second antigen nucleic acid is operably linked to the one or more second promoters.

In some aspects, the first promoter or the one or more second promoters is selected from the group consisting of: a cytomegalovirus (CMV) promoter, a Rouse sarcoma virus (RSV) promoter, a Moloney murine leukemia virus (Mo-MuLV) long terminal repeat (LTR) promoter, a mammalian elongation factor 1 (EF1) promoter, a cytokeratin 18 (CK18) promoter, a cytokeratin 19 (CK19) promoter, a simian virus 40 (SV40) promoter, a murine U6 promoter, a skeletal α-actin promoter, a β-actin promoter, a murine phosphoglycerate kinase 1 (PGK1) promoter, a human PGK1 promoter, a CBA promoter, a CAG promoter, and any combination thereof. In some aspects, the mammalian EF1 promoter is a hEF1-HTLV promoter. In some aspects, the one or more second promoters is the CMV promoter.

In some aspects, the second antigen nucleic acid is under the control of a promoter selected from the group consisting of a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CBA promoter, a CAG promoter, and any combination thereof. In some aspects, the mammalian EF1 promoter is a hEF1-HTLV promoter.

In some aspects, the first antigen nucleic acid encodes a full-length SARS-CoV-2 S protein or an antigenic fragment thereof. In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the first antigen nucleic acid encodes a polypeptide having at least 70%, at least 75%, at least 80%, at least 85%, at least 90%, at least 95%, or at least 99% sequence identity to the amino acid sequence of SEQ ID NO: 2 or SEQ ID NO: 4. In some aspects, the first antigen nucleic acid encodes a polypeptide comprising the amino acid sequence of SEQ ID NO: 2 or SEQ ID NO: 4. In some aspects, the first antigen nucleic acid comprises a nucleic acid sequence having at least 70%, at least 75%, at least 80%, at least 85%, at least 90%, at least 95%, at least 99%, or 100% sequence identity to SEQ ID NO: 1 or SEQ ID NO: 3. In some aspects, the first antigen nucleic acid is operably linked to a mammalian EF1 promoter. In some aspects, the mammalian EF1 promoter is a hEF1-HTLV promoter.

In some aspects, the second antigen nucleic acid encodes a full-length SARS-CoV-2 S protein or an antigenic fragment thereof. In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the second antigen nucleic acid encodes a polypeptide having at least 70%, at least 75%, at least 80%, at least 85%, at least 90%, at least 95%, or at least 99% sequence identity to the amino acid sequence of SEQ ID NO: 2 or SEQ ID NO: 4. In some aspects, the second antigen nucleic acid encodes a polypeptide comprising the amino acid sequence of SEQ ID NO: 2 or SEQ ID NO: 4. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 1 or SEQ ID NO: 3. In some aspects, the second antigen nucleic acid is operably linked to a mammalian EF1 promoter through an IRES sequence. In some aspects, the mammalian EF1 promoter is a hEF1-HTLV promoter.

In some aspects, the first antigen nucleic acid encodes a full-length SARS-CoV-2 S protein or an antigenic fragment thereof, and wherein the second antigen nucleic acid encodes a SARS-CoV-2 membrane (M) protein or an antigenic fragment thereof. In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4, and wherein the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the first antigen nucleic acid encodes a polypeptide having at least 70%, at least 75%, at least 80%, at least 85%, at least 90%, at least 95%, or at least 99% sequence identity to the amino acid sequence of SEQ ID NO: 2 or SEQ ID NO: 4, and wherein the second antigen nucleic acid encodes a polypeptide having at least 70%, at least 75%, at least 80%, at least 85%, at least 90%, at least 95%, or at least 99% sequence identity to the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the first antigen nucleic acid encodes a polypeptide comprising the amino acid sequence of SEQ ID NO: 2 or SEQ ID NO: 4, and wherein the second antigen nucleic acid encodes a polypeptide comprising the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the first antigen nucleic acid comprises a nucleic acid sequence having at least 70%, at least 75%, at least 80%, at least 85%, at least 90%, at least 95%, at least 99%, or 100% sequence identity to SEQ ID NO: 1 or SEQ ID NO: 3, and wherein the second antigen nucleic acid comprises a nucleic acid sequence having at least 70%, at least 75%, at least 80%, at least 85%, at least 90%, at least 95%, at least 99%, or 100% sequence identity to SEQ ID NO: 7, SEQ ID NO: 9, SEQ ID NO: 11, SEQ ID NO: 13, SEQ ID NO: 15, SEQ ID NO: 17, or SEQ ID NO: 19. In some aspects, the first antigen nucleic acid is operably linked to a mammalian EF1 promoter, and wherein the second antigen nucleic acid is operably linked to a CMV promoter. In some aspects, the mammalian EF1 promoter is a hEF1-HTLV promoter.

In some aspects, the first antigen nucleic acid encodes the receptor binding domain (RBD) of the SARS-CoV-2 S protein or an antigenic fragment thereof. In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the first antigen nucleic acid encodes a polypeptide having at least 70%, at least 75%, at least 80%, at least 85%, at least 90%, at least 95%, or at least 99% sequence identity to the amino acid sequence of SEQ ID NO: 6. In some aspects, the first antigen nucleic acid encodes a polypeptide comprising the amino acid sequence of SEQ ID NO: 6. In some aspects, the first antigen nucleic acid comprises a nucleic acid sequence having at least 70%, at least 75%, at least 80%, at least 85%, at least 90%, at least 95%, at least 99%, or 100% sequence identity to SEQ ID NO: 5.

In some aspects, the second antigen nucleic acid encodes the receptor binding domain (RBD) of the SARS-CoV-2 S protein or an antigenic fragment thereof. In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the second antigen nucleic acid encodes a polypeptide having at least 70%, at least 75%, at least 80%, at least 85%, at least 90%, at least 95%, or at least 99% sequence identity to the amino acid sequence of SEQ ID NO: 6. In some aspects, the second antigen nucleic acid encodes a polypeptide comprising the amino acid sequence of SEQ ID NO: 6. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 5.

In some aspects, the first antigen nucleic acid encodes the S1 subunit of the SARS-CoV-2 S protein or an antigenic fragment thereof. In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the first antigen nucleic acid encodes a polypeptide having at least 70%, at least 75%, at least 80%, at least 85%, at least 90%, at least 95%, or at least 99% sequence identity to the amino acid sequence of SEQ ID NO: 40. In some aspects, the first antigen nucleic acid encodes a polypeptide comprising the amino acid sequence of SEQ ID NO: 40. In some aspects, the first antigen nucleic acid comprises a nucleic acid sequence having at least 70%, at least 75%, at least 80%, at least 85%, at least 90%, at least 95%, at least 99%, or 100% sequence identity to SEQ ID NO: 39. In some aspects, the first antigen nucleic acid is operably linked to a mammalian EF1 promoter. In some aspects, the mammalian EF1 promoter is a hEF1-HTLV promoter.

In some aspects, the second antigen nucleic acid encodes the S1 subunit of the SARS-CoV-2 S protein or an antigenic fragment thereof. In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the second antigen nucleic acid encodes a polypeptide having at least 70%, at least 75%, at least 80%, at least 85%, at least 90%, at least 95%, or at least 99% sequence identity to the amino acid sequence of SEQ ID NO: 40. In some aspects, the second antigen nucleic acid encodes a polypeptide comprising the amino acid sequence of SEQ ID NO: 40. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 39. In some aspects, the second antigen nucleic acid is operably linked to a mammalian EF1 promoter through an IRES sequence. In some aspects, the mammalian EF1 promoter is a hEF1-HTLV promoter.

In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the second antigen nucleic acid encodes a polypeptide having at least 70%, at least 75%, at least 80%, at least 85%, at least 90%, at least 95%, or at least 99% sequence identity to the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the second antigen nucleic acid encodes a polypeptide comprising the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 7, SEQ ID NO: 9, SEQ ID NO: 11, SEQ ID NO: 13, SEQ ID NO: 15, SEQ ID NO: 17, or SEQ ID NO: 19. In some aspects, the second antigen nucleic acid encodes at least 8, at least 10, at least 15, at least 20, at least 25, at least 30, at least 35, at least 40, at least 45, at least 50, at least 55, at least 60, at least 65, at least 70, or at least 75 contiguous amino acids of SEQ ID NO: 22, SEQ ID NO: 24, or SEQ ID NO: 26. In some aspects, the second antigen nucleic acid encodes a polypeptide having at least 70%, at least 75%, at least 80%, at least 85%, at least 90%, at least 95%, or at least 99% sequence identity to the amino acid sequence of SEQ ID NO: 22, SEQ ID NO: 24, or SEQ ID NO: 26. In some aspects, the second antigen nucleic acid encodes a polypeptide comprising the amino acid sequence of SEQ ID NO: 22, SEQ ID NO: 24, or SEQ ID NO: 26. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 21, SEQ ID NO: 23, or SEQ ID NO: 25. In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, or at least 400 contiguous amino acids of SEQ ID NO: 28. In some aspects, the second antigen nucleic acid encodes a polypeptide having at least 70%, at least 75%, at least 80%, at least 85%, at least 90%, at least 95%, or at least 99% sequence identity to the amino acid sequence of SEQ ID NO: 28. In some aspects, the second antigen nucleic acid encodes a polypeptide comprising the amino acid sequence of SEQ ID NO: 28. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 27.

In some aspects, the polypeptide(s) encoded by the first antigen nucleic acid (e.g., a first antigen nucleic acid encoding a SARS-CoV-2 full-length S protein, the RBD of a SARS-Cov-2 S protein, or the S1 subunit of a SARS-CoV-2 S protein) and/or the second antigen nucleic acid (e.g., a second antigen nucleic acid encoding a SARS-CoV-2 full-length S protein, the RBD of a SARS-Cov-2 S protein, or the S1 subunit of a SARS-CoV-2 S protein) comprise one or more mutations. In some aspects, the one or more mutations in the polypeptide(s) encoded by the first antigen nucleic acid and/or the second antigen nucleic acid comprise one or more mutations previously reported in Li, T. et al., Emerg Microbes Infect. 9(1):2076-90 (2020); Lee, P. et al., Immune Netw. 21(1):e4 (2021); Yu, J. et al., Science 369(6505):806-11 (2020); Cattin-Ortola, J. et al., Nat Commun. 12(1):5333 (2021); Corbett, K. et al., Nature 586(7830):567-71 (2020); Hsieh, C. et al., Science 369(6510):1501-5 (2020); and Harvey, W. et al., Nat Rev Microbiol. 19(7):409-24 (2021), each of which is incorporated by reference herein in its entirety.

In some aspects, the one or more mutations in the polypeptide(s) encoded by the first antigen nucleic acid and/or the second antigen nucleic acid are selected from: ΔM1-S13, S12P, S131, L5F, L18F, T19R, T20N, P26S, Q52R, A67V, ΔH69-V70, G75V, T76I, D80A, T95I, R102I, ΔD119-F120, C136Y, D138Y, ΔF140, ΔL141-Y144, AY144, Y144S, Y145N, ΔH146, N148S, K150R, K150E, K150T, K150Q, S151P, W152C, E154K, AE156-F157, F157L, F157A, R158G, R190S, ΔI210, D215G, A222V, ΔL241-S243, ΔL242-L244, ΔA243-L244, AR246-G252, R246I, 11-amino acid residue insertion between Y248 and L249, D253G, D253N, R346K, V367F, E406W, K417E, K417V, K417N, K417T, N439K, K444A, K444R, K444N, K444Q, V445A, V445E, G447A, N450D, L452R, L452Q, Y453F, L455F, N460I, S477G, S477N, S477R, T478I, T478K, V483I, E484K, E484Q, G485R, F486A, F486V, F486L, N487A, F490S, Q493E, Q493K, S494P, N501Y, A570D, Q613H, D614G, H655Y, Q677H, Δ678-679, Δ681-682, Δ681-684, Δ682-685, P681H, P681R, R682S, R682A, R682Q, R683S, R683G, R683Q, R685G, R685Q, I692V, A701V, T716I, F817P, T859N, F888L, A892P, A899P, A942P, D950N, S982A, K986P, V987P, T1027I, Q1071H, D1118H, V1176F, M1229I, ΔC1253-T1273, ΔC1254-T1273, ΔK1255-T1273, D1257A, E1258A, D1259A, D1260A, E1262A, K1269A, H1271K, T1273A, or any combination thereof, wherein the amino acid locations correspond to SEQ ID NO: 2 or SEQ ID NO: 4.

In some aspects, the one or more mutations in the polypeptide(s) encoded by the first antigen nucleic acid and/or the second antigen nucleic acid comprise one or more mutations in the N-terminal signal peptide of the full-length SARS-CoV-2 S protein, which corresponds to amino acids 1-13 of SEQ ID NO: 2 or SEQ ID NO: 4. In some aspects, the one or more mutations in the N-terminal signal peptide is ΔM1-S13, wherein the amino acid locations correspond to SEQ ID NO: 2 or SEQ ID NO: 4.

In some aspects, the one or mutations in the polypeptide(s) encoded by the first antigen nucleic acid and/or the second antigen nucleic acid comprise one or more mutations in the C-terminus of the full-length SARS-CoV-2 S protein. In some aspects, the one or more mutations in the C-terminus of the full-length SARS-CoV-2 S protein comprise one or more mutations in the C-terminal endoplasmic reticulum (ER) retention peptide, which corresponds to amino acids 1254-1273 of SEQ ID NO: 2 or SEQ ID NO: 4. In some aspects, the one or more mutations in the C-terminal ER retention peptide are selected from D1257A, E1258A, D1259A, D1260A, E1262A, K1269A, H1271K, T1273A, or any combination thereof, wherein the amino acid locations correspond to SEQ ID NO: 2 or SEQ ID NO: 4. In some aspects, the one or more mutations in the C-terminal ER retention peptide comprise D1257A+E1258A+D1259A+D1260A+E1262A (i.e., a D/E to A mutant), wherein the amino acid locations correspond to SEQ ID NO: 2 or SEQ ID NO: 4. In some aspects, the one or mutations in the C-terminal ER retention peptide is ΔC1253-T1273, ΔC1254-T1273, or ΔK1255-T1273.

In some aspects, the one or more mutations in the polypeptide(s) encoded by the first antigen nucleic acid and/or the second antigen nucleic acid comprise K986P+V987P (i.e., a S-2P mutation), wherein the amino acid locations correspond to SEQ ID NO: 2 or SEQ ID NO: 4. In some aspects, the one or more mutations in the polypeptide(s) encoded by the first antigen nucleic acid and/or the second antigen nucleic acid comprise F817P+A892P+A899P+A942P (i.e., a hexa-proline S mutation), wherein the amino acid locations correspond to SEQ ID NO: 2 or SEQ ID NO: 4. In some aspects, the one or more mutations in the polypeptide(s) encoded by the first antigen nucleic acid and/or the second antigen nucleic acid comprise one or more mutations in the 681-PRRAR/SVA-688 S1/S2 furin cleavage site, wherein the amino acid locations correspond to SEQ ID NO: 2 or SEQ ID NO: 4. In some aspects, the one or more mutations in the 681-PRRAR/SVA-688 S1/S2 furin cleavage site are: (a) R682S+R683S (i.e., a SSAR mutation), (b) Δ681-684 (i.e., a ΔPRRA mutation), (c) Δ678-679+Δ681-682, (d) R682A+R683G+R685G (i.e., a 682-AGAG-685 mutation), (e) R682Q+R683Q+R685Q, (f) R682S+R685G, or (g) Δ682-685 (i.e., a ΔRRAR mutation), wherein the amino acid locations correspond to SEQ ID NO: 2 or SEQ ID NO: 4.

In some aspects, the one or more mutations in the polypeptide(s) encoded by the first antigen nucleic acid and/or the second antigen nucleic acid comprise: (a) F817P+A892P+A899P+A942P (i.e., a hexa-proline S mutation) and (b) K986P+V987P (i.e., a S-2P mutation), wherein the amino acid locations correspond to SEQ ID NO: 2 or SEQ ID NO: 4. In some aspects, the one or more mutations in the polypeptide(s) encoded by the first antigen nucleic acid and/or the second antigen nucleic acid comprise: (a) R682A+R683G+R685G (i.e., a 682-AGAG-685 mutation) and (b) K986P+V987P (i.e., a S-2P mutation), wherein the amino acid locations correspond to SEQ ID NO: 2 or SEQ ID NO: 4. In some aspects, the one or more mutations in the polypeptide(s) encoded by the first antigen nucleic acid and/or the second antigen nucleic acid comprise: (a) R682A+R683G+R685G (i.e., a 682-AGAG-685 mutation), (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations in the polypeptide(s) encoded by the first antigen nucleic acid and/or the second antigen nucleic acid comprise: (a) R682Q+R683Q+R685Q and (b) K986P+V987P (i.e., a S-2P mutation). In some aspects, the one or more mutations in the polypeptide(s) encoded by the first antigen nucleic acid and/or the second antigen nucleic acid comprise:: (a) R682Q+R683Q+R685Q, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations in the polypeptide(s) encoded by the first antigen nucleic acid and/or the second antigen nucleic acid comprise: (a) R682S+R685G and (b) K986P+V987P (i.e., a S-2P mutation), wherein the amino acid locations correspond to SEQ ID NO: 2 or SEQ ID NO: 4. In some aspects, the one or more mutations in the polypeptide(s) encoded by the first antigen nucleic acid and/or the second antigen nucleic acid comprise: (a) R682S+R685G, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4.

In some aspects, the polynucleotide further comprises one or more post-transcriptional regulatory elements. In some aspects, the post-transcriptional regulatory element is a wood chuck hepatitis virus post-transcriptional regulatory element (WPRE).

In some aspects, the polynucleotide further comprises at least one 3′ UTR poly(a) tail sequence operably linked to the first antigen nucleic acid, the second antigen nucleic acid, the nucleic acid encoding an immune modifier, or any combination thereof. In some aspects, the 3′ UTR poly(a) tail sequence is a 3′ UTR SV40 poly(a) tail sequence, a 3′ UTR bovine growth hormone (bGH) poly(A) sequence, a 3′ UTR actin poly(A) tail sequence, a 3′ UTR hemoglobin poly(A) sequence, or combinations thereof.

In some aspects, the polynucleotide further comprises at least one enhancer sequence. In some aspects, the enhancer sequence is a human actin enhancer sequence, a human myosin enhancer sequence, a human hemoglobin enhancer sequence, a human muscle creatine enhancer sequence, a viral enhancer sequence, a polynucleotide function enhancer sequence, or any combination thereof. In some aspects, the enhancer sequence is a CMV intronic sequence, a j-actin intronic sequence, or the combination thereof.

In some aspects, the polynucleotide further comprises an inverted terminal repeat (ITR). In some aspects, the polynucleotide comprises a first ITR and a second ITR. In some aspects, the first ITR and the second ITR are both derived from an adeno-associated virus (AAV).

In some aspects, the vector is a DNA plasmid vector. In some aspects, the DNA plasmid vector is selected from the group consisting of: pVac 1, pVac 4, and pVac 7. In some aspects, the vector is a viral vector, a bacterial vector, a cosmid, or an artificial chromosome. In some aspects, the viral vector is selected from an AAV vector, adenoviral vector, retroviral vector, poxvirus vector, baculovirus vector, herpes viral vector, or combinations thereof.

In some aspects, the composition, pharmaceutical composition, or vaccine further comprises an adjuvant. In some aspects, the adjuvant comprises one or more concatamers of non-coding 5′-C-phosphate-G-3′ (CpG) dinucleotides. In some aspects, the one or more concatamers of non-coding CpG dinucleotides activate the Toll-like receptor 9 (TLR9) signaling pathway. In some aspects, the one or more concatamers of non-coding CpG dinucleotides comprise one or more concatamers of non-coding CpG dinucleotides previously reported in Bauer, A. et al., Nucleic Acids Research 38(12):3891-908 (2010); Cornelie, S. et al., Journal of Biological Chemistry 279(15):15124-9 (2004); Klinman, D. et al., J Immunol. 158(8):3635-9 (1997); Klinman, D. et al., Immunological Reviews 199(1):201-16 (2004); Luo, Z. et al., Mol Med Rep. 6(6):1309-14 (2012); Bode, C. et al., Expert Rev Vaccines 10(4):499-511 (2011); and Kuo, T. et al., Scientific Reports 10:20085 (2020), each of which is incorporated by reference herein in its entirety.

In some aspects, the composition, pharmaceutical composition, or vaccine further comprises a pharmaceutically a pharmaceutically acceptable carrier. In some aspects, the composition, pharmaceutical composition, or vaccine is lyophilized.

In some aspects, the delivery component of the composition, pharmaceutical composition, or vaccine is a cationic polymer. In some aspects, the cationic polymer is a synthetic functionalized polymer, a lipid, a lipopolymer, or a chemical derivative thereof. In some aspects, the synthetic functionalized polymer is a biodegradable cross-linked cationic multi-block copolymer.

In some aspects, the biodegradable cross-linked cationic multi-block copolymer is represented by the formula: (CP)ₓL_{y}Y_{z}, wherein: (a) CP represents a cationic polymer containing at least one secondary amine group, wherein the cationic polymer has a number averaged molecular weight within the range of 1,000 to 25,000 Dalton; (b) Y represents a bifunctional biodegradable linker containing ester, amide, disulfide, or phosphate linages; (c) L represents a ligand; (d) x is an integer in the range from 1 to 20; (e) y is an integer in the range from 0 to 100; and (f) z is an integer in the range from 0 to 40. In some aspects, the cationic polymer comprises linear polyethyleneimine (LPEI). In some aspects, the cationic polymer comprising linear polyethyleneimine (LPEI) is BD15K-12, which has the following formula:

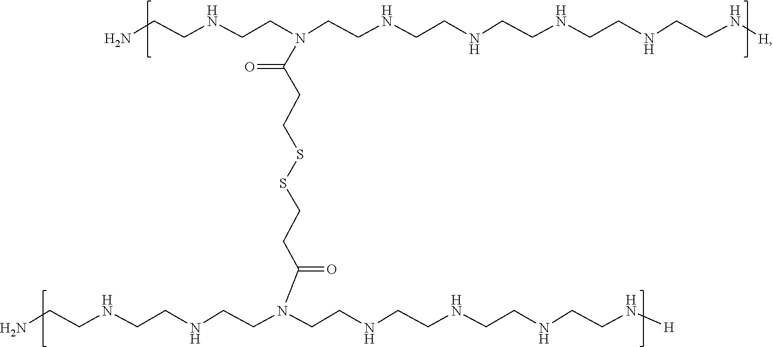

wherein the PEIs are approximately 15,000 Da, and wherein there is an average of 12 crosslinkers per PEI. In some aspects, the bifunctional biodegradable linker is hydrophilic and comprises a biodegradable linkage comprising a disulfide bond. In some aspects, the bifunctional biodegradable linker is a dithiodipropionyl linker.

In some aspects, the biodegradable cross-linked cationic multi-block copolymer comprises LPEI and a dithiodipropionyl linker for cross-linking the multi-block copolymer, wherein the LPEI has an average molecular weight of 1,000 to 25,000 Dalton. In some aspects, the biodegradable cross-linked cationic multi-block copolymer is covalently linked to at least one ligand.

In some aspects, the ligand is a targeting ligand selected from the group consisting of: a sugar moiety, a polypeptide, folate, and an antigen. In some aspects, the sugar moiety is a monosaccharide. In some aspects, the monosaccharide is galactose. In some aspects, the sugar moiety is an oligosaccharide. In some aspects, the polypeptide is a glycoprotein, an antibody, an antibody fragment, a cell receptor, a cytokine receptor, or a growth factor receptor. In some aspects, the growth factor receptor is an epidermal growth factor receptor. In some aspects, the glycoprotein is transferrin or asialoorosomucoid (ASOR). In some aspects, the antigen is a viral antigen, a bacterial antigen, or a parasite antigen.

In some aspects, the biodegradable cross-linked cationic multi-block copolymer is covalently linked to polyethylene glycol (PEG) of molecular weight ranging from 500 to 20,000 Dalton. In some aspects, the biodegradable cross-linked cationic multi-block copolymer is covalently linked to a fatty acyl chain selected from the group consisting of: oleic acid, palmitic acid, and stearic acid. In some aspects, the biodegradable cross-linked cationic multi-block copolymer comprises at least one amine group that is electrostatically attracted to a polyanionic compound. In some aspects, the polyanionic compound is a nucleic acid, wherein the biodegradable cross-linked cationic multi-block copolymer condenses the nucleic acid to form a compact structure.

In some aspects, the delivery component of the composition, pharmaceutical composition, or vaccine is a cationic lipopolymer comprising a PEI backbone covalently linked to a lipid or a PEG. In some aspects, the PEI backbone is covalently linked to a lipid and a PEG. In some aspects, the lipid and the PEG are directly attached to the PEI backbone by covalent bonds. In some aspects, the lipid is attached to the PEI backbone through a PEG spacer. In some aspects, the PEG has a molecular weight of between 50 to 20,000 Dalton. In some aspects, the molar ratio of PEG to PEI is within a range of 0.1:1 to 500:1. In some aspects, the molar ratio of the lipid to the PEI is within a range of 0.1:1 to 500:1. In some aspects, the lipid is a cholesterol, a cholesterol derivative, a C12 to Cis fatty acid, or a fatty acid derivative. In some aspects, the PEI is covalently linked to cholesterol and PEG, and wherein the average PEG:PEI:cholesterol molar ratio in the cationic lipopolymer is within the range of 1-5 PEG:1 PEI:0.4-1.5 cholesterol. In some aspects, the PEI has a linear or branch configuration with a molecular weight of 100 to 500,000 Dalton.

In some aspects, the cationic lipopolymer further comprises a pendant functional moiety selected from the group consisting of: a receptor ligand, a membrane permeating agent, an endosomolytic agent, a nuclear localization sequence, and a pH sensitive endosomolytic peptide.

In some aspects, the cationic lipopolymer further comprises a targeting ligand, wherein the targeting ligand is directly attached to the PEI backbone or is attached through a PEG linker. In some aspects, the targeting ligand is selected from the group consisting of: a sugar moiety, a polypeptide, folate, and an antigen. In some aspects, the sugar moiety is a monosaccharide. In some aspects, the monosaccharide is galactose. In some aspects, the sugar moiety is an oligosaccharide. In some aspects, the polypeptide is a glycoprotein, an antibody, an antibody fragment, a cell receptor, a cytokine receptor, or a growth factor receptor. In some aspects, the growth factor receptor is an epidermal growth factor receptor. In some aspects, the glycoprotein is transferrin or asialoorosomucoid (ASOR). In some aspects, the antigen is a viral antigen, a bacterial antigen, or a parasite antigen.

In some aspects, the cationic polymer of the composition, pharmaceutical composition, or vaccine is present in an amount sufficient to produce a ratio of amine nitrogen in the cationic polymer to phosphate in the polynucleotide, multicistronic mRNA vector, or DNA plasmid vector from about 0.01:1 to about 50:1 (e.g., about 0.01:1 to about 40:1; about 0.01:1 to about 30:1; about 0.01:1 to about 20:1; about 0.01:1 to about 10:1, or about 0.01:1 to about 5:1). In some aspects, the ratio of amine nitrogen in the cationic polymer to phosphate in the polynucletide, multicistronic mRNA vector, or DNA plasmid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the cationic polymer to phosphate in the polynucleotide, multicistronic mRNA vector, or DNA plasmid vector is from about 1:10 to about 10:1.

In some aspects, the composition, pharmaceutical composition, or vaccine comprises about 0.1 mg/ml to about 10 mg/ml (e.g., about 0.1 mg/ml to about 5 mg/ml; about 0.5 mg/ml to about 10 mg/ml; or about 0.5 mg/ml to about 5 mg/ml) nucleic acid (e.g., DNA plasmid vector or multicistronic mRNA vector) complexed with the cationic polymer. In some aspects, the composition, pharmaceutical composition, or vaccine comprises about 1 mg/ml to about 10 mg/ml (e.g., about 1 mg/ml to about 6 mg/ml; about 2 mg/ml to about 6 mg/ml; about 5 mg/ml to about 10 mg/ml; or about 6 mg/ml to about 10 mg/ml) nucleic acid (e.g., DNA plasmid vector or multicistronic mRNA vector) complexed with the cationic polymer.

In some aspects, the delivery component of the composition, pharmaceutical composition, or vaccine comprises a lipopolyamine with the following formula:

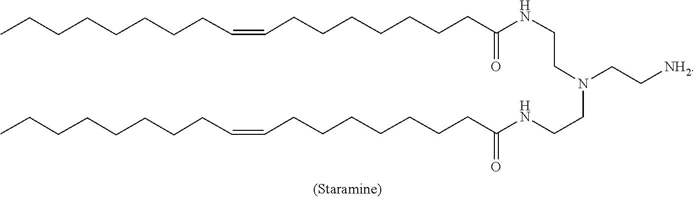 [SMILES: CCCCCCCC/C=C\CCCCCCCC(=O)NCCN(CCN)CCNC(=O)CCCCCCC/C=C\CCCCCCCC]

In some aspects, the delivery component comprises a mixture of the lipopolyamine and an alkylated derivative of the lipopolyamine. In some aspects, the alkylated derivative of the lipopolyamine is a polyoxyalkylene, polyvinylpyrrolidone, polyacrylamide, polydimethylacrylamide, polyvinyl alcohol, dextran, poly (L-glutamic acid), styrene maleic anhydride, poly-N-(2-hydroxypropyl) methacrylamide, or polydivinylether maleic anhydride. In some aspects, the alkylated derivative of the lipopolyamine has the following formula:

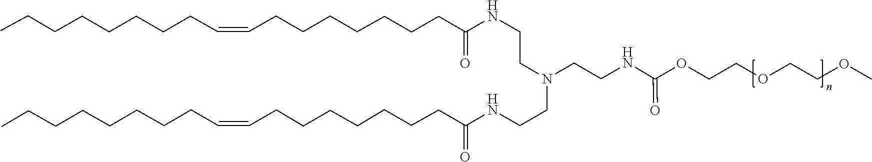

(methoxypolyethylene glycol (mPEG) modified Staramine), wherein n represents an integer from 10 to 100 repeating units containing 2-5 carbon atoms each. In some aspects, the alkylated derivative of the lipopolyamine has the following formula:

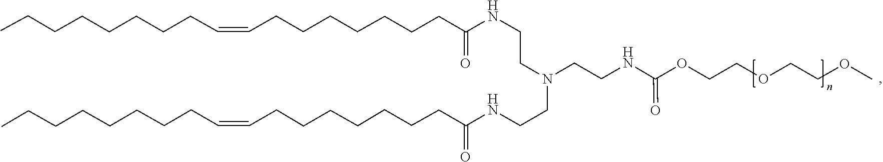

wherein n=11 (Staramine-mPEG515). In some aspects, the alkylated derivative of the lipopolyamine has the following formula:

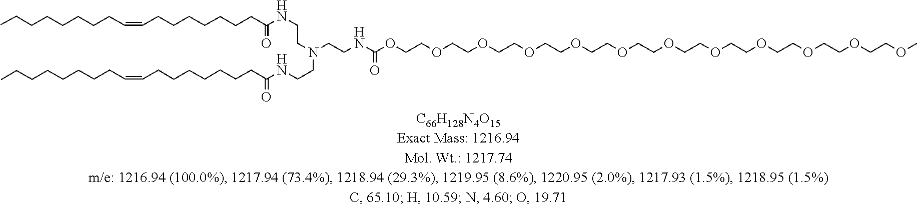 [SMILES: CCCCCCCC/C=C\CCCCCCCC(=O)NCCN(CCNC(=O)CCCCCCC/C=C\CCCCCCCC)CCNC(=O)OCCOCCOCCOCCOCCOCCOCCOCCOCCOCCOCCOC]

In some aspects, the ratio of the lipopolyamine to the alkylated derivative of the lipopolyamine in the mixture is 1:1 to 10:1. In some aspects, the lipopolyamine is present in an amount sufficient to produce a ratio of amine nitrogen in the lipopolyamine to phosphate in the polynucleotide, multicistronic mRNA vector, or DNA plasmid vector from about 0.01:1 to about 50:1 (e.g., about 0.01:1 to about 40:1; about 0.01:1 to about 30:1; about 0.01:1 to about 20:1; about 0.01:1 to about 10:1, or about 0.01:1 to about 5:1). In some aspects, the ratio of amine nitrogen in the lipopolyamine to phosphate in the polynucleotide, multicistronic mRNA vector, or DNA plasmid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the lipopolyamine to phosphate in the polynucleotide, multicistronic mRNA vector, or DNA plasmid vector is from about 1:10 to about 10:1.

In some aspects, the delivery component of the composition, pharmaceutical composition, or vaccine comprises a lipopolyamine with the following formula:

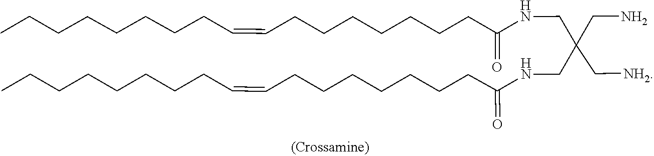 [SMILES: CCCCCCCC/C=C\CCCCCCCC(=O)NCC(CN)(CN)CNC(=O)CCCCCCC/C=C\CCCCCCCC]

In some aspects, the delivery component comprises a mixture of the lipopolyamine and an alkylated derivative of the lipopolyamine. In some aspects, the alkylated derivative of the lipopolyamine is a polyoxyalkylene, polyvinylpyrrolidone, polyacrylamide, polydimethylacrylamide, polyvinyl alcohol, dextran, poly (L-glutamic acid), styrene maleic anhydride, poly-N-(2-hydroxypropyl) methacrylamide, or polydivinylether maleic anhydride. In some aspects, the ratio of the lipopolyamine to the alkylated derivative of the lipopolyamine in the mixture is 1:1 to 10:1. In some aspects, the lipopolyamine is present in an amount sufficient to produce a ratio of amine nitrogen in the lipopolyamine to phosphate in the polynucleotide, multicistronic mRNA vector, or DNA plasmid vector from about 0.01:1 to about 50:1 (e.g., about 0.01:1 to about 40:1; about 0.01:1 to about 30:1; about 0.01:1 to about 20:1; about 0.01:1 to about 10:1, or about 0.01:1 to about 5:1). In some aspects, the ratio of amine nitrogen in the lipopolyamine to phosphate in the polynucleotide, multicistronic mRNA vector, or DNA plasmid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the lipopolyamine to phosphate in the polynucleotide, multicistronic mRNA vector, or DNA plasmid vector is from about 1:10 to about 10:1.

In some aspects, the delivery component of the composition, pharmaceutical composition, or vaccine comprises a poloxamer back-bone having a metal chelator covalently coupled to at least one terminal end of the poloxamer backbone. In some aspects, the metal chelator is coupled to at least two terminal ends of the poloxamer backbone. In some aspects, the poloxamer backbone is a poloxamer backbone disclosed in U.S. Publ. No. 2010/0004313, which is herein incorporated by reference in its entirety. In some aspects, the metal chelator is a metal chelator disclosed in U.S. Publ. No. 2010/0004313. In some aspects, the delivery component of the composition, pharmaceutical composition, or vaccine comprises a polymer having the following formula:

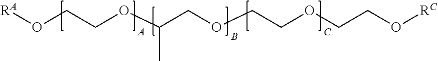

and pharmaceutically acceptable salts thereof, wherein:
   A represents an integer from 2 to 141;
   B represents an integer from 16 to 67;
   C represents an integer from 2 to 141;
   R^{A} and R^{C} are the same or different, and are R′-L- or H, wherein at least one of R^{A} and R^{C} is R′-L-;
   L is a bond, —CO—, CH₂—O, or —O—CO—; and
   R′ is a metal chelator.

In some aspects, the metal chelator is RNNH—, RN2N—, or (R″—(N(R″) CH2CH2)x)2-N—CH2CO—, wherein each x is independently 0-2, and wherein R″ is HO₂C—CH₂—. In some aspects, the metal chelator is a crown ether selected from the group consisting of 12-crown-4, 15-crown-5, 18-crown-6, 20-crown-6, 21-crown-7, and 24-crown-8. In some aspects, the crown ether is a substituted-crown ether, wherein the substituted crown ether has:
(1) one or more of the crown ether oxygens independently replaced by NH or S,
(2) one or more of the crown ether —CH₂—CH₂— moieties replaced by C₆H₄—, —C₁₀H₆—, or C₆H₁₀—,
(3) one or more of the crown ether —CH₂—O—CH₂— moieties replaced by C₄H₂O— or —C₅H₃N—, or
(4) any combination thereof.

In some aspects, the metal chelator is a cryptand, wherein the cryptand is selected from the group consisting of (1,2,2) cryptand, (2,2,2) cryptand, (2,2,3) cryptand, and (2,3,3) cryptand. In some aspects, the cryptand is a substituted-cryptand, wherein the substituted cryptand has:
(1) one or more of the crypthand ether oxygens independently replaced by NH or S,
(2) one or more of the crown ether CH₂—CH₂— moieties replaced by —C₆H₄—, —C₁₀H₆—, or C₆H₁₀,
(3) one or more of the crown ether —CH₂—O—CH₂— moieties replaced by —C₄H₂O— or —C₅H₃N—, or
(4) any combination thereof.

In some aspects, the delivery component is Crown Poloxamer (aza-crown-linked poloxamer), wherein the Crown Poloxamer comprises a polymer having the following formula:

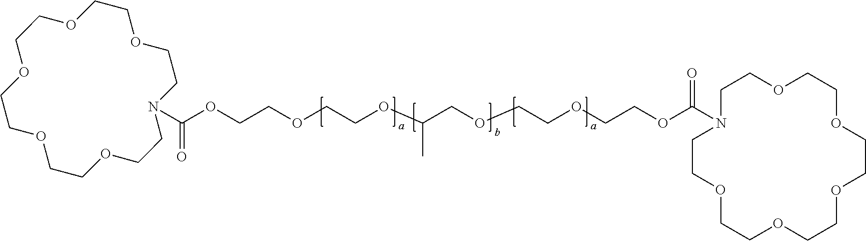

or pharmaceutically acceptable salts thereof, wherein:
   a represents an integer of about 10 units; and
   b represents an integer of about 21 units; and
wherein the total molecular weight of the polymer is about 2,000 Da to about 2,200 Da.

In some aspects, the polymer is present in a solution with the polynucleotide, multicistronic mRNA vector, or DNA plasmid vector from about 0.1%—about 5% or about 0.5%—about 5%.

In some aspects, the delivery component of the composition, pharmaceutical composition, or vaccine is a 3-amino ester. In some aspects, the polymer is present in a solution with the polynucleotide, multicistronic mRNA vector, or DNA plasmid vector from about 0.1%—about 5% or about 0.5%—about 5%.

In some aspects, the delivery component of the composition, pharmaceutical composition, or vaccine is a poly-inosinic-polycytidylic acid. In some aspects, the poly-inosinic-polycytidylic acid is present in a solution with the polynucleotide, multicistronic mRNA vector, or DNA plasmid vector from about 0.1% —about 5% or about 0.5%—about 5%.

In some aspects, the delivery component further comprises benzalkonium chloride.

In some aspects, the delivery component comprises BD15-12. In some aspects, the ratio of nucleotide to BD15-12 polymer (N:P) is 5:1.

In some aspects, the delivery component comprises Omnifect. In some aspects, the ratio of nucleotide to Omnifect polymer (N:P) is 10:1.

In some aspects, the delivery component comprises Crown Poloxamer (aza-crown-linked poloxamer). In some aspects, the ratio of nucleotide to Crown Poloxamer (N:P) is 5:1. In some aspects, the delivery component comprises Crown Poloxamer and a PEG-PEI-cholesterol (PPC) lipopolymer. In some aspects, the delivery component comprises Crown Poloxamer and benzalkonium chloride. In some aspects, the delivery component comprises Crown Poloxamer and Omnifect. In some aspects, the delivery component comprises Crown Poloxamer and a linear polyethyleneimine (LPEI). In some aspects, the delivery component comprises Crown Poloxamer and BD15-12.

In some aspects, the delivery component comprises Staramine and mPEG modified Staramine. In some aspects, the mPEG modified Staramine is Staramine-mPEG515. In some aspects, the mPEG modified Staramine is Staramine-mPEG11. In some aspects, the ratio of Staramine to mPEG modified Staramine is 10:1. In some aspects, the nucleotide to polymer (N:P) ratio is 5:1. In some aspects, the delivery component comprises Staramine, mPEG modified Staramine, and Crown Poloxamer. In some aspects, the delivery component comprises Staramine, Staramine-mPEG515, and Crown Poloxamer. In some aspects, the delivery component comprises Staramine, Staramine-mPEG11, and Crown Poloxamer.

In some aspects, the composition, pharmaceutical composition, or vaccine is lyophilized.

Also provided herein is a host cell comprising any polynucleotide, vector, multicistronic mRNA vector, DNA plasmid vector, composition, pharmaceutical composition, or vaccine described or exemplified herein. In some aspects, the host cell is a eukaryotic host cell. In some aspects, the host cell is a human host cell.

Also provided herein is a kit comprising any polynucleotide, vector, multicistronic mRNA vector, DNA plasmid vector, composition, pharmaceutical composition, vaccine, or lyophilized composition described or exemplified herein. In some aspects, the kit further comprises a glass vial. In some aspects, the kit further comprises instructions for using the polynucleotide, vector, multicistronic mRNA vector, DNA plasmid vector, composition, or pharmaceutical composition in a method for inducing an immune response in a subject. In some aspects, the kit further comprise instruction for reconstituting the composition, pharmaceutical composition, vaccine or lyophilized vaccine. In some aspects, the kit further comprises instructions for using the polynucleotide, vector, multicistronic mRNA vector, DNA plasmid vector, composition, pharmaceutical composition, vaccine, or lyophilized composition in a method for preventing, reducing the incidence of, attenuating or treating a virus (e.g., SARS-CoV-2), a bacteria or a parasite infection in a subject.

Also provided herein is a method of inducing an immune response in a subject, the method comprising administering an effective amount of any polynucleotide, vector, multicistronic mRNA vector, DNA plasmid vector, composition, pharmaceutical composition, or vaccine described or exemplified herein to the subject. In some aspects, the immune response is to one or more antigens disclosed herein. In some aspects, the immune response is a protective immune response. In some aspects, the polynucleotide, vector, multicistronic mRNA vector, DNA plasmid vector, composition, pharmaceutical composition, or vaccine is administered to the subject by an intramuscular, subcutaneous, intralymphatic, intranasal, or intraperitoneal route of administration.

In some aspects, the immune response is to one or more antigens comprising one or more viral antigens, one or more bacterial antigens, or one or more parasite antigens.

In some aspects, the immune response is to a bacterial antigen selected from the group consisting of a Yersinia pestis antigen or a Mycobacterium tuberculosis antigen. In some aspects, the Yersinia pestis antigen is a Yersinia pestis capsular antigen. In some aspects, the Yersinia pestis capsular antigen is F1-Ag or virulence antigen (V-Ag). In some aspects, the Mycobacterium tuberculosis antigen is an Apa antigen, an HP65 antigen, or a rAg85A antigen.

In some aspects, the immune response is to a viral antigen is selected from the group consisting of: an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a dengue virus antigen, an Ebola virus antigen, a Zika virus, a chikungunya virus antigen, a measles virus antigen, a Middle East Respiratory Syndrome Coronavirus (MERS-CoV) antigen, or a SARS-CoV antigen. In some aspects, the enterovirus antigen is an enterovirus 71 (E71) antigen or a coxsackievirus (Cox) protein antigen. In some aspects, the E71 antigen is an E71-VP1 antigen or a glutathione S-transferase (GST)-tagged E71-VP1 antigen. In some aspects, the Cox protein antigen is GST-tagged Cox protein antigen. In some aspects, the HSV antigen is an HSV-1 envelope antigen, an HSV-2 envelope antigen, or an HSV-2 surface glycoprotein antigen. In some aspects, the HSV-2 surface glycoprotein antigen is a gB2 antigen, a gC2 antigen, a gD2 antigen, or a gE2 antigen. In some aspects, the HIV antigen is an Env antigen, a Gag antigen, a Nef antigen, or a Pol antigen. In some aspects, the HPV antigen is a minor capsid protein L2 antigen. In some aspects, the minor capsid protein L2 antigen comprises one or more epitope domains (amino acids 10-36 and/or amino acids 65-89) of minor capsid protein L2. In some aspects, the HCV antigen is a nonstructural 3 (NS3) antigen. In some aspects, the RSV antigen is an F antigen or a G antigen. In some aspects, the Dengue virus antigen is an E protein antigen, an E protein domain III (EDIII) antigen, a non-structural protein 1 (NS1) antigen, or a DEN-80E antigen. In some aspects, the Ebola virus antigen is a spike glycoprotein (GB) antigen, a VP24 antigen, a VP40 antigen, a nucleoprotein (NP) antigen, a VP30 antigen, or a VP35 antigen. In some aspects the Zika virus antigen is an envelope domain III antigen or a CKD antigen. In some aspects, the Chikungunya virus antigen is an E1 glycoprotein subunit antigen, the MHC class I epitope PPFGAGRPGQFGDI (SEQ ID NO: 34), the MHC class I epitope TAECKDKNL (SEQ ID NO: 35), or the MHC class II epitope VRYKCNCGG (SEQ ID NO: 36). In some aspects, the measles virus antigen is a hemagglutinin protein MV-H antigen or a fusion protein MV-F antigen. In some aspects, the MERS-CoV antigen is a spike (S) protein antigen, an antigen from the receptor-binding domain of the S protein, or an antigen from the membrane fusion domain of the S protein. In some aspects, the SARS-CoV antigen is a spike (S) protein antigen, an antigen from the receptor binding domain of the S protein, an antigen from the membrane fusion domain of the S protein, an envelope (E) protein antigen, or an M protein antigen.

In some aspects, the immune response is to one or more influenza virus antigens from any influenza virus type or subtype. In some aspects, the one or more influenza virus antigens are selected from the group consisting of: an influenza virus hemagglutinin (HA) antigen, an influenza virus neuraminidase (NA) antigen, an influenza virus matrix-2 (M2) protein antigen, antigenic fragments thereof, and any combination thereof. In some aspects, the one or more influenza virus antigens are derived from influenza virus type A, type B, type C, type D, or any combination thereof. In some spects, the one or more influenza virus antigens are derived from influenza virus type A. In some aspects, the one or more influenza virus antigens derived from influenza virus type A have (a) a HA subtype selected from H1 through H18 or any combination thereof and (b) a NA subtype selected from N1 through N11 or any combination thereof. In some aspects, the one or more influenza virus antigens derived from influenza virus type A, subtype H1N1; influenza virus type A, subtype H2N2; influenza virus type A, subtype H3N2; influenza virus type A, subtype H5N1; influenza virus type A, subtype H7N7; influenza virus type A, subtype H7N9; influenza virus type A, subtype H9N2; or any combination thereof. In some aspects, the one or more influenza virus antigens are derived from influenza virus type A, subtype H1N1; influenza virus type A, subtype H3N2; or the combination thereof. In some spects, the one or more influenza virus antigens are derived from influenza virus type B. In some aspects, the immune response is to one or more SARS-CoV-2 antigens or antigenic fragments thereof disclosed herein and one or more influenza virus antigens or antigenic fragments thereof disclosed herein. In some aspects, the immune response is to two or more SARS-CoV-2 antigens or antigenic fragments thereof disclosed herein. In some aspects, the immune response is to two or more SARS-CoV-2 antigens or antigenic fragments thereof disclosed herein that are from different strains of SARS-CoV-2. In some aspects, the immune response is to two or more different variants of the same SARS-CoV-2 protein or antigenic fragment thereof, wherein the different variants of the same SARS-CoV2 protein or antigenic fragment thereof are derived from different strains of SARS-CoV-2. In some aspects, the immune response is to two or more SARS-CoV-2 S proteins or antigenic fragments thereof from different strains of SARS-CoV-2. In some aspects, the SARS-CoV-2 S proteins or antigenic fragments thereof from different strains of SARS-CoV-2 comprise one or more mutations previously reported in Li, T. et al., Emerg Microbes Infect. 9(1):2076-90 (2020); Lee, P. et al., Immune Netw. 21(1):e4 (2021); Yu, J. et al., Science 369(6505):806-11 (2020); Cattin-Ortola, J. et al., Nat Commun. 12(1):5333 (2021); Corbett, K. et al., Nature 586(7830):567-71 (2020); Hsieh, C. et al., Science 369(6510):1501-5 (2020); and Harvey, W. et al., Nat Rev Microbiol. 19(7):409-24 (2021), each of which is incorporated by reference herein in its entirety. In some aspects, the different strains of SARS-CoV-2 are selected from the group consisting of: an Alpha SARS-CoV-2 strain (e.g., strains B.1.1.7 and Q.1-Q.8); a Beta SARS-CoV-2 strain (e.g., strains B.1.351, B.1.351.2, and B.1.351.3); a Delta SARS-CoV-2 strain (e.g., strain B.1.617.2 and AY.1 sublineages); a SARS-CoV-2 strain Gamma strain (e.g., strains P.1, P.1.1, and P.1.2); an Epsilon SARS-CoV-2 strain (e.g., strains B.1.427 and B.1.429); an Eta SARS-CoV-2 strain (e.g., strain B.1.525); an Iota SARS-CoV-2 strain (e.g., strain B.1.526); a Kappa SARS-CoV-2 strain (e.g., strain B.1.617.1); a Lambda SARS-CoV-2 strain; a B.1.617.3 SARS-CoV-2 strain; a Mu SARS-CoV-2 strain (e.g., strains B.1.621 and B.1.621.1); a Zeta strain (e.g., strain P.2); and any combination thereof. In some aspects, the different strains of SARS-CoV-2 are selected from the group consisting of: B.1.1.7, Q.1, Q.2, Q.3, Q.4, Q.5, Q.6, Q.7, Q.8, B.1.351, B.1.351.2, B.1.351.3, B.1.617.2, AY.1 sublineages, P.1, P.1.1, P.1.2, B.1.427, B.1.429, B.1.525, B.1.526, B.1.617.1, B.1.617.3, B.1.621, B.1.621.1, P.2, and any combination thereof.

In some aspects, the immune response is to a parasite antigen, wherein the parasite antigen is a protozoan antigen. In some aspects, the immune response is to a parasite antigen selected from the group consisting of a Toxoplasma gondii antigen or a Plasmodium falciparum: antigen. In some aspects, the Toxoplasma gondii antigen is antigen MIC8. In some aspects, the Plasmodium falciparum antigen is a SERA5 polypeptide antigen, or a circumsporozite protein antigen. In some aspects, the immune response is to a parasite antigen, wherein the parasite antigen is a parasitic or pathogenic fungus antigen. In some aspects, the immune response is to a parasite selected from the group consisting of a Candida spp. antigen (e.g., a Candida albicans antigen, a Candida glabrata antigen, a Candida parapsilosis antigen, a Candida tropicalis antigen, a Candida lusitaniae antigen, a Candida krusei antigen), a Pneumocystis spp. antigen, a Malassezia spp. antigen (e.g., a Malassezia furfur antigen), an Aspergillus fumigatus antigen, a Cryptococcus spp. antigen (e.g., a Cryptococcus neoformans antigen, a Cryptococcus gattii antigen), a Histoplasma capsulatum antigen, a Blastomyces dermatitidis antigen, a Paracoccidioides spp. antigen (e.g., a Paracoccidioides brasiliensis antigen, a Paracoccidioides lutzii antigen), a Coccidioides spp. antigen (e.g., a Coccidioides immitis antigen, a Coccidioides posadasii antigen), a Penicillium marneffei antigen, a Sporothrix schenckii antigen, a Trichosporon asahii antigen, a Fusarium spp. antigen (e.g., a Fusarium solanum antigen, a Fusarium oxysporum antigen), a Nectria spp. antigen, a Pseudoallescheria boydii antigen, a Cladophialphora bantianum antigen, a Ramichloridium spp. antigen, a Dactylaria gallopava antigen, an Exophiala spp. antigen (e.g., an Exophiala jeanselmei antigen, an Exophiala dermatitidis antigen), a Curvularia spp. antigen, a Bipolaris spp. antigen, an Alternaria spp. antigen, a Lacazia loboi antigen, a Conidiobolus spp. antigen (e.g., a Conidiobolus coronatus antigen, a Conidiobolus incongruus antigen), and any combination thereof.

Also provided herein are methods of preventing, reducing the incidence of, attenuating or treating a virus (e.g., SARS-CoV-2), a bacteria or a parasite infection in a subject, wherein the methods comprise administering an effective amount of any polynucleotide, vector, multicistronic mRNA vector, DNA plasmid vector, composition, pharmaceutical composition, or vaccine described or exemplified herein to the subject. In some aspects, the polynucleotide, vector, multicistronic mRNA vector, DNA plasmid vector, composition, pharmaceutical composition, or vaccine is administered to the subject by an intramuscular, subcutaneous, intralymphatic, or intraperitoneal route of administration.

Also provided herein is a method of making any composition, pharmaceutical composition, or vaccine described or exemplified herein, the method comprising the steps of: (a) combining a delivery component disclosed herein with a polynucleotide disclosed herein, (b) lyophilizing the combined delivery component and polynucleotide to a powder, and (c) reconstituting the powder with a diluent to form a solution of nucleic acid complexed with the delivery component.

Also provided herein is a composition, pharmaceutical composition, or vaccine for use in accordance with any method disclosed herein.

### BRIEF DESCRIPTION OF THE DRAWINGS

FIG. 1 (FIG. 1) provides a schematic of a vector construct comprising polynucleotide sequences of a gene encoding a SARS-CoV-2 spike (S) protein under the control of promoter 1 (Prom-1), a gene encoding a second SARS-CoV-2 protein under the control of promoter 2 (Prom-2), and genes encoding interleukin 12 (IL-12) p35 and IL-12 p40 under the control of two cytomegalovirus (CMV) promoters in a plasmid backbone.

FIG. 2 (FIG. 2) provides a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), genes encoding IL-12 p35 and IL-12 p40 under the control of two CMV promoters, and a gene encoding major histocompatibility complex class I (MHC I) under the control of promoter Z (Prom-Z) in a plasmid backbone.

FIG. 3 (FIG. 3) provides a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), genes encoding IL-12 p35 and IL-12 p40 under the control of two CMV promoters, and a gene encoding major histocompatibility complex class II (MHC II) under the control of promoter Z (Prom-Z) in a plasmid backbone.

FIG. 4 (FIG. 4) provides a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), and a gene encoding interleukin 2 (IL-2) under the control of a CMV promoter in a plasmid backbone.

FIG. 5 (FIG. 5) provides a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), a gene encoding IL-2 under the control of a CMV promoter, and a gene encoding MHC I under the control of promoter Z (Prom-Z) in a plasmid backbone.

FIG. 6 (FIG. 6) provides a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), a gene encoding IL-2 under the control of a CMV promoter, and a gene encoding MHC II under the control of promoter Z (Prom-Z) in a plasmid backbone.

FIG. 7 (FIG. 7) provides a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), a gene encoding IL-2 under the control of a CMV promoter, a gene encoding C-C motif chemokine ligand (CCL) 3 (CCL3) under the control of promoter X (Prom-X), and a gene encoding CCL4 under the control of promoter Y (Prom-Y) in a plasmid backbone.

FIG. 8 (FIG. 8) provides a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), and a gene encoding interleukin 15 (IL-15) under the control of a CMV promoter in a plasmid backbone.

FIG. 9 (FIG. 9) provides a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), a gene encoding IL-15 under the control of a CMV promoter, and a gene encoding MHC I under the control of promoter Z (Prom-Z) in a plasmid backbone.

FIG. 10 (FIG. 10) provides a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), a gene encoding IL-15 under the control of a CMV promoter, and a gene encoding MHC II under the control of promoter Z (Prom-Z) in a plasmid backbone.

FIG. 11 (FIG. 11) provides a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), a gene encoding IL-15 under the control of a CMV promoter, a gene encoding CCL3 under the control of promoter X (Prom-X), and a gene encoding CCL4 under the control of promoter Y (Prom-Y) in a plasmid backbone.

FIG. 12 (FIG. 12) provides a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), a gene encoding CCL3 under the control of promoter X (Prom-X), and a gene encoding CCL4 under the control of promoter Y (Prom-Y) in a plasmid backbone.

FIG. 13A-13B (FIG. 13A-13B) provides a schematic of the full-length SARS-CoV-2 S protein with S1 and S2 subunits demarcated (FIG. 13A) and overview of constructs pVac1-pVac 5 (FIG. 13B).

FIG. 14A-14I (FIG. 14A-14I) provides schematics of vectors, including pVac vectors. All constructs express partial (S1, amino acids 15-695) or complete SARS-CoV-2 Spike Protein (S, amino acids 1-1273), with a D614G amino acid substitution. S proteins are under the control of a mammalian EF-1α promoter. pUNO vector is provided comprising genes expressing the full-length SARS-CoV-2 Spike Protein (FIG. 14A). FIG. 14B provides pVac1 expressing the SARS-CoV-2 Spike Protein S1 Subunit. FIG. 14C provides pVac2 expressing the SARS-CoV-2 Spike Protein S1 Subunit and IL-12; the single gene (p35 and p40) under the control of two distinct CMV promoters. FIG. 14D provides pVac3 expressing the SARS-CoV-2 Spike Protein S1 Subunit, co- the CoV-2 M antigen under the control of an Internal Ribosome Entry Site (IRES) sequence, and the human heterodimeric cytokine IL12; the single gene (p35 and p40) under the control of two distinct CMV promoters. FIG. 14E (FIG. 14E) provides pVac4 expressing the full-length SARS-CoV-2 Spike Protein with D614G mutation. FIG. 14F (FIG. 14F) provides pVac5 expressing the full-length SARS-CoV-2 Spike Protein with D614G mutation and co-expressing the human heterodimeric cytokine IL12 (the single genes p35 and p40 under the control of two distinct CMV promoters). (pVac5; FIG. 14F), FIG. 14G (FIG. 14G) provides pVac6 expressing the full-length SARS-CoV-2 Spike Protein with D614G mutation with M antigen under the control of an Internal Ribosome Entry Site (IRES) sequence and co-expressing the human heterodimeric cytokine IL12; the single gene (p35 and p40) under the control of two distinct CMV promoters. FIG. 14H (FIG. 14H) provides pVac7 expressing the full-length SARS-CoV-2 Spike Protein with D614G mutation with M antigen under the control of a CMV promoter. FIG. 14I (FIG. 14I) provides the p2CMV-V4 backbone vector used to construct the pVac 1 vector.

FIG. 15A-15F (FIG. 15A-15F) provides Spike S1 protein expression in 293T cell lysates following pVac1 transfection (FIG. 15A), Spike S1 protein expression in 293T cell lysates following transfection with either pVac1, pVac2, or pVac3 (FIG. 15B), Spike protein expression in cell lysate (FIG. 15C) and cell media (FIG. 15D), mIL12 expression in vitro following transfection of both pVac2 and pVac 3 (FIG. 15E), and Spike protein expression level in 293T cell lysates and supernatant following transfection with spike protein plasmids and Omnifect (FIG. 15F).

FIG. 16 (FIG. 16) shows that CP delivery of plasmid DNA results in expression of antigen in vivo. Blood from mice injected with plasmid expressing the reporter gene secreted alkaline phosphatase (SEAP), by IM delivery with CP, were collected at days 1, 3, and 7 following inoculation and tested for SEAP enzyme activity. SEAP enzyme activity is represented by relative light units (RLU). The data are represented as arithmetic mean titers of five mice per group.

FIG. 17A (FIG. 17A) shows that sera from mice immunized with pVac1 delivered by electroporation (EP) can partially neutralize the SARS-CoV-2 D614G S eGFP reporter pseudotyped lentivirus. FIG. 17B (FIG. 17B) shows that sera from mice immunized with plasmid DNA (pVac1 or pUNO) expressing SARS-CoV-2 D614G S protein, delivered by an intramuscular (IM) route with Crown Poloxamer (CP; aza-crown-linked-poloxamer), produce IgG antigen-specific antibodies. FIG. 17C (FIG. 17C) shows that spleens from mice immunized with plasmid DNA (pVac1 or pUNO) expressing SARS-CoV-2 D614G S protein, delivered IM with CP, produce interferon gamma (IFNγ) in the presense of 15mer S overlapping peptides library. Each column in FIG. 17C represents the average of a duplicate of a pool of four spleens. The error bars represent the standard deviation.

FIG. 18 (FIG. 18) provides that sera from mice immunized with pUNO delivered by CP can effectively neutralize the SARS-CoV-2 D614G S eGFP reporter pseudotyped lentivirus.

FIG. 19 (FIG. 19) provides that spleens from mice immunized with plasmid DNA (pUNO, pVac2 or pVac3) expressing SARS-CoV-2 D614G S protein, delivered IM with CP, produce interferon gamma (IFNγ) in the presense of 15mer S overlapping peptides library. Each column represents the average of a duplicate of a pool of four spleens. The error bars represent the standard deviation.

FIG. 20A (FIG. 20A) shows T cell responses to CP DNA vaccine formulations (pVac1 and pUNO 25 0ug, IM) following two or three immunizations (FIG. 19A). Each column represents the average of a duplicate of a pool of four spleens. The error bar represents the standard deviation. FIG. 20B (FIG. 20B) shows B cell responses to CP DNA vaccine formulations (pVac1 and pUNO 25 0ug, IM) following two or three immunizations. The antibody titers are higher in the groups immunized three times compared to the groups immunized two times.

### DETAILED DESCRIPTION OF THE DISCLOSURE

### 5.1 Overview

The polynucleotides (e.g., DNA or mRNA), vectors, multicistronic mRNA vectors, DNA plasmid vectors, compositions, pharmaceutical compositions, and vaccines of the disclosure address, for example, the issue of suboptimal immunogenicity often associated with DNA-based vaccine approaches on one or more levels. First, in some aspects, the polynucleotides can co-express powerful immune modifiers such as cytokines and chemokines that augment the immune responses to the viral antigens. Second, in some aspects, the polynucleotides can include multiple viral antigens and/or multiple epitomes of a viral antigen instead of a single viral antigen, which can be co-expressed from the vectors disclosed herein to expand the spectrum of immunogenicity. Third, in some aspects, to further improve vaccine efficiency, the vectors disclosed herein can be formulated with delivery systems (e.g., a delivery component disclosed herein) that protects the vector or polynucleotide from nuclease degradation and promote its translocation through cell compartments. In some aspects, the delivery systems disclosed herein can also be formulated to exhibit an adjuvant property to promote mobilization of antigen presenting cells to the site of vaccine delivery and antigen expression, thereby augmenting the uptake of the vaccine vector and the expressed viral antigens into professional antigen presenting cells to elicit MIC Class I and MIC Class II presentation. See Greenland, J. R., et al., Molecular Therapy, 12(1):164-70 (2005) (hereafter “Greenland 2005”) and Suschak, J. J., et al., Human Vaccines & Immunotherapeutics, 13(12):2837-48 (2017) (hereafter “Suschak 2017”).

In some aspects, the polynucleotide can comprise a nucleic acid sequence encoding one or more viral antigens (e.g., a SARS CoV-2 antigen). In some aspects, the one or more viral antigens comprise viral antigens (e.g., a S protein, a S1 subunit of a S protein, a RBD of a S protein, a membrane fusion domain of a S protein, a M protein, an E protein, or an antigenic fragment thereof) from two or more SARS-CoV-2 strains. In some aspects, the vector further comprises a nucleic acid sequence encoding one or more immune modifiers. In some aspects, the vector comprises a nucleic acid sequence encoding a SARS CoV-2 antigen and, optionally, a second viral antigen. In some aspects, the vector comprises a nucleic acid sequence encoding a SARS CoV-2 antigen and a second SARS CoV-2 antigen from a different SARS CoV-2 strain. In some aspects, the vector comprises a nucleic acid sequence encoding a SARS CoV-2 S protein antigen and a second SARS CoV-2 S protein antigen from a different SARS CoV-2 strain.

In some aspects, the present disclosure is directed to polynucleotides (e.g., a multicistronic DNA plasmid or multicistronic mRNA) comprising: (a) a first antigen nucleic acid which encodes a first pathogen antigen (e.g., a SARS-CoV-2 S protein or an antigenic fragment thereof); and (b) a nucleic acid encoding an immune modifier. In some aspects, the first antigen nucleic acid is operably linked to a first promoter. In some aspects, the polynucleotide comprises two or more nucleic acids encoding an immune modifier. In some aspects, the polynucleotide further comprises: (c) a second antigen nucleic acid which encodes a second pathogen antigen (e.g., a second SARS-CoV-2 protein antigen or an antigenic fragment thereof). As described herein, the nucleic acid molecules of the present disclosure comprise one or more features that distinguish the present nucleic acid molecules form those that exist in nature (e.g., comprising at least one gene encoding a SARS-CoV-2 S protein and a gene encoding an immune modifier). Not to be bound by any theory, in some aspects, the expression of multiple pathogen antigens (e.g., SARS-CoV-2 antigens), expands the spectrum of immunogenicity, while the expression of at least one immune modifier augments the immune responses to the multiple pathogen antigens. In some aspects, the pathogen antigens are viral pathogen antigens, bacterial pathogen antigens, or parasite pathogen antigens.

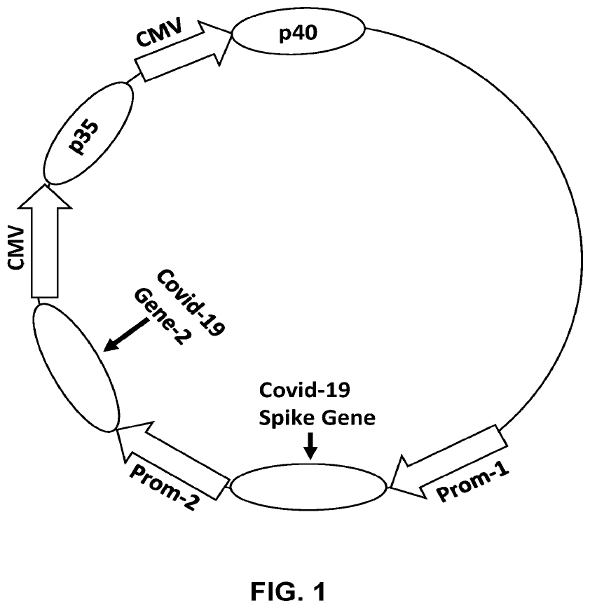

In some aspects, the polynucleotide can comprise: (a) a first nucleic acid encoding a first pathogen antigen (e.g., SARS-CoV-2 spike (S) protein or an antigenic fragment thereof), wherein the first nucleic acid is operably linked to a first promoter; (b) a second nucleic acid encoding a second pathogen antigen (e.g., SARS-CoV-2 protein or an antigenic fragment thereof), wherein the second nucleic acid is operably linked to a second promoter; and (c) a third nucleic acid encoding an immune modifier, wherein the third nucleic acid is operably linked to a third promoter. In some aspects, the polynucleotide can include the elements as disclosed in any of FIGS. 1-12. In some aspects, the vector constructs illustrated in any of FIGS. 1-12 can modified to replace the “Covid-19 Spike Gene” (a first nucleotide sequence encoding a SARS-CoV-2 protein) and the “Covid-19 Gene-2” (a second nucleotide sequence encoding a SARS-CoV-2 protein) with nucleotide sequences encoding any combinations of pathogen antigen or antigenic fragment thereof disclosed herein.

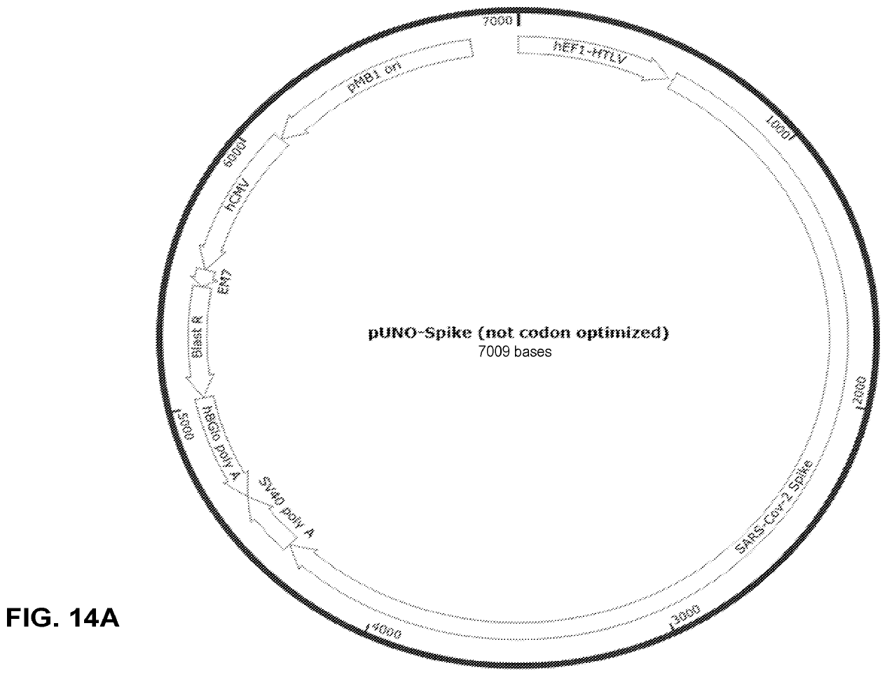

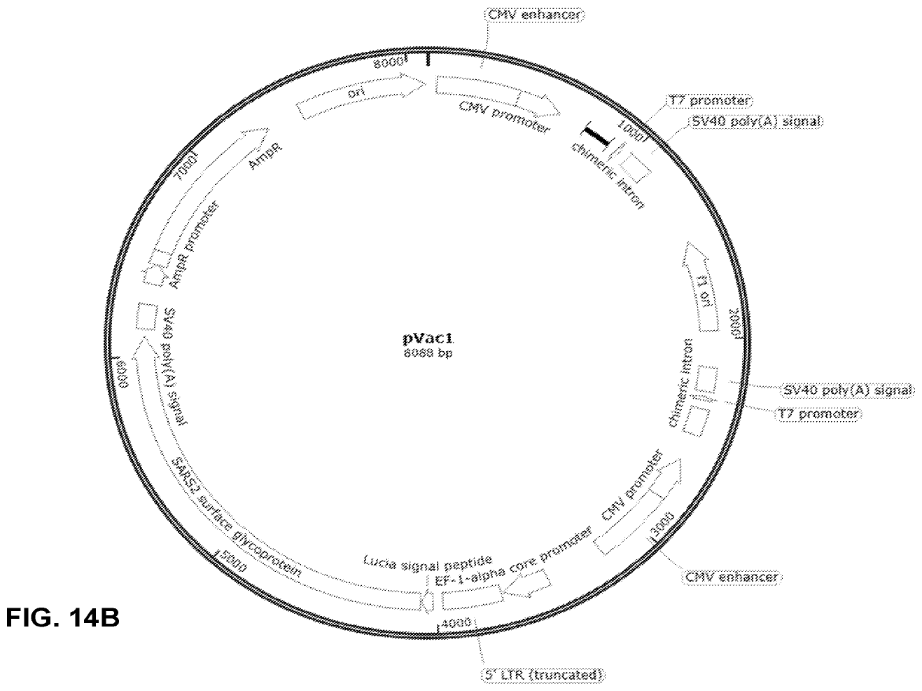

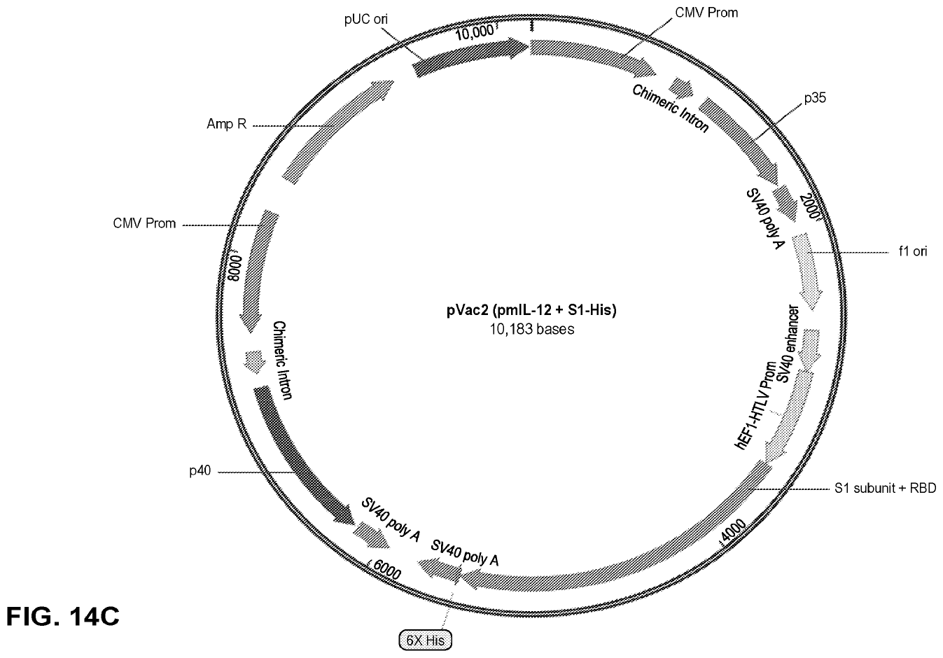

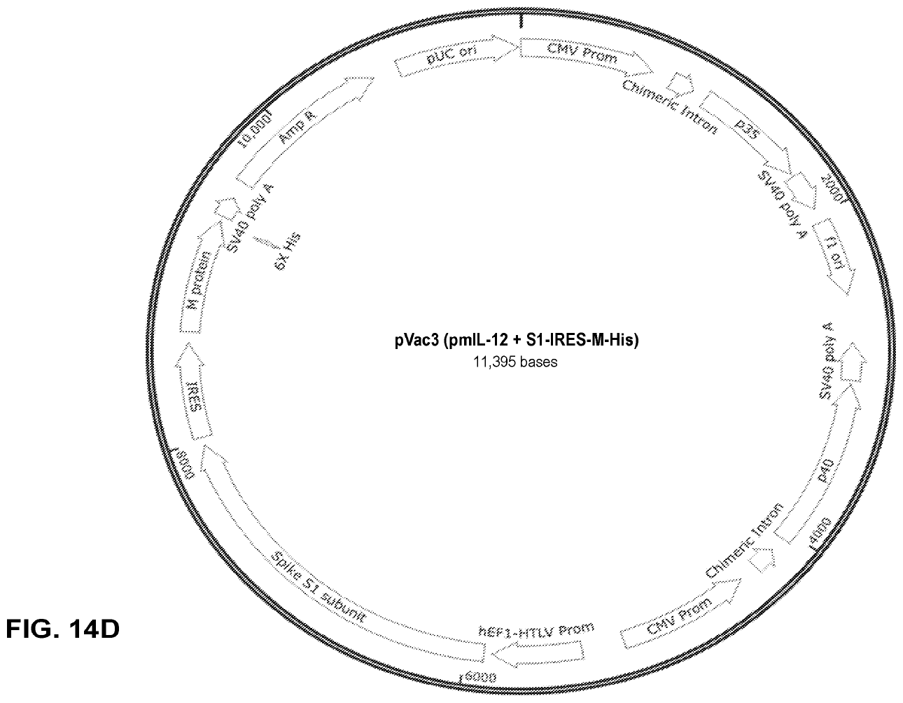

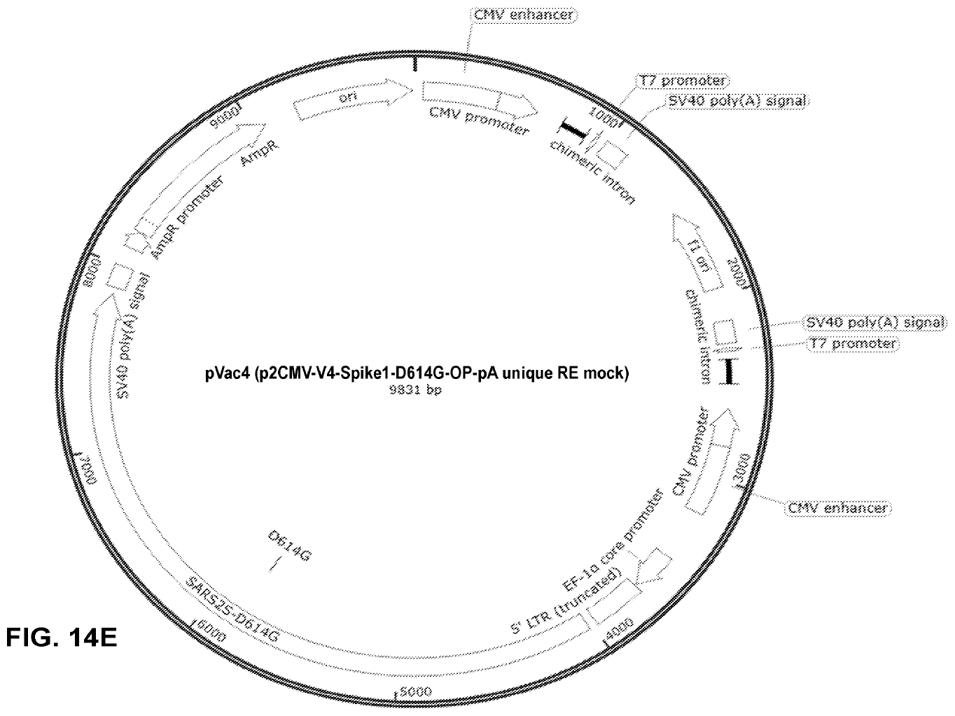

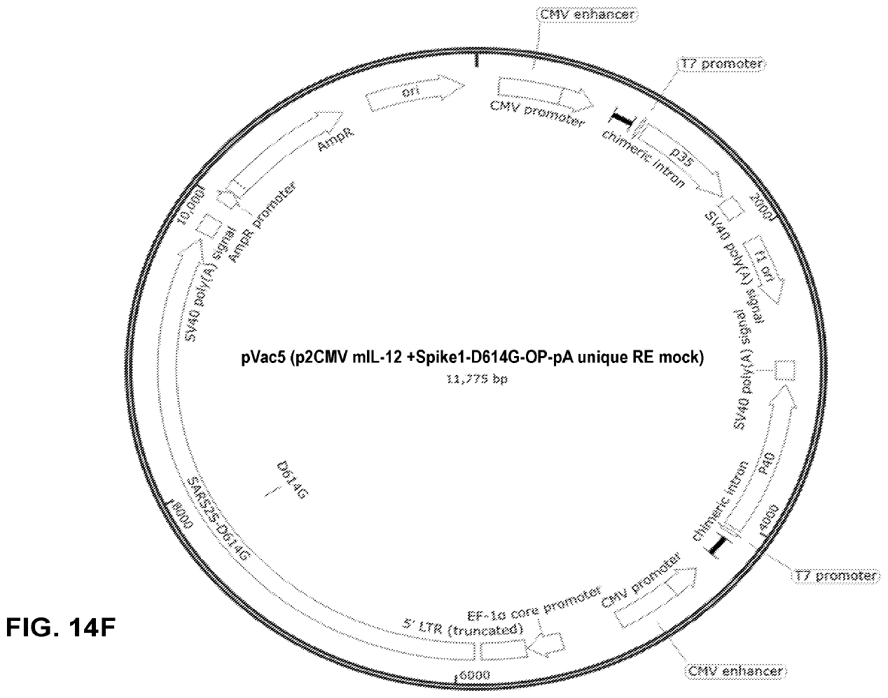

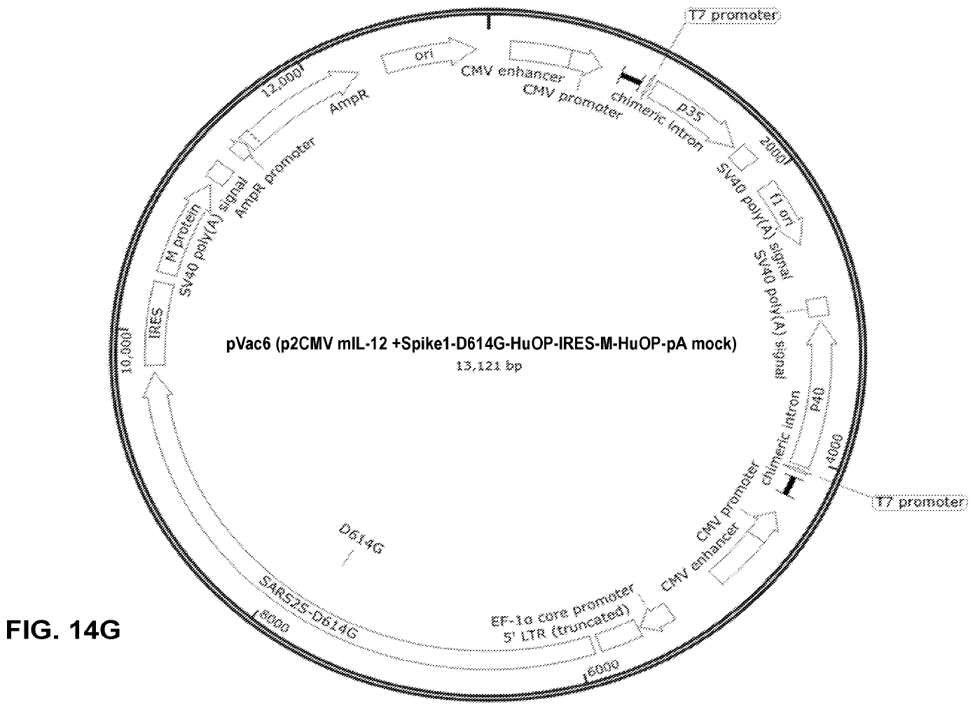

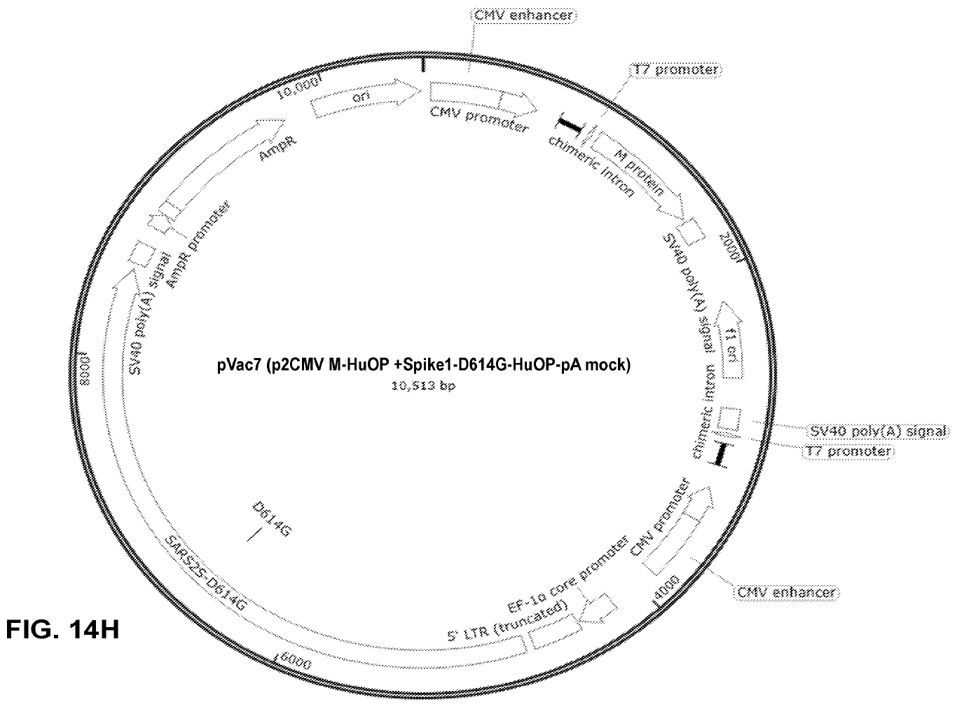

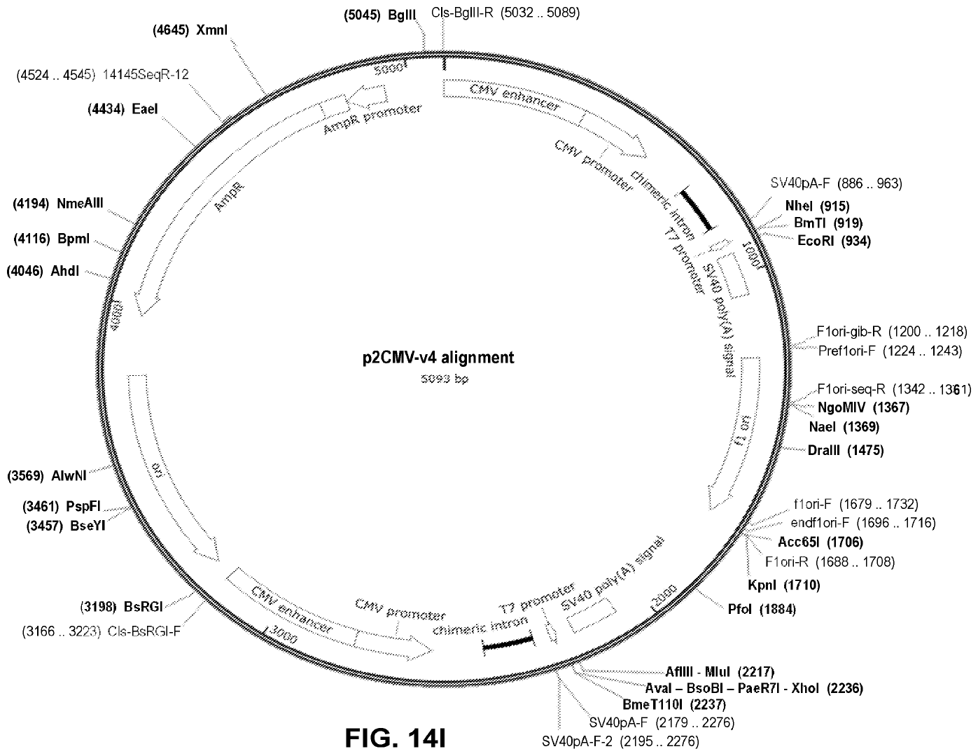

In some aspects, the polynucleotide can comprise: (a) a first nucleic acid encoding a first pathogen antigen (e.g., SARS-CoV-2 spike (S) protein or an antigenic fragment thereof), wherein the first nucleic acid is operably linked to a first promoter; (b) a second nucleic acid encoding a second pathogen antigen (e.g., SARS-CoV-2 membrane (M) protein or an antigenic fragment thereof), wherein the second nucleic acid is operably linked to the first promoter through an IRES sequence; and (c) a third nucleic acid encoding a first immune modifier, wherein the third nucleic acid is operably linked to a third promoter. In some aspects, the polynucleotide further comprises a fourth nucleic acid encoding a second immune modifier, wherein the fourth nucleic acid is operably linked to a fourth promoter. In some aspects, the polynucleotide can include the elements as disclosed in any of FIGS. 14C (pVac 2), 14D (pVac 3), 14F (pVac 5), or 14G (pVac 6). In some aspects, the vector constructs illustrated in any of 14C (pVac 2), 14D (pVac 3), 14F (pVac 5), or 14G (pVac 6) can be modified to replace the SARS-CoV-2 full-length surface (S) protein, the SARS-CoV-2 full-length D614G S protein, or the S1 subunit of the SARS-CoV-2 S protein (a first nucleotide sequence encoding a SARS-CoV-2 protein) and/or the SARS-CoV-2 membrane (M) protein (a second nucleic acid encoding a SARS-CoV-2 protein) with nucleotide sequences encoding any combinations of pathogen antigen or antigenic fragment thereof disclosed herein.

In some aspects, the polynucleotide can comprise a first nucleic acid encoding a first pathogen antigen (e.g., SARS-CoV-2 spike (S) protein or an antigenic fragment thereof), wherein the first nucleic acid is operably linked to a first promoter. In some aspects, the polynucleotide further comprises a second nucleic acid encoding a second pathogen antigen (e.g., SARS-CoV-2 membrane (M) protein or an antigenic fragment thereof). In some aspects, the second nucleic acid is operably linked to the first promoter through an IRES sequence. In some aspects, the first pathogen antigen and the second pathogen antigen are SARS-CoV-2 antigens from different SARS CoV-2 strains. In some aspects, the first pathogen antigen and the second pathogen antigen are different variants of the same SARS-CoV-2 antigen, wherein the different variants of the same SARS-CoV-2 antigen are derived from different strains of SARS CoV-2. In some aspects, the first pathogen antigen and the second pathogen antigen are different variants of a SARS-CoV-2 S protein antigen, wherein the different variants of the SARS-CoV-2 S protein antigen are derived from different strains of SARS CoV-2. In some aspects, the polynucleotide further comprises a second promoter, and the second nucleic acid is operably linked to the second promoter. In some aspects, the polynucleotide can include the elements as disclosed in any of FIGS. 14B (pVac 1), 14 E (pVac 4), or 14H (pVac 7). In some aspects, the vector constructs illustrated in any of FIGS. 14B (pVac 1), 14 E (pVac 4), or 14H (pVac 7) can be modified to replace the SARS-CoV-2 full-length surface (S) protein, the SARS-CoV-2 full-length D614G S protein, or the S1 subunit of the SARS-CoV-2 S protein (a first nucleotide sequence encoding a SARS-CoV-2 protein) and/or the SARS-CoV-2 membrane (M) protein (a second nucleic acid encoding a SARS-CoV-2 protein) with nucleotide sequences encoding any combinations of pathogen antigen or antigenic fragment thereof disclosed herein.

In some aspects, the first pathogen antigen and/or the second pathogen antigen comprise a SARS-CoV-2 S protein or antigenic fragment thereof from an Alpha SARS-CoV-2 strain, wherein the SARS-CoV-2 S protein or antigenic fragment thereof comprises the amino acid sequence of SEQ ID NO: 2 or SEQ ID NO: 4 with one or mutations selected from ΔH69-V70, Δ144, E484K, N501Y, A570D, D614G, P681H, T716I, S982A, and D1118H wherein the amino acid locations correspond to SEQ ID NO: 2 or SEQ ID NO: 4. In some aspects, the first pathogen antigen and/or the second pathogen antigen comprise a SARS-CoV-2 S protein or antigenic fragment thereof from a SARS-CoV-2 S protein or antigenic fragment thereof from a Beta SARS-CoV-2 strain, wherein the SARS-CoV-2 S protein or antigenic fragment thereof comprises the amino acid sequence of SEQ ID NO: 2 or SEQ ID NO: 4 with one or mutations selected from L18F, D80A, D215G, ΔL241-S243, K417N, E484K, N501Y, D614G, and A701V, wherein the amino acid locations correspond to SEQ ID NO: 2 or SEQ ID NO: 4. In some aspects, the first pathogen antigen and/or the second pathogen antigen comprise a SARS-CoV-2 S protein or antigenic fragment thereof from a SARS-CoV-2 S protein or antigenic fragment thereof from a Gamma SARS-CoV-2 strain, wherein the SARS-CoV-2 S protein or antigenic fragment thereof comprises the amino acid sequence of SEQ ID NO: 2 or SEQ ID NO: 4 with one or mutations selected from L18F, T20N, P26S, D138Y, R190S, K417T, E484K, N501Y, D614G, H655Y, T1027I, and VI 176F, wherein the amino acid locations correspond to SEQ ID NO: 2 or SEQ ID NO: 4. In some aspects, the first pathogen antigen and/or the second pathogen antigen comprise a SARS-CoV-2 S protein or antigenic fragment thereof from a Delta SARS-CoV-2 strain, wherein the SARS-CoV-2 S protein or antigenic fragment thereof comprises the amino acid sequence of SEQ ID NO: 2 or SEQ ID NO: 4 with one or mutations selected from T19R, ΔD119-F120, AE156-F157, R158G, L452R, T478K, D614G, P681R, and D950N, wherein the amino acid locations correspond to SEQ ID NO: 2 or SEQ ID NO: 4. In some aspects, the first pathogen antigen and/or the second pathogen antigen comprise a SARS-CoV-2 S protein or antigenic fragment thereof from a Kappa SARS-CoV-2 strain, wherein the SARS-CoV-2 S protein or antigenic fragment thereof comprises the amino acid sequence of SEQ ID NO: 2 or SEQ ID NO: 4 with one or mutations selected from E154K, L452R, E484Q, D614G, P681R, and Q1071H, wherein the amino acid locations correspond to SEQ ID NO: 2 or SEQ ID NO: 4. In some aspects, the first pathogen antigen and/or the second pathogen antigen comprise a SARS-CoV-2 S protein or antigenic fragment thereof from an Eta SARS-CoV-2 strain, wherein the SARS-CoV-2 S protein or antigenic fragment thereof comprises the amino acid sequence of SEQ ID NO: 2 or SEQ ID NO: 4 with one or mutations selected from Q52R, A67V, ΔH69-V70, AY144, E484K, D614G, Q677H, and F888L, wherein the amino acid locations correspond to SEQ ID NO: 2 or SEQ ID NO: 4. In some aspects, the first pathogen antigen and/or the second pathogen antigen comprise a SARS-CoV-2 S protein or antigenic fragment thereof from an Iota SARS-CoV-2 strain, wherein the SARS-CoV-2 S protein or antigenic fragment thereof comprises the amino acid sequence of SEQ ID NO: 2 or SEQ ID NO: 4 with one or mutations selected from L5F, T95I, D253G, E484K, D614G, and A701V, wherein the amino acid locations correspond to SEQ ID NO: 2 or SEQ ID NO: 4. In some aspects, the first pathogen antigen and/or the second pathogen antigen comprise a SARS-CoV-2 S protein or antigenic fragment thereof from a Lambda SARS-CoV-2 strain, wherein the SARS-CoV-2 S protein or antigenic fragment thereof comprises the amino acid sequence of SEQ ID NO: 2 or SEQ ID NO: 4 with one or mutations selected from G75V, T76I, AR246-G252, D253N, L452Q, F490S, D614G, and T859N, wherein the amino acid locations correspond to SEQ ID NO: 2 or SEQ ID NO: 4. In some aspects, the first pathogen antigen and/or the second pathogen antigen comprise a SARS-CoV-2 S protein or antigenic fragment thereof from a Mu SARS-CoV-2 strain, wherein the SARS-CoV-2 S protein or antigenic fragment thereof comprises the amino acid sequence of SEQ ID NO: 2 or SEQ ID NO: 4 with one or mutations selected from T95I, Y144S, Y145N, R346K, E484K, N501Y, D614G, P681H, and D950N, wherein the amino acid locations correspond to SEQ ID NO: 2 or SEQ ID NO: 4. In some aspects, the first pathogen antigen and/or the second pathogen antigen comprise a SARS-CoV-2 S protein or antigenic fragment thereof from an Epsilon SARS-CoV-2 strain, wherein the SARS-CoV-2 S protein or antigenic fragment thereof comprises the amino acid sequence of SEQ ID NO: 2 or SEQ ID NO: 4 with one or mutations selected from S13I, W152C, L452R, and D614G, wherein the amino acid locations correspond to SEQ ID NO: 2 or SEQ ID NO: 4.

In some aspects, the present disclosure is also directed to vectors, vaccines, compositions, or pharmaceutical compositions comprising a polynucleotide disclosed herein. In some aspects, the vectors, vaccines, compositions, or pharmaceutical compositions further comprise a delivery component (e.g., a cationic polymer such as a biodegradable cross-linked cationic multi-block copolymer, a PEG-PEI-cholesterol (PPC) lipopolymer, a lipopolyamine, or a lipopolyamine derivative). In some aspects, the PEG-PEI-cholesterol (PPC) lipopolymer has an average PEG:PEI:cholesterol ratio of 2.5:1:0.6.

In some aspects, the delivery component exhibits an adjuvant property. Not to be bound by any theory, in some aspects, the adjuvant property of the delivery component promotes mobilization of antigen presenting cells to the site of vaccine delivery and antigen expression, thereby augmenting the uptake of the polynucleotide and the expressed antigens into professional antigen presenting cells to elicit MHC Class I and MHC Class II presentation. In some aspects, the present disclosure is directed to vaccines or compositions comprising (i) a vector (e.g., a multicistronic DNA plasmid vector or a multicistronic messenger RNA (mRNA) vector) comprising a nucleic acid sequence encoding one or more viral antigens (e.g., a SARS CoV-2 antigen) and (ii) a delivery component (e.g., a cationic polymer, a poly-inosinic-polycytidylic acid, or a poloxamer). In some aspects, the vector further comprises a nucleic acid sequence encoding one or more immune modifiers. In some aspects, the vector comprises a nucleic acid sequence encoding a SARS CoV-2 antigen and, optionally, a second viral antigen. In some aspects, the vector comprises a nucleic acid sequence encoding a SARS CoV-2 antigen and a second SARS CoV-2 antigen from a different SARS CoV-2 strain. In some aspects, the vector comprises a nucleic acid sequence encoding a SARS CoV-2 S protein antigen and a second SARS CoV-2 S protein antigen from a different SARS CoV-2 strain.

Some aspects relate to methods of eliciting humoral and/or cellular immune response against a pathogen (e.g., SARS-CoV-2) challenge or infection following in vivo administration of a vector or composition of the disclosure.

The present disclosure is also directed to methods of inducing an immune response in a subject comprising administering an effective amount of any polynucleotide, vector, composition, pharmaceutical composition, or vaccine disclosed herein to the subject. The present disclosure is also directed to methods of preventing, reducing the incidence of, attenuating or treating a viral, a bacterial or a parasite infection in a subject comprising administering an effective amount of any polynucleotide, vector, composition, pharmaceutical composition, or vaccine disclosed herein to the subject. In some aspects, the infection is a SARS-CoV-2 viral infection. The present disclosure is also directed to methods of making any composition, pharmaceutical composition, or vaccine disclosed herein.

### 5.2 Definitions

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

As used herein, the term “approximately” or “about,” as applied to one or more values of interest, refers to a value that is similar to a stated reference value and within a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). When the term “approximately” or “about” is applied herein to a particular value, the value without the term “approximately” or “about is also disclosed herein.

As described herein, any concentration range, percentage range, ratio range, or integer range is to be understood to include the value of any integer within the recited range and, when appropriate, fractions thereof (such as one tenth and one hundredth of an integer), unless otherwise indicated.

As used herein, the terms “ug” and “uM” are used interchangeably with “μg” and “μM,” respectively.

Units, prefixes, and symbols are denoted in their Syst\tilde{e}me International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As used herein, the term “coronavirus” or “CoV” refers to the common name for Coronaviridae. In humans, CoV causes respiratory infections, which are typically mild but can be lethal in rare forms such as SARS (severe acute respiratory syndrome)-CoV, MERS (Middle East Respiratory Syndrome)-CoV, and SARS-CoV-2. CoV has a nucleocapsid of helical symmetry and the genome size ranges from about 26 to about 32 kilobases. Other exemplary human CoV include CoV 229E, CoV NL63, CoV OC43, CoV HKU1, and CoV HKU20. The envelope of CoV carries three glycoproteins: spike (S) protein (receptor binding, cell fusion, major antigen); envelope (E) protein (small, envelope-associated protein); and membrane (M) protein (budding and envelope formation). In a few types of CoV, there is a fourth glycoprotein: hemagglutinin-esterase (HE) protein. The genome has a 5′ methylated cap and 3′ poly-A and functions directly as mRNA. Entry of the CoV into a human cell occurs via endocytosis and membrane fusion; and replication occurs in the cell's cytoplasm. CoV are transmitted by aerosols of respiratory secretions, by the faecal-oral route, and by mechanical transmission. Most virus growth occurs in epithelial cells. Occasionally, the liver, kidneys, heart, or eyes can be infected, as well as other cell types such as macrophages.

As used herein, the term “SARS-CoV-2” refers to the strain of coronavirus that causes coronavirus disease 2019 (COVID-19), the respiratory illness responsible for the COVID-19 pandemic. Taxonomically, SARS-CoV-2 is a member of the subgenus Sarbecovirus (beta-CoV lineage B) and is a strain of SARS-CoV. It is believed to have zoonotic origins and has close genetic similarity to bat coronaviruses, suggesting it emerged from a bat-borne virus. Its RNA sequence is approximately 30,000 bases in length. SARS-CoV-2 is unique among known betacoronaviruses in its incorporation of a polybasic cleavage site, a characteristic known to increase pathogenicity and transmissibility in other viruses. Like other coronaviruses, SARS-CoV-2 has four structural proteins, the S (spike), E (envelope), M (membrane), and N (nucleocapsid) proteins. The N protein holds the RNA genome, and the S, E, and M proteins together create the viral envelope. The spike protein is the protein responsible for allowing the virus to attach to and fuse with the membrane of a host cell; specifically, its S1 subunit catalyzes attachment, the S2 subunit fusion. Protein modeling experiments on the spike protein of the virus have suggested that SARS-CoV-2 has sufficient affinity to the receptor angiotensin converting enzyme 2 (ACE2) on human cells to use them as a mechanism of cell entry. See Xu, X, et al., Science China Life Sciences, 63(3):457-60 (2020). SARS-CoV-2 can also use basigin to assist in cell entry. See Wang, K., et al., bioRxiv, doi:10.1101/2020.03.14.988345 (2020).

The terms “nucleic acids,” “nucleic acid molecules, “nucleotides,” “nucleotide(s) sequence,” and “polynucleotide” can be used interchangeably and refer to the phosphate ester polymeric form of ribonucleosides (adenosine, guanosine, uridine or cytidine; “RNA molecules”, including mRNA) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, DNA plasmid, synthetic DNA, and semi-synthetic DNA. A “nucleic acid composition” of the disclosure comprises one or more nucleic acids as described herein.

RNA can be obtained by transcription of a DNA-sequence, e.g., inside a cell. In eularyotic cells, transcription is typically performed inside the nucleus or the mitochondria. In vivo, transcription of DNA usually results in premature RNA, which has to be processed into messenger RNA (mRNA). Processing of the premature RNA, e.g., in eukaryotic organisms, comprises a variety of different posttranscriptional-modifications such as splicing, 5′-capping, polyadenylation, export from the nucleus or the mitochondria and the like. The sum of these processes is also called maturation of RNA. The mature mRNA usually provides the nucleotide sequence that can be translated into an amino acid sequence of a particular peptide, protein, or protein antigen. Typically a mature mRNA comprises a 5′ cap, optionally a 5′-UTR, an open reading frame, optionally a 3′-UTR, and a poly(A) sequence.

The term “multicistronic mRNA” or “multicistronic mRNA vector,” as used herein, refers to an mRNA having two or more open reading frames. An open reading frame in this context is a sequence of codons that is translatable into a polypeptide or protein.

The term “5′-cap,” as used herein, refers to an entity, typically a modified nucleotide entity, which generally “caps” the 5′-end of a mature mRNA. A 5′-cap can typically be formed by a modified nucleotide, particularly by a derivative of a guanine nucleotide. In some aspects, the 5′-cap is linked to the 5′-terminus via a 5′-5′-triphosphate linkage. A 5′-cap can be methylated, e.g., m7GpppN, wherein N is the terminal 5′ nucleotide of the nucleic acid carrying the 5′-cap, typically the 5′-end of an RNA. The naturally occurring 5′-cap is m7GpppN.

As used herein, a “poly(A) sequence,” also called “poly(A) tail” or “3′-poly(A) tail,” is typically understood to be a sequence of adenine nucleotides, e.g., of up to about 400 adenine nucleotides. A poly(A) sequence can be located at the 3′ end of an mRNA. In some aspects, a poly(A) sequence can also be located within an mRNA or any other nucleic acid molecule, such as, e.g., in a vector, for example, in a vector serving as template for the generation of an RNA, preferably an mRNA, e.g., by transcription of the vector. In some aspects, a poly(A) sequence is present in the 3′-UTR of the mRNA as defined herein.

In some aspects, a 3′-UTR sequence is part of an mRNA, which is located between the protein coding region (i.e. the open reading frame) and the 3′ terminus of the mRNA molecule. If a 3′-terminal poly(A) sequence (‘poly(A) tail’) was added to the RNA (e.g. by polyadenylation), then the term 3′-UTR can refer to that part of the molecule, which is located between the protein coding region and the 3′-terminal poly(A) sequence. In some aspects, a 3′-UTR can also comprise a poly(A) sequence (e.g., a poly(A) sequence which is not located at the very 3′ terminus of the RNA molecule). A 3′-UTR of the mRNA is not translated into an amino acid sequence. The 3′-UTR sequence is generally encoded by the gene, which is transcribed into the respective mRNA during the gene expression process. The genomic sequence is first transcribed into pre-mature mRNA, which comprises optional introns. The pre-mature mRNA is then further processed into mature mRNA in a maturation process. This maturation process comprises the steps of 5′ capping, splicing the pre-mature mRNA to excise optional introns and modifications of the 3′-end, such as polyadenylation of the 3′-end of the pre-mature mRNA and optional endo-/or exonuclease cleavages etc. In some aspects, a 3′-UTR corresponds to the sequence of a mature mRNA, which is located 3′ to the stop codon of the protein coding region (e.g., immediately 3′ to the stop codon of the protein coding region), and which extends to the 3′ terminus of the RNA molecule or to the 5′-side of a 3′ terminal poly(A) sequence (e.g., to the nucleotide immediately 5′ to the 3′ terminus or immediately 5′ to the 3′ terminal poly(A) sequence). The term “corresponds to” means that the 3′-UTR sequence can be an RNA sequence, such as in the mRNA sequence used for defining the 3′-UTR sequence, or a DNA sequence, which corresponds to such RNA sequence. In some aspects, the term “a 3′-UTR of a gene”, such as “3′-UTR of alpha or beta globin”, is the sequence, which corresponds to the 3′-UTR of the mature mRNA derived from this gene, i.e. the mRNA obtained by transcription of the gene and maturation of the pre-mature mRNA. The term “3′-UTR of a gene” encompasses the DNA sequence and the RNA sequence of the 3′-UTR. In some aspects, the 3′-UTR is derived from a gene that relates to an mRNA with an enhanced half-like (i.e., that provides a stable mRNA), for example a 3′-UTR of a gene selected from the group consisting of: albumin gene, an α-globin gene, a α-globin gene, a tyrosine hydroxylase gene, a lipoxygenase gene, and a collagen alpha gene, such as a collagen alpha 1(I) gene.

A 5′-UTR is typically understood to be a particular section of messenger RNA (mRNA). It is located 5′ of the open reading frame of the mRNA. In some aspect, the 5′-UTR starts with the transcriptional start site and ends one nucleotide before the start codon of the open reading frame. The 5′-UTR can comprise elements for controlling gene expression, also called regulatory elements. Such regulatory elements can be, for example, ribosomal binding sites or a 5′-Terminal Oligopyrimidine Tract. The 5′-UTR can be posttranscriptionally modified, for example by addition of a 5′-cap. In some aspects, a 5′-UTR corresponds to the sequence of a mature mRNA which is located between the 5′ cap and the start codon. In some aspects, the 5′-UTR corresponds to the sequence which extends from a nucleotide located 3′ to the 5′-cap (e.g., from the nucleotide located immediately 3′ to the 5′cap) to a nucleotide located 5′ to the start codon of the protein coding region (e.g., to the nucleotide located immediately 5′ to the start codon of the protein coding region). The nucleotide located immediately 3′ to the 5′ cap of a mature mRNA typically corresponds to the transcriptional start site. The term “corresponds to” means that the 5′-UTR sequence can be an RNA sequence, such as in the mRNA sequence used for defining the 5′-UTR sequence, or a DNA sequence which corresponds to such RNA sequence. In some aspects, the term “a 5′-UTR of a gene”, is the sequence, which corresponds to the 5′-UTR of the mature mRNA derived from this gene.

As used herein, the term “transfecting” or “transfection” refers to the transport of nucleic acids from the environment external to a cell to the internal cellular environment, with particular reference to the cytoplasm and/or cell nucleus. Without being bound by any particular theory, it is to be understood that nucleic acids can be delivered to cells either after being encapsulated within or adhering to one or more cationic polymer/nucleic acid complexes or being entrained therewith. Particular transfecting instances deliver a nucleic acid to a cell nucleus. Nucleic acids include DNA and RNA as well as synthetic congeners thereof. Such nucleic acids include missense, antisense, nonsense, as well as protein producing nucleotides, on and off and rate regulatory nucleotides that control protein, peptide, and nucleic acid production. In particular, but not limited to, they can be genomic DNA, cDNA, mRNA, tRNA, rRNA, hybrid sequences or synthetic or semi-synthetic sequences, and of natural or artificial origin. In addition, the nucleic acid can be variable in size, ranging from oligonucleotides to chromosomes. These nucleic acids can be of human, animal, vegetable, bacterial, viral, or synthetic origin. They can be obtained by any technique known to a person skilled in the art.

As used herein, the term “biodegradable” or “biodegradation” is defined as the conversion of materials into less complex intermediates or end products by solubilization hydrolysis, or by the action of biologically formed entities which can be enzymes and other products of the organism.

As used herein, “peptide” means peptides of any length and includes proteins. The terms “polypeptide” and “oligopeptide” are used herein without any particular intended size limitation, unless a particular size is otherwise stated.

As used herein, a “derivative” of a carbohydrate includes, for example, an acid form of a sugar, e.g. glucuronic acid; an amine of a sugar, e.g. galactosamine; a phosphate of a sugar, e.g. mannose-6-phosphate; and the like.

As used herein, the term “inverted terminal repeat” (or “ITR”) refers to a single stranded sequence of nucleotides followed downstream by its reverse complement. The intervening sequence of nucleotides between the initial sequence and the reverse complement can be any length including zero.

“Administering” and similar terms refer to the physical introduction of a therapeutic agent (e.g., nucleic acid molecules, vectors, compositions, and pharmaceutical compositions described herein) to a subject, using any of the various methods and delivery systems known to those skilled in the art. Exemplary routes of administration include intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural, intrasterna, oral, rectal, topical, epidermal, mucosal, intranasal, vaginal, rectal, sublingual administration, and combinations thereof. Administering can also be performed, for example, once, a plurality of times, and/or over one or more extended periods.

“Treatment” or “therapy” of a subject refers to any type of intervention or process performed on, or the administration of an active agent to, a subject with the objective of reversing, alleviating, ameliorating, inhibiting, slowing down, or preventing the onset, progression, development, severity, or recurrence of a symptom, complication, condition, or biochemical indicia associated with a disease.

A “therapeutically effective amount,” “effective amount,” “therapeutic dose,” “effective dose,” or “effective dosage,” as used herein, means an amount or a dose that achieves a therapeutic goal, as described herein. One of ordinary skill in the art will further understand that a therapeutically effective amount etc. can be administered in a single dose, or can be achieved by administration of multiple doses (i.e., 1, 2, 3, 4, 5, 6, 7, 8, 9, 10 or more doses). The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

As used herein, the terms “prevent,” “preventing,” “prevention,” “prophylactic treatment,” and the like, refer to reducing the probability of developing a disease or condition in a subject, who does not have, but is at risk of or susceptible to developing a disease or condition.

As used herein, the term “adjuvant” refers to any component which improves the body's response to a vaccine.

As used herein, the term “vaccine” or “vaccine composition” refers to an immunogenically active composition for the prophylaxis and/or treatment of diseases. Accordingly, in some aspects, vaccines are medicaments which comprise or deliver antigens and are intended to be used in humans or animals for generating specific defense and protective substance by vaccination.

A used herein, the term “inducing immunity” or “immunogenically active” refers to the ability to stimulate an immune response, i.e., to stimulate the production of antibodies, particularly humoral antibodies, or to stimulate a cell-mediated response. For example, the ability to stimulate the production of circulating or secretory antibodies or the production of a cell-mediated response in local mucosal regions, peripheral blood, cerebral spinal fluid or the like. In some aspects, the effective immunizing amount of the immunogenically active component(s) of this disclosure can vary and can be any amount sufficient to evoke an immune response and provide a protective immune response against SARS-CoV-2 virus infection. A dosage unit comprising a polynucleotide (e.g., plasmid DNA) of the disclosure is contemplated. At least one dosage unit per patient is contemplated herein as a vaccination regimen. In some embodiments, two or more dosage units can be useful. The skilled artisan will quickly recognize that a particular quantity of vaccine composition per dosage unit, as well as the total number of dosage units per vaccination regimen, can be optimized, so long as an effective immunizing amount of the virus or a component thereof is ultimately delivered to the subject.

An “immunological response” to a substance such as a composition or vaccine is the development in the subject of a cellular and/or antibody-mediated immune response to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. In some aspects, the subject can display either a therapeutic or protective immunological response so resistance to new infection will be enhanced and/or the clinical severity of the disease reduced. In some aspects, such protection can be demonstrated by either a reduction or lack of symptoms normally displayed by an infected subject, a quicker recovery time and/or a lowered viral titer in the infected subject.

It is recognized that the antigenic polypeptides of the disclosure can be full length polypeptides or active fragments or variants thereof. In some aspects, the term “active fragments” or “active variants” or “antigenic fragments” refers to fragments or variants that retain all or some of the antigenic nature of the polypeptide. Thus, in some aspects, the present disclosure encompasses any SARS-CoV-2 polypeptide, antigen, epitope or immunogen that elicits an immunogenic response in a subject. The SARS-CoV-2 polypeptide, antigen, epitope or immunogen can be any SARS-CoV-2 polypeptide, antigen, epitope or immunogen, such as, but not limited to, a protein, peptide or fragment or variant thereof, that elicits, induces or stimulates a response in a subject. The SARS-CoV-2 polypeptide, antigen, epitope or immunogen can be derived from any strain of SARS-CoV-2 including, but not limited to, an Alpha SARS-CoV-2 strain (e.g., strains B.1.1.7 and Q.1-Q.8); a Beta SARS-CoV-2 strain (e.g., strains B.1.351, B.1.351.2, and B.1.351.3); a Delta SARS-CoV-2 strain (e.g., strain B.1.617.2 and AY.1 sublineages); a SARS-CoV-2 strain Gamma strain (e.g., strains P.1, P.1.1, and P.1.2); an Epsilon SARS-CoV-2 strain (e.g., strains B.1.427 and B.1.429); an Eta SARS-CoV-2 strain (e.g., strain B.1.525); an Iota SARS-CoV-2 strain (e.g., strain B.1.526); a Kappa SARS-CoV-2 strain (e.g., strain B.1.617.1); a Lambda SARS-CoV-2 strain; a B.1.617.3 SARS-CoV-2 strain; a Mu SARS-CoV-2 strain (e.g., strains B.1.621 and B.1.621.1); or a Zeta strain (e.g., strain P.2). Mutations and viral sequence data for the SARS-CoV-2 variants are publically available at the CoVariants website (https://covariants.org/) and the National Center for Biotechnology Information (NCBI) website (https://www.ncbi.nlm.nih.gov/labs/virus/vssi/#/sars-cov-2), each of which is incorporated by reference in its entirety.

The term “epitope” refers to the site on an antigen or hapten to which specific B cells and/or T cells respond. The term is also used interchangeably with “antigenic determinant” or “antigenic determinant site.” Antibodies that recognize the same epitope can be identified in a simple immunoassay showing the ability of one antibody to block the binding of another antibody to a target antigen.

As used herein, the term “pharmaceutical agent,” “pharmaceutical composition,” or “drug” or any other similar term means any chemical or biological material or compound suitable for administration by the methods previously known in the art and/or by the methods taught in the present disclosure, which induce a desired biological or pharmacological effect, which can include but are not limited to (1) having a prophylactic effect on the organism and preventing an undesired biological effect such as preventing an infection, (2) alleviating a condition caused by a disease, for example, alleviating pain or inflammation caused as a result of disease, and/or (3) either alleviating, reducing, or completely eliminating a disease from the organism. The effect can be local or it can be systemic.

A “pharmaceutically acceptable carrier” refers to a carrier that can be administered to a subject, together with an agent, and which does not destroy the pharmacological activity thereof and is nontoxic when administered in doses sufficient to deliver a therapeutic amount of the agent. In certain aspects, the pharmaceutically acceptable carrier is an aqueous solvent, i.e., a solvent comprising water, optionally with additional co-solvents. Exemplary pharmaceutically acceptable carriers include water, buffer solutions in water (such as phosphate-buffered saline (PBS), and 5% dextrose in water (D5W). In certain embodiments, the aqueous solvent further comprises dimethyl sulfoxide (DMSO), e.g., in an amount of about 1-4%, or 1-3%. In certain aspects, the pharmaceutically acceptable carrier is isotonic (i.e., has substantially the same osmotic pressure as a body fluid such as plasma).

A “subject” includes any human or non-human animal. The term “nonhuman animal” includes, but is not limited to, vertebrates such as nonhuman primates, sheep, dogs, and rodents such as mice, rats, and guinea pigs. In some aspects, the subject is a human. The terms “subject” and “patient” are used interchangeably herein.

The term “expression” as used herein refers to a process by which a polynucleotide produces a gene product, for example, a SARS-CoV-2 S protein or antigenic fragment thereof. In some aspects, it includes, without limitation, transcription of the polynucleotide into messenger RNA (mRNA) and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein can further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

As used herein, the term “5′” or “5 prime” refers to the 5′ end of a nucleic acid or nucleic acid sequence, and the term “3” or “3 prime” refer to the 3′ end of nucleic acid or nucleic acid sequence.

The terms “identical” or percent “identity” in the context of two or more nucleic acids refer to two or more sequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

As used herein, the term “promoter” refers to DNA sequence capable of controlling the expression of a coding sequence or functional RNA. In some aspects, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The term “operably linked” refers to genetic elements that are joined together in a manner that enables them to carry out their normal functions. For example, a gene is operably linked to a promoter when its transcription is under the control of the promoter and this transcription results in the production of the product encoded by the gene.

As used herein, the term “immune modifier” refers to a protein that augments the immune response to a one or more antigens. In some aspects, immune modifiers include, but are not limited to, a cytokine, a chemokine, major histocompatibility complex (MHC) class I (MHC I), MHC class II (MHC II), human leukocyte antigen (HLA)-DR isotype (HLA-DR), CD80, CD86, and any combination thereof. Cytokine immune modifiers include, but are not limited to, interleukin (IL) 2 (IL-2), IL-12 p35, IL-12 p40, IL-12 p70, IL-15, IL-18, tumor necrosis factor alpha (TNFα), granulocyte-macrophage colony-stimulating factor (GM-CSF), interferon (IFN) α (IFN-α), and IFN-β. Chemokine immune modifiers include, but are not limited to, C-C motif chemokine ligand (CCL) 3 (CCL3), CCL4, CCL5, CCL21, CCL28, C-X-C motif chemokine ligand (CXCL) 10 (CXCL10), and any combination thereof. In some aspects, the immune modifiers include a viral protein (e.g., SARS-CoV-2 non-structural protein 1 (Nsp1), SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b) that attenuates a local inflammatory response and/or interferon response. In some aspects, the viral protein is from the same virus as a viral antigen encoded by an antigen nucleic acid. In some aspects, In some aspects, the viral protein is from a different virus than a viral antigen encoded by an antigen nucleic acid. In some aspects, the viral protein attenuates a local inflammatory response and/or interferon response elicited by a pathogen antigen disclosed herein. In some aspects, the immune modifiers include SARS-CoV-2 Nsp1, SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b, and any combination thereof. In some aspects, the immune modifiers include one or more concatamers of non-coding 5′-C-phosphate-G-3′ (CpG) dinucleotides. In some aspects, the one or more concatamers of non-coding CpG dinucleotides activate the Toll-like receptor 9 (TLR9) signaling pathway. In some aspects, the one or more concatamers of non-coding CpG dinucleotides comprise one or more concatamers of non-coding CpG dinucleotides previously reported in Bauer, A. et al., Nucleic Acids Research 38(12):3891-908 (2010); Cornelie, S. et al., Journal of Biological Chemistry 279(15):15124-9 (2004); Klinman, D. et al., J Immunol. 158(8):3635-9 (1997); Klinman, D. et al., Immunological Reviews 199(1):201-16 (2004); Luo, Z. et al., Mol Med Rep. 6(6):1309-14 (2012); Bode, C. et al., Expert Rev Vaccines 10(4):499-511 (2011); and Kuo, T. et al., Scientific Reports 10:20085 (2020), each of which is incorporated by reference herein in its entirety. In some aspects, an immune modifier as disclosed herein can include a combination of immune modifiers, e.g., a cytokine or chemokine protein or their coding sequence either co-expressed from the same plasmid as the antigen or from a different plasmid. See, e.g., Hirao, L. A., et al, Vaccine, 26:3112-20 (2008); Kanagavelu, S. K., et al., Vaccine, 30:691-702 (2012); Ahlers, D. J., et al., CurrMol Med., 3:285-301 (2003); Jafarfzade, B. S., et al., Bratisl Lek Listy, 118:564-9 (2017); Ahler, J. D. and Belyakov, I. M., Eur J Immunol, 39:2657-69 (2009); Moore, A. C., et al., J Virol. 76:243-50 (2002); Barouch, D. H., et al., PNAS, 97:4192-7 (2000); Kalams, S. A., et al., J Infectious Disease, 208:818-29 (2013); Buchbinder, S., et al., PLOS One, 12(7):e0179597 (2017); Henke, A, et al., Intervirology, 49:249-52 (2006); and Yang, S. H., et al., Gen Ther., 13:1110-7 (2006).

The term “targeting ligand,” as used herein, is intended to refer to ligands conjugated to a polymer either directly or via one or more spacer molecules. In some aspects, only a small portion of the available amino groups of the polymer is coupled to the ligand. In some aspects, the targeting ligands conjugated to the polymers direct the polymers-nucleic acid complex to bind to specific target cells and penetrate into such cells (e.g., epithelial cells, endothelial cells, hematopoietic cells, and the like). In some aspects, the target ligands can also be an intracellular targeting element, enabling the transfer of the nucleic acid/drug to be guided towards certain favored cellular compartments (mitochondria, nucleus, and the like). In some aspects, the ligand is a polypeptide, folate, and an antigen. In some aspects, the polypeptide ligand is a glycoprotein (e.g., transferrin or asialoorosomucoid (ASOR)), an antibody, an antibody fragment, a cell receptor, a cytokine receptor, or a growth factor receptor (e.g., epidermal growth factor receptor). In some aspects, the antigen ligand is a viral antigen, a bacterial antigen, or a parasite antigen. In some aspects, the ligand is a fusogenic agent (e.g., polymixin B and hemaglutinin HA2), a lysosomotrophic agent, or a nucleus localization signal (NLS) (e.g., T-antigen, and the like). In some aspects, the ligand is a sugar moiety coupled to an amino group. In some aspects, the sugar moiety is a mono- or oligo-saccharide, such as galactose, glucose, fucose, fructose, lactose, sucrose, mannose, cellobiose, nytrose, triose, dextrose, trehalose, maltose, galactosamine, glucosamine, galacturonic acid, glucuronic acid, and gluconic acid.

The term “antibody” includes molecules or active fragments (i.e., antigen binding fragments) of molecules that bind to antigens. These active fragments can be derived from an antibody of the present disclosure by a number of techniques. For further description of general techniques for the isolation of active fragments of antibodies, see for example, Khaw, B. A. et al. J. Nucl. Med. 23:1011-1019 (1982). The term “antibody” also includes bispecific and chimeric antibodies and antibodies in nonmammalian species.

The term “biodegradable linker” or “biofunctional biodegradable linker,” as used herein, refers to a biodegradable linker containing ester, amide, disulfide, and/or phosphate linkages that is used to cross-link cationic multi-block copolymers. In some aspects, the biodegradable linker is hydrophilic and comprises a biodegradable linkage comprising a disulfide bond. In some aspects, the biodegradable linker is a dithiodipropionyl linker.

The term “vector,” as used herein, is intended to refer to a nucleic acid molecule capable of transporting another nucleic acid to which it has been linked; or an entity comprising such a nucleic acid molecule capable of transporting another nucleic acid. In some aspects, the vector is a “plasmid,” which refers to a circular double stranded DNA loop into which additional DNA segments can be ligated. In some aspects, the vector is a viral vector, wherein additional DNA segments can be ligated into the viral genome. In some aspects, such vectors include, but are not limited to: an adenoviral vector, an adeno-associated virus (AAV) vector, retroviral vector, a lentiviral vector, poxvirus vector, a baculovirus vector, a herpes viral vector, simian virus 40 (SV40), cytomegalovirus (CMV), mouse mammary tumor virus (MMTV), and Moloney murine leukemia virus. Certain vectors, or polynucleotides that are part of vectors, are capable of autonomous replication in a host cell into which they are introduced (e.g., bacterial vectors having a bacterial origin of replication, and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can sometimes be used interchangeably, depending on the context, as the plasmid is the most commonly used form of vector. However, also disclosed herein are other forms of expression vectors, such as viral vectors (e.g., replication defective retroviruses, poxviruses, herpesviruses, baculoviruses, adenoviruses and adeno-associated viruses), which can serve equivalent functions.

As used herein, the term “poloxamer” or “poloxamer backbone” refers to molecules having the general formula HO—(C₂H₄O)_{α}(C₃H₆O)_{b}(C₂H₄O)_{c}—H in which α and c are approximately equal. See, Handbook of Biodegradable Polymers, Chapter 12′ “The Poloxamers: Their Chemistry and Medical Applicationss” authored by Lorraine E. Reeve. See also U.S. Publ. No. 2010/0004313, which is herein incorporated by reference in its entirety. Because the poloxamers are the products of a sequential series of reactions, the chain lengths of individual poloxamer blocks are statistical distributions about the average chain length. Thus, the number of ethyleneoxy groups (a and c) and the number of propylenoxy groups (b) are meant to be averages. Poloxamers are generally based on an amphiphilic triblock copolymer of ethylene oxide and propylene oxide, having a central hydrophobic chain of polypropylene oxide flanked by two hydrophilic chains of polyethylene oxide. Because the lengths of the polymer blocks of a poloxamer backbone can vary between various polymeric constructs, many different poloxamers are considered to be within the scope of the present disclosure. In one aspect, for example, the average molecular weight of the poloxamer backbone can range from about 100 to about 100,000 Dalton. In another aspect, the average molecular weight of the poloxamer backbone can range from about 500 to about 50,000 Dalton. In yet another aspect, the average molecular weight of the poloxamer backbone can range from about 1000 to about 20,000 Dalton. The poloxamer backbone can also be described in terms of a ratio of ethylene oxide to propylene oxide. In another aspect, the ratio of ethylene oxide to propylene eoxide is from about 20:1 to about 1:20. For example, in one aspect the ratio of ethylene oxide to propylene oxide is from about 5:1 to about 1:5.

Various additional aspects of the disclosure are described, disclosed or illustrated in further detail in the following subsections.

### 5.3 Polynucleotides

In some aspects, the polynucleotides of the disclosure can include DNA or mRNA sequences (e.g., multicistronic DNA or multicistronic mRNA) for use in the compositions (e.g., pharmaceutical compostions and vaccines) disclosed herein. In some aspects, the present disclosure is directed to a polynucleotide can comprise a nucleic acid sequence encoding one or more viral antigens (e.g., a SARS CoV-2 antigen). In some aspects, the vector further comprises a nucleic acid sequence encoding one or more immune modifiers. In some aspects, the vector comprises a nucleic acid sequence encoding a SARS CoV-2 antigen and, optionally, a second viral antigen. In some aspects, the vector comprises a nucleic acid sequence encoding a SARS-CoV-2 antigen and a second SARS-CoV-2 antigen. In some aspects, the nucleic acid sequence encodes a SARS-CoV-2 antigen and a second SARS-CoV-2 antigen from a different SARS-CoV-2 strain. In some aspects, the nucleic acid sequence encodes different variants of the same SARS-CoV-2 antigen, wherein the different variants of the same SARS-CoV-2 antigen are derived from different strains of SARS CoV-2. In some aspects, the nucleic acid sequence encodes different variants of a SARS-CoV-2 S protein antigen, wherein the different variants of the SARS-CoV-2 S protein antigens are derived from different strains of SARS CoV-2.

In some aspects, the present disclosure is directed to a polynucleotide comprising: (a) a first antigen nucleic acid which encodes a pathogen protein antigen (e.g., a viral antigen, a bacterial antigen, or a parasite antigen) or an antigenic fragment thereof; and (b) a nucleic acid encoding an immune modifier. In some aspects, the first antigen nucleic acid is operably linked to a first promoter. In some aspects, the polynucleotide comprises two or more nucleic acids encoding an immune modifier. In some aspects, each of the nucleic acids encoding an immune modifier encodes a different immune modifier.

The polynucleotides disclosed herein can further comprise: (c) a second antigen nucleic acid which encodes a second pathogen protein antigen (e.g., a viral antigen, a bacterial antigen, or a parasite antigen) or an antigenic fragment thereof. In some aspects, the second pathogen protein or antigenic fragment thereof comprises one or more viral antigens, one or more bacterial antigens, or one or more parasite antigens. In some aspects, the first pathogen antigen and the second pathogen antigen are SARS-CoV-2 antigens from different strains of SARS-CoV-2. In some aspects, the first pathogen antigen and the second pathogen antigens are different variants of the same SARS-CoV-2 antigen, wherein the different variants of the same SARS-CoV-2 antigen are derived from different strains of SARS-CoV-2. In some aspects, the first pathogen antigen and the second pathogen antigen are different variants of a SARS-CoV-2 S protein antigen, wherein the different variants of the SARS-CoV-2 S protein antigen are derived from different strains of SARS CoV-2. In some aspects, the second antigen nucleic acid is operably linked to the first promoter through an internal ribosome entry site (IRES) sequence. In some aspects, the IRES sequence comprises a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 41.

In some aspects, the first and/or second pathogen protein is a bacterial antigen selected from the group consisting of a Yersinia pestis antigen, a Mycobacterium tuberculosis antigen, antigenic fragments thereof, and any combinations thereof. In some aspects, the Yersinia pestis antigen is a Yersinia pestis capsular antigen. In some aspects, the Yersinia pestis capsular antigen is F1-Ag or virulence antigen (V-Ag). In some aspects, the Mycobacterium tuberculosis antigen is an Apa antigen, an HP65 antigen, a rAg85A antigen, any antigenic fragments thereof, or any combinations thereof.

In some aspects, the first and/or second pathogen protein is a viral antigen selected from the group consisting of: an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, antigenic fragments thereof, or any combinations thereof. In some aspects, the enterovirus antigen is an enterovirus 71 (E71) antigen, a coxsackievirus (Cox) protein antigen, antigenic fragments thereof, or any combinations thereof. In some aspects, the E71 antigen is an E71-VP1 antigen, a glutathione S-transferase (GST)-tagged E71-VP1 antigen, antigenic fragments thereof, or any combinations thereof. In some aspects, the Cox protein antigen is GST-tagged Cox protein antigen. In some aspects, the HSV antigen is an HSV-1 envelope antigen, an HSV-2 envelope antigen, an HSV-2 surface glycoprotein antigen, antigenic fragments thereof, or any combinations thereof. In some aspects, the HSV-2 surface glycoprotein antigen is a gB2 antigen, a gC2 antigen, a gD2 antigen, a gE2 antigen, or antigenic fragments thereof, or any combinations thereof. In some aspects, the HIV antigen is an Env antigen, a Gag antigen, a Nef antigen, a Pol antigen, antigenic fragments thereof, and or combinations thereof. In some aspects, the HPV antigen is a minor capsid protein L2 antigen. In some aspects, the minor capsid protein L2 antigen comprises one or more epitope domains (amino acids 10-36 and/or amino acids 65-89) of minor capsid protein L2. In some aspects, the HCV antigen is a nonstructural 3 (NS3) antigen. In some aspects, the RSV antigen is an F antigen, a G antigen, antigenic fragments thereof, or any combinations thereof. In some aspects, the Dengue virus antigen is an E protein antigen, an E protein domain III (EDIII) antigen, a non-structural protein 1 (NS1) antigen, a DEN-80E antigen, antigenic fragments thereof, or any combinations thereof. In some aspects, the Ebola virus antigen is a spike glycoprotein (GB) antigen, a VP24 antigen, a VP40 antigen, a nucleoprotein (NP) antigen, a VP30 antigen, a VP35 antigen, antigenic fragments thereof, or any combinations thereof. In some aspects the Zika virus antigen is an envelope domain III antigen, a CKD antigen, antigenic fragments thereof, or any combinations thereof. In some aspects, the Chikungunya virus antigen is an E1 glycoprotein subunit antigen, the MHC class I epitope PPFGAGRPGQFGDI (SEQ ID NO: 34), the MHC class I epitope TAECKDKNL (SEQ ID NO: 35), the MHC class II epitope VRYKCNCGG (SEQ ID NO: 36), antigenic fragments thereof, or any combinations thereof. In some aspects, the measles virus antigen is a hemagglutinin protein MV-H antigen, a fusion protein MV-F antigen, antigenic fragments thereof, or any combinations thereof. In some aspects, the MERS-CoV antigen is a spike (S) protein antigen, an antigen from the receptor-binding domain of the S protein, an antigen from the membrane fusion domain of the S protein, antigenic fragments thereof, or any combinations thereof. In some aspects, the SARS-CoV antigen is a spike (S) protein antigen, an antigen from the receptor binding domain of the S protein, an antigen from the membrane fusion domain of the S protein, an envelope (E) protein antigen, an M protein antigen, antigenic fragments thereof, or any combinations thereof.

In some aspects, the first and/or second pathogen protein comprises one or more influenza virus antigens from any influenza virus type or subtype. In some aspects, the one or more influenza virus antigens are selected from the group consisting of: an influenza virus hemagglutinin (HA) antigen, an influenza virus neuraminidase (NA) antigen, an influenza virus matrix-2 (M2) protein antigen, antigenic fragments thereof, and any combination thereof. In some aspects, the one or more influenza virus antigens are derived from influenza virus type A, type B, type C, type D, or any combination thereof. In some spects, the one or more influenza virus antigens are derived from influenza virus type A. In some aspects, the one or more influenza virus antigens derived from influenza virus type A have (a) a HA subtype selected from H1 through H18 or any combination thereof and (b) a NA subtype selected from N1 through N11 or any combination thereof. In some aspects, the one or more influenza virus antigens derived from influenza virus type A, subtype H1N1; influenza virus type A, subtype H2N2; influenza virus type A, subtype H3N2; influenza virus type A, subtype H5N1; influenza virus type A, subtype H7N7; influenza virus type A, subtype H7N9; influenza virus type A, subtype H9N2; or any combination thereof. In some aspects, the one or more influenza virus antigens are derived from influenza virus type A, subtype H1N1; influenza virus type A, subtype H3N2; or the combination thereof. In some spects, the one or more influenza virus antigens are derived from influenza virus type B. In some aspects, the first pathogen protein comprises one or more SARS-CoV-2 antigens or antigenic fragments thereof disclosed herein, and the second pathogen protein comprises one or more influenza virus antigens or antigenic fragments thereof disclosed herein. In some aspects, the pathogen protein is a parasite antigen, wherein the parasite antigen is a protozoan antigen. In some aspects, the pathogen protein is a parasite antigen selected from the group consisting of a Toxoplasma gondii antigen, a Plasmodium falciparum antigen, antigenic fragments thereof, and any combinations thereof. In some aspects, the Toxoplasma gondii antigen is antigen MIC8. In some aspects, the Plasmodium falciparum antigen is a SERA5 polypeptide antigen, a circumsporozite protein antigen, antigenic fragments thereof, or any combinations thereof. In some aspects, the pathogen protein is a parasite antigen, wherein the parasite antigen is a parasitic or pathogenic fungus antigen. In some aspects, the parasitic or pathogenic fungus antigen is selected from the group consisting of a Candida spp. antigen (e.g., a Candida albicans antigen, a Candida glabrata antigen, a Candida parapsilosis antigen, a Candida tropicalis antigen, a Candida lusitaniae antigen, a Candida krusei antigen), a Pneumocystis spp. antigen, a Malassezia spp. antigen (e.g., a Malassezia furfur antigen), an Aspergillus fumigatus antigen, a Cryptococcus spp. antigen (e.g., a Cryptococcus neoformans antigen, a Cryptococcus gattii antigen), a Histoplasma capsulatum antigen, a Blastomyces dermatitidis antigen, a Paracoccidioides spp. antigen (e.g., a Paracoccidioides brasiliensis antigen, a Paracoccidioides lutzii antigen), a Coccidioides spp. antigen (e.g., a Coccidioides immitis antigen, a Coccidioides posadasii antigen), a Penicillium marneffei antigen, a Sporothrix schenckii antigen, a Trichosporon asahii antigen, a Fusarium spp. antigen (e.g., a Fusarium solanum antigen, a Fusarium oxysporum antigen), a Nectria spp. antigen, a Pseudoallescheria boydii antigen, a Cladophialphora bantianum antigen, a Ramichloridium spp. antigen, a Dactylaria gallopava antigen, an Exophiala spp. antigen (e.g., an Exophiala jeanselmei antigen, an Exophiala dermatitidis antigen), a Curvularia spp. antigen, a Bipolaris spp. antigen, an Alternaria spp. antigen, a Lacazia loboi antigen, a Conidiobolus spp. antigen (e.g., a Conidiobolus coronatus antigen, a Conidiobolus incongruus antigen), and any combination thereof.

In some aspects, the present disclosure is directed to a polynucleotide (e.g., multicistronic DNA or multicistronic mRNA) comprising: (a) a first antigen nucleic acid which encodes a SARS-CoV-2 spike (S) protein or an antigenic fragment thereof, and (b) a nucleic acid encoding an immune modifier. In some aspects, the first antigen nucleic acid is operably linked to a first promoter. In some aspects, the polynucleotide comprises two or more nucleic acids encoding an immune modifier. In some aspects, each of the nucleic acids encoding an immune modifier encodes a different immune modifier.

The polynucleotides disclosed herein can further comprise: (c) a second antigen nucleic acid which encodes a SARS-CoV-2 protein or an antigenic fragment thereof. In some aspects, the SARS-CoV-2 protein or antigenic fragment thereof is selected from the group consisting of: a SARS-CoV-2 membrane (M) protein or an antigenic fragment thereof, a SARS-CoV-2 envelope (E) protein or an antigenic fragment thereof, a SARS-CoV-2 nucleocapsid (N) protein or an antigenic fragment thereof, and any combination thereof. In some aspects, the first antigen nucleic acid encodes a SARS-CoV-2 protein or an antigenic fragment thereof and the second antigen nucleic acid encodes a SARS-CoV-2 protein or an antigenic fragment thereof from a different strain of SARS-CoV-2. In some aspects, the first antigen nucleic acid encodes a SARS-CoV-2 S protein or an antigenic fragment thereof and the second antigen nucleic acid encodes a SARS-CoV-2 S protein or an antigenic fragment thereof from a different strain of SARS-CoV-2. In some aspects, the second antigen nucleic acid is operably linked to the first promoter through an internal ribosome entry site (IRES) sequence. In some aspects, the IRES sequence comprises a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 41.

The polynucleotides disclosed herein can further comprise one or more second promoters. In some aspects, the second antigen nucleic acid is operably linked to the one or more second promoters. In some aspects, one or more nucleic acids encoding an immune modifier is operably linked to the one or more second promoters. In some aspects, one or more of the nucleic acids encoding an immune modifier is operably linked to the first promoter or the one or more second promoters through an internal ribosome entry site (IRES) sequence. In some aspects, the IRES sequence comprises a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 41.

In some aspects, the first promoter or the one or more second promoters is selected from the group consisting of: a cytomegalovirus (CMV) promoter (SEQ ID NO: 31), a Rouse sarcoma virus (RSV) promoter, a Moloney murine leukemia virus (Mo-MuLV) long terminal repeat (LTR) promoter, a mammalian elongation factor 1 (EF1) promoter, a cytokeratin 18 (CK18) promoter, a cytokeratin 19 (CK19) promoter, a simian virus 40 (SV40) promoter (SEQ ID NO: 32), a murine U6 promoter, a skeletal β-actin promoter, a β-actin promoter, a murine phosphoglycerate kinase 1 (PGK1) promoter, a human PGK1 promoter, a CMV enhancer/chicken β-actin (CAG) promoter (SEQ ID NO: 33), and any combination thereof. In some aspects, the one or more second promoters is the CMV promoter. In some aspects, the one or more second promoters is a mammalian EF1 promoter. In some aspects, the mammalian EF1 promoter is a hEF1-HTLV promoter (SEQ ID NO: 38).

In some aspects, each of the nucleic acids which encodes an immune modifier is under the control of a promoter selected from the group consisting of a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CAG promoter, and any combination thereof. In some aspects, the mammalian EF1 promoter is a hEF1-HTLV promoter.

In some aspects, each of the second antigen nucleic acids is under the control of a promoter selected from the group consisting of a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CAG promoter, and any combination thereof. In some aspects, the mammalian EF1 promoter is a hEF1-HTLV promoter.

In some aspects, the immune modifier is selected from the group consisting of: interleukin (IL) 2 (IL-2), IL-12 p35, IL-12 p40, IL-12 p70, IL-15, IL-18, tumor necrosis factor alpha (TNFα), granulocyte-macrophage colony-stimulating factor (GM-CSF), interferon (IFN) α (IFN-α), IFN-β, a chemokine, major histocompatibility complex (MHC) class I (MHC I), MHC class II (MHC II), human leukocyte antigen (HLA)-DR isotype (HLA-DR), CD80, CD86, and any combination thereof. In some aspects, the chemokine is selected from the group consisting of: C-C motif chemokine ligand (CCL) 3 (CCL3), CCL4, CCL5, CCL21, CCL28, C-X-C motif chemokine ligand (CXCL) 10 (CXCL10), and any combination thereof.

In some aspects, the immune modifier is an interleukin, e.g., IL-12. IL-12 is composed of four alpha helices. It is a heterodimeric cytokine encoded by two separate genes, IL-12A (p35) and IL-12B (p40). The active heterodimer (referred to as p70), and a homodimer of p40 are formed following protein synthesis. In some aspects, the immune modifier is an IL-12 heterodimer (IL-12 p70) or an IL-12 homodimer (e.g., IL-12 p35 or IL-12 p40).

In some aspects, the IL-12 p35 immune modifier comprises an amino acid sequence having at least 70%, at least 75%, at least 80%, at least 85%, at least 90%, at least 95%, at least 99%, or 100% sequence identity to SEQ ID NO: 43 (mouse IL-12 p35) or SEQ ID NO: 47 (human IL-12 p35). In some aspects, the IL-12 p40 immune modifier comprises an amino acid sequence having at least 70%, at least 75%, at least 80%, at least 85%, at least 90%, at least 95%, at least 99%, or 100% sequence identity to SEQ ID NO: 45 (mouse IL-12 p40) or SEQ ID NO: 49 (human IL-12 p40).

In some aspects, the nucleic acid encoding IL-12 p35 has at least 70%, at least 75%, at least 80%, at least 85%, at least 90%, at least 95%, at least 99%, or 100% sequence identity to SEQ ID NO: 42 (nucleic acid sequence encoding mouse IL-12 p35) or SEQ ID NO: 46 (nucleic acid sequence encoding human IL-12 p35). In some aspects, the nucleic acid encoding IL-12 p40 has at least 70%, at least 75%, at least 80%, at least 85%, at least 90%, at least 95%, at least 99%, or 100% sequence identity to SEQ ID NO: 44 (nucleic acid sequence encoding mouse IL-12 p40) or SEQ ID NO: 48 (nucleic acid sequence encoding human IL-12 p40).

In some aspects, the immune modifier is a viral protein (e.g., SARS-CoV-2 non-structural protein 1 (Nsp1), SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b) that attenuates a local inflammatory response and/or interferon response. In some aspects, the viral protein is from the same virus as a viral antigen encoded by an antigen nucleic acid. In some aspects, In some aspects, the viral protein is from a different virus than a viral antigen encoded by an antigen nucleic acid. In some aspects, the viral protein attenuates a local inflammatory response and/or interferon response elicited by a pathogen antigen disclosed herein. In some aspects, the immune modifier is selected from the group consisting of SARS-CoV-2 Nsp1, SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b, and any combination thereof.

In some aspects, the immune modifier comprises one or more concatamers of non-coding 5′-C-phosphate-G-3′ (CpG) dinucleotides. In some aspects, the one or more concatamers of non-coding CpG dinucleotides activate the Toll-like receptor 9 (TLR9) signaling pathway. In some aspects, the one or more concatamers of non-coding CpG dinucleotides comprise one or more concatamers of non-coding CpG dinucleotides previously reported in Bauer, A. et al., Nucleic Acids Research 38(12):3891-908 (2010); Cornelie, S. et al., Journal of Biological Chemistry 279(15):15124-9 (2004); Klinman, D. et al., J Immunol. 158(8):3635-9 (1997); Klinman, D. et al., Immunological Reviews 199(1):201-16 (2004); Luo, Z. et al., Mol Med Rep. 6(6):1309-14 (2012); Bode, C. et al., Expert Rev Vaccines 10(4):499-511 (2011); and Kuo, T. et al., Scientific Reports 10:20085 (2020), each of which is incorporated by reference herein in its entirety.

In some aspects, the nucleic acid encoding an immune modifier comprises a combination (i) a nucleic acid encoding an interleukin, and (ii) a nucleic acid encoding a major histocompatibility complex and/or a chemokine.

In some aspects, the nucleic acid encoding an immune modifier comprises a nucleic acid encoding IL-12 p35, a nucleic acid encoding IL-12 p40, or the combination thereof. In some aspects, the nucleic acid encoding IL-12 p35 has at least 70%, at least 75%, at least 80%, at least 85%, at least 90%, at least 95%, at least 99%, or 100% sequence identity to SEQ ID NO: 42 (nucleic acid sequence encoding mouse IL-12 p35) or SEQ ID NO: 46 (nucleic acid sequence encoding human IL-12 p35). In some aspects, the nucleic acid encoding IL-12 p40 has at least 70%, at least 75%, at least 80%, at least 85%, at least 90%, at least 95%, at least 99%, or 100% sequence identity to SEQ ID NO: 44 (nucleic acid sequence encoding mouse IL-12 p40) or SEQ ID NO: 48 (nucleic acid sequence encoding human IL-12 p40). In some aspects, the nucleic acid encoding an immune modifier further comprises a nucleic acid encoding MHC I, a nucleic acid encoding MHC II, or the combination thereof.

In some aspects, the nucleic acid encoding an immune modifier comprises a nucleic acid encoding IL-12, a nucleic acid encoding IL-15, or the combination thereof.

In some aspects, the nucleic acid encoding an immune modifier comprises a combination of a nucleic acid encoding IL-12 and a nucleic acid encoding IL-15.

In some aspects, the nucleic acid encoding an immune modifier comprises a nucleic acid encoding IL-2, a nucleic acid encoding IL-15, or the combination thereof. In some aspects, the nucleic acid encoding an immune modifier further comprises a nucleic acid encoding MHC I, a nucleic acid encoding MHC II, a nucleic acid encoding CCL3, a nucleic acid encoding CCL4, any the combination thereof.

In some aspects, the nucleic acid encoding an immune modifier comprises a nucleic acid encoding CCL3, a nucleic acid encoding CCL4, or the combination thereof.

The first antigen nucleic acid of the polynucleotides disclosed herein can encode a SARS-CoV-2 full length polypeptide or antigenic fragment thereof. For example, in some aspects, the first antigen nucleic acid of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the first antigen nucleic acid of the polynucleotide encodes a polypeptide having at least 70%, at least 75%, at least 80%, at least 85%, at least 90%, at least 95%, or at least 99% sequence identity to the amino acid sequence of SEQ ID NO: 2 or SEQ ID NO: 4. In some aspects, the first antigen nucleic acid of the polynucleotide encodes a polypeptide comprising the amino acid sequence of SEQ ID NO: 2 or SEQ ID NO: 4. In some aspects, the first antigen nucleic acid of the polynucleotide comprises a nucleic acid sequence having at least 70%, at least 75%, at least 80%, at least 85%, at least 90%, at least 95%, at least 99%, or 100% sequence identity to SEQ ID NO: 1 or SEQ ID NO: 3.

In some aspects, the second antigen nucleic acid of the polynucleotides disclosed herein can encode a SARS-CoV-2 full length polypeptide or antigenic fragment thereof. For example, in some aspects, the second antigen nucleic acid of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the second antigen nucleic acid of the polynucleotide encodes a polypeptide having at least 70%, at least 75%, at least 80%, at least 85%, at least 90%, at least 95%, or at least 99% sequence identity to the amino acid sequence of SEQ ID NO: 2 or SEQ ID NO: 4. In some aspects, the second antigen nucleic acid of the polynucleotide encodes a polypeptide comprising the amino acid sequence of SEQ ID NO: 2 or SEQ ID NO: 4. In some aspects, the second antigen nucleic acid of the polynucleotide comprises a nucleic acid sequence having at least 70%, at least 75%, at least 80%, at least 85%, at least 90%, at least 95%, at least 99%, or 100% sequence identity to SEQ ID NO: 1 or SEQ ID NO: 3.

In some aspects, the first antigen nucleic acid of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4, wherein the contiguous amino acids of SEQ ID NO: 2 or SEQ ID NO: 4 comprise one or more mutations (i.e., one or more substitutions, deletions, insertions, or any combination thereof). In some aspects, the first antigen nucleic acid of the polynucleotide encodes a polypeptide comprising the amino acid sequence of SEQ ID NO: 2 or SEQ ID NO: 4, wherein the polypeptide comprises one or more mutations (i.e., one or more substitutions, deletions, insertions, or any combination thereof).

In some aspects, the second antigen nucleic acid of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4, wherein the contiguous amino acids of SEQ ID NO: 2 or SEQ ID NO: 4 comprise one or more mutations (i.e., one or more substitutions, deletions, insertions, or any combination thereof). In some aspects, the second antigen nucleic acid of the polynucleotide encodes a polypeptide comprising the amino acid sequence of SEQ ID NO: 2 or SEQ ID NO: 4, wherein the polypeptide comprises one or more mutations (i.e., one or more substitutions, deletions, insertions, or any combination thereof).

In some aspects, the first antigen nucleic acid of the polynucleotides disclosed herein can encode the receptor binding domain (RBD) of the SARS-Cov-2 S protein or an antigenic fragment thereof. For example, in some aspects, the first antigen nucleic acid of the polynucleotide encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the first antigen nucleic acid of the polynucleotide encodes a polypeptide having at least 70%, at least 75%, at least 80%, at least 85%, at least 90%, at least 95%, or at least 99% sequence identity to the amino acid sequence of SEQ ID NO: 6. In some aspects, the first antigen nucleic acid of the polynucleotide encodes encodes a polypeptide comprising the amino acid sequence of SEQ ID NO: 6. In some aspects, the first antigen nucleic acid of the polynucleotide comprises a nucleic acid sequence having at least 70%, at least 75%, at least 80%, at least 85%, at least 90%, at least 95%, at least 99%, or 100% sequence identity to SEQ ID NO: 5.

In some aspects, the second antigen nucleic acid of the polynucleotides disclosed herein can encode the receptor binding domain (RBD) of the SARS-Cov-2 S protein or an antigenic fragment thereof. For example, in some aspects, the second antigen nucleic acid of the polynucleotide encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the second antigen nucleic acid of the polynucleotide encodes a polypeptide having at least 70%, at least 75%, at least 80%, at least 85%, at least 90%, at least 95%, or at least 99% sequence identity to the amino acid sequence of SEQ ID NO: 6. In some aspects, the second antigen nucleic acid of the polynucleotide encodes a polypeptide comprising the amino acid sequence of SEQ ID NO: 6. In some aspects, the second antigen nucleic acid of the polynucleotide comprises a nucleic acid sequence having at least 70%, at least 75%, at least 80%, at least 85%, at least 90%, at least 95%, at least 99%, or 100% sequence identity to SEQ ID NO: 5.

In some aspects, the first antigen nucleic acid of the polynucleotides disclosed herein can encode the RBD of the SARS-Cov-2 S protein or an antigenic fragment thereof. For example, in some aspects, the first antigen nucleic acid of the polynucleotide encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6, wherein the contiguous amino acids of SEQ ID NO: 6 comprise one or more mutations (i.e., one or more substitutions, deletions, insertions, or any combination thereof). In some aspects, the first antigen nucleic acid of the polynucleotide encodes encodes a polypeptide comprising the amino acid sequence of SEQ ID NO: 6, wherein the polypeptide comprises one or more mutations (i.e., one or more substitutions, deletions, insertions, or any combination thereof).

In some aspects, the second antigen nucleic acid of the polynucleotides disclosed herein can encode the RBD of the SARS-Cov-2 S protein or an antigenic fragment thereof. For example, in some aspects, the second antigen nucleic acid of the polynucleotide encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6, wherein the contiguous amino acids of SEQ ID NO: 6 comprise one or more mutations (i.e., one or more substitutions, deletions, insertions, or any combination thereof). In some aspects, the second antigen nucleic acid of the polynucleotide encodes a polypeptide comprising the amino acid sequence of SEQ ID NO: 6, wherein the polypeptide comprises one or more mutations (i.e., one or more substitutions, deletions, insertions, or any combination thereof).

In some aspects, the first antigen nucleic acid of the polynucleotides disclosed herein can encode the S1 subunit of the SARS-Cov-2 S protein or an antigenic fragment thereof. For example, in some aspects, the first antigen nucleic acid of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the first antigen nucleic acid encodes a polypeptide having at least 70%, at least 75%, at least 80%, at least 85%, at least 90%, at least 95%, or at least 99% sequence identity to the amino acid sequence of SEQ ID NO: 40. In some aspects, the first antigen nucleic acid of the polynucleotide encodes a polypeptide comprising the amino acid sequence of SEQ ID NO: 40. In some aspects, the first antigen nucleic acid of the polynucleotide comprises a nucleic acid sequence having at least 70%, at least 75%, at least 80%, at least 85%, at least 90%, at least 95%, at least 99%, or 100% sequence identity to SEQ ID NO: 39.

In some aspects, the second antigen nucleic acid of the polynucleotides disclosed herein can encode the S1 subunit of the SARS-Cov-2 S protein or an antigenic fragment thereof. For example, in some aspects, the second antigen nucleic acid of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the second antigen nucleic acid encodes a polypeptide having at least 70%, at least 75%, at least 80%, at least 85%, at least 90%, at least 95%, or at least 99% sequence identity to the amino acid sequence of SEQ ID NO: 40. In some aspects, the second antigen nucleic acid of the polynucleotide encodes a polypeptide comprising the amino acid sequence of SEQ ID NO: 40. In some aspects, the second antigen nucleic acid of the polynucleotide comprises a nucleic acid sequence having at least 70%, at least 75%, at least 80%, at least 85%, at least 90%, at least 95%, at least 99%, or 100% sequence identity to SEQ ID NO: 39.

In some aspects, the first antigen nucleic acid of the polynucleotides disclosed herein can encode the S1 subunit of the SARS-Cov-2 S protein or an antigenic fragment thereof. For example, in some aspects, the first antigen nucleic acid of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40, wherein the contiguous amino acids of SEQ ID NO: 40 comprise one or more mutations (i.e., one or more substitutions, deletions, insertions, or any combination thereof). In some aspects, the first antigen nucleic acid of the polynucleotide encodes encodes a polypeptide comprising the amino acid sequence of SEQ ID NO: 40, wherein the polypeptide comprises one or more mutations (i.e., one or more substitutions, deletions, insertions, or any combination thereof).

In some aspects, the second antigen nucleic acid of the polynucleotides disclosed herein can encode the S1 subunit of the SARS-Cov-2 S protein or an antigenic fragment thereof. For example, in some aspects, the second antigen nucleic acid of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40, wherein the contiguous amino acids of SEQ ID NO: 40 comprise one or more mutations (i.e., one or more substitutions, deletions, insertions, or any combination thereof). In some aspects, the second antigen nucleic acid of the polynucleotide encodes a polypeptide comprising the amino acid sequence of SEQ ID NO: 40, wherein the polypeptide comprises one or more mutations (i.e., one or more substitutions, deletions, insertions, or any combination thereof).

In some aspects, the one or more mutations in the SARS-CoV-2 full-length S protein, the RBD of the SARS-Cov-2 S protein, the S1 subunit of the SARS-CoV-2 S protein, or antigenic fragments thereof comprise one or more mutations previously reported in Li, T. et al., Emerg Microbes Infect. 9(1):2076-90 (2020); Lee, P. et al., Immune Netw. 21(1):e4 (2021); Yu, J. et al., Science 369(6505):806-11 (2020); Cattin-Ortola, J. et al., Nat Commun. 12(1):5333 (2021); Corbett, K. et al., Nature 586(7830):567-71 (2020); Hsieh, C. et al., Science 369(6510):1501-5 (2020); and Harvey, W. et al., Nat Rev Microbiol. 19(7):409-24 (2021), each of which is incorporated by reference herein in its entirety.

In some aspects, the one or more mutations in the SARS-CoV-2 full-length S protein, the RBD of the SARS-Cov-2 S protein, the S1 subunit of the SARS-CoV-2 S protein, or antigenic fragments thereof are selected from: ΔM1-S13, S12P, S13I, L5F, L18F, T19R, T20N, P26S, Q52R, A67V, ΔH69-V70, G75V, T76I, D80A, T95I, R102I, ΔD119-F120, C136Y, D138Y, ΔF140, ΔL141-Y144, AY144, Y144S, Y145N, ΔH146, N148S, K150R, K150E, K150T, K150Q, S151P, W152C, E154K, ΔE156-F157, F157L, F157A, R158G, R190S, ΔI210, D215G, A222V, ΔL241-S243, ΔL242-L244, ΔA243-L244, ΔR246-G252, R246I, 11-amino acid residue insertion between Y248 and L249, D253G, D253N, R346K, V367F, E406W, K417E, K417V, K417N, K417T, N439K, K444A, K444R, K444N, K444Q, V445A, V445E, G447A, N450D, L452R, L452Q, Y453F, L455F, N460I, S477G, S477N, S477R, T478I, T478K, V483I, E484K, E484Q, G485R, F486A, F486V, F486L, N487A, F490S, Q493E, Q493K, S494P, N501Y, A570D, Q613H, D614G, H655Y, Q677H, Δ678-679, Δ681-682, Δ681-684, Δ682-685, P681H, P681R, R682S, R682A, R682Q, R683S, R683G, R683Q, R685G, R685Q, I692V, A701V, T716I, F817P, T859N, F888L, A892P, A899P, A942P, D950N, S982A, K986P, V987P, T1027I, Q1071H, D1118H, V1176F, M1229I, ΔC1253-T1273, ΔC1254-T1273, ΔK1255-T1273, D1257A, E1258A, D1259A, D1260A, E1262A, K1269A, H1271K, T1273A, or any combination thereof, wherein the amino acid locations correspond to SEQ ID NO: 2 or SEQ ID NO: 4.

In some aspects, the one or more mutations comprise one or more mutations in the N-terminal signal peptide, which corresponds to amino acids 1-13 of SEQ ID NO: 2 or SEQ ID NO: 4. In some aspects, the one or more mutations in the N-terminal signal peptide is ΔM1-S13, wherein the amino acid locations correspond to SEQ ID NO: 2 or SEQ ID NO: 4.

In some aspects, the one or mutations comprise one or more mutations in the C-terminus of the full-length SARS-CoV-2 S protein. In some aspects, the one or more mutations in the C-terminus of the full-length SARS-CoV-2 S protein comprise one or more mutations in the C-terminal endoplasmic reticulum (ER) retention peptide, which corresponds to amino acids 1254-1273 of SEQ ID NO: 2 or SEQ ID NO: 4. In some aspects, the one or more mutations in the C-terminal ER retention peptide are selected from D1257A, E1258A, D1259A, D1260A, E1262A, K1269A, H1271K, T1273A, or any combination thereof, wherein the amino acid locations correspond to SEQ ID NO: 2 or SEQ ID NO: 4. In some aspects, the one or more mutations in the C-terminal ER retention peptide comprise D1257A+E1258A+D1259A+D1260A+E1262A (i.e., a D/E to A mutant), wherein the amino acid locations correspond to SEQ ID NO: 2 or SEQ ID NO: 4. In some aspects, the one or mutations in the C-terminal ER retention peptide is ΔC1253-T1273, ΔC1254-T1273, or ΔK1255-T1273.

In some aspects, the one or more mutations comprise K986P+V987P (i.e., a S-2P mutation), wherein the amino acid locations correspond to SEQ ID NO: 2 or SEQ ID NO: 4. In some aspects, the one or more mutations comprise F817P+A892P+A899P+A942P (i.e., a hexa-proline S mutation), wherein the amino acid locations correspond to SEQ ID NO: 2 or SEQ ID NO: 4. In some aspects, the one or more mutations comprise one or more mutations in the 681-PRRAR/SVA-688 S1/S2 furin cleavage site, wherein the amino acid locations correspond to SEQ ID NO: 2 or SEQ ID NO: 4. In some aspects, the one or more mutations in the 681-PRRAR/SVA-688 S1/S2 furin cleavage site are: (a) R682S+R683S (i.e., a SSAR mutation), (b) Δ681-684 (i.e., a ΔPRRA mutation), (c) Δ678-679+Δ681-682, (d) R682A+R683G+R685G (i.e., a 682-AGAG-685 mutation), (e) R682Q+R683Q+R685Q, (f) R682S+R685G, or (g) Δ682-685 (i.e., a ΔRRAR mutation), wherein the amino acid locations correspond to SEQ ID NO: 2 or SEQ ID NO: 4.

In some aspects, the one or more mutations comprise: (a) F817P+A892P+A899P+A942P (i.e., a hexa-proline S mutation) and (b) K986P+V987P (i.e., a S-2P mutation), wherein the amino acid locations correspond to SEQ ID NO: 2 or SEQ ID NO: 4. In some aspects, the one or more mutations comprise: (a) R682A+R683G+R685G (i.e., a 682-AGAG-685 mutation) and (b) K986P+V987P (i.e., a S-2P mutation), wherein the amino acid locations correspond to SEQ ID NO: 2 or SEQ ID NO: 4. In some aspects, the one or more mutations comprise: (a) R682A+R683G+R685G (i.e., a 682-AGAG-685 mutation), (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations comprise: (a) R682Q+R683Q+R685Q and (b) K986P+V987P (i.e., a S-2P mutation). In some aspects, the one or more mutations comprise: (a) R682Q+R683Q+R685Q, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations comprise: (a) R682S+R685G and (b) K986P+V987P (i.e., a S-2P mutation), wherein the amino acid locations correspond to SEQ ID NO: 2 or SEQ ID NO: 4. In some aspects, the one or more mutations comprise: (a) R682S+R685G, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4.

The second antigen nucleic acid of the polynucleotides disclosed herein can encode a SARS-CoV-2 M protein or an antigenic fragment thereof, a SARS-CoV-2 E protein or an antigenic fragment thereof, a SARS-CoV-2 N protein or an antigenic fragment thereof, or any combination thereof. In some aspects, the first antigen nucleic acid and the second antigen nucleic acid of the polynucleotides encode SARS-CoV-2 proteins or antigenic fragments thereof from different strains of SARS-CoV-2. In some aspects, the first antigen nucleic acid and the second antigen nucleic acid of the polynucleotides encode variants of the same SARS-CoV-2 protein or antigenic fragment thereof, wherein the variants of the same SARS-CoV-2 protein or antigenic fragment thereof are derived from different strains of SARS-CoV-2. In some aspects, the first antigen nucleic acid and the second antigen nucleic acid of the polynucleotides encode variants of a SARS-CoV-2 S protein or antigenic fragment thereof, wherein the variants of the SARS-CoV-2 S protein or antigenic fragment thereof are derived from different strains of SARS-CoV-2.

For example, in some aspects, the second antigen nucleic acid of the polynucleotide encodes at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the second antigen nucleic acid encodes a polypeptide having at least 70%, at least 75%, at least 80%, at least 85%, at least 90%, at least 95%, or at least 99% sequence identity to the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the second antigen nucleic acid of the polynucleotide encodes a polypeptide comprising the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the second antigen nucleic acid of the polynucleotide comprises a nucleic acid sequence having at least 70%, at least 75%, at least 80%, at least 85%, at least 90%, at least 95%, at least 99%, or 100% sequence identity to SEQ ID NO: 7, SEQ ID NO: 9, SEQ ID NO: 11, SEQ ID NO: 13, SEQ ID NO: 15, SEQ ID NO: 17, or SEQ ID NO: 19.

In some aspects, the second antigen nucleic acid of the polynucleotide encodes at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20, wherein the contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20 comprise one or more mutations selected from A2S, F28L, I48V, V70L, I82T, M84T, or any combination thereof, wherein the amino acid locations correspond to SEQ ID NO: 8. In some aspects, the second antigen nucleic acid of the polynucleotide encodes a polypeptide comprising the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20, wherein the polypeptide comprises one or more mutations selected from A2S, F28L, I48V, V70L, I82T, M84T, or any combination thereof, wherein the amino acid locations correspond to SEQ ID NO: 8.

In some aspects, the second antigen nucleic acid of the polynucleotide encodes at least 8, at least 10, at least 15, at least 20, at least 25, at least 30, at least 35, at least 40, at least 45, at least 50, at least 55, at least 60, at least 65, at least 70, or at least 75 contiguous amino acids of SEQ ID NO: 22, SEQ ID NO: 24, or SEQ ID NO: 26. In some aspects, the second antigen nucleic acid encodes a polypeptide having at least 70%, at least 75%, at least 80%, at least 85%, at least 90%, at least 95%, or at least 99% sequence identity to the amino acid sequence of SEQ ID NO: 22, SEQ ID NO: 24, or SEQ ID NO: 26. In some aspects, the second antigen nucleic acid of the polynucleotide encodes a polypeptide comprising the amino acid sequence of SEQ ID NO: 22, SEQ ID NO: 24, or SEQ ID NO: 26. In some aspects, the second antigen nucleic acid of the polynucleotide comprises a nucleic acid sequence having at least 70%, at least 75%, at least 80%, at least 85%, at least 90%, at least 95%, at least 99%, or 100% sequence identity to SEQ ID NO: 21, SEQ ID NO: 23, or SEQ ID NO: 25.

In some aspects, the second antigen nucleic acid of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, or at least 400 contiguous amino acids of SEQ ID NO: 28. In some aspects, the second antigen nucleic acid encodes a polypeptide having at least 70%, at least 75%, at least 80%, at least 85%, at least 90%, at least 95%, or at least 99% sequence identity to the amino acid sequence of SEQ ID NO: 28. In some aspects, the second antigen nucleic acid of the polynucleotide encodes a polypeptide comprising the amino acid sequence of SEQ ID NO: 28. In some aspects, the second antigen nucleic acid of the polynucleotide comprises a nucleic acid sequence having at least 70%, at least 75%, at least 80%, at least 85%, at least 90%, at least 95%, at least 99%, or 100% sequence identity to SEQ ID NO: 27.

In some aspects, the present disclosure is directed to a polynucleotide (e.g., multicistronic DNA or multicistronic mRNA) comprising a first antigen nucleic acid which encodes a first pathogen protein or an antigenic fragment thereof, wherein the first antigen nucleic acid is operably linked to a first promoter. In some aspects, the first antigen nucleic acid which encodes a first pathogen protein is selected from the group consisting of a viral protein, a bacterial protein, a parasite protein, and any antigenic fragment thereof.

In some aspects, the polynucleotide further comprises a second antigen nucleic acid which encodes a second pathogen protein or an antigenic fragment thereof. In some aspects, the second antigen nucleic acid which encodes a second pathogen protein is selected from the group consisting of a viral protein, a bacterial protein, a parasite protein, and any antigenic fragment thereof.

In some aspects, the first pathogen protein and/or the second pathogen protein is/are selected from the group consisting of a Yersinia pestis antigen, a Mycobacterium tuberculosis antigen, an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, a Toxoplasma gondii antigen, a Plasmodium falciparum: antigen, an influenza virus antigen, antigenic fragments thereof, and any combinations thereof.

In some aspects, the first pathogen protein and/or the second pathogen protein is/are selected from the group consisting of: a Yersiniapestis F1-Ag, a Yersiniapestis V-Ag, a Mycobacterium tuberculosis Apa antigen, a Mycobacterium tuberculosis H₁P65 antigen, a Mycobacterium tuberculosis rAg85A antigen, an E71 VP1 antigen, a GST-tagged E71-VP1 antigen, a Cox protein antigen, a GST-tagged Cox protein antigen, an HSV-1 envelope antigen, an HSV-2 envelope antigen, an HSV-2 gB2 antigen, an HSV-2 gC2 antigen, an HSV-2 gD2 antigen, an HSV-2 gE2 antigen, an HIV Env antigen, an HIV Gag antigen, an HIV Nef antigen, an HIV Pol antigen, an HPV minor capsid protein L2 antigen, an HCV NS3 antigen, a RSV F antigen, a RSV G antigen, a Dengue virus E protein antigen, a Dengue virus EDIII antigen, a Dengue virus NS1 antigen, a Dengue virus DEN-80E antigen, an Ebola virus GB antigen, an Ebola virus VP24 antigen, an Ebola virus VP40 antigen, an Ebola virus NP antigen, an Ebola virus VP30 antigen, an Ebola virus VP35 antigen, a Zika virus envelope domain III antigen, a Zika virus CKD antigen, a Chikungunya virus E1 glycoprotein subunit antigen, the MHC class I epitope PPFGAGRPGQFGDI (SEQ ID NO: 34), the MHC class I epitope TAECKDKNL (SEQ ID NO: 35), the MHC class II epitope VRYKCNCGG (SEQ ID NO: 36), a measles virus hemagglutinin protein MV-H antigen, a measles virus fusion protein MV-F antigen, a MERS-CoV S protein antigen, an antigen from the receptor-binding domain of the MERS-CoV S protein, an antigen from the membrane fusion domain of the MERS-CoV S protein, a SARS-CoV S protein antigen, an antigen from the receptor binding domain of the SARS-CoV S protein, an antigen from the membrane fusion domain of the SARS-CoV S protein, a SARS-CoV E protein antigen, a SARS-CoV M protein antigen, a Toxoplasma gondii MIC8 antigen, a Plasmodium falciparum: SERA5 polypeptide antigen, a Plasmodium falciparum: circumsporozite protein antigen, an influenza virus hemagglutinin (HA) antigen, an influenza virus neuraminidase (NA) antigen, an influenza virus matrix-2 (M2) protein antigen, antigenic fragments thereof, and any combination thereof.

In some aspects, the first antigen nucleic acid encodes a SARS CoV-2 spike (S) protein or an antigenic fragment thereof. In some aspects, the second pathogen protein or antigenic fragment thereof is selected from the group consisting of: a SARS-CoV-2 membrane (M) protein or an antigenic fragment thereof, a SARS-CoV-2 envelope (E) protein or an antigenic fragment thereof, a SARS-CoV-2 nucleocapsid (N) protein or an antigenic fragment thereof, and any combination thereof. In some aspects, the SARS-CoV-2 S protein or antigenic fragment thereof and the second pathogen protein or antigenic fragment thereof are derived from different strains of SARS-CoV-2. In some aspects, the first antigen nucleic acid encodes a SARS CoV-2 S protein or antigenic fragment thereof and a second SARS CoV-2 S protein or antigenic fragment thereof from a different strain of SARS-CoV-2.

In some aspects, the second antigen nucleic acid is operably linked to the first promoter through an internal ribosome entry site (IRES) sequence. In some aspects, the IRES sequence comprises a nucleic acid sequence having at least 70%, at least 75%, at least 80%, at least 85%, at least 90%, at least 95%, at least 99%, or 100% sequence identity to SEQ ID NO: 41.

In some aspects, the polynucleotide comprises one or more second promoters. In some aspects, the second antigen nucleic acid is operably linked to the one or more second promoters.

In some aspects, the first promoter or the one or more second promoters is selected from the group consisting of: a cytomegalovirus (CMV) promoter, a Rouse sarcoma virus (RSV) promoter, a Moloney murine leukemia virus (Mo-MuLV) long terminal repeat (LTR) promoter, a mammalian elongation factor 1 (EF1) promoter, a cytokeratin 18 (CK18) promoter, a cytokeratin 19 (CK19) promoter, a simian virus 40 (SV40) promoter, a murine U6 promoter, a skeletal α-actin promoter, a β-actin promoter, a murine phosphoglycerate kinase 1 (PGK1) promoter, a human PGK1 promoter, a CBA promoter, a CAG promoter, and any combination thereof. In some aspects, the mammalian EF1 promoter is a hEF1-HTLV promoter. In some aspects, the one or more second promoters is the CMV promoter.

In some aspects, the second antigen nucleic acid is under the control of a promoter selected from the group consisting of a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CBA promoter, a CAG promoter, and any combination thereof. In some aspects, the mammalian EF1 promoter is a hEF1-HTLV promoter.

In some aspects, the first antigen nucleic acid encodes a full-length SARS-CoV-2 S protein or an antigenic fragment thereof. In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the first antigen nucleic acid encodes a polypeptide comprising the amino acid sequence of SEQ ID NO: 2 or SEQ ID NO: 4. In some aspects, the first antigen nucleic acid comprises a nucleic acid sequence having at least 70%, at least 75%, at least 80%, at least 85%, at least 90%, at least 95%, at least 99%, or 100% sequence identity to SEQ ID NO: 1 or SEQ ID NO: 3. In some aspects, the first antigen nucleic acid is operably linked to a mammalian EF1 promoter. In some aspects, the mammalian EF1 promoter is a hEF1-HTLV promoter.

In some aspects, the second antigen nucleic acid encodes a full-length SARS-CoV-2 S protein or an antigenic fragment thereof. In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the second antigen nucleic acid encodes a polypeptide comprising the amino acid sequence of SEQ ID NO: 2 or SEQ ID NO: 4. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 1 or SEQ ID NO: 3. In some aspects, the second antigen nucleic acid is operably linked to a mammalian EF1 promoter through an IRES sequence. In some aspects, the mammalian EF1 promoter is a hEF1-HTLV promoter.

In some aspects, the first antigen nucleic acid encodes a full-length SARS-CoV-2 S protein or an antigenic fragment thereof, and wherein the second antigen nucleic acid encodes a SARS-CoV-2 membrane (M) protein or an antigenic fragment thereof. In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4, and wherein the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the first antigen nucleic acid encodes a polypeptide comprising the amino acid sequence of SEQ ID NO: 2 or SEQ ID NO: 4, and wherein the second antigen nucleic acid encodes a polypeptide comprising the amino acid sequence of SEQ ID NO: 8, SEQ ID NO: 10 SEQ ID NO: 12, SEQ ID NO: 14, SEQ ID NO: 16, SEQ ID NO: 18, or SEQ ID NO: 20. In some aspects, the first antigen nucleic acid comprises a nucleic acid sequence having at least 70%, at least 75%, at least 80%, at least 85%, at least 90%, at least 95%, at least 99%, or 100% sequence identity to SEQ ID NO: 1 or SEQ ID NO: 3, and wherein the second antigen nucleic acid comprises a nucleic acid sequence having at least 70%, at least 75%, at least 80%, at least 85%, at least 90%, at least 95%, at least 99%, or 100% sequence identity to SEQ ID NO: 7, SEQ ID NO: 9, SEQ ID NO: 11, SEQ ID NO: 13, SEQ ID NO: 15, SEQ ID NO: 17, or SEQ ID NO: 19. In some aspects, the first antigen nucleic acid is operably linked to a mammalian EF1 promoter, and wherein the second antigen nucleic acid is operably linked to a CMV promoter. In some aspects, the mammalian EF1 promoter is a hEF1-HTLV promoter.

In some aspects, the first antigen nucleic acid encodes the receptor binding domain (RBD) of the SARS-CoV-2 S protein or an antigenic fragment thereof. In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the first antigen nucleic acid encodes a polypeptide comprising the amino acid sequence of SEQ ID NO: 6. In some aspects, the first antigen nucleic acid comprises a nucleic acid sequence having at least 70%, at least 75%, at least 80%, at least 85%, at least 90%, at least 95%, at least 99%, or 100% sequence identity to SEQ ID NO: 5.

In some aspects, the second antigen nucleic acid encodes the receptor binding domain (RBD) of the SARS-CoV-2 S protein or an antigenic fragment thereof. In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the second antigen nucleic acid encodes a polypeptide comprising the amino acid sequence of SEQ ID NO: 6. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 5.

In some aspects, the first antigen nucleic acid encodes the S1 subunit of the SARS-CoV-2 S protein or an antigenic fragment thereof. In some aspects, the first antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the first antigen nucleic acid encodes a polypeptide comprising the amino acid sequence of SEQ ID NO: 40. In some aspects, the first antigen nucleic acid comprises a nucleic acid sequence having at least 70%, at least 75%, at least 80%, at least 85%, at least 90%, at least 95%, at least 99%, or 100% sequence identity to SEQ ID NO: 39. In some aspects, the first antigen nucleic acid is operably linked to a mammalian EF1 promoter. In some aspects, the mammalian EF1 promoter is a hEF1-HTLV promoter.

In some aspects, the second antigen nucleic acid encodes the S1 subunit of the SARS-CoV-2 S protein or an antigenic fragment thereof. In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the second antigen nucleic acid encodes a polypeptide comprising the amino acid sequence of SEQ ID NO: 40. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 39. In some aspects, the second antigen nucleic acid is operably linked to a mammalian EF1 promoter through an TRES sequence. In some aspects, the mammalian EF1 promoter is a hEF1-HTLV promoter.

In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the second antigen nucleic acid encodes a polypeptide comprising the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 7, SEQ ID NO: 9, SEQ ID NO: 11, SEQ ID NO: 13, SEQ ID NO: 15, SEQ ID NO: 17, or SEQ ID NO: 19. In some aspects, the second antigen nucleic acid encodes at least 8, at least 10, at least 15, at least 20, at least 25, at least 30, at least 35, at least 40, at least 45, at least 50, at least 55, at least 60, at least 65, at least 70, or at least 75 contiguous amino acids of SEQ ID NO: 22, SEQ ID NO: 24, or SEQ ID NO: 26. In some aspects, the second antigen nucleic acid encodes a polypeptide comprising the amino acid sequence of SEQ ID NO: 22, SEQ ID NO: 24, or SEQ ID NO: 26. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 21, SEQ ID NO: 23, or SEQ ID NO: 25. In some aspects, the second antigen nucleic acid encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, or at least 400 contiguous amino acids of SEQ ID NO: 28. In some aspects, the second antigen nucleic acid encodes a polypeptide comprising the amino acid sequence of SEQ ID NO: 28. In some aspects, the second antigen nucleic acid comprises a nucleic acid sequence having at least 70%, at least 75%, at least 80%, at least 85%, at least 90%, at least 95%, at least 99%, or 100% sequence identity to SEQ ID NO: 27.

In some aspects, the polypeptide(s) encoded by the first antigen nucleic acid (e.g., a first antigen nucleic acid encoding a SARS-CoV-2 full-length S protein, the RBD of a SARS-Cov-2 S protein, or the S1 subunit of a SARS-CoV-2 S protein) and/or the second antigen nucleic acid (e.g., a first antigen nucleic acid encoding a SARS-CoV-2 full-length S protein, the RBD of a SARS-Cov-2 S protein, or the S1 subunit of a SARS-CoV-2 S protein) comprise one or more mutations. In some aspects, the one or more mutations in the polypeptide(s) encoded by the first antigen nucleic acid or the second antigen nucleic acid comprise one or more mutations previously reported in Li, T. et al., Emerg Microbes Infect. 9(1):2076-90 (2020); Lee, P. et al., Immune Netw. 21(1):e4 (2021); Yu, J. et al., Science 369(6505):806-11 (2020); Cattin-Ortola, J. et al., Nat Commun. 12(1):5333 (2021); Corbett, K. et al., Nature 586(7830):567-71 (2020); Hsieh, C. et al., Science 369(6510):1501-5 (2020); and Harvey, W. et al., Nat Rev Microbiol. 19(7):409-24 (2021), each of which is incorporated by reference herein in its entirety.

In some aspects, the one or more mutations in the polypeptide(s) encoded by the first antigen nucleic acid and/or the second antigen nucleic acid are selected from: ΔM1-S13, S12P, S131, L5F, L18F, T19R, T20N, P26S, Q52R, A67V, ΔH69-V70, G75V, T76I, D80A, T95I, R102I, ΔD119-F120, C136Y, D138Y, ΔF140, ΔL141-Y144, AY144, Y144S, Y145N, ΔH146, N148S, K150R, K150E, K150T, K150Q, S151P, W152C, E154K, ΔE156-F157, F157L, F157A, R158G, R190S, ΔI210, D215G, A222V, ΔL241-S243, ΔL242-L244, ΔA243-L244, ΔR246-G252, R246I, 11-amino acid residue insertion between Y248 and L249, D253G, D253N, R346K, V367F, E406W, K417E, K417V, K417N, K417T, N439K, K444A, K444R, K444N, K444Q, V445A, V445E, G447A, N450D, L452R, L452Q, Y453F, L455F, N460I, S477G, S477N, S477R, T478I, T478K, V483I, E484K, E484Q, G485R, F486A, F486V, F486L, N487A, F490S, Q493E, Q493K, S494P, N501Y, A570D, Q613H, D614G, H655Y, Q677H, Δ678-679, Δ681-682, Δ681-684, Δ682-685, P681H, P681R, R682S, R682A, R682Q, R683S, R683G, R683Q, R685G, R685Q, I692V, A701V, T716I, F817P, T859N, F888L, A892P, A899P, A942P, D950N, S982A, K986P, V987P, T1027I, Q1071H, D1118H, V1176F, M1229I, ΔC1253-T1273, ΔC1254-T1273, ΔK1255-T1273, D1257A, E1258A, D1259A, D1260A, E1262A, K1269A, H1271K, T1273A, or any combination thereof, wherein the amino acid locations correspond to SEQ ID NO: 2 or SEQ ID NO: 4.

In some aspects, the one or more mutations in the polypeptide(s) encoded by the first antigen nucleic acid and/or the second antigen nucleic acid comprise one or more mutations in the N-terminal signal peptide of the full-length SARS-CoV-2 S protein, which corresponds to amino acids 1-13 of SEQ ID NO: 2 or SEQ ID NO: 4. In some aspects, the one or more mutations in the N-terminal signal peptide is ΔM1-S13, wherein the amino acid locations correspond to SEQ ID NO: 2 or SEQ ID NO: 4.

In some aspects, the one or mutations in the polypeptide(s) encoded by the first antigen nucleic acid and/or the second antigen nucleic acid comprise one or more mutations in the C-terminus of the full-length SARS-CoV-2 S protein. In some aspects, the one or more mutations in the C-terminus of the full-length SARS-CoV-2 S protein comprise one or more mutations in the C-terminal endoplasmic reticulum (ER) retention peptide, which corresponds to amino acids 1254-1273 of SEQ ID NO: 2 or SEQ ID NO: 4. In some aspects, the one or more mutations in the C-terminal ER retention peptide are selected from D1257A, E1258A, D1259A, D1260A, E1262A, K1269A, H1271K, T1273A, or any combination thereof, wherein the amino acid locations correspond to SEQ ID NO: 2 or SEQ ID NO: 4. In some aspects, the one or more mutations in the C-terminal ER retention peptide comprise D1257A+E1258A+D1259A+D1260A+E1262A (i.e., a D/E to A mutant), wherein the amino acid locations correspond to SEQ ID NO: 2 or SEQ ID NO: 4. In some aspects, the one or mutations in the C-terminal ER retention peptide is ΔC1253-T1273, ΔC1254-T1273, or ΔK1255-T1273.

In some aspects, the one or more mutations in the polypeptide(s) encoded by the first antigen nucleic acid and/or the second antigen nucleic acid comprise K986P+V987P (i.e., a S-2P mutation), wherein the amino acid locations correspond to SEQ ID NO: 2 or SEQ ID NO: 4. In some aspects, the one or more mutations in the polypeptide(s) encoded by the first antigen nucleic acid and/or the second antigen nucleic acid comprise F817P+A892P+A899P+A942P (i.e., a hexa-proline S mutation), wherein the amino acid locations correspond to SEQ ID NO: 2 or SEQ ID NO: 4. In some aspects, the one or more mutations in the polypeptide(s) encoded by the first antigen nucleic acid and/or the second antigen nucleic acid comprise one or more mutations in the 681-PRRAR/SVA-688 S1/S2 furin cleavage site, wherein the amino acid locations correspond to SEQ ID NO: 2 or SEQ ID NO: 4. In some aspects, the one or more mutations in the 681-PRRAR/SVA-688 S1/S2 furin cleavage site are: (a) R682S+R683S (i.e., a SSAR mutation), (b) Δ681-684 (i.e., a ΔPRRA mutation), (c) Δ678-679+Δ681-682, (d) R682A+R683G+R685G (i.e., a 682-AGAG-685 mutation), (e) R682Q+R683Q+R685Q, (f) R682S+R685G, or (g) Δ682-685 (i.e., a ΔRRAR mutation), wherein the amino acid locations correspond to SEQ ID NO: 2 or SEQ ID NO: 4.

In some aspects, the one or more mutations in the polypeptide(s) encoded by the first antigen nucleic acid and/or the second antigen nucleic acid comprise: (a) F817P+A892P+A899P+A942P (i.e., a hexa-proline S mutation) and (b) K986P+V987P (i.e., a S-2P mutation), wherein the amino acid locations correspond to SEQ ID NO: 2 or SEQ ID NO: 4. In some aspects, the one or more mutations in the polypeptide(s) encoded by the first antigen nucleic acid and/or the second antigen nucleic acid comprise: (a) R682A+R683G+R685G (i.e., a 682-AGAG-685 mutation) and (b) K986P+V987P (i.e., a S-2P mutation), wherein the amino acid locations correspond to SEQ ID NO: 2 or SEQ ID NO: 4. In some aspects, the one or more mutations in the polypeptide(s) encoded by the first antigen nucleic acid and/or the second antigen nucleic acid comprise: (a) R682A+R683G+R685G (i.e., a 682-AGAG-685 mutation), (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations in the polypeptide(s) encoded by the first antigen nucleic acid and/or the second antigen nucleic acid comprise: (a) R682Q+R683Q+R685Q and (b) K986P+V987P (i.e., a S-2P mutation). In some aspects, the one or more mutations in the polypeptide(s) encoded by the first antigen nucleic acid and/or the second antigen nucleic acid comprise:: (a) R682Q+R683Q+R685Q, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations in the polypeptide(s) encoded by the first antigen nucleic acid and/or the second antigen nucleic acid comprise: (a) R682S+R685G and (b) K986P+V987P (i.e., a S-2P mutation), wherein the amino acid locations correspond to SEQ ID NO: 2 or SEQ ID NO: 4. In some aspects, the one or more mutations in the polypeptide(s) encoded by the first antigen nucleic acid and/or the second antigen nucleic acid comprise: (a) R682S+R685G, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4.

The polynucleotides disclosed herein can further comprise one or more post-transcriptional regulatory elements. In some aspects, the post-translational regulatory element is positioned 3′ to a coding region of the polynucleotide. Non-limiting examples of post-transcriptional regulatory elements that are useful for the present disclosure include a mutated woodchuck hepatitis virus post-transcriptional regulatory element (WPRE), microRNA binding site, DNA nuclear targeting sequence, or combinations thereof. In some aspects, the post-transcriptional regulatory element is a WPRE.

The polynucleotide can also comprise one or more polyadenylation (poly(a)) signals, which can be downstream of any protein coding sequence. Examples of polyadenylation signals include but are not limited to a SV40 poly(a) tail (SEQ ID NO: 29), LTR poly(a) tail, bovine growth hormone (bGH) poly(a) tail (SEQ ID NO: 30), human growth hormone (hGH) poly(a) tail, or human β-globin poly(a) tail. The SV40 polyadenylation signal can be a polyadenylation signal from a pCEP4 vector (Invitrogen, San Diego, CA). In some aspects, the polynucleotides disclosed herein further comprise at least one 3′ UTR poly(a) tail sequence operably linked to the first antigen nucleic acid, the second antigen nucleic acid, the nucleic acid encoding an immune modifier, or any combination thereof. In some aspects, the 3′ UTR poly(a) tail sequence is a 3′ UTR SV40 poly(a) tail sequence, a 3′ UTR bovine growth hormone (bGH) poly(A) sequence, a 3′ UTR actin poly(A) tail sequence, a 3′ UTR hemoglobin poly(A) sequence, or combinations thereof.

The polynucleotides disclosed herein can further comprise at least one enhancer sequence upstream of any protein coding sequence. The enhancer can be necessary for DNA expression. In some aspects, the enhancer is a human actin enhancer, human myosin enhancer, human hemoglobin enhancer, human muscle creatine enhancer, or a viral enhancer such as one from CMV, HA, RSV or EBV. In some aspects, the enhancer is a polynucleotide function enhancer as described in U.S. Pat. Nos. 5,593,972, 5,962,428, and WO 94/016737. In some aspects, the enhancer sequence is a CMV intronic sequence or a β-actin intronic sequence. In aspects, the enhancer sequence is a SV40 enhancer sequence (SEQ ID NO: 37).

The polynucleotides disclosed herein can further comprise one or more inverted terminal repeats (ITRs). In some aspects, the polynucleotide comprises a first ITR and a second ITR. In some aspects, the polynucleotide comprises a first ITR, e.g., a 5′ ITR, and a second ITR, e.g., a 3′ ITR. Typically, ITRs are involved in parvovirus (e.g., adeno-associated virus (AAV)) DNA replication and rescue, or excision, from prokaryotic plasmids (Samulski et al., 1983, 1987; Senapathy et al., 1984; Gottlieb and Muzyczka, 1988). In addition, ITRs appear to be the minimum sequences required for AAV proviral integration and for packaging of AAV DNA into virions (McLaughlin et al., 1988; Samulski et al., 1989). These elements are essential for efficient multiplication of a parvovirus genome. In some aspects, the ITRs fold into a hairpin T-shaped structure. In some aspects, the ITRs fold into non-T-shaped hairpin structures, e.g., into a U-shaped hairpin structure.

In some aspects, the ITRs that are useful for the present disclosure comprise an ITR from an AAV genome. In certain aspects, the ITR is an ITR of an AAV genome selected from AAV1, AAV2, AAV3, AAV4, AAV5, AAV6, AAV7, AAV8, AAV9, AAV10, AAV11, and any combination thereof. In some aspects, the ITR is an ITR of the AAV2 genome. In some aspects, the ITR is a synthetic sequence genetically engineered to include at its 5′ and 3′ ends ITRs derived from one or more of AAV genomes.

In some aspects, the ITR is not derived from an AAV genome. In some aspects, the ITR is an ITR of a non-AAV. In some aspects, the ITR is an ITR of a non-AAV genome from the viral family Parvoviridae selected from, but not limited to, the group consisting of Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, Penstyldensovirus and any combination thereof. In certain aspects, the ITR is derived from erythrovirus parvovirus B19 (human virus). In some aspects, the ITR is derived from a Muscovy duck parvovirus (MDPV) strain. In certain aspects, the MDPV strain is attenuated, e.g., MDPV strain FZ91-30. In some aspects, the MDPV strain is pathogenic, e.g., MDPV strain YY. In some aspects, the ITR is derived from a porcine parvovirus, e.g., porcine parvovirus U44978. In some aspects, the ITR is derived from a mice minute virus, e.g., mice minute virus U34256. In some aspects, the ITR is derived from a canine parvovirus, e.g., canine parvovirus M19296. In some aspects, the ITR is derived from a mink enteritis virus, e.g., mink enteritis virus D00765. In some aspects, the ITR is derived from a Dependoparvovirus. In certain aspects, the Dependoparvovirus is a Dependovirus Goose parvovirus (GPV) strain. In some aspects, the GPV strain is attenuated, e.g., GPV strain 82-0321V. In some aspects, the GPV strain is pathogenic, e.g., GPV strain.

The polynucleotides disclosed herein can also comprise a mammalian origin of replication (e.g., an Epstein Barr virus origin of replication) in order to maintain the vector extrachromosomally and produce multiple copies of the vector in a cell.

In some aspects, the polynucleotide is a multicistronic mRNA. In some aspects, the multicistronic mRNA comprises a 5′ cap and a 3′ UTR poly(A) tail sequence. In some aspects, the 3′ UTR poly(a) tail sequence is a 3′ UTR SV40 poly(a) tail sequence (SEQ ID NO: 29), a 3′ UTR bovine growth hormone (bGH) poly(A) sequence (SEQ ID NO: 30), a 3′ UTR actin poly(A) tail sequence, a 3′ UTR hemoglobin poly(A) sequence, or any combinations thereof. In some aspects, the multicistronic mRNA comprises a 5′ UTR and/or a 3′ UTR.

In some aspects, the polynucleotides disclosed herein can comprise: a first nucleotide sequence, wherein the first nucleotide sequence encodes IL-12 p35 and is operably linked to a CMV promoter; a second nucleotide sequence, wherein the second nucleotide sequence encodes IL-12 p40 and is operably linked to a CMV promoter; a third nucleotide sequence, wherein the third nucleotide sequence encodes a first pathogen protein (e.g., a SARS-CoV-2 protein) and is operably linked to promoter 1; and a fourth nucleotide sequence, wherein the fourth nucleotide sequence encodes a second pathogen protein (e.g., a SARS-CoV-2 protein) and is operably linked to promoter 2. For example, in some aspects, the polynucleotide comprises: a 5′ first nucleotide sequence, wherein the first nucleotide sequence encodes IL-12 p35 and is operably linked to a CMV promoter; a second nucleotide sequence positioned 3′ to the first nucleotide sequence, wherein the second nucleotide sequence encodes IL-12 p40 and is operably linked to a CMV promoter; a third nucleotide sequence positioned 3′ to the second nucleotide sequence, wherein the third nucleotide sequence encodes a first pathogen protein (e.g., a SARS-CoV-2 protein) and is operably linked to promoter 1; and a fourth nucleotide sequence positioned 3′ to the third nucleotide sequence, wherein the fourth nucleotide sequence encodes a second pathogen protein (e.g., a SARS-CoV-2 protein) and is operably linked to promoter 2. In some aspects, the first, second, third, and fourth nucleotide sequences of the polynucleotide are configured as shown in FIG. 1.

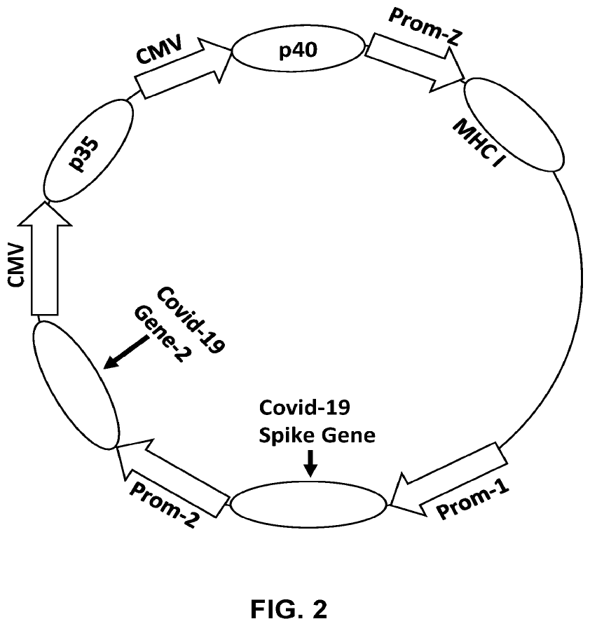

In some aspects, the polynucleotides disclosed herein can comprise: a first nucleotide sequence, wherein the first nucleotide sequence encodes IL-12 p35 and is operably linked to a CMV promoter; a second nucleotide sequence, wherein the second nucleotide sequence encodes IL-12 p40 and is operably linked to a CMV promoter; a third nucleotide sequence, wherein the third nucleotide sequence encodes MHC I and is operably linked to promoter Z; a fourth nucleotide sequence, wherein the fourth nucleotide sequence encodes a first pathogen protein (e.g., a SARS-CoV-2 protein) and is operably linked to promoter 1, and a fifth nucleotide sequence, wherein the fifth nucleotide sequence encodes a second pathogen protein (e.g., a SARS-CoV-2 protein) and is operably linked to promoter 2. For example, in some aspects, the polynucleotide comprises: a 5′ first nucleotide sequence, wherein the first nucleotide sequence encodes IL-12 p35 and is operably linked to a CMV promoter; a second nucleotide sequence positioned 3′ to the first nucleotide sequence, wherein the second nucleotide sequence encodes IL-12 p40 and is operably linked to a CMV promoter; a third nucleotide sequence positioned 3′ to the second nucleotide sequence, wherein the third nucleotide sequence encodes MHC I and is operably linked to promoter Z; a fourth nucleotide sequence positioned 3′ to the third nucleotide sequence, wherein the fourth nucleotide sequence encodes a first pathogen protein (e.g., a SARS-CoV-2 protein) and is operably linked to promoter 1, and a fifth nucleotide sequence positioned 3′ to the fourth nucleotide sequence, wherein the fifth nucleotide sequence encodes a second pathogen protein (e.g., a SARS-CoV-2 protein) and is operably linked to promoter 2. In some aspects, the first, second, third, fourth, and fifth nucleotide sequences of the polynucleotide are configured as shown in FIG. 2.

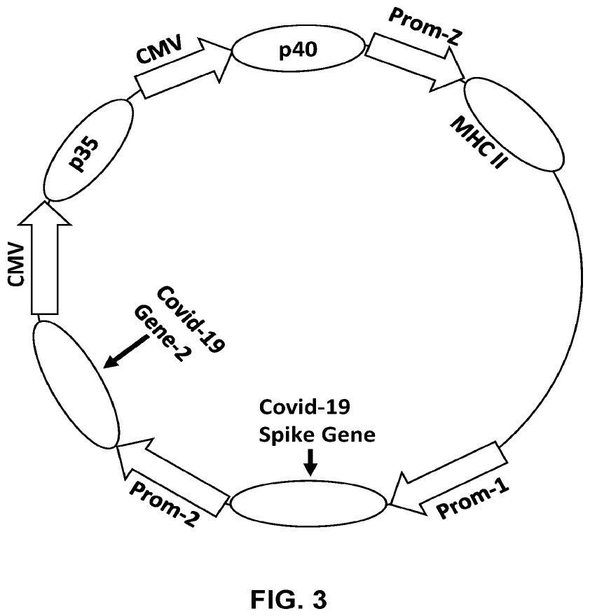

In some aspects, the polynucleotides disclosed herein can comprises: a first nucleotide sequence, wherein the first nucleotide sequence encodes IL-12 p35 and is operably linked to a CMV promoter; a second nucleotide sequence, wherein the second nucleotide sequence encodes IL-12 p40 and is operably linked to a CMV promoter; a third nucleotide sequence, wherein the third nucleotide sequence encodes MHC II and is operably linked to promoter Z; a fourth nucleotide sequence, wherein the fourth nucleotide sequence encodes a first pathogen protein (e.g., a SARS-CoV-2 protein) and is operably linked to promoter 1, and a fifth nucleotide sequence, wherein the fifth nucleotide sequence encodes a second pathogen protein (e.g., a SARS-CoV-2 protein) and is operably linked to promoter 2. For example, in some aspects, the polynucleotide comprises: a 5′ first nucleotide sequence, wherein the first nucleotide sequence encodes IL-12 p35 and is operably linked to a CMV promoter; a second nucleotide sequence positioned 3′ to the first nucleotide sequence, wherein the second nucleotide sequence encodes IL-12 p40 and is operably linked to a CMV promoter; a third nucleotide sequence positioned 3′ to the second nucleotide sequence, wherein the third nucleotide sequence encodes MHC II and is operably linked to promoter Z; a fourth nucleotide sequence positioned 3′ to the third nucleotide sequence, wherein the fourth nucleotide sequence encodes a first pathogen protein (e.g., a SARS-CoV-2 protein) and is operably linked to promoter 1, and a fifth nucleotide sequence positioned 3′ to the fourth nucleotide sequence, wherein the fifth nucleotide sequence encodes a second pathogen protein (e.g., a SARS-CoV-2 protein) and is operably linked to promoter 2. In some aspects, the first, second, third, fourth, and fifth nucleotide sequences of the polynucleotide are configured as shown in FIG. 3.

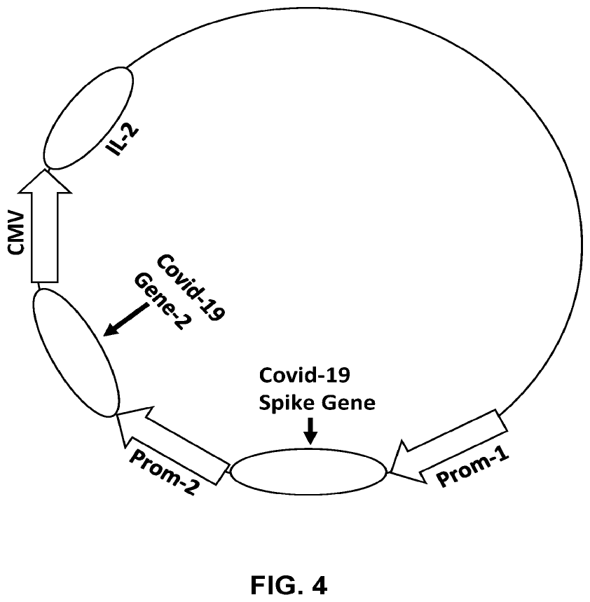

In some aspects, the polynucleotides disclosed herein can comprise: a first nucleotide sequence, wherein the first nucleotide sequence encodes IL-2 and is operably linked to a CMV promoter; a second nucleotide sequence, wherein the second nucleotide sequence encodes a first pathogen protein (e.g., a SARS-CoV-2 protein) and is operably linked to promoter 1; and a third nucleotide sequence, wherein the third nucleotide sequence encodes a second pathogen protein (e.g., a SARS-CoV-2 protein) and is operably linked to promoter 2. For example, in some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes a first pathogen protein (e.g., a SARS-CoV-2 protein) and is operably linked to promoter 1; and a third nucleotide sequence positioned 3′ to the second nucleotide sequence, wherein the third nucleotide sequence encodes a second pathogen protein (e.g., a SARS-CoV-2 protein) and is operably linked to promoter 2. In some aspects, the first, second, and third nucleotide sequences of the polynucleotide are configured as shown in FIG. 4.

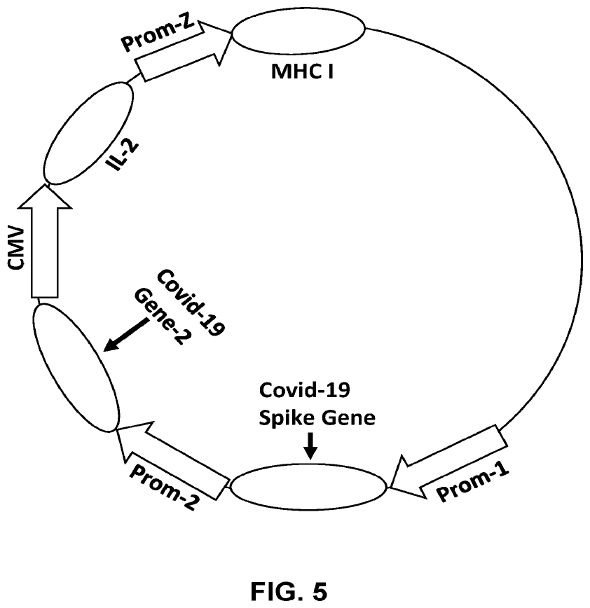

In some aspects, the polynucleotides disclosed herein can comprise: a first nucleotide sequence, wherein the first nucleotide sequence encodes IL-2 and is operably linked to a CMV promoter; a second nucleotide sequence, wherein the second nucleotide sequence encodes MHC I and is operably linked to promoter Z; a third nucleotide sequence, wherein the third nucleotide sequence encodes a first pathogen protein (e.g., a SARS-CoV-2 protein) and is operably linked to promoter 1; and a fourth nucleotide sequence, wherein the fourth nucleotide sequence encodes a second pathogen protein (e.g., a SARS-CoV-2 protein) and is operably linked to promoter 2. For example, in some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes MHC I and is operably linked to promoter Z; a third nucleotide sequence positioned 3′ to the second nucleotide sequence, wherein the third nucleotide sequence encodes a first pathogen protein (e.g., a SARS-CoV-2 protein) and is operably linked to promoter 1; and a fourth nucleotide sequence positioned 3′ to the third nucleotide sequence, wherein the fourth nucleotide sequence encodes a second pathogen protein (e.g., a SARS-CoV-2 protein) and is operably linked to promoter 2. In some aspects, the first, second, third, and fourth nucleotide sequences of the polynucleotide are configured as shown in FIG. 5.

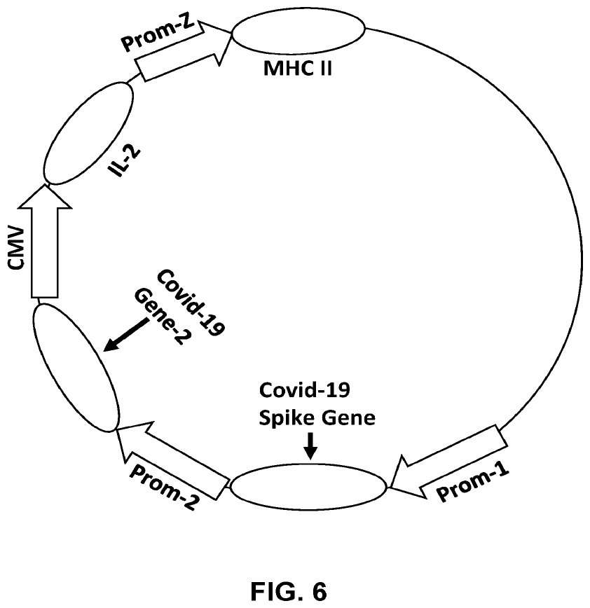

In some aspects, the polynucleotides disclosed herein can comprise: a first nucleotide sequence, wherein the first nucleotide sequence encodes IL-2 and is operably linked to a CMV promoter; a second nucleotide sequence, wherein the second nucleotide sequence encodes MHC II and is operably linked to promoter Z; a third nucleotide sequence, wherein the third nucleotide sequence encodes a first pathogen protein (e.g., a SARS-CoV-2 protein) and is operably linked to promoter 1; and a fourth nucleotide sequence, wherein the fourth nucleotide sequence encodes a second pathogen protein (e.g., a SARS-CoV-2 protein) and is operably linked to promoter 2. For example, in some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes MHC II and is operably linked to promoter Z; a third nucleotide sequence positioned 3′ to the second nucleotide sequence, wherein the third nucleotide sequence encodes a first pathogen protein (e.g., a SARS-CoV-2 protein) and is operably linked to promoter 1; and a fourth nucleotide sequence positioned 3′ to the third nucleotide sequence, wherein the fourth nucleotide sequence encodes a second pathogen protein (e.g., a SARS-CoV-2 protein) and is operably linked to promoter 2. In some aspects, the first, second, third, and fourth nucleotide sequences of the polynucleotide are configured as shown in FIG. 6.

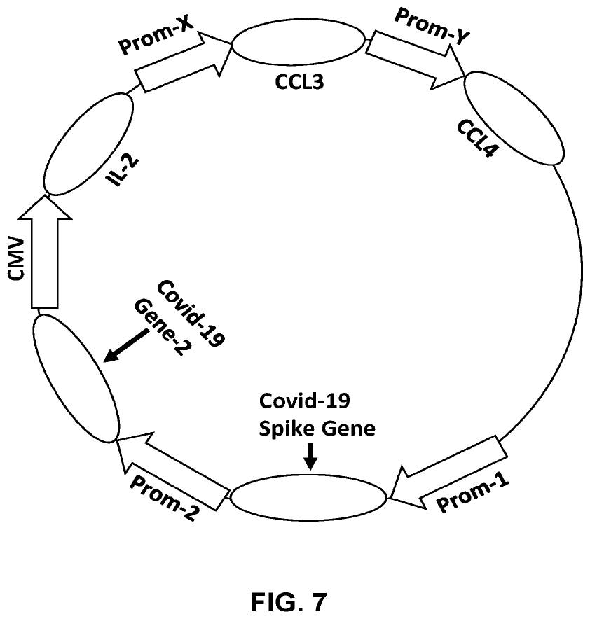

In some aspects, the polynucleotides disclosed herein can comprise: a first nucleotide sequence, wherein the first nucleotide sequence encodes IL-2 and is operably linked to a CMV promoter; a second nucleotide sequence, wherein the second nucleotide sequence encodes CCL3 and is operably linked to promoter X; a third nucleotide sequence, wherein the third nucleotide sequence encodes CCL4 and is operably linked to promoter Y; a fourth nucleotide sequence, wherein the fourth nucleotide sequence encodes a first pathogen protein (e.g., a SARS-CoV-2 protein) and is operably linked to promoter 1, and a fifth nucleotide sequence, wherein the fifth nucleotide sequence encodes a second pathogen protein (e.g., a SARS-CoV-2 protein) and is operably linked to promoter 2. For example, in some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes CCL3 and is operably linked to promoter X; a third nucleotide sequence positioned 3′ to the second nucleotide sequence, wherein the third nucleotide sequence encodes CCL4 and is operably linked to promoter Y; a fourth nucleotide sequence positioned 3′ to the third nucleotide sequence, wherein the fourth nucleotide sequence encodes a first pathogen protein (e.g., a SARS-CoV-2 protein) and is operably linked to promoter 1, and a fifth nucleotide sequence positioned 3′ to the fourth nucleotide sequence, wherein the fifth nucleotide sequence encodes a second pathogen protein (e.g., a SARS-CoV-2 protein) and is operably linked to promoter 2. In some aspects, the first, second, third, fourth, and fifth nucleotide sequences of the polynucleotide are configured as shown in FIG. 7.

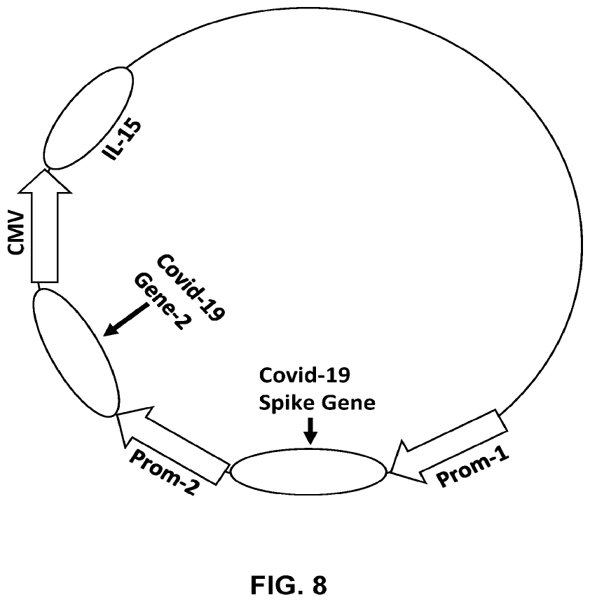

In some aspects, the polynucleotides disclosed herein can comprise: a first nucleotide sequence, wherein the first nucleotide sequence encodes IL-15 and is operably linked to a CMV promoter; a second nucleotide sequence, wherein the second nucleotide sequence encodes a first pathogen protein (e.g., a SARS-CoV-2 protein) and is operably linked to promoter 1; and a third nucleotide sequence, wherein the third nucleotide sequence encodes a second pathogen protein (e.g., a SARS-CoV-2 protein) and is operably linked to promoter 2. For example, in some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes a first pathogen protein (e.g., a SARS-CoV-2 protein) and is operably linked to promoter 1; and a third nucleotide sequence positioned 3′ to the second nucleotide sequence, wherein the third nucleotide sequence encodes a second pathogen protein (e.g., a SARS-CoV-2 protein) and is operably linked to promoter 2. In some aspects, the first, second, and third nucleotide sequences of the polynucleotide are configured as shown in FIG. 8.

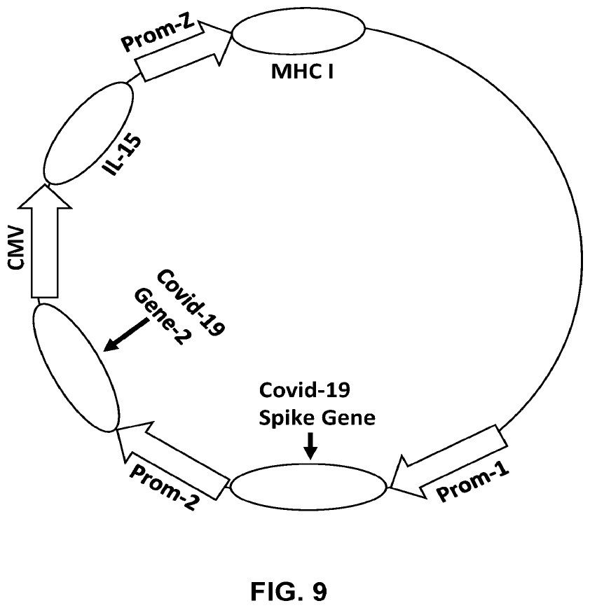

In some aspects, the polynucleotides disclosed herein can comprise: a first nucleotide sequence, wherein the first nucleotide sequence encodes IL-15 and is operably linked to a CMV promoter; a second nucleotide sequence, wherein the second nucleotide sequence encodes MHC I and is operably linked to promoter Z; a third nucleotide sequence, wherein the third nucleotide sequence encodes a first pathogen protein (e.g., a SARS-CoV-2 protein) and is operably linked to promoter 1; and a fourth nucleotide sequence, wherein the fourth nucleotide sequence encodes a second pathogen protein (e.g., a SARS-CoV-2 protein) and is operably linked to promoter 2. For example, in some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes MHC I and is operably linked to promoter Z; a third nucleotide sequence positioned 3′ to the second nucleotide sequence, wherein the third nucleotide sequence encodes a first pathogen protein (e.g., a SARS-CoV-2 protein) and is operably linked to promoter 1; and a fourth nucleotide sequence positioned 3′ to the third nucleotide sequence, wherein the fourth nucleotide sequence encodes a second pathogen protein (e.g., a SARS-CoV-2 protein) and is operably linked to promoter 2. In some aspects, the first, second, third, and fourth nucleotide sequences of the polynucleotide are configured as shown in FIG. 9.

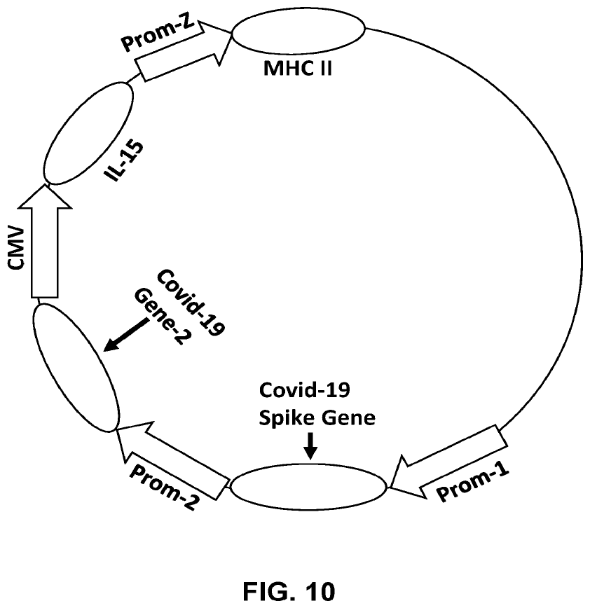

In some aspects, the polynucleotides disclosed herein can comprise: a first nucleotide sequence, wherein the first nucleotide sequence encodes IL-15 and is operably linked to a CMV promoter; a second nucleotide sequence, wherein the second nucleotide sequence encodes MHC II and is operably linked to promoter Z; a third nucleotide sequence, wherein the third nucleotide sequence encodes a first pathogen protein (e.g., a SARS-CoV-2 protein) and is operably linked to promoter 1; and a fourth nucleotide sequence, wherein the fourth nucleotide sequence encodes a second pathogen protein (e.g., a SARS-CoV-2 protein) and is operably linked to promoter 2. For example, in some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes MHC II and is operably linked to promoter Z; a third nucleotide sequence positioned 3′ to the second nucleotide sequence, wherein the third nucleotide sequence encodes a first pathogen protein (e.g., a SARS-CoV-2 protein) and is operably linked to promoter 1; and a fourth nucleotide sequence positioned 3′ to the third nucleotide sequence, wherein the fourth nucleotide sequence encodes a second pathogen protein (e.g., a SARS-CoV-2 protein) and is operably linked to promoter 2. In some aspects, the first, second, third, and fourth nucleotide sequences of the polynucleotide are configured as shown in FIG. 10.

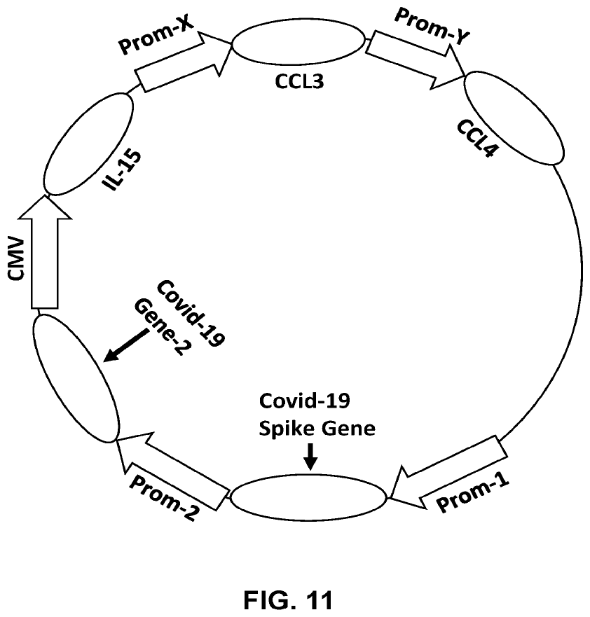

In some aspects, the polynucleotides disclosed herein can comprise: a first nucleotide sequence, wherein the first nucleotide sequence encodes IL-15 and is operably linked to a CMV promoter; a second nucleotide sequence, wherein the second nucleotide sequence encodes CCL3 and is operably linked to promoter X; a third nucleotide sequence, wherein the third nucleotide sequence encodes CCL4 and is operably linked to promoter Y; a fourth nucleotide sequence, wherein the fourth nucleotide sequence encodes a first pathogen protein (e.g., a SARS-CoV-2 protein) and is operably linked to promoter 1, and a fifth nucleotide sequence, wherein the fifth nucleotide sequence encodes a second pathogen protein (e.g., a SARS-CoV-2 protein) and is operably linked to promoter 2. For example, in some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes CCL3 and is operably linked to promoter X; a third nucleotide sequence positioned 3′ to the second nucleotide sequence, wherein the third nucleotide sequence encodes CCL4 and is operably linked to promoter Y; a fourth nucleotide sequence positioned 3′ to the third nucleotide sequence, wherein the fourth nucleotide sequence encodes a first pathogen protein (e.g., a SARS-CoV-2 protein) and is operably linked to promoter 1, and a fifth nucleotide sequence positioned 3′ to the fourth nucleotide sequence, wherein the fifth nucleotide sequence encodes a second pathogen protein (e.g., a SARS-CoV-2 protein) and is operably linked to promoter 2. In some aspects, the first, second, third, fourth, and fifth nucleotide sequences of the polynucleotide are configured as shown in FIG. 11.

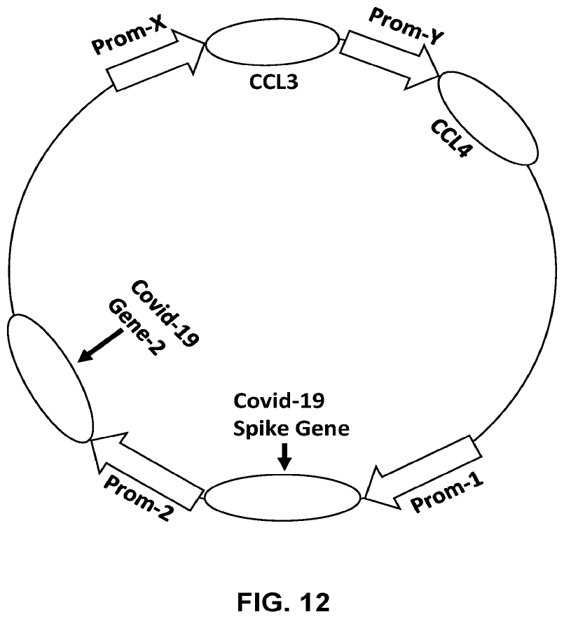

In some aspects, the polynucleotides disclosed herein can comprise: a first nucleotide sequence, wherein the first nucleotide sequence encodes CCL3 and is operably linked to promoter X; a second nucleotide sequence, wherein the second nucleotide sequence encodes CCL4 and is operably linked to promoter Y; a third nucleotide sequence, wherein the third nucleotide sequence encodes a first pathogen protein (e.g., a SARS-CoV-2 protein) and is operably linked to promoter 1; and a fourth nucleotide sequence, wherein the fourth nucleotide sequence encodes a second pathogen protein (e.g., a SARS-CoV-2 protein) and is operably linked to promoter 2. For example, in some aspects, the polynucleotide comprises: a 5′ first nucleotide sequence, wherein the first nucleotide sequence encodes CCL3 and is operably linked to promoter X; a second nucleotide sequence positioned 3′ to the first nucleotide sequence, wherein the second nucleotide sequence encodes CCL4 and is operably linked to promoter Y; a third nucleotide sequence positioned 3′ to the second nucleotide sequence, wherein the third nucleotide sequence encodes a first pathogen protein (e.g., a SARS-CoV-2 protein) and is operably linked to promoter 1; and a fourth nucleotide sequence positioned 3′ to the third nucleotide sequence, wherein the fourth nucleotide sequence encodes a second pathogen protein (e.g., a SARS-CoV-2 protein) and is operably linked to promoter 2. In some aspects, the first, second, third, and fourth nucleotide sequences of the polynucleotide are configured as shown in FIG. 12.

In some aspects, the vector constructs illustrated in any of FIGS. 1-12 can modified to replace the “Covid-19 Spike Gene” (a first nucleotide sequence encoding a SARS-CoV-2 protein) and the “Covid-19 Gene-2” (a second nucleotide sequence encoding a SARS-CoV-2 protein) with nucleotide sequences encoding any combinations of pathogen antigen or antigenic fragment thereof disclosed herein. In some aspect, the nucleotide sequences encode antigens to a virus, a bacteria or a parasite. In some aspects, the nucleotide sequences encode one or more antigens comprise one or more viral antigens, one or more bacterial antigens, or one or more parasite antigens. In some aspects the vector constructs disclosed herein can be used to express DNA or mRNA.

In some aspects, the polynucleotides disclosed herein can comprise: a nucleotide sequence, wherein the nucleotide sequence encodes a pathogen protein (e.g., a SARS-CoV-2 antigen) and the nucleotide sequence is operably linked to a promoter. In some aspects, the polynucleotide further comprises a nucleic acid sequence encoding one or more immune modifiers. In some aspects, the polynucleotide comprises a nucleic acid sequence encoding a SARS CoV-2 antigen and, optionally, a second viral antigen (e.g., a second SARS CoV-2 antigen and/or an influenza virus antigen).

In some aspects, the polynucleotides disclosed herein can comprise: a first nucleotide sequence, wherein the first nucleotide sequence encodes a first pathogen protein (e.g., a S1 subunit of the SARS-CoV-2 S protein) and is operably linked to a first promoter (e.g., a hEF1-HTLV promoter); a second nucleotide sequence, wherein the second nucleotide sequence encodes IL-12 p35 and is operably linked to a second promoter (e.g., a CMV promoter); and a third nucleotide sequence, wherein the third nucleotide sequence encodes IL-12 p40 and is operably linked to a second promoter (e.g., a CMV promoter). In some aspects, the first, second, and third nucleotide sequences of the polynucleotide are configured as shown in FIG. 14C (pVac 2).

In some aspects, the polynucleotides disclosed herein can comprise: a first nucleotide sequence, wherein the first nucleotide sequence encodes a first pathogen protein (e.g., a S1 subunit of the SARS-CoV-2 S protein) and is operably linked to a first promoter (e.g., a hEF1-HTLV promoter); a second nucleotide sequence, wherein the second nucleotide sequence encodes a second pathogen protein (e.g., a SARS-CoV-2 M protein) and is operably linked to the first promoter through an IRES sequence; a third nucleotide sequence, wherein the third nucleotide sequence encodes IL-12 p35 and is operably linked to a second promoter (e.g., a CMV promoter); and a fourth nucleotide sequence, wherein the fourth nucleotide sequence encodes IL-12 p340 and is operably linked to a second promoter (e.g., a CMV promoter). In some aspects, the first, second, third, and fourth nucleotide sequences of the polynucleotide are configured as shown in FIG. 14D (pVac 3).

In some aspects, the polynucleotides disclosed herein can comprise: a first nucleotide sequence, wherein the first nucleotide sequence encodes a first pathogen protein (e.g., a SARS-CoV-2 full-length D614G S protein) and is operably linked to a first promoter (e.g., an EF-1α promoter); a second nucleotide sequence, wherein the second nucleotide sequence encodes IL-12 p35 and is operably linked to a second promoter (e.g., a CMV promoter); and a third nucleotide sequence, wherein the third nucleotide sequence encodes IL-12 p40 and is operably linked to a second promoter (e.g., a CMV promoter). In some aspects, the first, second, and third nucleotide sequences of the polynucleotide are configured as shown in FIG. 14F (pVac 5).

In some aspects, the polynucleotides disclosed herein can comprise: a first nucleotide sequence, wherein the first nucleotide sequence encodes a first pathogen protein (e.g., a full-length SARS-CoV-2 D614G S protein) and is operably linked to a first promoter (e.g., an EF-1α promoter); a second nucleotide sequence, wherein the second nucleotide sequence encodes a second pathogen protein (e.g., a SARS-CoV-2 M protein) and is operably linked to the first promoter through an IRES sequence; a third nucleotide sequence, wherein the third nucleotide sequence encodes IL-12 p35 and is operably linked to a second promoter (e.g., a CMV promoter); and a fourth nucleotide sequence, wherein the fourth nucleotide sequence encodes IL-12 p340 and is operably linked to a second promoter (e.g., a CMV promoter). In some aspects, the first, second, third, and fourth nucleotide sequences of the polynucleotide are configured as shown in FIG. 14G (pVac 6).

In some aspects, the polynucleotides disclosed herein can comprise a first nucleotide sequence, wherein the first nucleotide sequence encodes a first pathogen protein (e.g., a S1 subunit of the SARS-CoV-2 S protein or a SARS-CoV-2 full-length D614G S protein) and is operably linked to a first promoter (e.g., an EF-1α promoter). In some aspects, the first nucleotide sequence of the polynucleotide is configured as shown in FIGS. 14B (pVac 1) or 14E (pVac 4).

In some aspects, the polynucleotides disclosed herein can comprise a first nucleotide sequence, wherein the first nucleotide sequence encodes a first pathogen protein (e.g., a SARS-CoV-2 full-length D614G S protein) and is operably linked to a first promoter (e.g., an EF-1α promoter); and a second nucleotide sequence, wherein the second nucleotide sequence encodes a second pathogen (e.g., a SARS-CoV-2 M protein) and is operably linked to a second promoter (e.g., a CMV promoter). In some aspects, the first nucleotide sequence of the polynucleotide is configured as shown in FIG. 14H (pVac 7).

In some aspects, the vector constructs illustrated in any of FIGS. 14A-14H can modified to replace the S1 subunit of the SARS-CoV-2 S protein or the SARS-CoV-2 full-length D614G S protein (a first nucleotide sequence encoding a first pathogen protein) and/or the SASRS-CoV-2 M protein (a second nucleotide sequence encoding a second pathogen protein) with nucleotide sequences encoding any combinations of pathogen antigen or antigenic fragment thereof disclosed herein. In some aspect, the nucleotide sequences encode antigens to a virus, a bacteria or a parasite. In some aspects, the nucleotide sequences encode one or more antigens comprise one or more viral antigens, one or more bacterial antigens, or one or more parasite antigens. In some aspects the vector constructs disclosed herein can be used to express DNA or mRNA.

In some aspects, the nucleotide sequences encode one or more bacterial antigens selected from a Yersinia pestis antigen, a Mycobacterium tuberculosis antigen, antigenic fragments thereof, or any combinations thereof. In some aspects, the Yersinia pestis antigen is a Yersinia pestis capsular antigen. In some aspects, the Yersinia pestis capsular antigen is F1-Ag or virulence antigen (V-Ag). In some aspects, the Mycobacterium tuberculosis antigen is an Apa antigen, an HP65 antigen, a rAg85A antigen, any antigenic fragments thereof, or any combinations thereof.

In some aspects, the nucleotide sequences encode one or more viral antigens selected from an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, antigenic fragments thereof, or any combinations thereof. In some aspects, the enterovirus antigen is an enterovirus 71 (E71) antigen, a coxsackievirus (Cox) protein antigen, antigenic fragments thereof, or any combinations thereof. In some aspects, the E71 antigen is an E71-VP1 antigen, a glutathione S-transferase (GST)-tagged E71-VP1 antigen, antigenic fragments thereof, or any combinations thereof. In some aspects, the Cox protein antigen is GST-tagged Cox protein antigen. In some aspects, the HSV antigen is an HSV-1 envelope antigen, an HSV-2 envelope antigen, an HSV-2 surface glycoprotein antigen, antigenic fragments thereof, or any combinations thereof. In some aspects, the HSV-2 surface glycoprotein antigen is a gB2 antigen, a gC2 antigen, a gD2 antigen, a gE2 antigen, or antigenic fragments thereof, or any combinations thereof. In some aspects, the HIV antigen is an Env antigen, a Gag antigen, a Nef antigen, a Pol antigen, antigenic fragments thereof, and or combinations thereof. In some aspects, the HPV antigen is a minor capsid protein L2 antigen. In some aspects, the minor capsid protein L2 antigen comprises one or more epitope domains (amino acids 10-36 and/or amino acids 65-89) of minor capsid protein L2. In some aspects, the HCV antigen is a nonstructural 3 (NS3) antigen. In some aspects, the RSV antigen is an F antigen, a G antigen, antigenic fragments thereof, or any combinations thereof. In some aspects, the Dengue virus antigen is an E protein antigen, an E protein domain III (EDIII) antigen, a non-structural protein 1 (NS1) antigen, a DEN-80E antigen, antigenic fragments thereof, or any combinations thereof. In some aspects, the Ebola virus antigen is a spike glycoprotein (GB) antigen, a VP24 antigen, a VP40 antigen, a nucleoprotein (NP) antigen, a VP30 antigen, a VP35 antigen, antigenic fragments thereof, or any combinations thereof. In some aspects, the Zika virus antigen is an envelope domain III antigen, a CKD antigen, antigenic fragments thereof, or any combinations thereof. In some aspects, the Chikungunya virus antigen is an E1 glycoprotein subunit antigen, the MHC class I epitope PPFGAGRPGQFGDI (SEQ ID NO: 34), the MHC class I epitope TAECKDKNL (SEQ ID NO: 35), the MHC class II epitope VRYKCNCGG (SEQ ID NO: 36), antigenic fragments thereof, or any combinations thereof. In some aspects, the measles virus antigen is a hemagglutinin protein MV-H antigen, a fusion protein MV-F antigen, antigenic fragments thereof, or any combinations thereof. In some aspects, the MERS-CoV antigen is a spike (S) protein antigen, an antigen from the receptor-binding domain of the S protein, an antigen from the membrane fusion domain of the S protein, antigenic fragments thereof, or any combinations thereof. In some aspects, the SARS-CoV antigen is a spike (S) protein antigen, an antigen from the receptor binding domain of the S protein, an antigen from the membrane fusion domain of the S protein, an envelope (E) protein antigen, an M protein antigen, antigenic fragments thereof, or any combinations thereof.

In some aspects, the nucleotide sequences encode one or more influenza virus antigens from any influenza virus type or subtype. In some aspects, the one or more influenza virus antigens are selected from the group consisting of: an influenza virus hemagglutinin (HA) antigen, an influenza virus neuraminidase (NA) antigen, an influenza virus matrix-2 (M2) protein antigen, antigenic fragments thereof, and any combination thereof. In some aspects, the one or more influenza virus antigens are derived from influenza virus type A, type B, type C, type D, or any combination thereof. In some spects, the one or more influenza virus antigens are derived from influenza virus type A. In some aspects, the one or more influenza virus antigens derived from influenza virus type A have (a) a HA subtype selected from H1 through H18 or any combination thereof and (b) a NA subtype selected from N1 through N11 or any combination thereof. In some aspects, the one or more influenza virus antigens derived from influenza virus type A, subtype H1N1; influenza virus type A, subtype H2N2; influenza virus type A, subtype H3N2; influenza virus type A, subtype H5N1; influenza virus type A, subtype H7N7; influenza virus type A, subtype H7N9; influenza virus type A, subtype H9N2; or any combination thereof. In some aspects, the one or more influenza virus antigens are derived from influenza virus type A, subtype H1N1; influenza virus type A, subtype H3N2; or the combination thereof. In some spects, the one or more influenza virus antigens are derived from influenza virus type B. In some aspects, the nucleotide sequences encode one or more SARS-CoV-2 antigens or antigenic fragments thereof disclosed herein and one or more influenza virus antigens or antigenic fragments thereof disclosed herein. In some aspects, the nucleotide sequences encode at least two SARS-CoV-2 antigens or antigenic fragments thereof disclosed herein. In some aspects, the at least two SARS-CoV-2 antigens or antigenic fragments thereof are derived from different strains of SARS-CoV-2. In some aspects, the at least two SARS-CoV-2 antigens or antigenic fragments thereof are different variants of the same SARS-CoV-2 antigen or antigenic fragment thereof, wherein the different variants of the same SARS-CoV-2 antigen or antigenic fragment thereof are derived from different strains of SARS-CoV-2. In some aspects, the nucleotide sequences encode one or more parasite antigens, wherein the one or more parasite antigens comprise one or more protozoan antigens. In some aspects, the nucleotide sequences encode one or more parasite antigens selected from Toxoplasma gondii antigen, a Plasmodium falciparum antigen, antigenic fragments thereof, or any combinations thereof. In some aspects, the Toxoplasma gondii antigen is antigen MIC8. In some aspects, the Plasmodium falciparum antigen is a SERA5 polypeptide antigen, a circumsporozite protein antigen, antigenic fragments thereof, or any combinations thereof. In some aspects, the nucleotide sequences encode one or more parasite antigens, wherein the one or more parasite antigens comprise one or more parasitic or pathogenic fungus antigens. In some aspects, the one or more parasitic or pathogenic fungus antigens are selected from the group consisting of a Candida spp. antigen (e.g., a Candida albicans antigen, a Candida glabrata antigen, a Candida parapsilosis antigen, a Candida tropicalis antigen, a Candida lusitaniae antigen, a Candida krusei antigen), a Pneumocystis spp. antigen, a Malassezia spp. antigen (e.g., a Malassezia furfur antigen), an Aspergillus fumigatus antigen, a Cryptococcus spp. antigen (e.g., a Cryptococcus neoformans antigen, a Cryptococcus gattii antigen), a Histoplasma capsulatum antigen, a Blastomyces dermatitidis antigen, a Paracoccidioides spp. antigen (e.g., a Paracoccidioides brasiliensis antigen, a Paracoccidioides lutzii antigen), a Coccidioides spp. antigen (e.g., a Coccidioides immitis antigen, a Coccidioides posadasii antigen), a Penicillium marneffei antigen, a Sporothrix schenckii antigen, a Trichosporon asahii antigen, a Fusarium spp. antigen (e.g., a Fusarium solanum antigen, a Fusarium oxysporum antigen), a Nectria spp. antigen, a Pseudoallescheria boydii antigen, a Cladophialphora bantianum antigen, a Ramichloridium spp. antigen, a Dactylaria gallopava antigen, an Exophiala spp. antigen (e.g., an Exophiala jeanselmei antigen, an Exophiala dermatitidis antigen), a Curvularia spp. antigen, a Bipolaris spp. antigen, an Alternaria spp. antigen, a Lacazia loboi antigen, a Conidiobolus spp. antigen (e.g., a Conidiobolus coronatus antigen, a Conidiobolus incongruus antigen), and any combination thereof.

In some aspects, the first pathogen protein is a SARS-CoV-2 S protein or an antigenic fragment thereof. In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the first SARS-CoV-2 protein comprises polypeptide having at least 70%, at least 75%, at least 80%, at least 85%, at least 90%, at least 95%, or at least 99% sequence identity to the amino acid sequence of SEQ ID NO: 2 or SEQ ID NO: 4. In some aspects, the first SARS-CoV-2 protein comprises the amino acid sequence of SEQ ID NO: 2 or SEQ ID NO: 4.

In some aspects, the second pathogen protein is a SARS-CoV-2 S protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the second SARS-CoV-2 protein comprises polypeptide having at least 70%, at least 75%, at least 80%, at least 85%, at least 90%, at least 95%, or at least 99% sequence identity to the amino acid sequence of SEQ ID NO: 2 or SEQ ID NO: 4. In some aspects, the second SARS-CoV-2 protein comprises the amino acid sequence of SEQ ID NO: 2 or SEQ ID NO: 4.

In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4, wherein the contiguous amino acids of SEQ ID NO: 2 or SEQ ID NO: 4 comprise one or more mutations (i.e., one or more substitutions, deletions, insertions, or any combination thereof). In some aspects, the first SARS-CoV-2 protein comprises the amino acid sequence of SEQ ID NO: 2 or SEQ ID NO: 4, wherein the polypeptide comprises one or more mutations (i.e., one or more substitutions, deletions, insertions, or any combination thereof).

In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4, wherein the contiguous amino acids of SEQ ID NO: 2 or SEQ ID NO: 4 comprise one or more mutations (i.e., one or more substitutions, deletions, insertions, or any combination thereof). In some aspects, the second SARS-CoV-2 protein comprises the amino acid sequence of SEQ ID NO: 2 or SEQ ID NO: 4, wherein the polypeptide comprises one or more mutations (i.e., one or more substitutions, deletions, insertions, or any combination thereof).

In some aspects, the first SARS-CoV-2 protein is the receptor binding domain (RBD) of the SARS-Cov-2 S protein or an antigenic fragment thereof. In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the first SARS-CoV-2 protein comprises polypeptide having at least 70%, at least 75%, at least 80%, at least 85%, at least 90%, at least 95%, or at least 99% sequence identity to the amino acid sequence of SEQ ID NO: 6. In some aspects, the first SARS-CoV-2 protein comprises the amino acid sequence of SEQ ID NO: 6.

In some aspects, the second SARS-CoV-2 protein is the receptor binding domain (RBD) of the SARS-Cov-2 S protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the second SARS-CoV-2 protein comprises polypeptide having at least 70%, at least 75%, at least 80%, at least 85%, at least 90%, at least 95%, or at least 99% sequence identity to the amino acid sequence of SEQ ID NO: 6. In some aspects, the second SARS-CoV-2 protein comprises the amino acid sequence of SEQ ID NO: 6.

In some aspects, the first SARS-CoV-2 protein is the receptor binding domain (RBD) of the SARS-Cov-2 S protein or an antigenic fragment thereof. In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6, wherein the contiguous amino acids of SEQ ID NO: 6 comprise one or more mutations (i.e., one or more substitutions, deletions, insertions, or any combination thereof). In some aspects, the first SARS-CoV-2 protein comprises the amino acid sequence of SEQ ID NO: 6, wherein the polypeptide comprises one or more mutations (i.e., one or more substitutions, deletions, insertions, or any combination thereof).

In some aspects, the second SARS-CoV-2 protein is the receptor binding domain (RBD) of the SARS-Cov-2 S protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6, wherein the contiguous amino acids of SEQ ID NO: 6 comprise one or more mutations (i.e., one or more substitutions, deletions, insertions, or any combination thereof). In some aspects, the second SARS-CoV-2 protein comprises the amino acid sequence of SEQ ID NO: 6, wherein the polypeptide comprises one or more mutations (i.e., one or more substitutions, deletions, insertions, or any combination thereof).

In some aspects, the first SARS-CoV-2 protein is the S1 subunit of the SARS-Cov-2 S protein or an antigenic fragment thereof. In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the first SARS-CoV-2 protein comprises polypeptide having at least 70%, at least 75%, at least 80%, at least 85%, at least 90%, at least 95%, or at least 99% sequence identity to the amino acid sequence of SEQ ID NO: 40. In some aspects, the first SARS-CoV-2 protein comprises the amino acid sequence of SEQ ID NO: 40.

In some aspects, the second SARS-CoV-2 protein is the S1 subunit of the SARS-Cov-2 S protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the second SARS-CoV-2 protein comprises polypeptide having at least 70%, at least 75%, at least 80%, at least 85%, at least 90%, at least 95%, or at least 99% sequence identity to the amino acid sequence of SEQ ID NO: 40. In some aspects, the second SARS-CoV-2 protein comprises the amino acid sequence of SEQ ID NO: 40.

In some aspects, the first SARS-CoV-2 protein is the S1 subunit of the SARS-Cov-2 S protein or an antigenic fragment thereof. In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40, wherein the contiguous amino acids of SEQ ID NO: 40 comprise one or more mutations (i.e., one or more substitutions, deletions, insertions, or any combination thereof). In some aspects, the first SARS-CoV-2 protein comprises the amino acid sequence of SEQ ID NO: 40, wherein the polypeptide comprises one or more mutations (i.e., one or more substitutions, deletions, insertions, or any combination thereof).

In some aspects, the second SARS-CoV-2 protein is the S1 subunit of the SARS-Cov-2 S protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40, wherein the contiguous amino acids of SEQ ID NO: 40 comprise one or more mutations (i.e., one or more substitutions, deletions, insertions, or any combination thereof). In some aspects, the second SARS-CoV-2 protein comprises the amino acid sequence of SEQ ID NO: 40, wherein the polypeptide comprises one or more mutations (i.e., one or more substitutions, deletions, insertions, or any combination thereof).

In some aspects, the one or more mutations in the SARS-CoV-2 full-length S protein, the RBD of the SARS-Cov-2 S protein, the S1 subunit of the SARS-CoV-2 S protein, or antigenic fragments thereof comprise one or more mutations previously reported in Li, T. et al., Emerg Microbes Infect. 9(1):2076-90 (2020); Lee, P. et al., Immune Netw. 21(1):e4 (2021); Yu, J. et al., Science 369(6505):806-11 (2020); Cattin-Ortola, J. et al., Nat Commun. 12(1):5333 (2021); Corbett, K. et al., Nature 586(7830):567-71 (2020); Hsieh, C. et al., Science 369(6510):1501-5 (2020); and Harvey, W. et al., Nat Rev Microbiol. 19(7):409-24 (2021), each of which is incorporated by reference herein in its entirety.

In some aspects, the one or more mutations in the SARS-CoV-2 full-length S protein, the RBD of the SARS-Cov-2 S protein, the S1 subunit of the SARS-CoV-2 S protein, or antigenic fragments thereof are selected from: ΔM1-S13, S12P, S131, L5F, L18F, T19R, T20N, P26S, Q52R, A67V, ΔH69-V70, G75V, T76I, D80A, T95I, R102I, ΔD119-F120, C136Y, D138Y, ΔF140, ΔL141-Y144, AY144, Y144S, Y145N, ΔH146, N148S, K150R, K150E, K150T, K150Q, S151P, W152C, E154K, ΔE156-F157, F157L, F157A, R158G, R190S, ΔI210, D215G, A222V, ΔL241-S243, ΔL242-L244, ΔA243-L244, ΔR246-G252, R246I, 11-amino acid residue insertion between Y248 and L249, D253G, D253N, R346K, V367F, E406W, K417E, K417V, K417N, K417T, N439K, K444A, K444R, K444N, K444Q, V445A, V445E, G447A, N450D, L452R, L452Q, Y453F, L455F, N460I, S477G, S477N, S477R, T478I, T478K, V483I, E484K, E484Q, G485R, F486A, F486V, F486L, N487A, F490S, Q493E, Q493K, S494P, N501Y, A570D, Q613H, D614G, H655Y, Q677H, Δ678-679, Δ681-682, Δ681-684, Δ682-685, P681H, P681R, R682S, R682A, R682Q, R683S, R683G, R683Q, R685G, R685Q, I692V, A701V, T716I, F817P, T859N, F888L, A892P, A899P, A942P, D950N, S982A, K986P, V987P, T10271, Q1071H, D1118H, V1176F, M1229I, ΔC1253-T1273, ΔC1254-T1273, ΔK1255-T1273, D1257A, E1258A, D1259A, D1260A, E1262A, K1269A, H1271K, T1273A, or any combination thereof, wherein the amino acid locations correspond to SEQ ID NO: 2 or SEQ ID NO: 4.

In some aspects, the one or more mutations comprise one or more mutations in the N-terminal signal peptide, which corresponds to amino acids 1-13 of SEQ ID NO: 2 or SEQ ID NO: 4. In some aspects, the one or more mutations in the N-terminal signal peptide is ΔM1-S13, wherein the amino acid locations correspond to SEQ ID NO: 2 or SEQ ID NO: 4.

In some aspects, the one or mutations comprise one or more mutations in the C-terminus of the full-length SARS-CoV-2 S protein. In some aspects, the one or more mutations in the C-terminus of the full-length SARS-CoV-2 S protein comprise one or more mutations in the C-terminal endoplasmic reticulum (ER) retention peptide, which corresponds to amino acids 1254-1273 of SEQ ID NO: 2 or SEQ ID NO: 4. In some aspects, the one or more mutations in the C-terminal ER retention peptide are selected from D1257A, E1258A, D1259A, D1260A, E1262A, K1269A, H1271K, T1273A, or any combination thereof, wherein the amino acid locations correspond to SEQ ID NO: 2 or SEQ ID NO: 4. In some aspects, the one or more mutations in the C-terminal ER retention peptide comprise D1257A+E1258A+D1259A+D1260A+E1262A (i.e., a D/E to A mutant), wherein the amino acid locations correspond to SEQ ID NO: 2 or SEQ ID NO: 4. In some aspects, the one or mutations in the C-terminal ER retention peptide is ΔC1253-T1273, ΔC1254-T1273, or ΔK1255-T1273.

In some aspects, the one or more mutations comprise K986P+V987P (i.e., a S-2P mutation), wherein the amino acid locations correspond to SEQ ID NO: 2 or SEQ ID NO: 4. In some aspects, the one or more mutations comprise F817P+A892P+A899P+A942P (i.e., a hexa-proline S mutation), wherein the amino acid locations correspond to SEQ ID NO: 2 or SEQ ID NO: 4. In some aspects, the one or more mutations comprise one or more mutations in the 681-PRRAR/SVA-688 S1/S2 furin cleavage site, wherein the amino acid locations correspond to SEQ ID NO: 2 or SEQ ID NO: 4. In some aspects, the one or more mutations in the 681-PRRAR/SVA-688 S1/S2 furin cleavage site are: (a) R682S+R683S (i.e., a SSAR mutation), (b) Δ681-684 (i.e., a ΔPRRA mutation), (c) Δ678-679+Δ681-682, (d) R682A+R683G+R685G (i.e., a 682-AGAG-685 mutation), (e) R682Q+R683Q+R685Q, (f) R682S+R685G, or (g) Δ682-685 (i.e., a ΔRRAR mutation), wherein the amino acid locations correspond to SEQ ID NO: 2 or SEQ ID NO: 4.

In some aspects, the one or more mutations comprise: (a) F817P+A892P+A899P+A942P (i.e., a hexa-proline S mutation) and (b) K986P+V987P (i.e., a S-2P mutation), wherein the amino acid locations correspond to SEQ ID NO: 2 or SEQ ID NO: 4. In some aspects, the one or more mutations comprise: (a) R682A+R683G+R685G (i.e., a 682-AGAG-685 mutation) and (b) K986P+V987P (i.e., a S-2P mutation), wherein the amino acid locations correspond to SEQ ID NO: 2 or SEQ ID NO: 4. In some aspects, the one or more mutations comprise: (a) R682A+R683G+R685G (i.e., a 682-AGAG-685 mutation), (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations comprise: (a) R682Q+R683Q+R685Q and (b) K986P+V987P (i.e., a S-2P mutation). In some aspects, the one or more mutations comprise: (a) R682Q+R683Q+R685Q, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations comprise: (a) R682S+R685G and (b) K986P+V987P (i.e., a S-2P mutation), wherein the amino acid locations correspond to SEQ ID NO: 2 or SEQ ID NO: 4. In some aspects, the one or more mutations comprise: (a) R682S+R685G, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4.

In some aspects, the second SARS-CoV-2 protein is a SARS-CoV-2 M protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the second SARS-CoV-2 protein comprises polypeptide having at least 70%, at least 75%, at least 80%, at least 85%, at least 90%, at least 95%, or at least 99% sequence identity to the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the second SARS-CoV-2 protein comprises the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20.

In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20, wherein the contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20 comprise one or more mutations selected from A2S, F28L, I48V, V70L, I82T, M84T, or any combination thereof, wherein the amino acid locations correspond to SEQ ID NO: 8. In some aspects, the second SARS-CoV-2 protein comprises the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20, wherein the polypeptide comprises one or more mutations selected from A2S, F28L, I48V, V70L, I82T, M84T, or any combination thereof, wherein the amino acid locations correspond to SEQ ID NO: 8.

In some aspects, the second SARS-CoV-2 protein is a SARS-CoV-2 E protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 55, at least 60, at least 65, at least 70, or at least 75 contiguous amino acids of SEQ ID NO: 22, SEQ ID NO: 24, or SEQ ID NO: 26. In some aspects, the second SARS-CoV-2 protein comprises polypeptide having at least 70%, at least 75%, at least 80%, at least 85%, at least 90%, at least 95%, or at least 99% sequence identity to the amino acid sequence of SEQ ID NO: 22, SEQ ID NO: 24, or SEQ ID NO: 26. In some aspects, the second SARS-CoV-2 protein comprises the amino acid sequence of SEQ ID NO: 22, SEQ ID NO: 24, or SEQ ID NO: 26.

In some aspects, the second SARS-CoV-2 protein is a SARS-CoV-2 N protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, or at least 400 contiguous amino acids of SEQ ID NO: 28. In some aspects, the second SARS-CoV-2 protein comprises polypeptide having at least 70%, at least 75%, at least 80%, at least 85%, at least 90%, at least 95%, or at least 99% sequence identity to the amino acid sequence of SEQ ID NO: 28. In some aspects, the second SARS-CoV-2 protein comprises the amino acid sequence of SEQ ID NO: 28.

In some aspects, promoter 1 is selected from the group consisting of: a CMV promoter, an RSV promoter, a Mo-MuLV LTR promoter, a mammalian EF1 promoter, a CK18 promoter, a CK19 promoter, and any combination thereof. In some aspects, promoter 2 is selected from the group consisting of: a CMV promoter, an RSV promoter, a Mo-MuLV LTR promoter, a mammalian EF1 promoter, a CK18 promoter, a CK19 promoter, and any combination thereof. In some aspects, promoter X is selected from the group consisting of: a CMV promoter, an RSV promoter, a Mo-MuLV LTR promoter, a mammalian EF1 promoter, a CK18 promoter, a CK19 promoter, and any combination thereof. In some aspects, promoter Y is selected from the group consisting of: a CMV promoter, an RSV promoter, a Mo-MuLV LTR promoter, a mammalian EF1 promoter, a CK18 promoter, a CK19 promoter, and any combination thereof. In some aspects, promoter Z is selected from the group consisting of: a CMV promoter, an RSV promoter, a Mo-MuLV LTR promoter, a mammalian EF1 promoter, a CK18 promoter, a CK19 promoter, and any combination thereof. In some aspects, the mammalian EF1 promoter is a hEF1-HTLV promoter.

In some aspects, the IRES sequence comprises a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 41.

### 5.4 Vectors, Vaccines, Compositions, and Pharmaceutical Compositions

Also provided herein are vectors or constructs comprising any polynucleotide described or exemplified herein, wherein the vector is a DNA plasmid vector, a multicistronic mRNA vector, a viral vector, a bacterial vector, a cosmid, or an artificial chromosome. Examples of vectors include but are not limited to AAV vectors, adenoviral vectors, retroviral vectors, poxvirus vectors, baculovirus vectors, herpes viral vectors, or combinations thereof. Also provided herein are DNA plasmid vectors and multicistronic mRNA vectors comprising any polynucleotide described or exemplified herein.

Also provided herein are compositions (e.g., pharmaceutical compositions and vaccines) comprising any polynucleotide or vector described or exemplified herein. In some aspects, the compositions (e.g., pharmaceutical composition or vaccine) further comprises a pharmaceutically acceptable carrier. In some aspects, the compositions (e.g., pharmaceutical composition or vaccine) further comprises a second polynucleotide encoding at least one immune modifier selected from the group consisting of: IL-2, IL-12 p35, IL-12 p40, IL-12 p70, IL-15, IL-18, TNFα, GM-CSF, IFN-α, IFN-β, a chemokine, MHC I, MHC II, HLA-DR, CD80, and CD86, wherein the polynucleotide encoding the at least one immune modifier is operably linked to a promoter. In some aspects, the compositions (e.g., pharmaceutical composition or vaccine) further comprises a delivery component (e.g., a cationic polymer, a poly-inosinic-polycytidylic acid, or a poloxamer). In some aspects, the delivery component further comprises benzalkonium chloride.

In some aspects, the IL-12 p35 immune modifier comprises an amino acid sequence having at least 70%, at least 75%, at least 80%, at least 85%, at least 90%, at least 95%, at least 99%, or 100% sequence identity to SEQ ID NO: 43 (mouse IL-12 p35) or SEQ ID NO: 47 (human IL-12 p35). In some aspects, the IL-12 p40 immune modifier comprises an amino acid sequence having at least 70%, at least 75%, at least 80%, at least 85%, at least 90%, at least 95%, at least 99%, or 100% sequence identity to SEQ ID NO: 45 (mouse IL-12 p40) or SEQ ID NO: 49 (human IL-12 p40).

In some aspects, the second polynucleotide encodes IL-12 p35, IL-12 p40, or the combination thereof. In some aspects, the IL-12 p35 immune modifier is encoded by a nucleic acid sequence having at least 70%, at least 75%, at least 80%, at least 85%, at least 90%, at least 95%, at least 99%, or 100% sequence identity to SEQ ID NO: 42 (nucleic acid sequence encoding mouse IL-12 p35) or SEQ ID NO: 46 (nucleic acid sequence encoding human IL-12 p35). In some aspects, the IL-12 p40 immune modifier is encoded by a nucleic acid sequence having at least 70%, at least 75%, at least 80%, at least 85%, at least 90%, at least 95%, at least 99%, or 100% sequence identity to SEQ ID NO: 44 (nucleic acid sequence encoding mouse IL-12 p40) or SEQ ID NO: 48 (nucleic acid sequence encoding human IL-12 p40).

In some aspects, the at least one immune modifier immune modifier encoded by the second polynucleotide comprises a viral protein (e.g., SARS-CoV-2 non-structural protein 1 (Nsp1), SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b) that attenuates a local inflammatory response and/or interferon response. In some aspects, the viral protein is from the same virus as a viral antigen encoded by an antigen nucleic acid. In some aspects, In some aspects, the viral protein is from a different virus than a viral antigen encoded by an antigen nucleic acid. In some aspects, the viral protein attenuates a local inflammatory response and/or interferon response elicited by a pathogen antigen disclosed herein. In some aspects, the at least one immune modifier immune modifier encoded by the second polynucleotide comprises a viral protein selected from the group consisting of SARS-CoV-2 Nsp1, SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b, and any combination thereof. In some aspects, the at least one immune modifier comprises one or more concatamers of non-coding 5′-C-phosphate-G-3′ (CpG) dinucleotides. In some aspects, the one or more concatamers of non-coding CpG dinucleotides activate the Toll-like receptor 9 (TLR9) signaling pathway. In some aspects, the one or more concatamers of non-coding CpG dinucleotides comprise one or more concatamers of non-coding CpG dinucleotides previously reported in Bauer, A. et al., Nucleic Acids Research 38(12):3891-908 (2010); Cornelie, S. et al., Journal of Biological Chemistry 279(15):15124-9 (2004); Klinman, D. et al., J Immunol. 158(8):3635-9 (1997); Klinman, D. et al., Immunological Reviews 199(1):201-16 (2004); Luo, Z. et al., Mol Med Rep. 6(6):1309-14 (2012); Bode, C. et al., Expert Rev Vaccines 10(4):499-511 (2011); and Kuo, T. et al., Scientific Reports 10:20085 (2020), each of which is incorporated by reference herein in its entirety.

In some aspects, the composition (e.g., pharmaceutical composition or vaccine) further comprises a third polynucleotide encoding at least one immune modifier selected from the group consisting of: IL-2, IL-12 p35, IL-12 p40, IL-12 p70, IL-15, IL-18, TNFα, GM-CSF, IFN-α, IFN-β, a chemokine, MHC I, MHC II, HLA-DR, CD80, and CD86, wherein the polynucleotide encoding the at least one immune modifier is operably linked to a promoter. In some aspects, the second polynucleotide encoding at least one immune modifier encodes the IL-12 p35 immune modifier, and the third polynucleotide encoding at least one immune modifier encodes the IL-12 p40 immune modifier. In some aspects, the second polynucleotide encoding the IL-12 p35 modifier comprises a nucleic acid sequence having at least 70%, at least 75%, at least 80%, at least 85%, at least 90%, at least 95%, at least 99%, or 100% sequence identity to SEQ ID NO: 42 (nucleic acid sequence encoding mouse IL-12 p35) or SEQ ID NO: 46 (nucleic acid sequence encoding human IL-12 p35). In some aspects, the third polynucleotide encoding the IL-12 p40 immune modifier comprises a nucleic acid sequence having at least 70%, at least 75%, at least 80%, at least 85%, at least 90%, at least 95%, at least 99%, or 100% sequence identity to SEQ ID NO: 44 (nucleic acid sequence encoding mouse IL-12 p40) or SEQ ID NO: 48 (nucleic acid sequence encoding human IL-12 p40).

In some aspects, the at least one immune modifier immune modifier encoded by the third polynucleotide comprises a viral protein (e.g., SARS-CoV-2 non-structural protein 1 (Nsp1), SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b) that attenuates a local inflammatory response and/or interferon response. In some aspects, the viral protein is from the same virus as a viral antigen encoded by an antigen nucleic acid. In some aspects, In some aspects, the viral protein is from a different virus than a viral antigen encoded by an antigen nucleic acid. In some aspects, the viral protein attenuates a local inflammatory response and/or interferon response elicited by a pathogen antigen disclosed herein. In some aspects, the at least one immune modifier immune modifier encoded by the third polynucleotide comprises a viral protein selected from the group consisting of SARS-CoV-2 Nsp1, SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b, and any combination thereof. In some aspects, the at least one immune modifier comprises one or more concatamers of non-coding 5′-C-phosphate-G-3′ (CpG) dinucleotides. In some aspects, the one or more concatamers of non-coding CpG dinucleotides activate the Toll-like receptor 9 (TLR9) signaling pathway. In some aspects, the one or more concatamers of non-coding CpG dinucleotides comprise one or more concatamers of non-coding CpG dinucleotides previously reported in Bauer, A. et al., Nucleic Acids Research 38(12):3891-908 (2010); Cornelie, S. et al., Journal of Biological Chemistry 279(15):15124-9 (2004); Klinman, D. et al., J Immunol. 158(8):3635-9 (1997); Klinman, D. et al., Immunological Reviews 199(1):201-16 (2004); Luo, Z. et al., Mol Med Rep. 6(6):1309-14 (2012); Bode, C. et al., Expert Rev Vaccines 10(4):499-511 (2011); and Kuo, T. et al., Scientific Reports 10:20085 (2020), each of which is incorporated by reference herein in its entirety. Also provided herein are compositions (e.g., pharmaceutical compositions or vaccines) comprising any polynucleotide, multicistronic mRNA vector, or DNA plasmid vector described or exemplified herein. In some aspects, the compositions (e.g., pharmaceutical composition or vaccine) comprises a second polynucleotide encoding at least one immune modifier selected from the group consisting of: IL-2, IL-12 p35, IL-12 p40, IL-12 p70, IL-15, IL-18, TNFα, GM-CSF, IFN-α, IFN-β, a chemokine, MHC I, MHC II, HLA-DR, CD80, and CD86, wherein the polynucleotide encoding the at least one immune modifier is operably linked to a promoter. In some aspects, the compositions (e.g., pharmaceutical composition or vaccine) further comprises a third polynucleotide encoding at least one immune modifier selected from the group consisting of: IL-2, IL-12 p35, IL-12 p40, IL-12 p70, IL-15, IL-18, TNFα, GM-CSF, IFN-α, IFN-β, a chemokine, MHC I, MHC II, HLA-DR, CD80, and CD86, wherein the third polynucleotide encoding the at least one immune modifier is operably linked to a promoter. In some aspects, the second polynucleotide encoding at least one immune modifier encodes IL-12 p35, and the third polynucleotide encoding at least one immune modifier encodies IL-12 p40.

In some aspects, the at least one immune modifier immune modifier encoded by the second polynucleotide comprises a viral protein (e.g., SARS-CoV-2 non-structural protein 1 (Nsp1), SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b) that attenuates a local inflammatory response and/or interferon response. In some aspects, the viral protein is from the same virus as a viral antigen encoded by an antigen nucleic acid. In some aspects, In some aspects, the viral protein is from a different virus than a viral antigen encoded by an antigen nucleic acid. In some aspects, the viral protein attenuates a local inflammatory response and/or interferon response elicited by a pathogen antigen disclosed herein. In some aspects, the at least one immune modifier immune modifier encoded by the second polynucleotide comprises a viral protein selected from the group consisting of SARS-CoV-2 Nsp1, SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b, and any combination thereof. In some aspects, the at least one immune modifier comprises one or more concatamers of non-coding 5′-C-phosphate-G-3′ (CpG) dinucleotides. In some aspects, the one or more concatamers of non-coding CpG dinucleotides activate the Toll-like receptor 9 (TLR9) signaling pathway. In some aspects, the one or more concatamers of non-coding CpG dinucleotides comprise one or more concatamers of non-coding CpG dinucleotides previously reported in Bauer, A. et al., Nucleic Acids Research 38(12):3891-908 (2010); Cornelie, S. et al., Journal of Biological Chemistry 279(15):15124-9 (2004); Klinman, D. et al., J Immunol. 158(8):3635-9 (1997); Klinman, D. et al., Immunological Reviews 199(1):201-16 (2004); Luo, Z. et al., Mol Med Rep. 6(6):1309-14 (2012); Bode, C. et al., Expert Rev Vaccines 10(4):499-511 (2011); and Kuo, T. et al., Scientific Reports 10:20085 (2020), each of which is incorporated by reference herein in its entirety.

In some aspects, the at least one immune modifier immune modifier encoded by the third polynucleotide comprises a viral protein (e.g., SARS-CoV-2 non-structural protein 1 (Nsp1), SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b) that attenuates a local inflammatory response and/or interferon response. In some aspects, the viral protein is from the same virus as a viral antigen encoded by an antigen nucleic acid. In some aspects, In some aspects, the viral protein is from a different virus than a viral antigen encoded by an antigen nucleic acid. In some aspects, the viral protein attenuates a local inflammatory response and/or interferon response elicited by a pathogen antigen disclosed herein. In some aspects, the at least one immune modifier immune modifier encoded by the third polynucleotide comprises a viral protein selected from the group consisting of SARS-CoV-2 Nsp1, SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b, and any combination thereof. In some aspects, the at least one immune modifier comprises one or more concatamers of non-coding 5′-C-phosphate-G-3′ (CpG) dinucleotides. In some aspects, the one or more concatamers of non-coding CpG dinucleotides activate the Toll-like receptor 9 (TLR9) signaling pathway. In some aspects, the one or more concatamers of non-coding CpG dinucleotides comprise one or more concatamers of non-coding CpG dinucleotides previously reported in Bauer, A. et al., Nucleic Acids Research 38(12):3891-908 (2010); Cornelie, S. et al., Journal of Biological Chemistry 279(15):15124-9 (2004); Klinman, D. et al., J Immunol. 158(8):3635-9 (1997); Klinman, D. et al., Immunological Reviews 199(1):201-16 (2004); Luo, Z. et al., Mol Med Rep. 6(6):1309-14 (2012); Bode, C. et al., Expert Rev Vaccines 10(4):499-511 (2011); and Kuo, T. et al., Scientific Reports 10:20085 (2020), each of which is incorporated by reference herein in its entirety.

In some aspects, the compositions (e.g., pharmaceutical compositions or vaccines) of the disclosure comprise a delivery component and a polynucleotide comprising: a first nucleotide sequence, wherein the first nucleotide sequence encodes IL-12 p35 and is operably linked to a CMV promoter; a second nucleotide sequence, wherein the second nucleotide sequence encodes IL-12 p40 and is operably linked to a CMV promoter; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-12 p35 and is operably linked to a CMV promoter; a second nucleotide sequence positioned 3′ to the first nucleotide sequence, wherein the second nucleotide sequence encodes IL-12 p40 and is operably linked to a CMV promoter; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 1.

In some aspects, the compositions (e.g., pharmaceutical compositions or vaccines) of the disclosure comprise a delivery component and a polynucleotide comprising: a first nucleotide sequence, wherein the first nucleotide sequence encodes IL-12 p35 and is operably linked to a CMV promoter; a second nucleotide sequence, wherein the second nucleotide sequence encodes IL-12 p40 and is operably linked to a CMV promoter; a third nucleotide sequence, wherein the third nucleotide sequence encodes MHC I and is operably linked to promoter Z; a fourth nucleotide sequence, wherein the fourth nucleotide sequence encodes a first SARS-CoV-2 protein and is operably linked to promoter 1, and a fifth nucleotide sequence, wherein the fifth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-12 p35 and is operably linked to a CMV promoter; a second nucleotide sequence positioned 3′ to the first nucleotide sequence, wherein the second nucleotide sequence encodes IL-12 p40 and is operably linked to a CMV promoter; a third nucleotide sequence positioned 3′ to the second nucleotide sequence, wherein the third nucleotide sequence encodes MHC I and is operably linked to promoter Z; a fourth nucleotide sequence positioned 3′ to the third nucleotide sequence, wherein the fourth nucleotide sequence encodes a first SARS-CoV-2 protein and is operably linked to promoter 1, and a fifth nucleotide sequence positioned 3′ to the fourth nucleotide sequence, wherein the fifth nucleotide sequence encodes a second SARS-CoV-2 protein and is operably linked to promoter 2. In some aspects, the first, second, third, fourth, and fifth nucleotide sequences of the polynucleotide are configured as shown in FIG. 2.

In some aspects, the compositions (e.g., pharmaceutical compositions or vaccines) of the disclosure comprise a delivery component and a polynucleotide comprising: a first nucleotide sequence, wherein the first nucleotide sequence encodes IL-12 p35 and is operably linked to a CMV promoter; a second nucleotide sequence, wherein the second nucleotide sequence encodes IL-12 p40 and is operably linked to a CMV promoter; a third nucleotide sequence, wherein the third nucleotide sequence encodes MHC II and is operably linked to promoter Z; a fourth nucleotide sequence, wherein the fourth nucleotide sequence encodes a first SARS-CoV-2 protein and is operably linked to promoter 1, and a fifth nucleotide sequence, wherein the fifth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-12 p35 and is operably linked to a CMV promoter; a second nucleotide sequence positioned 3′ to the first nucleotide sequence, wherein the second nucleotide sequence encodes IL-12 p40 and is operably linked to a CMV promoter; a third nucleotide sequence positioned 3′ to the second nucleotide sequence, wherein the third nucleotide sequence encodes MHC II and is operably linked to promoter Z; a fourth nucleotide sequence positioned 3′ to the third nucleotide sequence, wherein the fourth nucleotide sequence encodes a first SARS-CoV-2 protein and is operably linked to promoter 1, and a fifth nucleotide sequence positioned 3′ to the fourth nucleotide sequence, wherein the fifth nucleotide sequence encodes a second SARS-CoV-2 protein and is operably linked to promoter 2. In some aspects, the first, second, third, fourth, and fifth nucleotide sequences of the polynucleotide are configured as shown in FIG. 3.

In some aspects, the compositions (e.g., pharmaceutical compositions or vaccines) of the disclosure comprise a delivery component and a polynucleotide comprising: a first nucleotide sequence, wherein the first nucleotide sequence encodes IL-2 and is operably linked to a CMV promoter; a second nucleotide sequence, wherein the second nucleotide sequence encodes a first SARS-CoV-2 protein and is operably linked to promoter 1; and a third nucleotide sequence, wherein the third nucleotide sequence encodes a second SARS-CoV-2 protein and is operably linked to promoter 2. In some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes a first SARS-CoV-2 protein and is operably linked to promoter 1; and a third nucleotide sequence positioned 3′ to the second nucleotide sequence, wherein the third nucleotide sequence encodes a second SARS-CoV-2 protein and is operably linked to promoter 2. In some aspects, the first, second, and third nucleotide sequences of the polynucleotide are configured as shown in FIG. 4.

In some aspects, the compositions (e.g., pharmaceutical compositions or vaccines) of the disclosure comprise a delivery component and a polynucleotide comprising: a first nucleotide sequence, wherein the first nucleotide sequence encodes IL-2 and is operably linked to a CMV promoter; a second nucleotide sequence, wherein the second nucleotide sequence encodes MHC I and is operably linked to promoter Z; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes MHC I and is operably linked to promoter Z; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 5.

In some aspects, the compositions (e.g., pharmaceutical compositions or vaccines) of the disclosure comprise a delivery component and a polynucleotide comprising: a first nucleotide sequence, wherein the first nucleotide sequence encodes IL-2 and is operably linked to a CMV promoter; a second nucleotide sequence, wherein the second nucleotide sequence encodes MHC II and is operably linked to promoter Z; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes MHC II and is operably linked to promoter Z; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 6.

In some aspects, the compositions (e.g., pharmaceutical compositions or vaccines) of the disclosure comprise a delivery component and a polynucleotide comprising: a first nucleotide sequence, wherein the first nucleotide sequence encodes IL-2 and is operably linked to a CMV promoter; a second nucleotide sequence, wherein the second nucleotide sequence encodes CCL3 and is operably linked to promoter X; a third nucleotide sequence, wherein the third nucleotide sequence encodes CCL4 and is operably linked to promoter Y; a fourth nucleotide sequence, wherein the fourth nucleotide sequence encodes a first SARS-CoV-2 protein and is operably linked to promoter 1, and a fifth nucleotide sequence, wherein the fifth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes CCL3 and is operably linked to promoter X; a third nucleotide sequence positioned 3′ to the second nucleotide sequence, wherein the third nucleotide sequence encodes CCL4 and is operably linked to promoter Y; a fourth nucleotide sequence positioned 3′ to the third nucleotide sequence, wherein the fourth nucleotide sequence encodes a first SARS-CoV-2 protein and is operably linked to promoter 1, and a fifth nucleotide sequence positioned 3′ to the fourth nucleotide sequence, wherein the fifth nucleotide sequence encodes a second SARS-CoV-2 protein and is operably linked to promoter 2. In some aspects, the first, second, third, fourth, and fifth nucleotide sequences of the polynucleotide are configured as shown in FIG. 7.

In some aspects, the compositions (e.g., pharmaceutical compositions or vaccines) of the disclosure comprise a delivery component and a polynucleotide comprising: a first nucleotide sequence, wherein the first nucleotide sequence encodes IL-15 and is operably linked to a CMV promoter; a second nucleotide sequence, wherein the second nucleotide sequence encodes a first SARS-CoV-2 protein and is operably linked to promoter 1; and a third nucleotide sequence, wherein the third nucleotide sequence encodes a second SARS-CoV-2 protein and is operably linked to promoter 2. In some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes a first SARS-CoV-2 protein and is operably linked to promoter 1; and a third nucleotide sequence positioned 3′ to the second nucleotide sequence, wherein the third nucleotide sequence encodes a second SARS-CoV-2 protein and is operably linked to promoter 2. In some aspects, the first, second, and third nucleotide sequences of the polynucleotide are configured as shown in FIG. 8.

In some aspects, the compositions (e.g., pharmaceutical compositions or vaccines) of the disclosure comprise a delivery component and a polynucleotide comprising: a first nucleotide sequence, wherein the first nucleotide sequence encodes IL-15 and is operably linked to a CMV promoter; a second nucleotide sequence, wherein the second nucleotide sequence encodes MHC I and is operably linked to promoter Z; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes MHC I and is operably linked to promoter Z; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 9.

In some aspects, the compositions (e.g., pharmaceutical compositions or vaccines) of the disclosure comprise a delivery component and a polynucleotide comprising: a first nucleotide sequence, wherein the first nucleotide sequence encodes IL-15 and is operably linked to a CMV promoter; a second nucleotide sequence, wherein the second nucleotide sequence encodes MHC II and is operably linked to promoter Z; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes MHC II and is operably linked to promoter Z; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 10.

In some aspects, the compositions (e.g., pharmaceutical compositions or vaccines) of the disclosure comprise a delivery component and a polynucleotide comprising: a first nucleotide sequence, wherein the first nucleotide sequence encodes IL-15 and is operably linked to a CMV promoter; a second nucleotide sequence, wherein the second nucleotide sequence encodes CCL3 and is operably linked to promoter X; a third nucleotide sequence, wherein the third nucleotide sequence encodes CCL4 and is operably linked to promoter Y; a fourth nucleotide sequence, wherein the fourth nucleotide sequence encodes a first SARS-CoV-2 protein and is operably linked to promoter 1, and a fifth nucleotide sequence, wherein the fifth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes CCL3 and is operably linked to promoter X; a third nucleotide sequence positioned 3′ to the second nucleotide sequence, wherein the third nucleotide sequence encodes CCL4 and is operably linked to promoter Y; a fourth nucleotide sequence positioned 3′ to the third nucleotide sequence, wherein the fourth nucleotide sequence encodes a first SARS-CoV-2 protein and is operably linked to promoter 1, and a fifth nucleotide sequence positioned 3′ to the fourth nucleotide sequence, wherein the fifth nucleotide sequence encodes a second SARS-CoV-2 protein and is operably linked to promoter 2. In some aspects, the first, second, third, fourth, and fifth nucleotide sequences of the polynucleotide are configured as shown in FIG. 11.

In some aspects, the compositions (e.g., pharmaceutical compositions or vaccines) of the disclosure comprise a delivery component and a polynucleotide comprising: a first nucleotide sequence, wherein the first nucleotide sequence encodes CCL3 and is operably linked to promoter X; a second nucleotide sequence, wherein the second nucleotide sequence encodes CCL4 and is operably linked to promoter Y; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes CCL3 and is operably linked to promoter X; a second nucleotide sequence positioned 3′ to the first nucleotide sequence, wherein the second nucleotide sequence encodes CCL4 and is operably linked to promoter Y; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 12.

In some aspects, the compositions (e.g., pharmaceutical compostions or vaccines) can comprise a delivery component and a polynucleotides configured as shown in the vector constructs illustrated in any of FIGS. 1-12, which can modified to replace the “Covid-19 Spike Gene” (a first nucleotide sequence encoding a SARS-CoV-2 protein) and the “Covid-19 Gene-2” (a second nucleotide sequence encoding a SARS-CoV-2 protein) with nucleotide sequences encoding any combinations of pathogen antigen or antigenic fragment thereof disclosed herein. In some aspect, the nucleotide sequences encode antigens to a virus, a bacteria or a parasite. In some aspects, the nucleotide sequences encode one or more antigens comprise one or more viral antigens, one or more bacterial antigens, or one or more parasite antigens.

In some aspects, the compositions (e.g., pharmaceutical compositions or vaccines) of the disclosure comprise a delivery component and a polynucleotide comprising: a first nucleotide sequence, wherein the first nucleotide sequence encodes a first pathogen protein (e.g., a S1 subunit of the SARS-CoV-2 S protein) and is operably linked to a first promoter (e.g., a hEF1-HTLV promoter); a second nucleotide sequence, wherein the second nucleotide sequence encodes IL-12 p35 and is operably linked to a second promoter (e.g., a CMV promoter); and a third nucleotide sequence, wherein the third nucleotide sequence encodes IL-12 p40 and is operably linked to a second promoter (e.g., a CMV promoter). In some aspects, the first, second, and third nucleotide sequences of the polynucleotide are configured as shown in FIG. 14C (pVac 2).

In some aspects, the compositions (e.g., pharmaceutical compositions or vaccines) of the disclosure comprise a delivery component and a polynucleotide comprising: a first nucleotide sequence, wherein the first nucleotide sequence encodes a first pathogen protein (e.g., a S1 subunit of the SARS-CoV-2 S protein) and is operably linked to a first promoter (e.g., a hEF1-HTLV promoter); a second nucleotide sequence, wherein the second nucleotide sequence encodes a second pathogen protein (e.g., a SARS-CoV-2 M protein) and is operably linked to the first promoter through an IRES sequence; a third nucleotide sequence, wherein the third nucleotide sequence encodes IL-12 p35 and is operably linked to a second promoter (e.g., a CMV promoter); and a fourth nucleotide sequence, wherein the fourth nucleotide sequence encodes IL-12 p340 and is operably linked to a second promoter (e.g., a CMV promoter). In some aspects, the first, second, third, and fourth nucleotide sequences of the polynucleotide are configured as shown in FIG. 14D (pVac 3).

In some aspects, the compositions (e.g., pharmaceutical compositions or vaccines) of the disclosure comprise a delivery component and a polynucleotide comprising: a first nucleotide sequence, wherein the first nucleotide sequence encodes a first pathogen protein (e.g., a SARS-CoV-2 full-length D614G S protein) and is operably linked to a first promoter (e.g., an EF-1α promoter); a second nucleotide sequence, wherein the second nucleotide sequence encodes IL-12 p35 and is operably linked to a second promoter (e.g., a CMV promoter); and a third nucleotide sequence, wherein the third nucleotide sequence encodes IL-12 p40 and is operably linked to a second promoter (e.g., a CMV promoter). In some aspects, the first, second, and third nucleotide sequences of the polynucleotide are configured as shown in FIG. 14F (pVac 5).

In some aspects, the compositions (e.g., pharmaceutical compositions or vaccines) of the disclosure comprise a delivery component and a polynucleotide comprising: a first nucleotide sequence, wherein the first nucleotide sequence encodes a first pathogen protein (e.g., a full-length SARS-CoV-2 D614G S protein) and is operably linked to a first promoter (e.g., an EF-1α promoter); a second nucleotide sequence, wherein the second nucleotide sequence encodes a second pathogen protein (e.g., a SARS-CoV-2 M protein) and is operably linked to the first promoter through an IRES sequence; a third nucleotide sequence, wherein the third nucleotide sequence encodes IL-12 p35 and is operably linked to a second promoter (e.g., a CMV promoter); and a fourth nucleotide sequence, wherein the fourth nucleotide sequence encodes IL-12 p340 and is operably linked to a second promoter (e.g., a CMV promoter). In some aspects, the first, second, third, and fourth nucleotide sequences of the polynucleotide are configured as shown in FIG. 14G (pVac 6).

In some aspects, the compositions (e.g., pharmaceutical compositions or vaccines) of the disclosure comprise a delivery component and a polynucleotide comprising a first nucleotide sequence, wherein the first nucleotide sequence encodes a first pathogen protein (e.g., a S1 subunit of the SARS-CoV-2 S protein or a SARS-CoV-2 full-length D614G S protein) and is operably linked to a first promoter (e.g., an EF-1α promoter). In some aspects, the first nucleotide sequence of the polynucleotide is configured as shown in FIG. 14B (pVac 1) or 14E (pVac 4).

In some aspects, the compositions (e.g., pharmaceutical compositions or vaccines) of the disclosure comprise a delivery component and a polynucleotide comprising a first nucleotide sequence, wherein the first nucleotide sequence encodes a first pathogen protein (e.g., a SARS-CoV-2 full-length D614G S protein) and is operably linked to a first promoter (e.g., an EF-1α promoter); and a second nucleotide sequence, wherein the second nucleotide sequence encodes a second pathogen (e.g., a SARS-CoV-2 M protein) and is operably linked to a second promoter (e.g., a CMV promoter). In some aspects, the first nucleotide sequence of the polynucleotide is configured as shown in FIG. 14H (pVac 7).

In some aspects, the compositions (e.g., pharmaceutical compostions or vaccines) can comprise a delivery component and a polynucleotides configured as shown in the vector constructs illustrated in any of FIG.S X (pVac 1-7) can modified to replace the S1 subunit of the SARS-CoV-2 S protein or the SARS-CoV-2 full-length D614G S protein (a first nucleotide sequence encoding a first pathogen protein) and/or the SASRS-CoV-2 M protein (a second nucleotide sequence encoding a second pathogen protein) with nucleotide sequences encoding any combinations of pathogen antigen or antigenic fragment thereof disclosed herein. In some aspect, the nucleotide sequences encode antigens to a virus, a bacteria or a parasite. In some aspects, the nucleotide sequences encode one or more antigens comprise one or more viral antigens, one or more bacterial antigens, or one or more parasite antigens.

In some aspects, the nucleotide sequences encode one or more bacterial antigens selected from a Yersinia pestis antigen, a Mycobacterium tuberculosis antigen, antigenic fragments thereof, or any combinations thereof. In some aspects, the Yersinia pestis antigen is a Yersinia pestis capsular antigen. In some aspects, the Yersinia pestis capsular antigen is F1-Ag or virulence antigen (V-Ag). In some aspects, the Mycobacterium tuberculosis antigen is an Apa antigen, an HP65 antigen, a rAg85A antigen, any antigenic fragments thereof, or any combinations thereof.

In some aspects, the nucleotide sequences encode one or more viral antigens selected from an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, antigenic fragments thereof, or any combinations thereof. In some aspects, the enterovirus antigen is an enterovirus 71 (E71) antigen, a coxsackievirus (Cox) protein antigen, antigenic fragments thereof, or any combinations thereof. In some aspects, the E71 antigen is an E71-VP1 antigen, a glutathione S-transferase (GST)-tagged E71-VP1 antigen, antigenic fragments thereof, or any combinations thereof. In some aspects, the Cox protein antigen is GST-tagged Cox protein antigen. In some aspects, the HSV antigen is an HSV-1 envelope antigen, an HSV-2 envelope antigen, an HSV-2 surface glycoprotein antigen, antigenic fragments thereof, or any combinations thereof. In some aspects, the HSV-2 surface glycoprotein antigen is a gB2 antigen, a gC2 antigen, a gD2 antigen, a gE2 antigen, or antigenic fragments thereof, or any combinations thereof. In some aspects, the HIV antigen is an Env antigen, a Gag antigen, a Nef antigen, a Pol antigen, antigenic fragments thereof, and or combinations thereof. In some aspects, the HPV antigen is a minor capsid protein L2 antigen. In some aspects, the minor capsid protein L2 antigen comprises one or more epitope domains (amino acids 10-36 and/or amino acids 65-89) of minor capsid protein L2. In some aspects, the HCV antigen is a nonstructural 3 (NS3) antigen. In some aspects, the RSV antigen is an F antigen, a G antigen, antigenic fragments thereof, or any combinations thereof. In some aspects, the Dengue virus antigen is an E protein antigen, an E protein domain III (EDIII) antigen, a non-structural protein 1 (NS1) antigen, a DEN-80E antigen, antigenic fragments thereof, or any combinations thereof. In some aspects, the Ebola virus antigen is a spike glycoprotein (GB) antigen, a VP24 antigen, a VP40 antigen, a nucleoprotein (NP) antigen, a VP30 antigen, a VP35 antigen, antigenic fragments thereof, or any combinations thereof. In some aspects the Zika virus antigen is an envelope domain III antigen, a CKD antigen, antigenic fragments thereof, or any combinations thereof. In some aspects, the Chikungunya virus antigen is an E1 glycoprotein subunit antigen, the MHC class I epitope PPFGAGRPGQFGDI (SEQ ID NO: 34), the MHC class I epitope TAECKDKNL (SEQ ID NO: 35), the MHC class II epitope VRYKCNCGG (SEQ ID NO: 36), antigenic fragments thereof, or any combinations thereof. In some aspects, the measles virus antigen is a hemagglutinin protein MV-H antigen, a fusion protein MV-F antigen, antigenic fragments thereof, or any combinations thereof. In some aspects, the MERS-CoV antigen is a spike (S) protein antigen, an antigen from the receptor-binding domain of the S protein, an antigen from the membrane fusion domain of the S protein, antigenic fragments thereof, or any combinations thereof. In some aspects, the SARS-CoV antigen is a spike (S) protein antigen, an antigen from the receptor binding domain of the S protein, an antigen from the membrane fusion domain of the S protein, an envelope (E) protein antigen, an M protein antigen, antigenic fragments thereof, or any combinations thereof.

In some aspects, the nucleotide sequences encode one or more influenza virus antigens from any influenza virus type or subtype. In some aspects, the one or more influenza virus antigens are selected from the group consisting of: an influenza virus hemagglutinin (HA) antigen, an influenza virus neuraminidase (NA) antigen, an influenza virus matrix-2 (M2) protein antigen, antigenic fragments thereof, and any combination thereof. In some aspects, the one or more influenza virus antigens are derived from influenza virus type A, type B, type C, type D, or any combination thereof. In some spects, the one or more influenza virus antigens are derived from influenza virus type A. In some aspects, the one or more influenza virus antigens derived from influenza virus type A have (a) a HA subtype selected from H1 through H18 or any combination thereof and (b) a NA subtype selected from N1 through N11 or any combination thereof. In some aspects, the one or more influenza virus antigens derived from influenza virus type A, subtype H1N1; influenza virus type A, subtype H2N2; influenza virus type A, subtype H3N2; influenza virus type A, subtype H5N1; influenza virus type A, subtype H7N7; influenza virus type A, subtype H7N9; influenza virus type A, subtype H9N2; or any combination thereof. In some aspects, the one or more influenza virus antigens are derived from influenza virus type A, subtype H1N1; influenza virus type A, subtype H3N2; or the combination thereof. In some spects, the one or more influenza virus antigens are derived from influenza virus type B. In some aspects, the nucleotide sequences encode one or more SARS-CoV-2 antigens or antigenic fragments thereof disclosed herein and one or more influenza virus antigens or antigenic fragments thereof disclosed herein. In some aspects, the nucleotide sequence encodes one or more parasite antigens, wherein the one or more parasite antigens comprise one or more protozoan antigens. In some aspects, the nucleotide sequences encode one or more parasite antigens selected from Toxoplasma gondii antigen, a Plasmodium falciparum antigen, antigenic fragments thereof, or any combinations thereof. In some aspects, the Toxoplasma gondii antigen is antigen MIC8. In some aspects, the Plasmodium falciparum antigen is a SERA5 polypeptide antigen, a circumsporozite protein antigen, antigenic fragments thereof, or any combinations thereof. In some aspects, the nucleotide sequence encodes one or more parasite antigens, wherein the one or more parasite antigens comprise one or more parasitic or pathogenic fungus antigens. In some aspects, the one or more parasitic or pathogenic fungus antigens are selected from the group consisting of a Candida spp. antigen (e.g., a Candida albicans antigen, a Candida glabrata antigen, a Candida parapsilosis antigen, a Candida tropicalis antigen, a Candida lusitaniae antigen, a Candida krusei antigen), a Pneumocystis spp. antigen, a Malassezia spp. antigen (e.g., a Malassezia furfur antigen), an Aspergillus fumigatus antigen, a Cryptococcus spp. antigen (e.g., a Cryptococcus neoformans antigen, a Cryptococcus gattii antigen), a Histoplasma capsulatum antigen, a Blastomyces dermatitidis antigen, a Paracoccidioides spp. antigen (e.g., a Paracoccidioides brasiliensis antigen, a Paracoccidioides lutzii antigen), a Coccidioides spp. antigen (e.g., a Coccidioides immitis antigen, a Coccidioides posadasii antigen), a Penicillium marneffei antigen, a Sporothrix schenckii antigen, a Trichosporon asahii antigen, a Fusarium spp. antigen (e.g., a Fusarium solanum antigen, a Fusarium oxysporum antigen), a Nectria spp. antigen, a Pseudoallescheria boydii antigen, a Cladophialphora bantianum antigen, a Ramichloridium spp. antigen, a Dactylaria gallopava antigen, an Exophiala spp. antigen (e.g., an Exophiala jeanselmei antigen, an Exophiala dermatitidis antigen), a Curvularia spp. antigen, a Bipolaris spp. antigen, an Alternaria spp. antigen, a Lacazia loboi antigen, a Conidiobolus spp. antigen (e.g., a Conidiobolus coronatus antigen, a Conidiobolus incongruus antigen), and any combination thereof.

In some aspects, the first or second antigen nucleic acid encodes a first SARS-CoV-2 protein and a second SARS-CoV-2 protein, respectively. In some aspects, the first SARS-CoV-2 protein is a SARS-CoV-2 S protein or an antigenic fragment thereof. In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the first SARS-CoV-2 protein has at least 70%, at least 75%, at least 80%, at least 85%, at least 90%, at least 95%, or at least 99% sequence identity to the amino acid sequence of SEQ ID NO: 2 or SEQ ID NO: 4. In some aspects, the first SARS-CoV-2 protein comprises the amino acid sequence of SEQ ID NO: 2 or SEQ ID NO: 4.

In some aspects, the second SARS-CoV-2 protein is a SARS-CoV-2 S protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the second SARS-CoV-2 protein has at least 70%, at least 75%, at least 80%, at least 85%, at least 90%, at least 95%, or at least 99% sequence identity to the amino acid sequence of SEQ ID NO: 2 or SEQ ID NO: 4. In some aspects, the second SARS-CoV-2 protein comprises the amino acid sequence of SEQ ID NO: 2 or SEQ ID NO: 4.

In some aspects, the first SARS-CoV-2 protein is a SARS-CoV-2 S protein or an antigenic fragment thereof. In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4, wherein the contiguous amino acids of SEQ ID NO: 2 or SEQ ID NO: 4 comprise one or more mutations (i.e., one or more substitutions, deletions, insertions, or any combination thereof). In some aspects, the first SARS-CoV-2 protein comprises the amino acid sequence of SEQ ID NO: 2 or SEQ ID NO: 4, wherein the polypeptide comprises one or more mutations (i.e., one or more substitutions, deletions, insertions, or any combination thereof).

In some aspects, the second SARS-CoV-2 protein is a SARS-CoV-2 S protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4, wherein the contiguous amino acids of SEQ ID NO: 2 or SEQ ID NO: 4 comprise one or more mutations (i.e., one or more substitutions, deletions, insertions, or any combination thereof). In some aspects, the second SARS-CoV-2 protein comprises the amino acid sequence of SEQ ID NO: 2 or SEQ ID NO: 4, wherein the polypeptide comprises one or more mutations (i.e., one or more substitutions, deletions, insertions, or any combination thereof).

In some aspects, the first SARS-CoV-2 protein is the receptor binding domain (RBD) of the SARS-Cov-2 S protein or an antigenic fragment thereof. In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the first SARS-CoV-2 protein has at least 70%, at least 75%, at least 80%, at least 85%, at least 90%, at least 95%, or at least 99% sequence identity to the amino acid sequence of SEQ ID NO: 6. In some aspects, the first SARS-CoV-2 protein comprises the amino acid sequence of SEQ ID NO: 6.

In some aspects, the second SARS-CoV-2 protein is the receptor binding domain (RBD) of the SARS-Cov-2 S protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the second SARS-CoV-2 protein has at least 70%, at least 75%, at least 80%, at least 85%, at least 90%, at least 95%, or at least 99% sequence identity to the amino acid sequence of SEQ ID NO: 6. In some aspects, the second SARS-CoV-2 protein comprises the amino acid sequence of SEQ ID NO: 6.

In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6, wherein the contiguous amino acids of SEQ ID NO: 6 comprise one or more mutations (i.e., one or more substitutions, deletions, insertions, or any combination thereof). In some aspects, the first SARS-CoV-2 protein comprises the amino acid sequence of SEQ ID NO: 6, wherein the polypeptide comprises one or more mutations (i.e., one or more substitutions, deletions, insertions, or any combination thereof).

In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6, wherein the contiguous amino acids of SEQ ID NO: 6 comprise one or more mutations (i.e., one or more substitutions, deletions, insertions, or any combination thereof). In some aspects, the second SARS-CoV-2 protein comprises the amino acid sequence of SEQ ID NO: 6, wherein the polypeptide comprises one or more mutations (i.e., one or more substitutions, deletions, insertions, or any combination thereof).

In some aspects, the first SARS-CoV-2 protein is the S1 subunit of the SARS-Cov-2 S protein or an antigenic fragment thereof. In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the first SARS-CoV-2 protein has at least 70%, at least 75%, at least 80%, at least 85%, at least 90%, at least 95%, or at least 99% sequence identity to the amino acid sequence of SEQ ID NO: 40. In some aspects, the first SARS-CoV-2 protein comprises the amino acid sequence of SEQ ID NO: 40.

In some aspects, the second SARS-CoV-2 protein is the S1 subunit of the SARS-Cov-2 S protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the second SARS-CoV-2 protein has at least 70%, at least 75%, at least 80%, at least 85%, at least 90%, at least 95%, or at least 99% sequence identity to the amino acid sequence of SEQ ID NO: 40. In some aspects, the second SARS-CoV-2 protein comprises the amino acid sequence of SEQ ID NO: 40.

In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40, wherein the contiguous amino acids of SEQ ID NO: 40 comprise one or more mutations (i.e., one or more substitutions, deletions, insertions, or any combination thereof). In some aspects, the first SARS-CoV-2 protein comprises the amino acid sequence of SEQ ID NO: 40, wherein the polypeptide comprises one or more mutations (i.e., one or more substitutions, deletions, insertions, or any combination thereof).

In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40, wherein the contiguous amino acids of SEQ ID NO: 40 comprise one or more mutations (i.e., one or more substitutions, deletions, insertions, or any combination thereof). In some aspects, the second SARS-CoV-2 protein comprises the amino acid sequence of SEQ ID NO: 40, wherein the polypeptide comprises one or more mutations (i.e., one or more substitutions, deletions, insertions, or any combination thereof).

In some aspects, the one or more mutations in the SARS-CoV-2 full-length S protein, the RBD of the SARS-Cov-2 S protein, the S1 subunit of the SARS-CoV-2 S protein, or antigenic fragments thereof comprise one or more mutations previously reported in Li, T. et al., Emerg Microbes Infect. 9(1):2076-90 (2020); Lee, P. et al., Immune Netw. 21(1):e4 (2021); Yu, J. et al., Science 369(6505):806-11 (2020); Cattin-Ortola, J. et al., Nat Commun. 12(1):5333 (2021); Corbett, K. et al., Nature 586(7830):567-71 (2020); Hsieh, C. et al., Science 369(6510):1501-5 (2020); and Harvey, W. et al., Nat Rev Microbiol. 19(7):409-24 (2021), each of which is incorporated by reference herein in its entirety.

In some aspects, the one or more mutations in the SARS-CoV-2 full-length S protein, the RBD of the SARS-Cov-2 S protein, the S1 subunit of the SARS-CoV-2 S protein, or antigenic fragments thereof are selected from: ΔM1-S13, S12P, S13I, L5F, L18F, T19R, T20N, P26S, Q52R, A67V, ΔH69-V70, G75V, T76I, D80A, T95I, R102I, ΔD119-F120, C136Y, D138Y, ΔF140, ΔL141-Y144, AY144, Y144S, Y145N, ΔH146, N148S, K150R, K150E, K150T, K150Q, S151P, W152C, E154K, ΔE156-F157, F157L, F157A, R158G, R190S, ΔI210, D215G, A222V, ΔL241-S243, ΔL242-L244, ΔA243-L244, ΔR246-G252, R246I, 11-amino acid residue insertion between Y248 and L249, D253G, D253N, R346K, V367F, E406W, K417E, K417V, K417N, K417T, N439K, K444A, K444R, K444N, K444Q, V445A, V445E, G447A, N450D, L452R, L452Q, Y453F, L455F, N460I, S477G, S477N, S477R, T478I, T478K, V483I, E484K, E484Q, G485R, F486A, F486V, F486L, N487A, F490S, Q493E, Q493K, S494P, N501Y, A570D, Q613H, D614G, H655Y, Q677H, Δ678-679, Δ681-682, Δ681-684, Δ682-685, P681H, P681R, R682S, R682A, R682Q, R683S, R683G, R683Q, R685G, R685Q, I692V, A701V, T716I, F817P, T859N, F888L, A892P, A899P, A942P, D950N, S982A, K986P, V987P, T1027I, Q1071H, D1118H, V1176F, M1229I, ΔC1253-T1273, ΔC1254-T1273, ΔK1255-T1273, D1257A, E1258A, D1259A, D1260A, E1262A, K1269A, H1271K, T1273A, or any combination thereof, wherein the amino acid locations correspond to SEQ ID NO: 2 or SEQ ID NO: 4.

In some aspects, the one or more mutations comprise one or more mutations in the N-terminal signal peptide, which corresponds to amino acids 1-13 of SEQ ID NO: 2 or SEQ ID NO: 4. In some aspects, the one or more mutations in the N-terminal signal peptide is ΔM1-S13, wherein the amino acid locations correspond to SEQ ID NO: 2 or SEQ ID NO: 4.

In some aspects, the one or mutations comprise one or more mutations in the C-terminus of the full-length SARS-CoV-2 S protein. In some aspects, the one or more mutations in the C-terminus of the full-length SARS-CoV-2 S protein comprise one or more mutations in the C-terminal endoplasmic reticulum (ER) retention peptide, which corresponds to amino acids 1254-1273 of SEQ ID NO: 2 or SEQ ID NO: 4. In some aspects, the one or more mutations in the C-terminal ER retention peptide are selected from D1257A, E1258A, D1259A, D1260A, E1262A, K1269A, H1271K, T1273A, or any combination thereof, wherein the amino acid locations correspond to SEQ ID NO: 2 or SEQ ID NO: 4. In some aspects, the one or more mutations in the C-terminal ER retention peptide comprise D1257A+E1258A+D1259A+D1260A+E1262A (i.e., a D/E to A mutant), wherein the amino acid locations correspond to SEQ ID NO: 2 or SEQ ID NO: 4. In some aspects, the one or mutations in the C-terminal ER retention peptide is ΔC1253-T1273, ΔC1254-T1273, or ΔK1255-T1273.

In some aspects, the one or more mutations comprise K986P+V987P (i.e., a S-2P mutation), wherein the amino acid locations correspond to SEQ ID NO: 2 or SEQ ID NO: 4. In some aspects, the one or more mutations comprise F817P+A892P+A899P+A942P (i.e., a hexa-proline S mutation), wherein the amino acid locations correspond to SEQ ID NO: 2 or SEQ ID NO: 4. In some aspects, the one or more mutations comprise one or more mutations in the 681-PRRAR/SVA-688 S1/S2 furin cleavage site, wherein the amino acid locations correspond to SEQ ID NO: 2 or SEQ ID NO: 4. In some aspects, the one or more mutations in the 681-PRRAR/SVA-688 S1/S2 furin cleavage site are: (a) R682S+R683S (i.e., a SSAR mutation), (b) Δ681-684 (i.e., a ΔPRRA mutation), (c) Δ678-679+Δ681-682, (d) R682A+R683G+R685G (i.e., a 682-AGAG-685 mutation), (e) R682Q+R683Q+R685Q, (f) R682S+R685G, or (g) Δ682-685 (i.e., a ΔRRAR mutation), wherein the amino acid locations correspond to SEQ ID NO: 2 or SEQ ID NO: 4.

In some aspects, the one or more mutations comprise: (a) F817P+A892P+A899P+A942P (i.e., a hexa-proline S mutation) and (b) K986P+V987P (i.e., a S-2P mutation), wherein the amino acid locations correspond to SEQ ID NO: 2 or SEQ ID NO: 4. In some aspects, the one or more mutations comprise: (a) R682A+R683G+R685G (i.e., a 682-AGAG-685 mutation) and (b) K986P+V987P (i.e., a S-2P mutation), wherein the amino acid locations correspond to SEQ ID NO: 2 or SEQ ID NO: 4. In some aspects, the one or more mutations comprise: (a) R682A+R683G+R685G (i.e., a 682-AGAG-685 mutation), (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations comprise: (a) R682Q+R683Q+R685Q and (b) K986P+V987P (i.e., a S-2P mutation). In some aspects, the one or more mutations comprise:: (a) R682Q+R683Q+R685Q, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations comprise: (a) R682S+R685G and (b) K986P+V987P (i.e., a S-2P mutation), wherein the amino acid locations correspond to SEQ ID NO: 2 or SEQ ID NO: 4. In some aspects, the one or more mutations comprise: (a) R682S+R685G, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4.

In some aspects, the second SARS-CoV-2 protein is a SARS-CoV-2 M protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the second SARS-CoV-2 protein has at least 70%, at least 75%, at least 80%, at least 85%, at least 90%, at least 95%, or at least 99% sequence identity to the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the second SARS-CoV-2 protein comprises the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20.

In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20, wherein the contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20 comprise one or more mutations selected from A2S, F28L, I48V, V70L, I82T, M84T, or any combination thereof, wherein the amino acid locations correspond to SEQ ID NO: 8. In some aspects, the second SARS-CoV-2 protein comprises the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20, wherein the polypeptide comprises one or more mutations selected from A2S, F28L, I48V, V70L, I82T, M84T, or any combination thereof, wherein the amino acid locations correspond to SEQ ID NO: 8.

In some aspects, the second SARS-CoV-2 protein is a SARS-CoV-2 E protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 55, at least 60, at least 65, at least 70, or at least 75 contiguous amino acids of SEQ ID NO: 22, SEQ ID NO: 24, or SEQ ID NO: 26. In some aspects, the second SARS-CoV-2 protein has at least 70%, at least 75%, at least 80%, at least 85%, at least 90%, at least 95%, or at least 99% sequence identity to the amino acid sequence of SEQ ID NO: 22, SEQ ID NO: 24, or SEQ ID NO: 26. In some aspects, the second SARS-CoV-2 protein comprises the amino acid sequence of SEQ ID NO: 22, SEQ ID NO: 24, or SEQ ID NO: 26.

In some aspects, the second SARS-CoV-2 protein is a SARS-CoV-2 N protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, or at least 400 contiguous amino acids of SEQ ID NO: 28. In some aspects, the second SARS-CoV-2 protein has at least 70%, at least 75%, at least 80%, at least 85%, at least 90%, at least 95%, or at least 99% sequence identity to the amino acid sequence of SEQ ID NO: 28. In some aspects, the second SARS-CoV-2 protein comprises the amino acid sequence of SEQ ID NO: 28.

In some aspects, promoter 1 is selected from the group consisting of: a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CAG promoter, and any combination thereof. In some aspects, the mammalian EF1 promoter is a hEF1-HTLV promoter.

In some aspects, promoter 2 is selected from the group consisting of: a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CAG promoter, and any combination thereof. In some aspects, the mammalian EF1 promoter is a hEF1-HTLV promoter.

In some aspects, promoter X is selected from the group consisting of: a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CAG promoter, and any combination thereof. In some aspects, the mammalian EF1 promoter is a hEF1-HTLV promoter.

In some aspects, promoter Y is selected from the group consisting of: a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CAG promoter, and any combination thereof. In some aspects, the mammalian EF1 promoter is a hEF1-HTLV promoter.

In some aspects, promoter Z is selected from the group consisting of: a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a R-actin promoter, a murine PGK1 promoter, a human PGK1 promoter, a CAG promoter, and any combination thereof. In some aspects, the mammalian EF1 promoter is a hEF1-HTLV promoter.

In some aspects, the IRES sequence comprises a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 41.

The polynucleotides and compositions of the present disclosure (e.g., pharmaceutical compositions, vaccines, vectors, and DNA plasmid vectors) can be formulated according to known methods for preparing pharmaceutically useful compositions.

Formulations are described in a number of sources which are well known and readily available to those skilled in the art. For example, Remington's Pharmaceutical Science (Martin E W [1995]Easton Pennsylvania, Mack Publishing Company, 19th Ed.) describes formulations which can be used in connection with the subject disclosure. Formulations suitable for parenteral administration include, for example, aqueous sterile injection solutions, which can contain antioxidants, buffers, bacteriostats, and solutes which render the formulation isotonic with the blood of the intended recipient; and aqueous and nonaqueous sterile suspensions which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the formulations of the subject disclosure can include other agents conventional in the art having regard to the type of formulation in question.

The disclosure also provides lyophilized (or freeze-dried) compositions or vaccines that can be safely stored for periods of time and reconstituted prior to use. In some aspects, the composition, pharmaceutical composition, or vaccine of the disclosure is a lyophilized product, e.g., substantially free of aqueous components. In some aspects, the lyophilized composition or vaccine is reconstituted in a diluent, e.g., prior to administration. In some aspects, the lyophilized composition or vaccine is reconstituted in water.

Some aspects are directed to a lyophilized composition or vaccine comprising: (1) any polynucleotide or DNA plasmid vector disclosed herein and (2) any delivery component disclosed herein, wherein the composition is substantially free of aqueous components. In some aspects, a composition or vaccine of the disclosure is lyophilized.

In some aspects, the lyophilized composition or vaccine is stable at 0° C. to 5° C. for at least about 1 month (or 30 days), 2 months, 3 months, 4 months, 5 months, 6 months, 7 months, 8 months, 9 months, 10 months, 11 months, or 12 months. In some aspects, the lyophilized composition or vaccine is stable at 0° C. to 5° C. for at least 1 year, at least 2 years, at least 3 years, at least 4 years or at least 5 years. In some aspects, the lyophilized composition or vaccine is stable at 25° C. for at least about 7 days, about 10 day, or about 14 days.

In some aspects, the lyophilized composition or vaccine is reconstituted into a reconstituted composition or vaccine formulation for administration. In some aspects, the reconstituted composition or vaccine is stable at 0° C. to 5° C. for at least about 1 month, about 2 months, about 3 months, about 4 months, about 5 months, or about 6 months after reconstitution of the lyophilized composition or vaccine with a diluent (e.g., water). In some aspects, the reconstituted composition or vaccine is stable at 25° C. for at least about 2 days, about 3 days, about 4 days, about 5 days, about 6 days, or about 7 days after reconstitution of the lyophilized composition with a diluent. In some aspects, the diluent is water.

The compositions of the subject disclosure can further comprise other components such as a pharmaceutically acceptable carrier and/or an adjuvant. The adjuvant can be other genes that are expressed in alternative polynucleotide, plasmid, or vector or are delivered as proteins in combination with the compositions of the subject disclosure. The adjuvant can be selected from the group consisting of: α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof. Other genes that can be useful adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, INK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2, functional fragments thereof, and combinations thereof.

In some aspects, compositions of the disclosure can be formulated according to the mode of administration to be used. For example, an injectable vaccine pharmaceutical composition can be sterile, pyrogen free and particulate free. An isotonic formulation or solution can be used. Additives for isotonicity can include sodium chloride, dextrose, mannitol, sorbitol, and lactose. The vaccine can comprise a vasoconstriction agent. The isotonic solutions can include phosphate buffered saline. Vaccine can further comprise stabilizers including gelatin and albumin. The stabilizers can allow the formulation to be stable at room or ambient temperature for extended periods of time, including LGS or polycations or polyanions

In some aspects, therapeutically effective and optimal dosage ranges for the compositions of the subject disclosure can be determined using methods known in the art. For example, volunteer subjects or test animals can be inoculated with varying dosages at scheduled intervals and test blood samples can be evaluated for levels of antibody and/or SARS-CoV-2 neutralizing activity present in the blood, for example, by Western blot analysis. Such results can be used to refine an optimized immunization dosage and schedule for effective immunization of mammalian, specifically human, subjects.

Also provided herein are recombinant host cells comprising any polynucleotide, vector, DNA plasmid vector, or vaccine described herein. Host cells include prokaryotic cells, lower eukaryotic cells such as yeast, other eukaryotic cells such as insect cells, and higher cukaryotic cells such as mammalian cells. Mammalian host cells include, but are not limited to, CHO, VERO, BHK, Hela, MDCK, HEK 293, NIH 3T3, W138, BT483, Hs578T, HTB2, BT20 and T47D, NSO (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10, HBK, NSO, HT1080 and HsS78Bst cells. In some aspects, the recombinant host cells arc prepared by introducing the vectors, polynucleotides, or vaccines described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook. et al. (Molecular Cloning: A Laboratory Manual. 2nd. ed. Cold Spring Harbor Laboratory. Cold Spring Harbor Laboratory Press. Cold Spring Harbor. NY (1989).

### 5.4.1 Delivery Components

In some aspects, the disclosed compositions, pharmaceutical compositions, vaccines, vectors, multicistronic mRNA vectors, or DNA plasmid vectors further comprise a delivery component. In some aspects, the delivery component is a non-viral delivery component or system based on “naked DNA” or formulated plasmid DNA. In some aspects, the delivery component or system can be used to deliver mRNA. In some aspects, the non-viral delivery component is a cationic polymer. In some aspects, the cationic polymer is a synthetic functionalized polymer, a lipid, a lipopolymer, or a chemical derivative thereof.

Non-viral gene delivery components or systems, based on “naked DNA” or formulated plasmid DNA, have potential benefits over viral vectors due to simplicity of use and lack of inciting a specific immune response. A number of synthetic gene delivery systems have been described to overcome the limitations of naked DNA, including cationic lipids, peptides, and polymers. Similarly, non-viral delivery systems can be used for delivery of mRNA.

Polymers have emerged as a viable alternative to current systems because their excellent molecular flexibility allows for complex modifications and incorporation of novel chemistries. Cationic polymers, such as poly(L-lysine) (PLL) and poly(L-arginine) (PLA), polyethyleneimine (PEI) have been widely studied as gene delivery candidates due to their ability to condense DNA, and promote DNA stability and transmembrane delivery. PEI efficiently condenses DNA into small narrowly distributed positively charged spherical complexes and can transfect cells in vitro and in vivo. PEI is similar to other cationic polymers in that the transfection activity of PEI increases with increasing polymer/DNA ratios. A distinct advantage of PEI over PLL is its endosomolytic activity which enables PEI to yield high transfection efficiency. Commercial branched PEI is composed of 25% primary amines, 50% secondary amines and 25% tertiary amines. The overall protonation level of PEI doubles from pH 7 to pH 5, which means in the endosome PEI becomes heavily protonated. Protonation of PEI triggers chloride influx across the endosomal membrane, and water follows to counter the high ion concentration inside the endosome, which eventually leads to endosomal disruption from osmotic swelling and release of the entrapped DNA. Because of its intrinsic endosomolytic activity, PEI generally does not require the addition of an endosomolytic agent for transfection. Due to these advantages PEI has been increasingly utilized in polymer functionalization strategies to create safer and more efficient delivery systems.

In some aspects, the delivery component is a cationic polymer. In some aspects, the cationic polymer is a synthetic functionalized polymer, a lipid, a lipopolymer, or a chemical derivative thereof. In some aspects, the cationic polymer is present in an amount sufficient to produce a ratio of amine nitrogen in the cationic polymer to phosphate in the DNA plasmid vector or multicistronic mRNA vector from about 0.1:1 to about 100:1. In some aspects, the ratio of amine nitrogen in the cationic polymer to phosphate in the DNA plasmid vector or multicistronic mRNA vector is from about 0.1:1 to about 10:1. In some aspects, the composition, pharmaceutical composition, or vaccine comprises about 0.5 mg/ml to about 5.0 mg/ml nucleic acid complexed with the cationic polymer. In some aspects, the delivery component is a poloxamer or a derivative thereof. In some aspects, the poloxamer or derivative thereof is present in a solution with the polynucleotide, multicistronic mRNA vector, or DNA plasmid vector from about 0.1% to about 5% or about 0.5%—about 5%. In some aspects, the delivery component is a β-amino ester. In some aspects, the polymer is present in a solution with the polynucleotide, multicistronic mRNA vector, or DNA plasmid vector from about 0.1% to about 5% or about 0.5%—-about 5%. In some aspects, the delivery component is a poly-inosinic-polycytidylic acid. In some aspects, the poly-inosinic-polycytidylic acid is present in a solution with the polynucleotide, multicistronic mRNA vector, or DNA plasmid vector from about 0.1% to about 5% or about 0.5% —about 5%.

### 5.4.1.1 Biodegradable Cross-linked Cationic Multi-block Copolymers

In some aspects, the delivery component is a biodegradable cross-linked cationic multi-block copolymer, for example any biodegradable cross-linked cationic multi-block copolymer disclosed in U.S. Pat. No. 8,445,017. In some aspects, the biodegradable cross-linked cationic multi-block copolymer is a biodegradable cross-linked cationic multi-block copolymer of linear poly(alkylenimine) (LPAI) and a hydrophilic linker, wherein said LPAI blocks are crossed linked together by said hydrophilic linker with biodegradable ester, amide, disulfide, or phosphate linkages bonds. In some aspects, the linear poly(alkylenimine) (LPAI) is a member selected from the group consisting of polyethyleneimine, polypropylenimine, aminoglycoside-polyamine, dideoxy-diamino-.beta.-cyclodextrin, spermine and spermidine. In some aspects, the linear poly(alkylenimine) (LPAI) is linear poly(ethylenimine) (LPEI).

In some aspects, the cross-linked cationic multi-block copolymer is linked by the biodegradable linkers to other moieties such as, for example, fluorescent markers, lipids anchors or their derivatives, i.e., cholesterol, fatty acids or their derivatives. In some aspects, the molecular weight of the linear PEI used in this dislosure is within the range of 1,000 to 25,000 Dalton. In some aspects, the linear PEI blocks are preferably linked to one another via a diamide linkage utilizing a biodegradable disulfidediacid-derived linker, i.e., dithiodipropionate derivatives. In some aspects, the molar ratio of the linker to the PEI is within a range of 1/1 to 5/1; the molar ratio of the lipid anchors to PEI is from 0/1 to 3/1. In some aspects, the polymer is formulated as a polyammonium salt, preferably with a chloride counterion. Since the toxicity of PEI increases with an increase in its molecular weight, the use of lower molecular weight PEIs as blocks in the polymer provides an improved gene carrier for use as a general reagent for transfection of mammalian cells, and for the in vivo application of gene therapy.

In some aspects, the biodegradable, cross-linked cationic multi-block copolymer comprises low molecular weight linear PEI blocks and a dithioacid moiety, i.e., dithiodipropionic acid, as biodegradable linkers. The biodegradable, cross-linked cationic multi-block copolymers are synthesized by cross-linking low molecular weight linear PEI units via a biodegradable disulfide linkage. These biodegradable cross-linked cationic multi-block copolymers are water soluble and transfectionally superior (68-70 fold higher activity) to single block polymers. See U.S. Pat. No. 8,445,017.

In some aspects, the linker to the polymer molecular weight ratio is <0.2 which minimizes the dilution of polyamine polymer backbone. In some aspects, the chemical bond between the linker and the polymer blocks is a disulfide bond which can be biodegraded more easily as compared to amide bonds. Other biodegradable bonds can also be used in the present disclosure includes: phosphoesters, hydrazone, cis-asotinyl, urethane and poly(ethyl). Since any linker reacts in stepwise fashion, it can link either different blocks or the different areas of the same block (loop formation). The latter will favor the formation of a lightly cross-linked material with poor solubility due to multiple looping. The process disclosed in U.S. Pat. No. 8,445,017 solves this problem by incorporating partial and reversible blocking/protection of nitrogen atoms in the LPEI blocks. Such LPEI functionalization also increases polymer solubility, facilitating the linking of LPEI blocks. This process also allows for convenient incorporation of pendant auxiliary ligands (for example, lipids, or fluorescent markers) onto a cationic polymer.

In some aspects, the cationic block copolymers are represented by the following formula: (CP)ₓL_{y}Y_{z} wherein CP represents a cationic polymer containing at least one secondary amine group, said CP polymer has a number averaged molecular weight within the range of 1,000 to 25,000 Dalton; Y represents a bifunctional biodegradable linker containing ester, amide, disulfide, or phosphate linkages; L represents a ligand; x is an integer in the range from 1 to 20; y is an integer from 0 to 100; and z is an integer in the range from 0 to 40. In some aspects, the cationic polymer comprises linear polyethyleneimine (LPEI). In some aspects, the LPEI is BD15-12, which has the following formula:

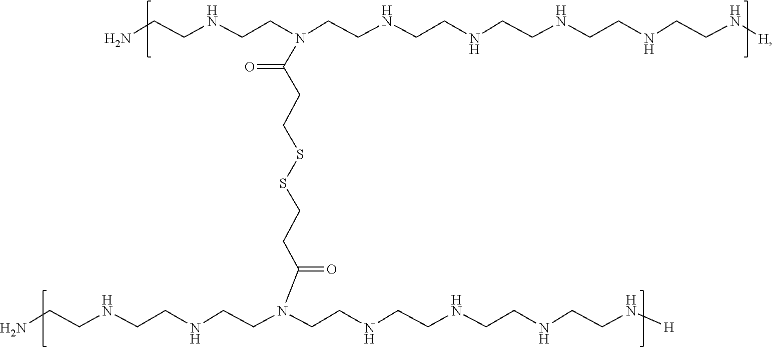

wherein the PEIs are approximately 15,000 Da, and wherein there is an average of 12 crosslinkers per PEI. In some aspects, the LPEI is Omnifect, which has the following formula:

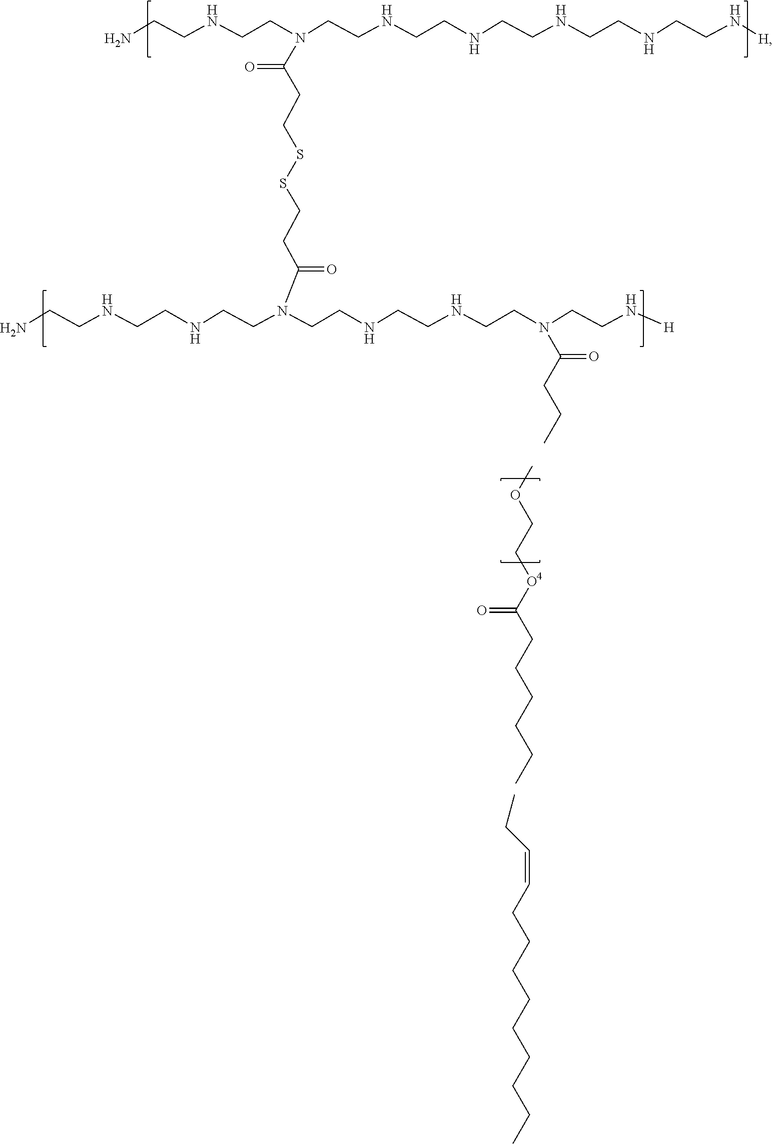

wherein the PEIs are approximately 3,600 Da, wherein there is an average of 3 crosslinkers per PEI, and wherein there is an average of 1 PEG-lipid per PEI. In some aspects, the bifunctional biodegradable linker is hydrophilic and comprises a biodegradable linkage comprising a disulfide bond. In some aspects, the bifunctional biodegradable linker is a dithiodipropionyl linker.

In some aspects, the biodegradable cross-linked cationic multi-block copolymer comprises LPEI and a dithiodipropionyl linker for cross-linking the multi-block copolymer, wherein the LPEI has an average molecular weight of 1,000 to 25,000 Dalton. In some aspects, the biodegradable cross-linked cationic multi-block copolymer is covalently linked to at least one ligand.

In some aspects, the biodegradable, cross-linked, cationic, multi-block copolymers of LPEI and lipopolymers of the present disclosure have amine groups that are electrostatically attracted to polyanionic compounds such as nucleic acids. In some aspects, the cationic copolymer condenses DNA and forms compact structures. In addition, low toxicity of monomeric degradation products after delivery of bioactive materials provides for gene carriers with reduced cytotoxicity and increased transfection efficiency.

In some aspects, the biodegradable cross-linked cationic multi-block copolymers are conjugated with tracers (for example, fluorescent markers) or ligands either directly or via spacer molecules. In some aspects, only a small portion of the available amino groups is coupled to the ligand. The ligands conjugated to the polymers are targeting ligands that direct the polymers-nucleic acid complex to bind to specific target cells and penetrate into such cells. The targeting ligands can also be an intracellular targeting element, enabling the transfer of the nucleic acid/drug to be guided towards certain favored cellular compartments (mitochondria, nucleus, and the like). The targeting ligands conjugated to the polymers direct the polymers-nucleic acid complex to bind to specific target cells and penetrate into such cells (e.g., epithelial cells, endothelial cells, hematopoietic cells, and the like). The target ligands can also be an intracellular targeting element, enabling the transfer of the nucleic acid/drug to be guided towards certain favored cellular compartments (mitochondria, nucleus, and the like).

In some aspects, the targeting ligand is a polypeptide, folate, and an antigen. In some aspects, the polypeptide is a glycoprotein (e.g., transferrin or asialoorosomucoid (ASOR)), an antibody, an antibody fragment, a cell receptor, a cytokine receptor, or a growth factor receptor (e.g., epidermal growth factor receptor). In some aspects, the antigen is a viral antigen, a bacterial antigen, or a parasite antigen. In some aspects, the ligand is a fusogenic agent (e.g., polymixin B and hemaglutinin HA2), a lysosomotrophic agent, or a nucleus localization signal (NLS) (e.g., T-antigen, and the like). In some aspects, the ligand is a sugar moiety coupled to an amino group. In some aspects, the sugar moiety is a mono- or oligo-saccharide, such as galactose, glucose, fucose, fructose, lactose, sucrose, mannose, cellobiose, nytrose, triose, dextrose, trehalose, maltose, galactosamine, glucosamine, galacturonic acid, glucuronic acid, and gluconic acid.

In some aspects, the biodegradable cross-linked cationic multi-block copolymer is covalently linked to polyethylene glycol (PEG) of molecular weight ranging from 500 to 20,000 Dalton. In some aspects, the biodegradable cross-linked cationic multi-block copolymer is covalently linked to a fatty acyl chain selected from the group consisting of: oleic acid, palmitic acid, and stearic acid. In some aspects, the biodegradable cross-linked cationic multi-block copolymer comprises at least one amine group that is electrostatically attracted to a polyanionic compound. In some aspects, the polyanionic compound is a nucleic acid, wherein the biodegradable cross-linked cationic multi-block copolymer condenses the nucleic acid to form a compact structure.

In some aspects, the biodegradable cross-linked cationic multi-block copolymer has the following formula.

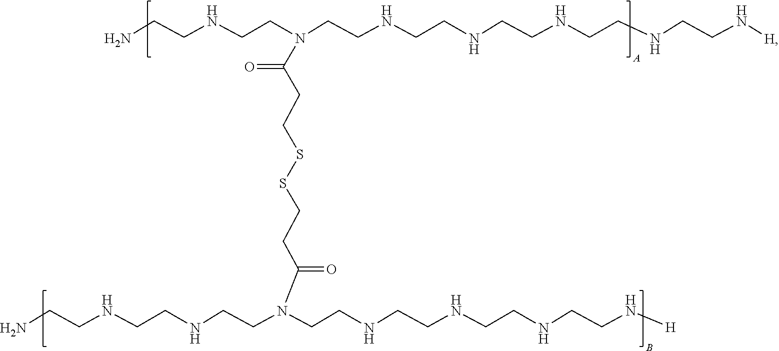

wherein A and B are such that the molecular weight of the individual linear polyethylenimine chains are from 5,000 to 25,000 Dalton; the intermolecular crosslinks connect approximately 5-10% of amines; the biodegradable crosslinks are dithiodipropionyl (each half composed of 3 carbon atoms) and can be from 1-10 carbon atoms. In some aspects, the biodegradable cross-linked cationic polymer comprises 10,000 to 15,000 Dalton linear PEI covalently connected with a dithiopropionyl linkage (see, e.g., U.S. Pat. No. 8,445,017). In some aspects, the biodegradable cross-linked cationic polymer comprises 10,000 to 15,000 Dalton linear PEI covalently connected with a dithiopropionyl linkage (see, e.g., U.S. Pat. No. 8,445,017) and is further conjugated to polyethyleneglycol (PEG) of molecular weight ranging from 500 to 20,000 Dalton. In some aspects, the biodegradable cross-linked cationic polymer comprises 10,000 to 15,000 Dalton linear PEI covalently connected with a dithiopropionyl linkage (see, e.g., U.S. Pat. No. 8,445,017) and is further conjugated to polyethyleneglycol (PEG) of molecular weight ranging from 500 to 20,000 Dalton. In some aspects, the biodegradable cross-linked cationic polymer comprises 15,000 to 20,000 Dalton linear PEI covalently connected with a dithiopropionyl linkage (see, e.g., U.S. Pat. No. 8,445,017). In some aspects, the biodegradable cross-linked cationic polymer comprises 15,000 to 20,000 Dalton linear PEI covalently connected with a dithiopropionyl linkage (see, e.g., U.S. Pat. No. 8,445,017) and is further conjugated to polyethyleneglycol (PEG) of molecular weight ranging from 500 to 20,000 Dalton.

In some aspects, the biodegradable cross-linked cationic multi-block copolymer is present in an amount sufficient to produce a ratio of amine nitrogen in the biodegradable cross-linked cationic multi-block copolymer to phosphate in the polynucleotide, multicistronic mRNA vector, or DNA plasmid vector from about 0.01:1 to about 50:1 (e.g., about 0.01:1 to about 40:1; about 0.01:1 to about 30:1; about 0.01:1 to about 20:1; about 0.01:1 to about 10:1, or about 0.01:1 to about 5:1). In some aspects, the ratio of amine nitrogen in the biodegradable cross-linked cationic multi-block copolymer to phosphate in the polynucleotide, multicistronic mRNA vector, or DNA plasmid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the biodegradable cross-linked cationic multi-block copolymer to phosphate in the polynucleotide, multicistronic mRNA vector, or DNA plasmid vector is from about 1:10 to about 10:1.

In some aspects, the composition, pharmaceutical composition, or vaccine comprises about 0.1 mg/ml to about 10 mg/ml (e.g., about 0.1 mg/ml to about 5 mg/ml; about 0.5 mg/ml to about 10 mg/ml; or about 0.5 mg/ml to about 5 mg/ml) nucleic acid complexed with the biodegradable cross-linked cationic multi-block copolymer. In some aspects, the composition, pharmaceutical composition, or vaccine comprises about 1 mg/ml to about 10 mg/ml (e.g., about 1 mg/ml to about 6 mg/ml; about 2 mg/ml to about 6 mg/ml; about 5 mg/ml to about 10 mg/ml; or about 6 mg/ml to about 10 mg/ml) nucleic acid complexed with the biodegradable cross-linked cationic multi-block copolymer.

### 5.4.1.2 Cationic Lipopolymers Comprising a PEI Backbone

In some aspects, the delivery component is a cationic lipopolymer comprising a PEI backbone covalently linked to a lipid or a polyethylene glycol (PEG) as disclosed in U.S. Pat. No. 7,964,571. In some aspects, the PEI backbone is covalently linked to a lipid and a PEG. In some aspects, the lipid and the PEG are directly attached to the PEI backbone by covalent bonds. In some aspects, the lipid is attached to the PEI backbone through a PEG spacer. In some aspects, the PEG has a molecular weight of between 50 to 20,000 Dalton. In some aspects, the molar ratio of PEG to PEI is within a range of 0.1:1 to 500:1. In some aspects, the molar ratio of the lipid to the PEI is within a range of 0.1:1 to 500:1. In some aspects, the lipid is a cholesterol, a cholesterol derivative, a C₁₂ to C₁₈ fatty acid, or a fatty acid derivative. The addition of PEG enhances the stability of the nucleic acid/polymer complexes in the biological milieu and allows for the incorporation of ligands (e.g., a targeting ligand) on to the PPC chain to improve the tissue selectivity of delivery. See U.S. Pat. No. 7,964,571.

In some aspects, the cationic lipopolymer is a PEG:PEI:cholesterol (PPC) lipopolymer, which comprises a PEI backbone covalently linked to cholesterol and PEG. In some aspects, the PEI is covalently linked to cholesterol and PEG, and wherein the average PEG:PEI:cholesterol molar ratio in the cationic lipopolymer is within the range of 1-5 PEG:1 PEI:0.4-1.5 cholesterol. In some aspects, the PEG-PEI-cholesterol (PPC) lipopolymer has an average PEG:PEI:cholesterol ratio of 2.5:1:0.6. In some aspects, the PEI has a linear or branch configuration with a molecular weight of 100 to 500,000 Dalton.

In some aspects, the cationic lipopolymer further comprises a pendant functional moiety selected from the group consisting of: a receptor ligand, a membrane permeating agent, an endosomolytic agent, a nuclear localization sequence, and a pH sensitive endosomolytic peptide.

In some aspects, the cationic lipopolymer further comprises a targeting ligand, wherein the targeting ligand is directly attached to the PEI backbone or is attached through a PEG linker. In some aspects, the targeting ligand is selected from the group consisting of: a sugar moiety, a polypeptide, folate, and an antigen. In some aspects, the sugar moiety is a monosaccharide. In some aspects, the monosaccharide is galactose. In some aspects, the sugar moiety is an oligosaccharide. In some aspects, the polypeptide is a glycoprotein, an antibody, an antibody fragment, a cell receptor, a cytokine receptor, or a growth factor receptor. In some aspects, the growth factor receptor is an epidermal growth factor receptor. In some aspects, the glycoprotein is transferrin or asialoorosomucoid (ASOR). In some aspects, the antigen is a viral antigen, a bacterial antigen, or a parasite antigen.

In some aspects, the cationic lipopolymer is present in an amount sufficient to produce a ratio of amine nitrogen in the cationic polymer to phosphate in the polynucleotide, multicistronic mRNA vector, or DNA plasmid vector from about 0.01:1 to about 50:1 (e.g., about 0.01:1 to about 40:1; about 0.01:1 to about 30:1; about 0.01:1 to about 20:1; about 0.01:1 to about 10:1, or about 0.01:1 to about 5:1). In some aspects, the ratio of amine nitrogen in the cationic lipopolymer to phosphate in the polynucleotide, multicistronic mRNA vector, or DNA plasmid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the cationic lipopolymer to phosphate in the polynucleotide, multicistronic mRNA vector, or DNA plasmid vector is from about 1:10 to about 10:1.

In some aspects, the composition, pharmaceutical composition, or vaccine comprises about 0.1 mg/ml to about 10.0 mg/ml (e.g., about 0.1 mg/ml to about 5 mg/ml; about 0.5 mg/ml to about 10 mg/ml; or about 0.5 mg/ml to about 5 mg/ml) nucleic acid complexed with the cationic polymer. In some aspects, the composition, pharmaceutical composition, or vaccine comprises about 1 mg/ml to about 10 mg/ml (e.g., about 1 mg/ml to about 6 mg/ml; about 2 mg/ml to about 6 mg/ml; about 5 mg/ml to about 10 mg/ml; or about 6 mg/ml to about 10 mg/ml) nucleic acid complexed with the cationic polymer.

### 5.4.1.3 Lipopolyamines and Derivatives Thereof

In some aspects, the delivery component of the composition, pharmaceutical composition, or vaccine comprises a lipopolyamine with the following formula:

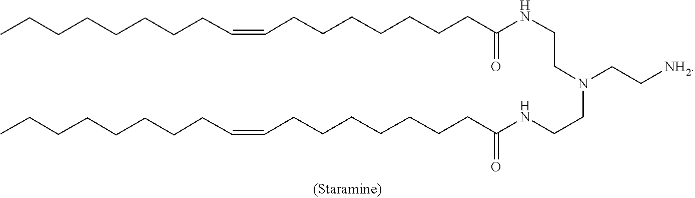 [SMILES: CCCCCCCC/C=C\CCCCCCCC(=O)NCCN(CCN)CCNC(=O)CCCCCCC/C=C\CCCCCCCC]

In some aspects, the delivery component comprises a mixture of the lipopolyamine and an alkylated derivative of the lipopolyamine. In some aspects, the alkylated derivative of the lipopolyamine is a polyoxyalkylene, polyvinylpyrrolidone, polyacrylamide, polydimethylacrylamide, polyvinyl alcohol, dextran, poly (L-glutamic acid), styrene maleic anhydride, poly-N-(2-hydroxypropyl) methacrylamide, or polydivinylether maleic anhydride. In some aspects, the alkylated derivative of the lipopolyamine has the following formula:

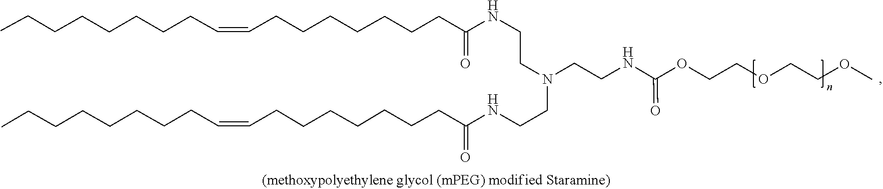

wherein n is an integer from 10 to 100 repeating units containing 2-5 carbon atoms each. In some aspects, the alkylated derivative of the lipopolyamine has the following formula:

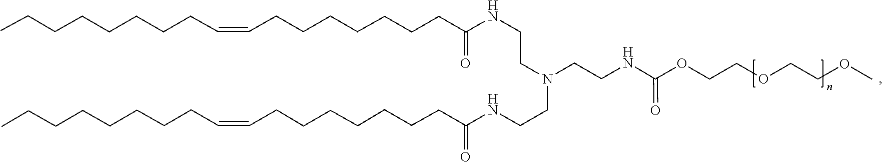

wherein n=11 (Staramine-mPEG515). In some aspects, the alkylated derivative of the lipopolyamine has the following formula:

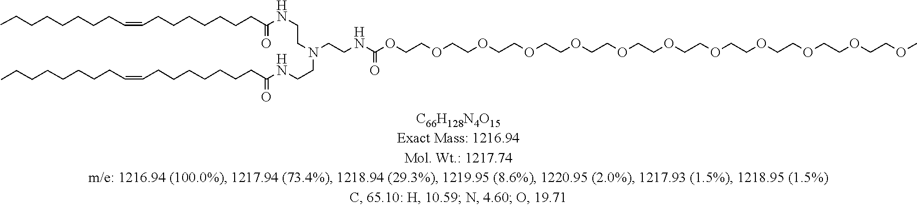 [SMILES: CCCCCCCC/C=C\CCCCCCCC(=O)NCCN(CCNC(=O)CCCCCCC/C=C\CCCCCCCC)CCNC(=O)OCCOCCOCCOCCOCCOCCOCCOCCOCCOCCOCCOC]

In some aspects, the ratio of the lipopolyamine to the alkylated derivative of the lipopolyamine in the mixture is 1:1 to 10:1. In some aspects, the lipopolyamine is present in an amount sufficient to produce a ratio of amine nitrogen in the lipopolyamine to phosphate in the polynucleotide, multicistronic mRNA vector, or DNA plasmid vector from about 0.01:1 to about 50:1 (e.g., about 0.01:1 to about 40:1; about 0.01:1 to about 30:1; about 0.01:1 to about 20:1; about 0.01:1 to about 10:1, or about 0.01:1 to about 5:1). In some aspects, the ratio of amine nitrogen in the lipopolyamine to phosphate in the polynucleotide, multicistronic mRNA vector, or DNA plasmid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the lipopolyamine to phosphate in the polynucleotide, multicistronic mRNA vector, or DNA plasmid vector is from about 1:10 to about 10:1.

In some aspects, the delivery component of the composition, pharmaceutical composition, or vaccine comprises a lipopolyamine with the following formula:

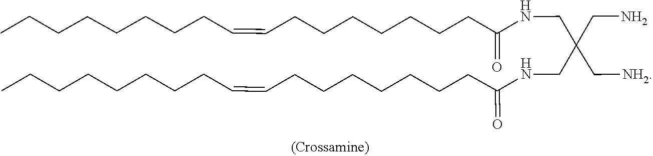 [SMILES: CCCCCCCC/C=C\CCCCCCCC(=O)NCC(CN)(CN)CNC(=O)CCCCCCC/C=C\CCCCCCCC]

In some aspects, the delivery component comprises a mixture of the lipopolyamine and an alkylated derivative of the lipopolyamine. In some aspects, the alkylated derivative of the lipopolyamine is a polyoxyalkylene, polyvinylpyrrolidone, polyacrylamide, polydimethylacrylamide, polyvinyl alcohol, dextran, poly (L-glutamic acid), styrene maleic anhydride, poly-N-(2-hydroxypropyl) methacrylamide, or polydivinylether maleic anhydride. In some aspects, the ratio of the lipopolyamine to the alkylated derivative of the lipopolyamine in the mixture is 1:1 to 10:1. In some aspects, the lipopolyamine is present in an amount sufficient to produce a ratio of amine nitrogen in the lipopolyamine to phosphate in the polynucleotide, multicistronic mRNA vector, or DNA plasmid vector from about 0.01:1 to about 50:1 (e.g., about 0.01:1 to about 40:1; about 0.01:1 to about 30:1; about 0.01:1 to about 20:1; about 0.01:1 to about 10:1, or about 0.01:1 to about 5:1). In some aspects, the ratio of amine nitrogen in the lipopolyamine to phosphate in the polynucleotide, multicistronic mRNA vector, or DNA plasmid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the lipopolyamine to phosphate in the polynucleotide, multicistronic mRNA vector, or DNA plasmid vector is from about 1:10 to about 10:1.

In some aspects, the composition, pharmaceutical composition, or vaccine comprises about 0.1 mg/ml to about 10.0 mg/ml (e.g., about 0.1 mg/ml to about 5 mg/ml; about 0.5 mg/ml to about 10 mg/ml; or about 0.5 mg/ml to about 5 mg/ml) nucleic acid complexed with the lipopolyamine or derivative thereof. In some aspects, the composition, pharmaceutical composition, or vaccine comprises about 1 mg/ml to about 10 mg/ml (e.g., about 1 mg/ml to about 6 mg/ml; about 2 mg/ml to about 6 mg/ml; about 5 mg/ml to about 10 mg/ml; or about 6 mg/ml to about 10 mg/ml) nucleic acid complexed with the lipopolyamine or derivative thereof.

In some aspects, the polynucleotide, vector, multicistronic mRNA vector, or DNA plasmid vector of the disclosure is complexed with or encapsulated by a delivery component, e.g., a lipopolymer. In some aspects, the polynucleotide, vector, multicistronic mRNA vector, or DNA plasmid vector is encapsulated by a delivery component, e.g., a lipopolymer. In some aspects, at least 20%, at least 30%, at least 40%, at least 50%, at least 60%, at least 70%, at least 75%, at least 80%, at least 85%, at least 90%, at least 95%, or at least 99% of the polynucleotide, vector, multicistronic mRNA vector, or DNA plasmid vector is encapsulated by the delivery component, e.g., a lipopolymer. In some aspects, about 20% to about 100%, about 20% to about 80%, about 20% to about 60%, or about 40% to about 80% of the polynucleotide, vector, multicistronic mRNA vector, or DNA plasmid vector of the disclosure is encapsulated by the delivery component, e.g., a lipopolymer. In some aspects, the delivery component forms a micellular structure.

### 5.4.1.4 Poloxamers and Derivatives Thereof

In some aspects, the delivery component comprises a poloxamer back-bone having a metal chelator covalently coupled to at least one terminal end of the poloxamer backbone (e.g. Crown Poloxamer). In some aspects, the metal chelator is coupled to at least two terminal ends of the poloxamer backbone. In some aspects, the poloxamer backbone is a poloxamer backbone disclosed in U.S. Publ. No. 2010/0004313, which is herein incorporated by reference in its entirety. In some aspects, the metal chelator is a metal chelator disclosed in U.S. Publ. No. 2010/0004313. In some aspects, the delivery component of the composition, pharmaceutical composition, or vaccine comprises a polymer having the following formula:

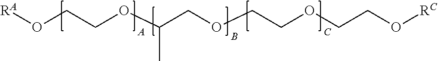

or pharmaceutically acceptable salts thereof, wherein:
   A represents an integer from 2 to 141;
   B represents an integer from 16 to 67;
   C represents an integer from 2 to 141;
   R^{A} and R^{C} are the same or different, and are R′-L- or H, wherein at least one of R^{A} and R^{C} is R′-L-;
   L is a bond, —CO—, —CH₂—O—, or —O—CO—; and
   R′ is a metal chelator.

In some aspects, the metal chelator is R^{N}NH—, R^{N}₂N, or (R″—(N(R″) CH₂CH₂)ₓ)₂—N—CH₂CO—, wherein each x is independently 0-2, and wherein R″ is HO₂C—CH₂—.

In some aspects, the metal chelator is a crown ether selected from the group consisting of 12-crown-4, 15-crown-5, 18-crown-6, 20-crown-6, 21-crown-7, and 24-crown-8. In some aspects, the crown ether is a substituted-crown ether, wherein the substituted-crown ether has:
(1) one or more of the crown ether oxygens independently replaced by NH or S,
(2) one or more of the crown ether CH₂—CH₂— moieties replaced by —C₆H₄—, —C₁₀H₆—, or C₆H₁₀—,
(3) one or more of the crown ether —CH₂—O—CH₂— moieties replaced by C₄H₂O— or —C₅H₃N—, or
(4) any combination thereof.

In some aspects, the metal chelator is a cryptand, wherein the cryptand is selected from the group consisting of (1,2,2) cryptand, (2,2,2) cryptand, (2,2,3) cryptand, and (2,3,3) cryptand. In some aspects, the cryptand is a substituted-cryptand, wherein the substituted cryptand has:
(1) one or more of the crypthand ether oxygens independently replaced by NH or S,
(2) one or more of the crown ether —CH₂—CH₂— moieties replaced by —C₆H₄—, C₁₀H₆—, or —C₆H₁₀—,
(3) one or more of the crown ether —CH₂—O—CH₂— moieties replaced by —C₄H₂O— or —C₅H₃N—, or
(4) any combination thereof.

In some aspects, the delivery component is Crown Poloxamer (aza-crown-linked poloxamer), wherein the Crown Poloxamer comprises a polymer having the following formula:

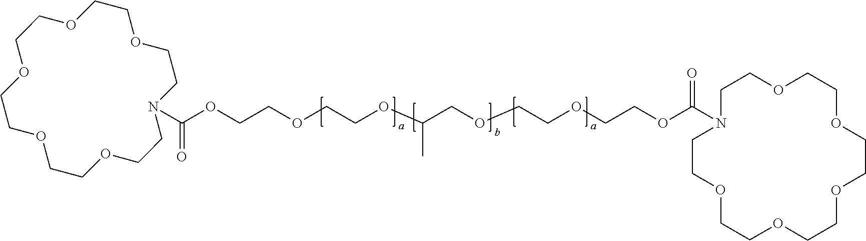

or pharmaceutically acceptable salts thereof, wherein:
   a represents an integer of about 10 units; and
   b represents an integer of about 21 units; and
wherein the total molecular weight of the polymer is about 2,000 Da to about 2,200 Da.

In some aspects, the polymer is present in a solution with the polynucleotide, multicistronic mRNA vector, or DNA plasmid vector from about 0.1%—about 5% or about 0.5%—about 5%.

In some aspects, the delivery component further comprises benzalkonium chloride (BAK).

In some aspects, the delivery component comprises BD15-12. In some aspects, the ratio of nucleotide to BD15-12 polymer (N:P) is 5:1.

In some aspects, the delivery component comprises Omnifect. In some aspects, the ratio of nucleotide to Omnifect polymer (N:P) is 10:1.

In some aspects, the delivery component comprises Crown Poloxamer (aza-crown-linked poloxamer). In some aspects, the ratio of nucleotide to Crown Poloxamer (N:P) is 5:1. In some aspects, the delivery component comprises Crown Poloxamer and a PEG-PEI-cholesterol (PPC) lipopolymer. In some aspects, the delivery component comprises Crown Poloxamer and benzalkonium chloride. In some aspects, the delivery component comprises Crown Poloxamer and Omnifect. In some aspects, the delivery component comprises Crown Poloxamer and a linear polyethyleneimine (LPEI). In some aspects, the delivery component comprises Crown Poloxamer and BD15-12.

In some aspects, the delivery component comprises Staramine and mPEG modified Staramine. In some aspects, the mPEG modified Staramine is Staramine-mPEG515. In some aspects, the mPEG modified Staramine is Staramine-mPEG11. In some aspects, the ratio of Staramine to mPEG modified Staramine is 10:1. In some aspects, the nucleotide to polymer (N:P) ratio is 5:1. In some aspects, the delivery component comprises Staramine, mPEG modified Staramine, and Crown Poloxamer. In some aspects, the delivery component comprises Staramine, Staramine-mPEG515, and Crown Poloxamer. In some aspects, the delivery component comprises Staramine, Staramine-mPEG11, and Crown Poloxamer.

### 5.5 Kits and Containers

The present disclosure also features containers comprising any polynucleotide, vector, vaccine, composition, or pharmaceutical composition described and exemplified herein. In some aspects, the container is a glass vial.

The present disclosure also features kits comprising any polynucleotide, vector, vaccine, composition, or pharmaceutical composition described and exemplified herein. The kits can be used to supply polynucleotides, vectors, vaccines, composition, pharmaceutical compositions, and other agents for use in diagnostic, basic research, or therapeutic methods, among others. In some aspects, the kits comprise any one or more of the polynucleotides, vectors, vaccines, composition, or pharmaceutical compositions described or exemplified herein and instructions for using the one or more polynucleotides, vectors, vaccines, composition, or pharmaceutical compositions in a method for inducing an immune response in a subject. In some aspects, the kits comprise any one or more of the polynucleotides, vectors, vaccines, composition, or pharmaceutical compositions described or exemplified herein and instructions for using the one or more polynucleotides, vectors, vaccines, composition, or pharmaceutical compositions in a method for preventing, reducing the incidence of, attenuating or treating a SARS-CoV-2 infection in a subject. 5.6 Uses and Methods

The present disclosure also features methods of inducing an immune response in a subject, the method comprising administering an effective amount of any polynucleotide, vector, multicistronic mRNA vector, DNA plasmid vector, composition, or pharmaceutical composition described or exemplified herein to the subject. In some aspects, the immune response is to one or more pathogen antigens disclosed herein, e.g., SARS-CoV-2 viral antigens.

In some aspects, the immune response is to one or more antigens comprising one or more viral antigens, one or more bacterial antigens, or one or more parasite antigens.

In some aspects, the immune response is to a bacterial antigen is selected from a Yersinia pestis antigen or a Mycobacterium tuberculosis antigen. In some aspects, the Yersinia pestis antigen is a Yersinia pestis capsular antigen. In some aspects, the Yersinia pestis capsular antigen is F1-Ag or virulence antigen (V-Ag). In some aspects, the Mycobacterium tuberculosis antigen is an Apa antigen, an HP65 antigen, or a rAg85A antigen.

In some aspects, the immune response is to a viral antigen selected from an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a dengue virus antigen, an Ebola virus antigen, a Zika virus, a chikungunya virus antigen, a measles virus antigen, a Middle East Respiratory Syndrome Coronavirus (MERS-CoV) antigen, or a SARS-CoV antigen. In some aspects, the enterovirus antigen is an enterovirus 71 (E71) antigen or a coxsackievirus (Cox) protein antigen. In some aspects, the E71 antigen is a E71-VP1 antigen or a glutathione S-transferase (GST)-tagged E71-VP1 antigen. In some aspects, the Cox protein antigen is GST-tagged Cox protein antigen. In some aspects, the HSV antigen is an HSV-1 envelope antigen, an HSV-2 envelope antigen, or an HSV-2 surface glycoprotein antigen. In some aspects, the HSV-2 surface glycoprotein antigen is a gB2 antigen, a gC2 antigen, a gD2 antigen, or a gE2 antigen. In some aspects, the HIV antigen is an Env antigen, a Gag antigen, a Nef antigen, or a Pol antigen. In some aspects, the HPV antigen is a minor capsid protein L2 antigen. In some aspects, the minor capsid protein L2 antigen comprises one or more epitope domains (amino acids 10-36 and/or amino acids 65-89) of minor capsid protein L2. In some aspects, the HCV antigen is a nonstructural 3 (NS3) antigen. In some aspects, the RSV antigen is an F antigen or a G antigen. In some aspects, the Dengue virus antigen is an E protein antigen, an E protein domain III (EDIII) antigen, a non-structural protein 1 (NS1) antigen, or a DEN-80E antigen. In some aspects, the Ebola virus antigen is a spike glycoprotein (GB) antigen, a VP24 antigen, a VP40 antigen, a nucleoprotein (NP) antigen, a VP30 antigen, or a VP35 antigen. In some aspects the Zika virus antigen is an envelope domain III antigen or a CKD antigen. In some aspects, the Chikungunya virus antigen is an E1 glycoprotein subunit antigen, the MHC class I epitope PPFGAGRPGQFGDI (SEQ ID NO: 34), the MHC class I epitope TAECKDKNL (SEQ ID NO: 35), or the MHC class II epitope VRYKCNCGG (SEQ ID NO: 36). In some aspects, the measles virus antigen is a hemagglutinin protein MV-H antigen or a fusion protein MV-F antigen. In some aspects, the MERS-CoV antigen is a spike (S) protein antigen, an antigen from the receptor-binding domain of the S protein, or an antigen from the membrane fusion domain of the S protein. In some aspects, the SARS-CoV antigen is a spike (S) protein antigen, an antigen from the receptor binding domain of the S protein, an antigen from the membrane fusion domain of the S protein, an envelope (E) protein antigen, or an M protein antigen.

In some aspects, the immune response is to one or more influenza virus antigens from any influenza virus type or subtype. In some aspects, the one or more influenza virus antigens are selected from the group consisting of: an influenza virus hemagglutinin (HA) antigen, an influenza virus neuraminidase (NA) antigen, an influenza virus matrix-2 (M2) protein antigen, antigenic fragments thereof, and any combination thereof. In some aspects, the one or more influenza virus antigens are derived from influenza virus type A, type B, type C, type D, or any combination thereof. In some spects, the one or more influenza virus antigens are derived from influenza virus type A. In some aspects, the one or more influenza virus antigens derived from influenza virus type A have (a) a HA subtype selected from H1 through H18 or any combination thereof and (b) a NA subtype selected from N1 through N11 or any combination thereof. In some aspects, the one or more influenza virus antigens derived from influenza virus type A, subtype H1N1; influenza virus type A, subtype H2N2; influenza virus type A, subtype H3N2; influenza virus type A, subtype H5N1; influenza virus type A, subtype H7N7; influenza virus type A, subtype H7N9; influenza virus type A, subtype H9N2; or any combination thereof. In some aspects, the one or more influenza virus antigens are derived from influenza virus type A, subtype H1N1; influenza virus type A, subtype H3N2; or the combination thereof. In some spects, the one or more influenza virus antigens are derived from influenza virus type B. In some aspects, the immune response is to one or more SARS-CoV-2 antigens or antigenic fragments thereof disclosed herein and one or more influenza virus antigens or antigenic fragments thereof disclosed herein. In some aspects, the immune response is to a parasite antigen, wherein the parasite antigen is a protozoan antigen. In some aspects, the immune response is to a parasite antigen selected from the group consisting of a Toxoplasma gondii antigen or a Plasmodium falciparum antigen. In some aspects, the Toxoplasma gondii antigen is antigen MIC8. In some aspects, the Plasmodium falciparum antigen is a SERA5 polypeptide antigen, or a circumsporozite protein antigen. In some aspects, the immune response is to a parasite antigen, wherein the parasite antigen is a parasitic or pathogenic fungus antigen. In some aspects, the parasitic or pathogenic fungus antigen is selected from the group consisting of a Candida spp. antigen (e.g., a Candida albicans antigen, a Candida glabrata antigen, a Candida parapsilosis antigen, a Candida tropicalis antigen, a Candida lusitaniae antigen, a Candida krusei antigen), a Pneumocystis spp. antigen, a Malassezia spp. antigen (e.g., a Malassezia furfur antigen), an Aspergillus fumigatus antigen, a Cryptococcus spp. antigen (e.g., a Cryptococcus neoformans antigen, a Cryptococcus gattii antigen), a Histoplasma capsulatum antigen, a Blastomyces dermatitidis antigen, a Paracoccidioides spp. antigen (e.g., a Paracoccidioides brasiliensis antigen, a Paracoccidioides lutzii antigen), a Coccidioides spp. antigen (e.g., a Coccidioides immitis antigen, a Coccidioides posadasii antigen), a Penicillium marneffei antigen, a Sporothrix schenckii antigen, a Trichosporon asahii antigen, a Fusarium spp. antigen (e.g., a Fusarium solanum antigen, a Fusarium oxysporum antigen), a Nectria spp. antigen, a Pseudoallescheria boydii antigen, a Cladophialphora bantianum antigen, a Ramichloridium spp. antigen, a Dactylaria gallopava antigen, an Exophiala spp. antigen (e.g., an Exophiala jeanselmei antigen, an Exophiala dermatitidis antigen), a Curvularia spp. antigen, a Bipolaris spp. antigen, an Alternaria spp. antigen, a Lacazia loboi antigen, a Conidiobolus spp. antigen (e.g., a Conidiobolus coronatus antigen, a Conidiobolus incongruus antigen), and any combination thereof.

Also provided herein is a method of preventing, reducing the incidence of, attenuating or treating a virus (e.g., SARS-CoV-2), a bacteria or a parasite infection in a subject.

The present disclosure also features methods of preventing, reducing the incidence of, attenuating or treating a virus (e.g., SARS-CoV-2), a bacteria or a parasite infection in a subject, the method comprising administering an effective amount of any polynucleotide, vector, multicistronic mRNA vector, DNA plasmid vector, composition, or pharmaceutical composition described or exemplified herein to the subject.

In some aspects, the methods of the disclosure are directed to administration of a composition comprising a delivery component and a polynucleotide comprising: a first nucleotide sequence, wherein the first nucleotide sequence encodes IL-12 p35 and is operably linked to a CMV promoter; a second nucleotide sequence, wherein the second nucleotide sequence encodes IL-12 p40 and is operably linked to a CMV promoter; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-12 p35 and is operably linked to a CMV promoter; a second nucleotide sequence positioned 3′ to the first nucleotide sequence, wherein the second nucleotide sequence encodes IL-12 p40 and is operably linked to a CMV promoter; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 1.

In some aspects, the methods of the disclosure are directed to administration of a composition comprising a delivery component and a polynucleotide comprising: a first nucleotide sequence, wherein the first nucleotide sequence encodes IL-12 p35 and is operably linked to a CMV promoter; a second nucleotide sequence, wherein the second nucleotide sequence encodes IL-12 p40 and is operably linked to a CMV promoter; a third nucleotide sequence, wherein the third nucleotide sequence encodes MHC I and is operably linked to promoter Z; a fourth nucleotide sequence, wherein the fourth nucleotide sequence encodes a first SARS-CoV-2 protein and is operably linked to promoter 1, and a fifth nucleotide sequence, wherein the fifth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-12 p35 and is operably linked to a CMV promoter; a second nucleotide sequence positioned 3′ to the first nucleotide sequence, wherein the second nucleotide sequence encodes IL-12 p40 and is operably linked to a CMV promoter; a third nucleotide sequence positioned 3′ to the second nucleotide sequence, wherein the third nucleotide sequence encodes MHC I and is operably linked to promoter Z; a fourth nucleotide sequence positioned 3′ to the third nucleotide sequence, wherein the fourth nucleotide sequence encodes a first SARS-CoV-2 protein and is operably linked to promoter 1, and a fifth nucleotide sequence positioned 3′ to the fourth nucleotide sequence, wherein the fifth nucleotide sequence encodes a second SARS-CoV-2 protein and is operably linked to promoter 2. In some aspects, the first, second, third, fourth, and fifth nucleotide sequences of the polynucleotide are configured as shown in FIG. 2.

In some aspects, the methods of the disclosure are directed to administration of a composition comprising a delivery component and a polynucleotide comprising: a first nucleotide sequence, wherein the first nucleotide sequence encodes IL-12 p35 and is operably linked to a CMV promoter; a second nucleotide sequence, wherein the second nucleotide sequence encodes IL-12 p40 and is operably linked to a CMV promoter; a third nucleotide sequence, wherein the third nucleotide sequence encodes MHC II and is operably linked to promoter Z; a fourth nucleotide sequence, wherein the fourth nucleotide sequence encodes a first SARS-CoV-2 protein and is operably linked to promoter 1, and a fifth nucleotide sequence, wherein the fifth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-12 p35 and is operably linked to a CMV promoter; a second nucleotide sequence positioned 3′ to the first nucleotide sequence, wherein the second nucleotide sequence encodes IL-12 p40 and is operably linked to a CMV promoter; a third nucleotide sequence positioned 3′ to the second nucleotide sequence, wherein the third nucleotide sequence encodes MHC II and is operably linked to promoter Z; a fourth nucleotide sequence positioned 3′ to the third nucleotide sequence, wherein the fourth nucleotide sequence encodes a first SARS-CoV-2 protein and is operably linked to promoter 1, and a fifth nucleotide sequence positioned 3′ to the fourth nucleotide sequence, wherein the fifth nucleotide sequence encodes a second SARS-CoV-2 protein and is operably linked to promoter 2. In some aspects, the first, second, third, fourth, and fifth nucleotide sequences of the polynucleotide are configured as shown in FIG. 3.

In some aspects, the methods of the disclosure are directed to administration of a composition comprising a delivery component and a polynucleotide comprising: a first nucleotide sequence, wherein the first nucleotide sequence encodes IL-2 and is operably linked to a CMV promoter; a second nucleotide sequence, wherein the second nucleotide sequence encodes a first SARS-CoV-2 protein and is operably linked to promoter 1; and a third nucleotide sequence, wherein the third nucleotide sequence encodes a second SARS-CoV-2 protein and is operably linked to promoter 2. In some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes a first SARS-CoV-2 protein and is operably linked to promoter 1; and a third nucleotide sequence positioned 3′ to the second nucleotide sequence, wherein the third nucleotide sequence encodes a second SARS-CoV-2 protein and is operably linked to promoter 2. In some aspects, the first, second, and third nucleotide sequences of the polynucleotide are configured as shown in FIG. 4.

In some aspects, the methods of the disclosure are directed to administration of a composition comprising a delivery component and a polynucleotide comprising: a first nucleotide sequence, wherein the first nucleotide sequence encodes IL-2 and is operably linked to a CMV promoter; a second nucleotide sequence, wherein the second nucleotide sequence encodes MHC I and is operably linked to promoter Z; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes MHC I and is operably linked to promoter Z; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 5.

In some aspects, the methods of the disclosure are directed to administration of a composition comprising a delivery component and a polynucleotide comprising: a first nucleotide sequence, wherein the first nucleotide sequence encodes IL-2 and is operably linked to a CMV promoter; a second nucleotide sequence, wherein the second nucleotide sequence encodes MHC II and is operably linked to promoter Z; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes MHC II and is operably linked to promoter Z; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 6.

In some aspects, the methods of the disclosure are directed to administration of a composition comprising a delivery component and a polynucleotide comprising: a first nucleotide sequence, wherein the first nucleotide sequence encodes IL-2 and is operably linked to a CMV promoter; a second nucleotide sequence, wherein the second nucleotide sequence encodes CCL3 and is operably linked to promoter X; a third nucleotide sequence, wherein the third nucleotide sequence encodes CCL4 and is operably linked to promoter Y; a fourth nucleotide sequence, wherein the fourth nucleotide sequence encodes a first SARS-CoV-2 protein and is operably linked to promoter 1, and a fifth nucleotide sequence, wherein the fifth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes CCL3 and is operably linked to promoter X; a third nucleotide sequence positioned 3′ to the second nucleotide sequence, wherein the third nucleotide sequence encodes CCL4 and is operably linked to promoter Y; a fourth nucleotide sequence positioned 3′ to the third nucleotide sequence, wherein the fourth nucleotide sequence encodes a first SARS-CoV-2 protein and is operably linked to promoter 1, and a fifth nucleotide sequence positioned 3′ to the fourth nucleotide sequence, wherein the fifth nucleotide sequence encodes a second SARS-CoV-2 protein and is operably linked to promoter 2. In some aspects, the first, second, third, fourth, and fifth nucleotide sequences of the polynucleotide are configured as shown in FIG. 7.

In some aspects, the methods of the disclosure are directed to administration of a composition comprising a delivery component and a polynucleotide comprising: a first nucleotide sequence, wherein the first nucleotide sequence encodes IL-15 and is operably linked to a CMV promoter; a second nucleotide sequence, wherein the second nucleotide sequence encodes a first SARS-CoV-2 protein and is operably linked to promoter 1; and a third nucleotide sequence, wherein the third nucleotide sequence encodes a second SARS-CoV-2 protein and is operably linked to promoter 2. In some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes a first SARS-CoV-2 protein and is operably linked to promoter 1; and a third nucleotide sequence positioned 3′ to the second nucleotide sequence, wherein the third nucleotide sequence encodes a second SARS-CoV-2 protein and is operably linked to promoter 2. In some aspects, the first, second, and third nucleotide sequences of the polynucleotide are configured as shown in FIG. 8.

In some aspects, the methods of the disclosure are directed to administration of a composition comprising a delivery component and a polynucleotide comprising: a first nucleotide sequence, wherein the first nucleotide sequence encodes IL-15 and is operably linked to a CMV promoter; a second nucleotide sequence, wherein the second nucleotide sequence encodes MHC I and is operably linked to promoter Z; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes MHC I and is operably linked to promoter Z; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 9.

In some aspects, the methods of the disclosure are directed to administration of a composition comprising a delivery component and a polynucleotide comprising: a first nucleotide sequence, wherein the first nucleotide sequence encodes IL-15 and is operably linked to a CMV promoter; a second nucleotide sequence, wherein the second nucleotide sequence encodes MHC II and is operably linked to promoter Z; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes MHC II and is operably linked to promoter Z; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 10.

In some aspects, the methods of the disclosure are directed to administration of a composition comprising a delivery component and a polynucleotide comprising: a first nucleotide sequence, wherein the first nucleotide sequence encodes IL-15 and is operably linked to a CMV promoter; a second nucleotide sequence, wherein the second nucleotide sequence encodes CCL3 and is operably linked to promoter X; a third nucleotide sequence, wherein the third nucleotide sequence encodes CCL4 and is operably linked to promoter Y; a fourth nucleotide sequence, wherein the fourth nucleotide sequence encodes a first SARS-CoV-2 protein and is operably linked to promoter 1, and a fifth nucleotide sequence, wherein the fifth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes CCL3 and is operably linked to promoter X; a third nucleotide sequence positioned 3′ to the second nucleotide sequence, wherein the third nucleotide sequence encodes CCL4 and is operably linked to promoter Y; a fourth nucleotide sequence positioned 3′ to the third nucleotide sequence, wherein the fourth nucleotide sequence encodes a first SARS-CoV-2 protein and is operably linked to promoter 1, and a fifth nucleotide sequence positioned 3′ to the fourth nucleotide sequence, wherein the fifth nucleotide sequence encodes a second SARS-CoV-2 protein and is operably linked to promoter 2. In some aspects, the first, second, third, fourth, and fifth nucleotide sequences of the polynucleotide are configured as shown in FIG. 11.

In some aspects, the methods of the disclosure are directed to administration of a composition comprising a delivery component and a polynucleotide comprising: a first nucleotide sequence, wherein the first nucleotide sequence encodes CCL3 and is operably linked to promoter X; a second nucleotide sequence, wherein the second nucleotide sequence encodes CCL4 and is operably linked to promoter Y; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes CCL3 and is operably linked to promoter X; a second nucleotide sequence positioned 3′ to the first nucleotide sequence, wherein the second nucleotide sequence encodes CCL4 and is operably linked to promoter Y; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 12.

In some aspects, the methods can comprise administering a pharmaceutical composition or vaccine comprising a delivery component and a polynucleotides configured as shown in the vector constructs illustrated in any of FIGS. 1-12, which can modified to replace the “Covid-19 Spike Gene” (a first nucleotide sequence encoding a SARS-CoV-2 protein) and the “Covid-19 Gene-2” (a second nucleotide sequence encoding a SARS-CoV-2 protein) with nucleotide sequences encoding any combinations of pathogen antigen or antigenic fragment thereof disclosed herein. In some aspect, the nucleotide sequences encode antigens to a virus, a bacteria or a parasite. In some aspects, the nucleotide sequences encode one or more antigens comprise one or more viral antigens, one or more bacterial antigens, or one or more parasite antigens.

In some aspects, the methods of the disclosure are directed to administration of a composition comprising a delivery component and a polynucleotide comprising: a first nucleotide sequence, wherein the first nucleotide sequence encodes a first pathogen protein (e.g., a S1 subunit of the SARS-CoV-2 S protein) and is operably linked to a first promoter (e.g., a hEF1-HTLV promoter); a second nucleotide sequence, wherein the second nucleotide sequence encodes IL-12 p35 and is operably linked to a second promoter (e.g., a CMV promoter); and a third nucleotide sequence, wherein the third nucleotide sequence encodes IL-12 p40 and is operably linked to a second promoter (e.g., a CMV promoter). In some aspects, the first, second, and third nucleotide sequences of the polynucleotide are configured as shown in FIG. 14C (pVac 2).

In some aspects, the methods of the disclosure are directed to administration of a composition comprising a delivery component and a polynucleotide comprising: a first nucleotide sequence, wherein the first nucleotide sequence encodes a first pathogen protein (e.g., a S1 subunit of the SARS-CoV-2 S protein) and is operably linked to a first promoter (e.g., a hEF1-HTLV promoter); a second nucleotide sequence, wherein the second nucleotide sequence encodes a second pathogen protein (e.g., a SARS-CoV-2 M protein) and is operably linked to the first promoter through an IRES sequence; a third nucleotide sequence, wherein the third nucleotide sequence encodes IL-12 p35 and is operably linked to a second promoter (e.g., a CMV promoter); and a fourth nucleotide sequence, wherein the fourth nucleotide sequence encodes IL-12 p340 and is operably linked to a second promoter (e.g., a CMV promoter). In some aspects, the first, second, third, and fourth nucleotide sequences of the polynucleotide are configured as shown in FIG. 14D (pVac 3).

In some aspects, the methods of the disclosure are directed to administration of a composition comprising a delivery component and a polynucleotide comprising: a first nucleotide sequence, wherein the first nucleotide sequence encodes a first pathogen protein (e.g., a SARS-CoV-2 full-length D614G S protein) and is operably linked to a first promoter (e.g., an EF-1α promoter); a second nucleotide sequence, wherein the second nucleotide sequence encodes IL-12 p35 and is operably linked to a second promoter (e.g., a CMV promoter); and a third nucleotide sequence, wherein the third nucleotide sequence encodes IL-12 p40 and is operably linked to a second promoter (e.g., a CMV promoter). In some aspects, the first, second, and third nucleotide sequences of the polynucleotide are configured as shown in FIG. 14F (pVac 5).

In some aspects, the methods of the disclosure are directed to administration of a composition comprising a delivery component and a polynucleotide comprising: a first nucleotide sequence, wherein the first nucleotide sequence encodes a first pathogen protein (e.g., a full-length SARS-CoV-2 D614G S protein) and is operably linked to a first promoter (e.g., an EF-1α promoter); a second nucleotide sequence, wherein the second nucleotide sequence encodes a second pathogen protein (e.g., a SARS-CoV-2 M protein) and is operably linked to the first promoter through an IRES sequence; a third nucleotide sequence, wherein the third nucleotide sequence encodes IL-12 p35 and is operably linked to a second promoter (e.g., a CMV promoter); and a fourth nucleotide sequence, wherein the fourth nucleotide sequence encodes IL-12 p340 and is operably linked to a second promoter (e.g., a CMV promoter). In some aspects, the first, second, third, and fourth nucleotide sequences of the polynucleotide are configured as shown in FIG. 14G (pVac 6).

In some aspects, the methods of the disclosure are directed to administration of a composition comprising a delivery component and a polynucleotide comprising a first nucleotide sequence, wherein the first nucleotide sequence encodes a first pathogen protein (e.g., a S1 subunit of the SARS-CoV-2 S protein or a SARS-CoV-2 full-length D614G S protein) and is operably linked to a first promoter (e.g., an EF-1α promoter). In some aspects, the first nucleotide sequence of the polynucleotide is configured as shown in FIG. 14B (pVac 1) or FIG. 14E (pVac 4).

In some aspects, the methods of the disclosure are directed to administration of a composition comprising a delivery component and a polynucleotide comprising a first nucleotide sequence, wherein the first nucleotide sequence encodes a first pathogen protein (e.g., a SARS-CoV-2 full-length D614G S protein) and is operably linked to a first promoter (e.g., an EF-1α promoter); and a second nucleotide sequence, wherein the second nucleotide sequence encodes a second pathogen (e.g., a SARS-CoV-2 M protein) and is operably linked to a second promoter (e.g., a CMV promoter). In some aspects, the first nucleotide sequence of the polynucleotide is configured as shown in FIG. 14H (pVac 7).

In some aspects, the methods can comprise administering a pharmaceutical composition or vaccine comprising a delivery component and a polynucleotides configured as shown in the vector constructs illustrated in any of FIGS. 14A-14H, which can modified to replace the S1 subunit of the SARS-CoV-2 S protein or the SARS-CoV-2 full-length D614G S protein (a first nucleotide sequence encoding a first pathogen protein) and/or the SASRS-CoV-2 M protein (a second nucleotide sequence encoding a second pathogen protein) with nucleotide sequences encoding any combinations of pathogen antigen or antigenic fragment thereof disclosed herein. In some aspect, the nucleotide sequences encode antigens to a virus, a bacteria or a parasite. In some aspects, the nucleotide sequences encode one or more antigens comprise one or more viral antigens, one or more bacterial antigens, or one or more parasite antigens.

In some aspects, the nucleotide sequences encode one or more bacterial antigens selected from a Yersinia pestis antigen, a Mycobacterium tuberculosis antigen, antigenic fragments thereof, or any combinations thereof. In some aspects, the Yersinia pestis antigen is a Yersinia pestis capsular antigen. In some aspects, the Yersinia pestis capsular antigen is F1-Ag or virulence antigen (V-Ag). In some aspects, the Mycobacterium tuberculosis antigen is an Apa antigen, an HP65 antigen, a rAg85A antigen, any antigenic fragments thereof, or any combinations thereof.

In some aspects, the nucleotide sequences encode one or more viral antigens selected from an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, antigenic fragments thereof, or any combinations thereof. In some aspects, the enterovirus antigen is an enterovirus 71 (E71) antigen, a coxsackievirus (Cox) protein antigen, antigenic fragments thereof, or any combinations thereof. In some aspects, the E71 antigen is an E71-VP1 antigen, a glutathione S-transferase (GST)-tagged E71-VP1 antigen, antigenic fragments thereof, or any combinations thereof. In some aspects, the Cox protein antigen is GST-tagged Cox protein antigen. In some aspects, the HSV antigen is an HSV-1 envelope antigen, an HSV-2 envelope antigen, an HSV-2 surface glycoprotein antigen, antigenic fragments thereof, or any combinations thereof. In some aspects, the HSV-2 surface glycoprotein antigen is a gB2 antigen, a gC2 antigen, a gD2 antigen, a gE2 antigen, or antigenic fragments thereof, or any combinations thereof. In some aspects, the HIV antigen is an Env antigen, a Gag antigen, a Nef antigen, a Pol antigen, antigenic fragments thereof, and or combinations thereof. In some aspects, the HPV antigen is a minor capsid protein L2 antigen. In some aspects, the minor capsid protein L2 antigen comprises one or more epitope domains (amino acids 10-36 and/or amino acids 65-89) of minor capsid protein L2. In some aspects, the HCV antigen is a nonstructural 3 (NS3) antigen. In some aspects, the RSV antigen is an F antigen, a G antigen, antigenic fragments thereof, or any combinations thereof. In some aspects, the Dengue virus antigen is an E protein antigen, an E protein domain III (EDIII) antigen, a non-structural protein 1 (NS1) antigen, a DEN-80E antigen, antigenic fragments thereof, or any combinations thereof. In some aspects, the Ebola virus antigen is a spike glycoprotein (GB) antigen, a VP24 antigen, a VP40 antigen, a nucleoprotein (NP) antigen, a VP30 antigen, a VP35 antigen, antigenic fragments thereof, or any combinations thereof. In some aspects the Zika virus antigen is an envelope domain III antigen, a CKD antigen, antigenic fragments thereof, or any combinations thereof. In some aspects, the Chikungunya virus antigen is an E1 glycoprotein subunit antigen, the MHC class I epitope PPFGAGRPGQFGDI (SEQ ID NO: 34), the MHC class I epitope TAECKDKNL (SEQ ID NO: 35), the MHC class II epitope VRYKCNCGG (SEQ ID NO: 36), antigenic fragments thereof, or any combinations thereof. In some aspects, the measles virus antigen is a hemagglutinin protein MV-H antigen, a fusion protein MV-F antigen, antigenic fragments thereof, or any combinations thereof. In some aspects, the MERS-CoV antigen is a spike (S) protein antigen, an antigen from the receptor-binding domain of the S protein, an antigen from the membrane fusion domain of the S protein, antigenic fragments thereof, or any combinations thereof. In some aspects, the SARS-CoV antigen is a spike (S) protein antigen, an antigen from the receptor binding domain of the S protein, an antigen from the membrane fusion domain of the S protein, an envelope (E) protein antigen, an M protein antigen, antigenic fragments thereof, or any combinations thereof.

In some aspects, the nucleotide sequences encode one or more influenza virus antigens from any influenza virus type or subtype. In some aspects, the one or more influenza virus antigens are selected from the group consisting of: an influenza virus hemagglutinin (HA) antigen, an influenza virus neuraminidase (NA) antigen, an influenza virus matrix-2 (M2) protein antigen, antigenic fragments thereof, and any combination thereof. In some aspects, the one or more influenza virus antigens are derived from influenza virus type A, type B, type C, type D, or any combination thereof. In some spects, the one or more influenza virus antigens are derived from influenza virus type A. In some aspects, the one or more influenza virus antigens derived from influenza virus type A have (a) a HA subtype selected from H1 through H18 or any combination thereof and (b) a NA subtype selected from N1 through N11 or any combination thereof. In some aspects, the one or more influenza virus antigens derived from influenza virus type A, subtype H1N1; influenza virus type A, subtype H2N2; influenza virus type A, subtype H3N2; influenza virus type A, subtype H5N1; influenza virus type A, subtype H7N7; influenza virus type A, subtype H7N9; influenza virus type A, subtype H9N2; or any combination thereof. In some aspects, the one or more influenza virus antigens are derived from influenza virus type A, subtype H1N1; influenza virus type A, subtype H3N2; or the combination thereof. In some spects, the one or more influenza virus antigens are derived from influenza virus type B. In some aspects, the nucleotide sequences encode one or more SARS-CoV-2 antigens or antigenic fragments thereof disclosed herein and one or more influenza virus antigens or antigenic fragments thereof disclosed herein.

In some aspects, the nucleotide sequences encode one or more parasite antigens, wherein the one or more parasite antigens comprise one or more protozoan antigens. In some aspects, the nucleotide sequences encode one or more parasite antigens selected from Toxoplasma gondii antigen, a Plasmodium falciparum antigen, antigenic fragments thereof, or any combinations thereof. In some aspects, the Toxoplasma gondii antigen is antigen MIC8. In some aspects, the Plasmodium falciparum antigen is a SERA5 polypeptide antigen, a circumsporozite protein antigen, antigenic fragments thereof, or any combinations thereof. In some aspects, the nucleotide sequences encode one or more parasite antigens, wherein the one or more parasite antigens comprise one or more parasitic or pathogenic fungus antigens. In some aspects, the one or more parasitic or pathogenic fungus antigens are selected from the group consisting of a Candida spp. antigen (e.g., a Candida albicans antigen, a Candida glabrata antigen, a Candida parapsilosis antigen, a Candida tropicalis antigen, a Candida lusitaniae antigen, a Candida krusei antigen), a Pneumocystis spp. antigen, a Malassezia spp. antigen (e.g., a Malassezia furfur antigen), an Aspergillus fumigatus antigen, a Cryptococcus spp. antigen (e.g., a Cryptococcus neoformans antigen, a Cryptococcus gattii antigen), a Histoplasma capsulatum antigen, a Blastomyces dermatitidis antigen, a Paracoccidioides spp. antigen (e.g., a Paracoccidioides brasiliensis antigen, a Paracoccidioides lutzii antigen), a Coccidioides spp. antigen (e.g., a Coccidioides immitis antigen, a Coccidioides posadasii antigen), a Penicillium marneffei antigen, a Sporothrix schenckii antigen, a Trichosporon asahii antigen, a Fusarium spp. antigen (e.g., a Fusarium solanum antigen, a Fusarium oxysporum antigen), a Nectria spp. antigen, a Pseudoallescheria boydii antigen, a Cladophialphora bantianum antigen, a Ramichloridium spp. antigen, a Dactylaria gallopava antigen, an Exophiala spp. antigen (e.g., an Exophiala jeanselmei antigen, an Exophiala dermatitidis antigen), a Curvularia spp. antigen, a Bipolaris spp. antigen, an Alternaria spp. antigen, a Lacazia loboi antigen, a Conidiobolus spp. antigen (e.g., a Conidiobolus coronatus antigen, a Conidiobolus incongruus antigen), and any combination thereof.

In some aspects, the first nucleic acid and the second nucleic acid encone a first SARS-CoV-2 antigen and a second SARS-CoV-2 antigen, respectively. In some aspects, the first SARS-CoV-2 protein is a SARS-CoV-2 S protein or an antigenic fragment thereof. In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the first SARS-CoV-2 protein comprises the amino acid sequence of SEQ ID NO: 2 or SEQ ID NO: 4.

In some aspects, the second SARS-CoV-2 protein is a SARS-CoV-2 S protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the second SARS-CoV-2 protein comprises the amino acid sequence of SEQ ID NO: 2 or SEQ ID NO: 4.

In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4, wherein the contiguous amino acids of SEQ ID NO: 2 or SEQ ID NO: 4 comprise one or more mutations (i.e., one or more substitutions, deletions, insertions, or any combination thereof). In some aspects, the first SARS-CoV-2 protein comprises the amino acid sequence of SEQ ID NO: 2 or SEQ ID NO: 4, wherein the polypeptide comprises one or more mutations (i.e., one or more substitutions, deletions, insertions, or any combination thereof).

In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4, wherein the contiguous amino acids of SEQ ID NO: 2 or SEQ ID NO: 4 comprise one or more mutations (i.e., one or more substitutions, deletions, insertions, or any combination thereof). In some aspects, the second SARS-CoV-2 protein comprises the amino acid sequence of SEQ ID NO: 2 or SEQ ID NO: 4, wherein the polypeptide comprises one or more mutations (i.e., one or more substitutions, deletions, insertions, or any combination thereof).

In some aspects, the first SARS-CoV-2 protein is the receptor binding domain (RBD) of the SARS-Cov-2 S protein or an antigenic fragment thereof. In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the first SARS-CoV-2 protein comprises the amino acid sequence of SEQ ID NO: 6.

In some aspects, the second SARS-CoV-2 protein is the receptor binding domain (RBD) of the SARS-Cov-2 S protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the second SARS-CoV-2 protein comprises the amino acid sequence of SEQ ID NO: 6.

In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6, wherein the contiguous amino acids of SEQ ID NO: 6 comprise one or more mutations (i.e., one or more substitutions, deletions, insertions, or any combination thereof). In some aspects, the first SARS-CoV-2 protein comprises the amino acid sequence of SEQ ID NO: 6, wherein the polypeptide comprises one or more mutations (i.e., one or more substitutions, deletions, insertions, or any combination thereof).

In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6, wherein the contiguous amino acids of SEQ ID NO: 6 comprise one or more mutations (i.e., one or more substitutions, deletions, insertions, or any combination thereof). In some aspects, the second SARS-CoV-2 protein comprises the amino acid sequence of SEQ ID NO: 6, wherein the polypeptide comprises one or more mutations (i.e., one or more substitutions, deletions, insertions, or any combination thereof).

In some aspects, the first SARS-CoV-2 protein is the S1 subunit of the SARS-Cov-2 S protein or an antigenic fragment thereof. In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the first SARS-CoV-2 protein comprises the amino acid sequence of SEQ ID NO: 40.

In some aspects, the second SARS-CoV-2 protein is the S1 subunit of the SARS-Cov-2 S protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the second SARS-CoV-2 protein comprises the amino acid sequence of SEQ ID NO: 40.

In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40, wherein the contiguous amino acids of SEQ ID NO: 40 comprise one or more mutations (i.e., one or more substitutions, deletions, insertions, or any combination thereof). In some aspects, the first SARS-CoV-2 protein comprises the amino acid sequence of SEQ ID NO: 40, wherein the polypeptide comprises one or more mutations (i.e., one or more substitutions, deletions, insertions, or any combination thereof).

In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40, wherein the contiguous amino acids of SEQ ID NO: 40 comprise one or more mutations (i.e., one or more substitutions, deletions, insertions, or any combination thereof). In some aspects, the second SARS-CoV-2 protein comprises the amino acid sequence of SEQ ID NO: 40, wherein the polypeptide comprises one or more mutations (i.e., one or more substitutions, deletions, insertions, or any combination thereof).

In some aspects, the one or more mutations in the SARS-CoV-2 full-length S protein, the RBD of the SARS-Cov-2 S protein, the S1 subunit of the SARS-CoV-2 S protein, or antigenic fragments thereof comprise one or more mutations previously reported in Li, T. et al., Emerg Microbes Infect. 9(1):2076-90 (2020); Lee, P. et al., Immune Netw. 21(1):e4 (2021); Yu, J. et al., Science 369(6505):806-11 (2020); Cattin-Ortola, J. et al., Nat Commun. 12(1):5333 (2021); Corbett, K. et al., Nature 586(7830):567-71 (2020); Hsieh, C. et al., Science 369(6510):1501-5 (2020); and Harvey, W. et al., Nat Rev Microbiol. 19(7):409-24 (2021), each of which is incorporated by reference herein in its entirety.

In some aspects, the one or more mutations in the SARS-CoV-2 full-length S protein, the RBD of the SARS-Cov-2 S protein, the S1 subunit of the SARS-CoV-2 S protein, or antigenic fragments thereof are selected from: ΔM1-S13, S12P, S131, L5F, L18F, T19R, T20N, P26S, Q52R, A67V, ΔH69-V70, G75V, T76I, D80A, T95I, R102I, ΔD119-F120, C136Y, D138Y, ΔF140, ΔL141-Y144, AY144, Y144S, Y145N, ΔH146, N148S, K150R, K150E, K150T, K150Q, S151P, W152C, E154K, ΔE156-F157, F157L, F157A, R158G, R190S, ΔI210, D215G, A222V, ΔL241-S243, ΔL242-L244, ΔA243-L244, ΔR246-G252, R246I, 11-amino acid residue insertion between Y248 and L249, D253G, D253N, R346K, V367F, E406W, K417E, K417V, K417N, K417T, N439K, K444A, K444R, K444N, K444Q, V445A, V445E, G447A, N450D, L452R, L452Q, Y453F, L455F, N460I, S477G, S477N, S477R, T478I, T478K, V483I, E484K, E484Q, G485R, F486A, F486V, F486L, N487A, F490S, Q493E, Q493K, S494P, N501Y, A570D, Q613H, D614G, H655Y, Q677H, Δ678-679, Δ681-682, Δ681-684, Δ682-685, P681H, P681R, R682S, R682A, R682Q, R683S, R683G, R683Q, R685G, R685Q, I692V, A701V, T716I, F817P, T859N, F888L, A892P, A899P, A942P, D950N, S982A, K986P, V987P, T1027I, Q1071H, D1118H, V1176F, M1229I, ΔC1253-T1273, ΔC1254-T1273, ΔK1255-T1273, D1257A, E1258A, D1259A, D1260A, E1262A, K1269A, H1271K, T1273A, or any combination thereof, wherein the amino acid locations correspond to SEQ ID NO: 2 or SEQ ID NO: 4.

In some aspects, the one or more mutations comprise one or more mutations in the N-terminal signal peptide, which corresponds to amino acids 1-13 of SEQ ID NO: 2 or SEQ ID NO: 4. In some aspects, the one or more mutations in the N-terminal signal peptide is ΔM1-S13, wherein the amino acid locations correspond to SEQ ID NO: 2 or SEQ ID NO: 4.

In some aspects, the one or mutations comprise one or more mutations in the C-terminus of the full-length SARS-CoV-2 S protein. In some aspects, the one or more mutations in the C-terminus of the full-length SARS-CoV-2 S protein comprise one or more mutations in the C-terminal endoplasmic reticulum (ER) retention peptide, which corresponds to amino acids 1254-1273 of SEQ ID NO: 2 or SEQ ID NO: 4. In some aspects, the one or more mutations in the C-terminal ER retention peptide are selected from D1257A, E1258A, D1259A, D1260A, E1262A, K1269A, H1271K, T1273A, or any combination thereof, wherein the amino acid locations correspond to SEQ ID NO: 2 or SEQ ID NO: 4. In some aspects, the one or more mutations in the C-terminal ER retention peptide comprise D1257A+E1258A+D1259A+D1260A+E1262A (i.e., a D/E to A mutant), wherein the amino acid locations correspond to SEQ ID NO: 2 or SEQ ID NO: 4. In some aspects, the one or mutations in the C-terminal ER retention peptide is ΔC1253-T1273, ΔC1254-T1273, or ΔK1255-T1273.

In some aspects, the one or more mutations comprise K986P+V987P (i.e., a S-2P mutation), wherein the amino acid locations correspond to SEQ ID NO: 2 or SEQ ID NO: 4. In some aspects, the one or more mutations comprise F817P+A892P+A899P+A942P (i.e., a hexa-proline S mutation), wherein the amino acid locations correspond to SEQ ID NO: 2 or SEQ ID NO: 4. In some aspects, the one or more mutations comprise one or more mutations in the 681-PRRAR/SVA-688 S1/S2 furin cleavage site, wherein the amino acid locations correspond to SEQ ID NO: 2 or SEQ ID NO: 4. In some aspects, the one or more mutations in the 681-PRRAR/SVA-688 S1/S2 furin cleavage site are: (a) R682S+R683S (i.e., a SSAR mutation), (b) Δ681-684 (i.e., a ΔPRRA mutation), (c) Δ678-679+Δ681-682, (d) R682A+R683G+R685G (i.e., a 682-AGAG-685 mutation), (e) R682Q+R683Q+R685Q, (f) R682S+R685G, or (g) Δ682-685 (i.e., a ΔRRAR mutation), wherein the amino acid locations correspond to SEQ ID NO: 2 or SEQ ID NO: 4.

In some aspects, the one or more mutations comprise: (a) F817P+A892P+A899P+A942P (i.e., a hexa-proline S mutation) and (b) K986P+V987P (i.e., a S-2P mutation), wherein the amino acid locations correspond to SEQ ID NO: 2 or SEQ ID NO: 4. In some aspects, the one or more mutations comprise: (a) R682A+R683G+R685G (i.e., a 682-AGAG-685 mutation) and (b) K986P+V987P (i.e., a S-2P mutation), wherein the amino acid locations correspond to SEQ ID NO: 2 or SEQ ID NO: 4. In some aspects, the one or more mutations comprise: (a) R682A+R683G+R685G (i.e., a 682-AGAG-685 mutation), (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations comprise: (a) R682Q+R683Q+R685Q and (b) K986P+V987P (i.e., a S-2P mutation). In some aspects, the one or more mutations comprise:: (a) R682Q+R683Q+R685Q, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations comprise: (a) R682S+R685G and (b) K986P+V987P (i.e., a S-2P mutation), wherein the amino acid locations correspond to SEQ ID NO: 2 or SEQ ID NO: 4. In some aspects, the one or more mutations comprise: (a) R682S+R685G, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4.

In some aspects, the second SARS-CoV-2 protein is a SARS-CoV-2 M protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the second SARS-CoV-2 protein comprises the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20.

In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20, wherein the contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20 comprise one or more mutations selected from A2S, F28L, I48V, V70L, I82T, M84T, or any combination thereof, wherein the amino acid locations correspond to SEQ ID NO: 8. In some aspects, the second SARS-CoV-2 protein comprises the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20, wherein the polypeptide comprises one or more mutations selected from A2S, F28L, I48V, V70L, I82T, M84T, or any combination thereof, wherein the amino acid locations correspond to SEQ ID NO: 8.

In some aspects, the second SARS-CoV-2 protein is a SARS-CoV-2 E protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 55, at least 60, at least 65, at least 70, or at least 75 contiguous amino acids of SEQ ID NO: 22, SEQ ID NO: 24, or SEQ ID NO: 26. In some aspects, the second SARS-CoV-2 protein comprises the amino acid sequence of SEQ ID NO: 22, SEQ ID NO: 24, or SEQ ID NO: 26.

In some aspects, the second SARS-CoV-2 protein is a SARS-CoV-2 N protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, or at least 400 contiguous amino acids of SEQ ID NO: 28. In some aspects, the second SARS-CoV-2 protein comprises the amino acid sequence of SEQ ID NO: 28.

In some aspects, promoter 1 is selected from the group consisting of: a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CAG promoter, and any combination thereof. In some aspects, the mammalian EF1 promoter is a hEF1-HTLV promoter.

In some aspects, promoter 2 is selected from the group consisting of: a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CAG promoter, and any combination thereof. In some aspects, the mammalian EF1 promoter is a hEF1-HTLV promoter.

In some aspects, promoter X is selected from the group consisting of: a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CAG promoter, and any combination thereof. In some aspects, the mammalian EF1 promoter is a hEF1-HTLV promoter.

In some aspects, promoter Y is selected from the group consisting of: a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CAG promoter, and any combination thereof. In some aspects, the mammalian EF1 promoter is a hEF1-HTLV promoter.

In some aspects, promoter Z is selected from the group consisting of: a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CAG promoter, and any combination thereof. In some aspects, the mammalian EF1 promoter is a hEF1-HTLV promoter.

In some aspects, the IRES sequence comprises a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 41.

The process of administration can be varied, depending on the agent, or agents, and the desired effect. Thus, the process of administration involves administering a therapeutic agent (e.g., any polynucleotide, vector, vaccine, composition, or pharmaceutical composition disclosed herein) to a patient in need of such treatment. Methods of delivering compositions comprising DNA vaccines are described in U.S. Pat. Nos. 4,945,050 and 5,036,006.

Administration can be accomplished by any means appropriate for the therapeutic agent, for example, by parenteral, mucosal, pulmonary, topical, catheter-based, or oral means of delivery. Parenteral delivery can include for example, subcutaneous, intravenous, intramuscular, intra-arterial, intraperitoneal, intralymphatic, and injection into the tissue of an organ. Mucosal delivery can include, for example, intranasal delivery, preferably administered into the airways of a patient, i.e., nose, sinus, throat, lung, for example, as nose drops, by nebulization, vaporization, or other methods known in the art. Oral or intranasal delivery can include the administration of a propellant. Pulmonary delivery can include inhalation of the agent. Catheter-based delivery can include delivery by iontropheretic catheter-based delivery. Oral delivery can include delivery of a coated pill, or administration of a liquid by mouth. Administration can generally also include delivery with a pharmaceutically acceptable carrier, such as, for example, a buffer, a polypeptide, a peptide, a polysaccharide conjugate, a liposome, and/or a lipid, according to methods known in the art.

Pulmonary delivery can include inhalation of the agent. Catheter-based delivery can include delivery by iontropheretic catheter-based delivery. Oral delivery can include delivery of a coated pill, or administration of a liquid by mouth. Administration can generally also include delivery with a pharmaceutically acceptable carrier, such as, for example, a buffer, a polypeptide, a peptide, a polysaccharide conjugate, a liposome, and/or a lipid, according to methods known in the art.

In some aspects, the viral vectors of the present disclosure are administered in an amount approximately corresponding to 10² to 10¹⁴ PFU, 10⁵ to 10¹² PFU, or 10⁶ to 10¹⁰ PFU per subject, calculated as the PFU of the viral vector. In some aspects, the viral vectors of the present disclosure are administered by directly injecting a viral vector suspension prepared by suspending the viral vector in PBS (phosphate buffered saline) or saline into a local site (e.g., into the lung tissue, liver, muscle or brain), by nasal or respiratory inhalation, or by intravascular (e.g., intra-arterial, intravenous, and portal venous), intralymphatic, subcutaneous, intracutaneous, intradermal, or intraperitoneal administration.

The present disclosure also features methods of making any composition, pharmaceutical composition, or vaccine described or exemplified herein, the method comprising the steps of: (a) combining a delivery component disclosed herein with a polynucleotide disclosed herein, (b) lyophilizing the combined delivery component and polynucleotide to a powder, and (c) reconstituting the powder with a diluent to form a solution of nucleic acid complexed with the delivery component

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); Crooks, Antisense drug Technology: Principles, strategies and applications, 2^{nd} Ed. CRC Press (2007) and in Ausubel et al. (1989) Current Protocols in Molecular Biology (John Wiley and Sons, Baltimore, Md.).

All of the references cited above, as well as all references cited herein and the amino acid or nucleotide sequences (e.g., GenBank numbers and/or Uniprot numbers), are incorporated herein by reference in their entireties.

The following examples are offered by way of illustration and not by way of limitation.

### EXAMPLES

### 6.1 Vector Construction

DNA plasmids vectors are constructed with the elements as shown in FIGS. 1-12. The starting plasmid backbone used vector construction will contain multiple restriction sites that will be utilized for subcloning the desired expression cassettes in order to generate the DNA plasmid constructs. Each expression cassette that is cloned into the vector will contain all of the necessary components required for expressing the function protein or antigen (e.g promoter sequence, gene sequence, poly A tail sequence). The general methodology will consist of digesting the starting DNA plasmid backbone with the appropriate restriction digest enzyme. The insert sequence will be obtained from isolating and gel purifying a genetic sequence from a second DNA plasmid using appropriate restriction digest enzymes. Alternatively, the sequences to be inserted into the plasmid vector will be generated synthetically or a combination of the previous two approaches can be used. The starting backbone and insert sequences will be mixed together and ligated using DNA ligase. The resulting DNA plasmids are then transformed into e. coli using standard protocols and streaked onto LB agar plates containing the appropriate antibiotics. Bacterial colonies will subsequently be isolated and grown in medium so that the plasmid can be purified and screened by restriction digest and gel electrophoresis in order to identify the colonies that have the correct plasmids.

6.2 Vector Formulation with the Delivery Component

The present disclosure can include a nucleic acid (e.g., a DNA plasmid vector or multicistronic mRNA vector) complexed with a biodegradable cross-linked cationic multi-block copolymer of the following formula:

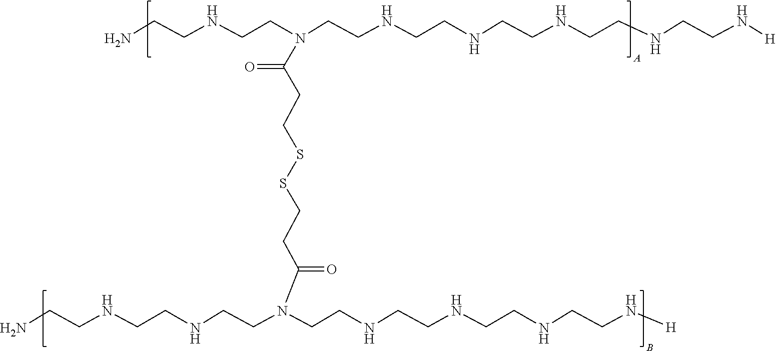

wherein: A and B are such that the molecular weight of the individual linear polyethylenimine chains are from 5,000 to 20,000 Dalton; the intermolecular crosslinks connect approximately 5-10% of amines; the biodegradable crosslinks are dithiodipropionyl (each half composed of 3 carbon atoms) and can be from 1-10 carbon atoms.

In some aspects, the biodegradable cross-linked cationic polymer comprises 10,000 to 15,000 Dalton linear PEI covalently connected with a dithiopropionyl linkage (see, e.g., U.S. Pat. No. 8,445,017). The polymer is dissolved in sterile water to give a final concentration of 3 mg/ml. The DNA is dissolved in sterile water to give a final concentration of 1 mg/ml. To make the polymer/DNA complex, the two components are diluted separately with 5% glucose to a volume of 150 uL each, and then the plasmid DNA solution is added to the polymer solution.

In some aspects, the biodegradable cross-linked cationic polymer comprises 10,000 to 15,000 Dalton linear PEI covalently connected with a dithiopropionyl linkage (see, e.g., U.S. Pat. No. 8,445,017) and is further conjugated to polyethyleneglycol (PEG) of molecular weight ranging from 500 to 20,000 Dalton. The resultant polymer is dissolved in sterile water to give a final concentration of 3 mg/ml. The DNA is dissolved in sterile water to give a final concentration of 1 mg/ml. To make the polymer/DNA complex, the two components are diluted separately with 5% glucose to a volume of 150 uL each, and then the plasmid DNA solution is added to the polymer solution.

In some aspects, the biodegradable cross-linked cationic polymer comprises 10,000 to 15,000 Dalton linear PEI covalently connected with a dithiopropionyl linkage (see, e.g., U.S. Pat. No. 8,445,017) and is further conjugated to polyethyleneglycol (PEG) of molecular weight ranging from 500 to 20,000 Dalton. The resultant polymer is dissolved in sterile water to give a final concentration of 3 mg/ml. The DNA is dissolved in sterile water to give a final concentration of 1 mg/ml. To make the polymer/DNA complex, the two components are diluted separately with 5% glucose to a volume of 150 uL each, and then the plasmid DNA solution is added to the polymer solution.

In some aspects, the biodegradable cross-linked cationic polymer comprises 15,000 to 20,000 Dalton linear PEI covalently connected with a dithiopropionyl linkage (see, e.g., U.S. Pat. No. 8,445,017). The polymer is dissolved in sterile water to give a final concentration of 3 mg/ml. The DNA is dissolved in sterile water to give a final concentration of 1 mg/ml. To make the polymer/DNA complex, the two components are diluted separately with 5% glucose to a volume of 150 uL each, and then the plasmid DNA solution is added to the polymer solution.

In some aspects, the biodegradable cross-linked cationic polymer comprises 15,000 to 20,000 Dalton linear PEI covalently connected with a dithiopropionyl linkage (see, e.g., U.S. Pat. No. 8,445,017) and is further conjugated to polyethyleneglycol (PEG) of molecular weight ranging from 500 to 20,000 Dalton. The resultant polymer is dissolved in sterile water to give a final concentration of 3 mg/ml. The DNA is dissolved in sterile water to give a final concentration of 1 mg/ml. To make the polymer/DNA complex, the two components are diluted separately with 5% glucose to a volume of 150 uL each, and then the plasmid DNA solution is added to the polymer solution.

Complex formation is allowed to proceed for 15 minutes at room temperature. To study the effect of the charge ratio on gene transfer, DNA complexes with biodegradable cross-linked cationic polymer can be prepared at different ratios 1/15/1, 10/1, and 20/1 nitrogen/phosphate (N/P). Following complex formation, the complexes are diluted in a cuvette for measurement of particle size and the potential of the complex. The electrophoretic mobility of the samples can be measured at 25° C., and at a wavelength of 657 nm and at a constant angle of 900 with a Particle sizer.

6.3 Vector Formulation with the Delivery Carrier PPC

Bench-scale production of highly concentrated liquid formulations of fully condensed nucleic Acid with a cationic lipopolymer is prepared. This involves preparation of nucleic acid complexes with a cationic polymer followed by lyophilization and reconstitution to isotonic solutions. The nucleic acid used is a plasmid DNA, and the polymer comprises a PEI backbone covalently linked to PEG and cholesterol (PEG-PEI-cholesterol (“PPC”)). The molar ratio between PEG and PEI and between cholesterol and PEI is 0.5-10 and 0.1-10, respectively. First, the DNA and PPC solutions are separately prepared at 5 mg/ml in water for injection and subsequently diluted to 0.3 mg/ml (DNA) and 1.108 mg/ml (PPC) in 3% lactose. The DNA in lactose solution is added to the PPC in lactose solution using a micropipette to a nitrogen to phosphate ratio (N:P ratio) of 11:1, and the formulation is incubated for 15 minutes at room temperature to allow the complexes to form. The PPC/DNA complexes in 3% lactose are lyophilized using a FREEZONE freeze dry System from LABCONCO Corp. Kansas City, Mo. 500 ul of prepared formulation is added to 2 ml borosilicate glass vials which were then lyophilized using a freeze drying program consisting of the following segments:
1) freezing segment (ramp 0.25° C./min, hold at 34° C. for 4 hours),
2) primary drying segment (hold at 34° C. for 24 hrs),
3) secondary drying segment (ramp to 20° C. and hold for 24 hrs), and
4) ramp to 4° C. at 0.25° C./min.

The resultant lyophilized powder is reconstituted with 150 μl water for injection to make 0.5 mg/ml DNA.

6.4 Vector Formulation with the Delivery Carrier Poloxamer

Poloxamers are gently mixed 1 mg/ml of nucleic acids in water or saline solution (0.15M) at variable concentrations. Formulated poloxamer (5%)/plasmid solutions are analyzed by gel electrophoresis in order to verify interaction between formulated plasmid and poloxamer. Comparison between unformulated plasmid DNA and DNA formulated with poloxamer have the similar movement through the gel and therefore indicate no binding between plasmid DNA and poloxamer. The formulated plasmid with poloxamer is used for gene transfer in mammalian cell or tissue.

### 6.5 Synthesis of Aza-Crown-Linked Poloxamer (Crown Poloxamer)

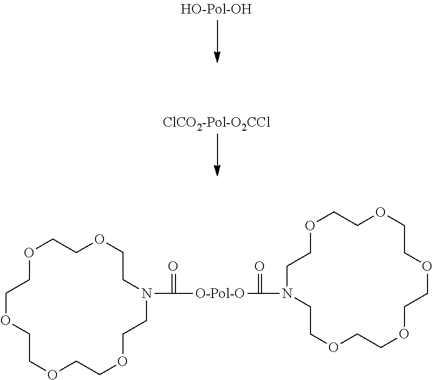

An aza-crown-linked poloxamer (crown poloxamer) is constructed as follows. Poloxamer 124 (Pluronic L-44; 500 mg, 220 mol) was dissolved in toluene (3 ml), and the resulting solution was treated with 2 ml (4 mmol) of 2M phosgene solution in toluene. After 3 hrs at room temperature, the mixture was concentrated in vacuum, the residue was re-dissolved in 3 ml toluene and concentrated again. The residue was dissolved in dry chloroform (5 ml). To this solution was added aza-18-crown-6 [1-aza-4, 7, 10, 13, 16-pentaoxacyclooctadecane (125 mg, 500 mol) and Hunig's base (100 μl, 574 mol). After 70 hrs the reaction mixture was concentrated in vacuum, the residue was re-dissolved in distilled water and dialyzed [membrane cutoff 1000 Da]against distilled water. Concentration of the dialyzate afforded 410 mg of the title compound. Proton NMR (D20): 4.20 ppm (t, CH₂OC═O); 3.7-3.5 ppm [(—CH₂—CH₂—O—), both crown and poloxamer)]; 3.4 ppm (m, crown CH₂N); 1.1 ppm (m, poloxamer-(CH₃)CH—CH₂—).

6.6 pVac Vector Construction

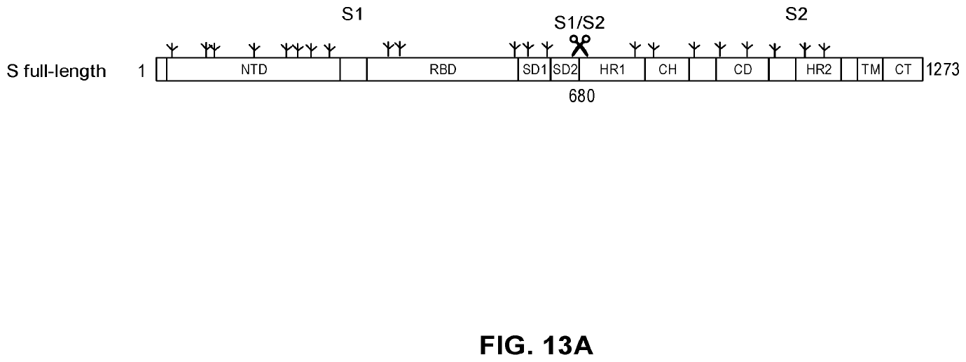

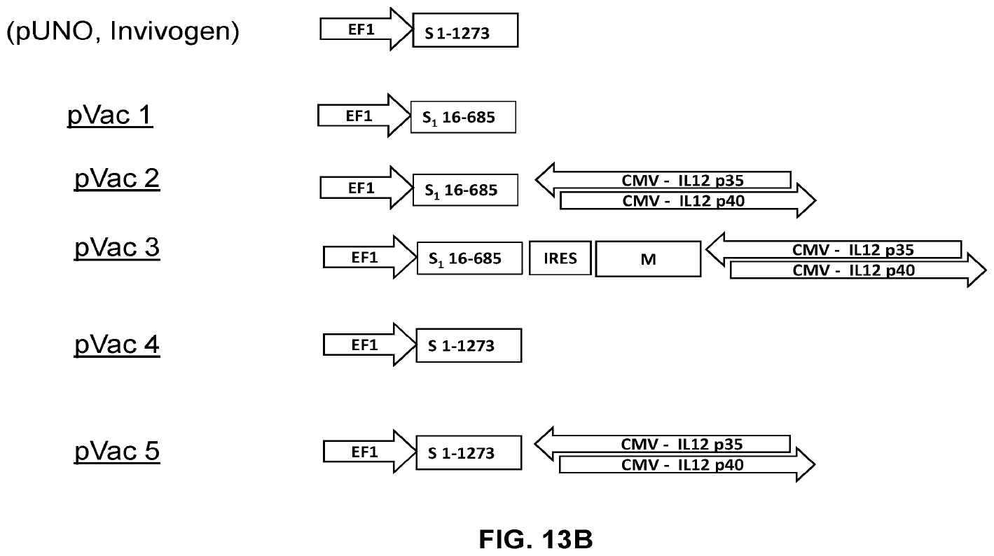

DNA vector constructs for expressing viral antigens were prepared. In particular, vectors referred to as pVac 1-7 were created to express the S1 subunit of the SARS-CoV-2 S protein (SP1 or Spike1, which corresponds to amino acids 15-695 of the full-length S protein) or a SARS-CoV-2 full-length S protein (SARS2S-D614G; 1273 amino acids in length), both of which contain a D614G substitution (FIG. 13A). The S protein coding sequence of each vector is under the control of a mammalian EF-1α promoter. The plasmids pVac3, pVac6, and pVac7 were designed to co-express the SARS-CoV-2 M antigen. The pVac3 and pVac6 plasmids included an Internal Ribosome Entry Site (IRES) sequence between the viral antigens. The pVac2, pVac3 and pVac5 plasmids were designed to further co-express the human heterodimeric cytokine IL12. The coding sequences for the IL-12 p35 and IL-12 p40 subunits are under the control of two CMV promoters (FIG. 13B). An unmodified pUNO Spike including the full-length SARS-CoV-2 S protein was also tested (Invivogen (San Diego, CA)).

Construction of pVac 3 (FIG. 14D) included using a TLO1001-SP1-UniRE vector and fragment MHisPA-unqRE (TWIST, San Francisco, CA; SEQ ID NO: 66). First, a p2CMV vector was engineered to express IL-12 p35 and IL-12 p40 from two CMV promoters and SARS-CoV-2 full-length D614G S protein from a hEF1-HTLV promoter (p2CMV mIL-12+SARS2S-D614G). The resulting p2CMV mIL-12+SARS2S-D614G vector was cut by AgeI/SapI (7677+4256 bp) as backbone. PCR of AgeImHisPA insert was completed using MHispA-unique RE template with primers AgelMhis-F (SEQ ID NO: 51) and pAcelsion-R (SEQ ID NO: 52). Following PCR, Gibson assembly cloning was used to assemble an intermediate vector including p2CMV mIL-12+SARS2S-D614G and MHispA-unique RE. To complete pVac 3 construction, the intermediate vector was digested by AgeI/SalI-HF (8653+46 bp). A AgeIspIRESSalI PCR product was amplified from a TLO1001-SP1-UniRE template using the primers CelsionAgeIspl-F (SEQ ID NO: 53) and IRESSalI-R (SEQ ID NO: 54). The AgeIspIRESSalI PCR product was then digested by AgeI/SalI-HF and ligated by T4 ligase.

Construction of pVac 2 (FIG. 14C) included digesting the pVac 3 vector (p2CMV mIL-12+Spike1-IRES-M-His-pA) by PspXI/SapI (9782+1613 bp), followed by CIP treatment and column purification. PCR for the insert was done using KOD Hot Start DNA Polymerase with the pVac3 template (p2CMV mIL-12+Spike1-IRES-M-His-pA) and PspXI-SV40 pA-F (SEQ ID NO: 56) and Cls-SapI-R (SEQ ID NO: 57) primers.

The pVac 1 vector (FIG. 14B) was constructed using a p2CMV-v4 vector as a backbone. The p2CMV-v4 vector is shown in FIG. 14I. The p2CMVv4 vector was cut with BglII/XmnI (sequential digestion: 4693+400 bp) then followed with CIP treatment and Column purification. Using a p2CMV mIL-12+Spike 1-pA unique RE vector as a template, PCR was performed with Cls-BglII-R-2 (SEQ ID NO: 58) and Cls-XmnI-R (SEQ ID NO: 59) primers using KOD Hot Start DNA Polymerase. The vector was then completed using Gibson Assembly.

To construct pVac 5 (FIG. 14F), an intermediate vector with full Spike1-D614G-OP, Spike1-KanR was synthesized. Next, the intermediate vector was digested by AgeI-HF/PspXI yielding constructs of 3848, 1772, and 445 bp. Additionally, the template from pVac3 (p2CMV mIL-12+Spike1-pA unique RE) step 1 was also digested by AgeI-HF/PspXI (Cutsmart) yielding constructs of 7927 and 2105 bp. The resulting constructs were ligated by T4 DNA ligase, and clones were verified by Sanger sequencing.

To construct pVac 4 (FIG. 14G), an intermediate vector with full Spike1-D614G-OP, Spike1-KanR was synthesized. Next the intermediate vector was digested by AgeI-HF/PspXI yielding constructs of 3848, 1772, and 445 bp. Additionally, the template from pVac3 p2CMV mIL-12+Spike1-IRES-M-His-pA unique RE was also digested by AgeI-HF/PspXI yielding constructs of 7927 and 2105 bp. The resulting constructs were ligated by T4 DNA ligase and clones were verified by Sanger sequencing.

To construct pVac6 (FIG. 14G) a human codon optimized M protein DNA fragment was synthesized. Vector p2CMV mIL-12+Spike1-D614G-OP-pA unique was digested by PspXI and treated by CIP. PCR for the IRES fragment from p2CMV mIL-12+Spike1-IRES-M-His-pA was completed using CLS-PspXI-TRES-F (SEQ. ID NO. 60) and IREScelsion-R (SEQ. ID NO. 61) primers. PCR of the M protein from synthesized fragment was done using Mhiscelsion-F (SEQ. ID NO. 62) and CLS-M-R (SEQ. ID NO. 63) primers. Gibson Assembly was used with the two inserts to create vector.

To construct pVac7 (FIG. 14H), p2CMV-V4-Spike1-D614G-OP-pA unique RE (the pVac3 intermediate vector) was digested by MluI-HF and treated by CIP. PCR of the M protein from synthesized fragment was completed using CLS-XbaI-M-F (SEQ. ID NO. 64) and CLS-MluI-M-R (SEQ. ID NO. 65) primers. Gibson Assembly was used with the two inserts to create vector.

### 6.7 In Vitro Expression

Expression level of spike protein and mIL-12 level was evaluated after in vitro transfection of COS-1 or 293T cells. Cell lines were plated in 12-well plate one day before transfection at 150,000 cell/well in 1 mL of DMEM with 10% FBS. On the following day, 1-1.5 mg of Plasmid DNA was mixed with Omnifect and plasmid DNA was mixed as in described in the examples; the Omnifect/plasmid mixture was add on to cells in culture plates in the presence of 10% FBS. Concentration of plasmid DNA in each well was adjusted to 100-200 pM by addition of extra cell culture media to each well. Transfected cells were incubated at 37° C. in a CO₂ incubator for 48 hours. When the incubation period was concluded, cell culture media was collected from each well in 1.5 mL tubes and cells were washed with phosphate-buffered saline and lysed with TENT buffer (50 mM Tris-Cl [pH8.0]2 mM EDTA, 150 mM NaCl, 1% Triton X-100) or RNA lysis buffer with betamercaptoethanol (Qiagen, Hilden, Germany). Spike protein expression level was evaluated in cell culture media and cell lysate using in house developed spike protein ELISA, western blot, and qPCR. mIL-12 protein expression was measured in cell culture media by mIL-12 ELISA purchased from R&D systems (Minneapolis, MN; cat #M1270). The total protein level was determined using a BCA protein assay kit (ThermoFisher, Waltham, MA; cat #23225).

### Western Blot

Total protein was isolated by centrifugation at 4° C., 10,000 rpm for 5 minutes. Lysate protein concentration was determined through a BCA assay kit (ThermoFisher, Waltham, MA; cat #23225). Total protein concentration was normalized, and protein was mixed with Leammli buffer and Betamercaptoethanol and boiled for 5 minutes at 95° C. Protein was loaded in a tris glycine 4-12% precast gel and run for 2.5 hours at 100 V. The gel was transferred to a PVDF membrane using the iBlot2 dry transfer system, following which the membrane was blocked with 5% nonfat milk in TBST for 1 hour at room temperature. After this, primary antibody was added at a desired concentration and the membrane was incubated overnight at 4° C. The membrane was washed 3 times with TBST, then incubated with recommended concentration of secondary antibody for 45 minutes at room temperature. Following this, the membrane was washed with TBST 3 times for 5 minutes, and the membrane was developed using ECL solution on the Licor C-DiGit Blot Scanner.

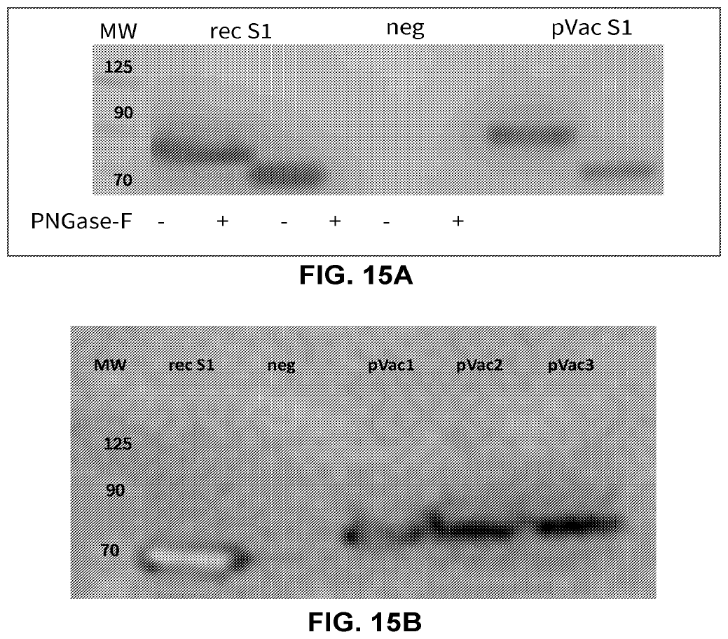

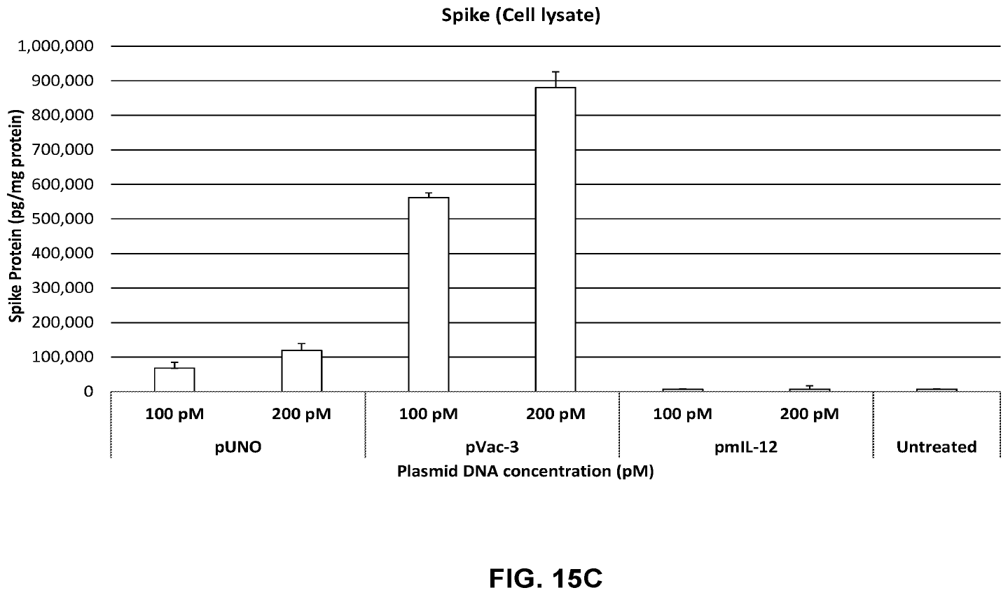

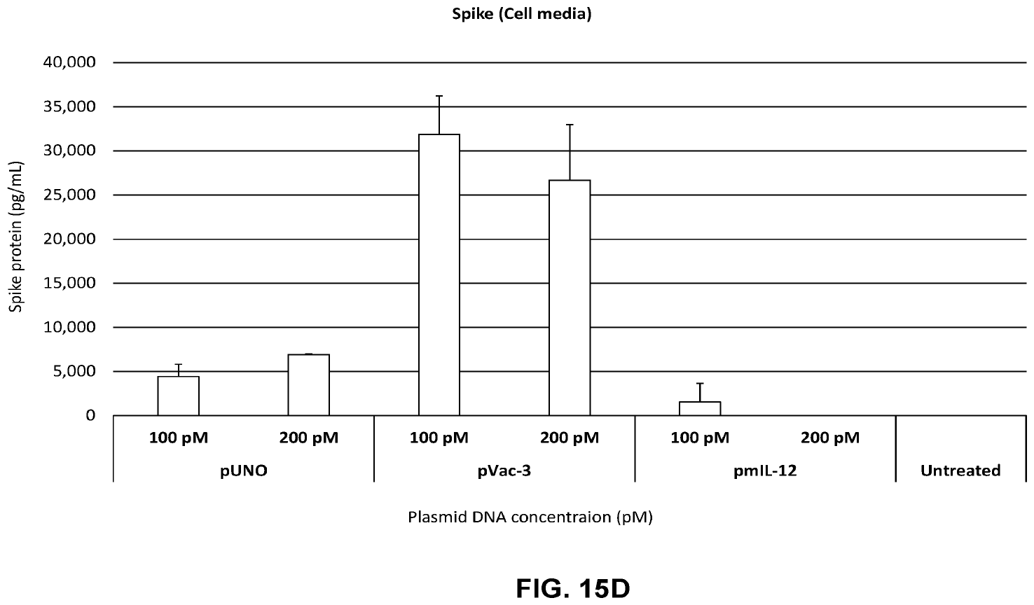

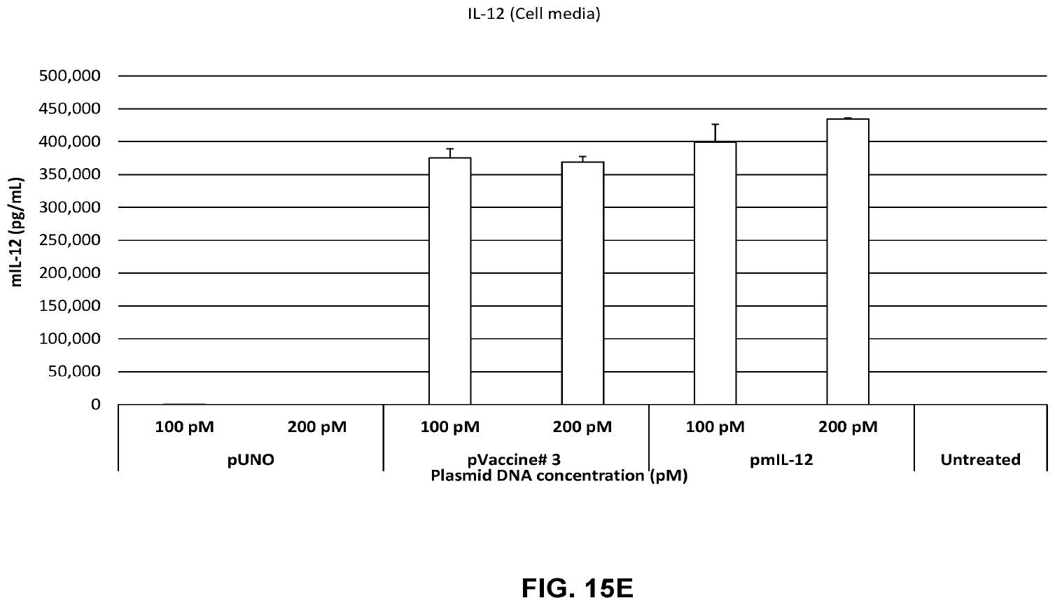

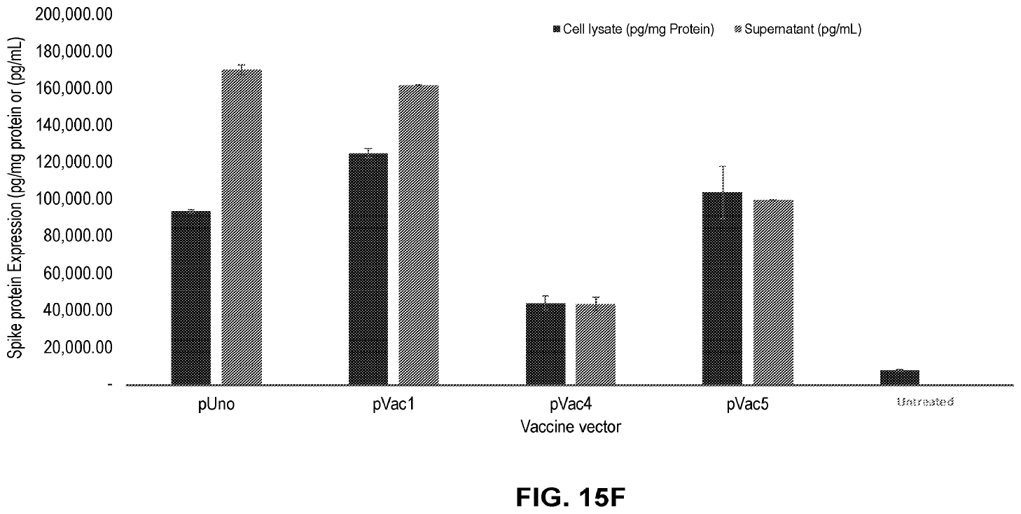

Expression of Spike S1 protein was confirmed after transfection of pVac1 in 293T cells (FIG. 15A). Further, digestion with PNGase-F resulted in expected decrease in molecular weight (FIG. 15A). Spike S1 protein expression in 293T cell lysates was also confirmed following transfection with either pVac1, pVac2, or pVac3 (FIG. 15B) qPCR

To assess the relative expression of viral mRNA following transfection with vectors of interest, a quantitative PCR assay was developed. In short, 293T cells were transfected for 24 hours with either a vehicle control or vector of interest. Next, cells were washed with PBS and lysed with RNA lysis buffer with added Betamercaptoethanol (Qiagen, Hilden, Germany). RNA was isolated using a RNeasy kit from Qiagen (Hilden, Germany). RNA quantification was performed using spectrophotometry, and cDNA was synthesized using a Verso cDNA synthesis kit (Thermo Scientific, Waltham, MA). Following this, cDNA was diluted 1:5 with molecular biology grade water, and 5 μL of sample was loaded with 25 μL of master mix using Taqman chemistry. The qPCR reaction was performed using a QuantStudio5 from Applied Biosystems using fast advanced taqman master-mix conditions measuring comparative ΔΔCt values against a normalized endogenous control, 18S. Along with RAW Ct analysis, statistical analysis was performed to calculate relative fold change. Spike S1 showed increased relative expression in 293T cell lysate following transfection with either pVac1 (3367.1 relative quantification [RQ]) or pVac2 (5538 RQ) (amplification data not shown). Additionally, Spike S1, Membrane, and 18S mRNA showed increased relative expression in 293T cell lysate following transfection with pVac3 (13412.5 RQ; data not shown).

### Spike Protein ELISA

Expression level of Spike protein was assessed in cell culture media and cell lysate. Cell culture media was collected in 1.5 mL Eppendorf tube and centrifuged at 10,000 rpm for 5 min, supernatant was collected and stored at 4° C. for short term storage and or −20° C. for a long-term storage (>24 hrs). Cells were washed phosphate-buffered saline and lysed with TENT buffer (50 mM Tris-Cl [pH 8.0]2 mM EDTA, 150 mM NaCl, 1% Triton X-100). Lysates were collected in 1.5 mL Eppendorf tubes and centrifuged at 10,000 rpm for 5 min, supernatant was collected and stored at 4° C. for short term storage and or −20° C. for a long-term storage (>24 hrs). To perform spike protein ELISA, 96-well, Nunc were coated overnight at 4° C. with 1 μg/ml of primary spike protein antibody form Sino biological (cat #40150-D003). After incubation for overnight, wells were aspirated and washed four times with 300 μl of wash buffer (0.05% Tween-20 in PBS, pH 7.2-7.4). Following washing steps, 300 μL of blocking buffer (2% BSA in Wash Buffer) was added to each well and incubated at room temperature for one hour then repeated the aspiration/wash steps. Serial dilution of spike protein standard from Sino Biological (40589-V08B1) was prepared in dilution buffer (40,000-625 μg/mL), 100 μl of each standard dilution, cell lysate and cell culture media were added to standard wells and sample wells and incubated for two hours at room temperature, then repeated the aspiration/wash steps. 100 μL of 1 g/mL secondary antibody (Sino Biological Cat #4150-D001H) added to each 96-well and incubated for one hour at room temperature, then repeated the aspiration/wash steps. 100 μL of substrate solution TMB (Tetramethylbenzidine) in DMSO with equal volume of substrate dilution buffer to each well and incubated in the dark for 20 minutes at room temperature. 100 μL of Stop Solution (1 M phosphoric acid) was added to each well. Optical density of each well was determined within 20 minutes, using a microplate reader set to 450 nm.

Using the above ELISA method, Spike protein was confirmed in vitro following transfection of both pVac2, and pVac 3, in both the cell lysate (FIG. 15C) and cell media (FIG. 15D), and mIL12 expression was confirmed in the cell media (FIG. 15E) Spike protein expression was confirmed in pVac1, pVac4 and pVac5 (FIG. 15F).

### 6.8 In Vivo Expression

### Secreted Alkaline Phosphatase (SEAP) Assay

To assess the kinetics and amount of in vivo antigen expression, a plasmid DNA with reporter gene encoding for SEAP was administered with 0.5% w/v crown poloxamer (CP) formulation to mice via intramuscular injection. Groups of five healthy female BALB/c mice aged 8-10 weeks were immunized with plasmid DNA (pDNA) encoding for SEAP and formulated with CP, or PBS. A 100 ul dose was administered to each mouse (50 ul per site) in the quadriceps muscle. Blood samples were taken 1-, 3—and 7-days post injection. Serum was separated from the blood immediately after collection and stored at −80° C. until use. A chemiluminescent SEAP assay Phospha-Light System (Applied Biosystems) was used to detect the SEAP enzymatic activity in the serum. Sera from immunized mice were diluted 1:20 in 1× Phospha-Light reaction buffer diluent. Samples were placed in a water bath sealed with aluminum sealing foil and heat inactivated for 30 minutes at 65° C. in a dry heat block. After cooling on ice for 3 minutes, and equilibrating to room temperature, 50 μL of PhosphaLight assay buffer was added to the wells and the samples were left at room temperature for 5 minutes. Then, 50 μL of reaction buffer containing 1:20 CSPDR (chemiluminescent alkaline phosphate Substrate) Substrate was added, and the luminescence was measured after 20 minutes of incubation at room temperature. Luminescence was measured on a EL808 ULTRA Microplate Reader luminometer Bio-Tek (Winooski, VT) with a 1 second integration per well. The activity of SEAP in each sample was measured in duplicate and the mean of these two measurements taken.

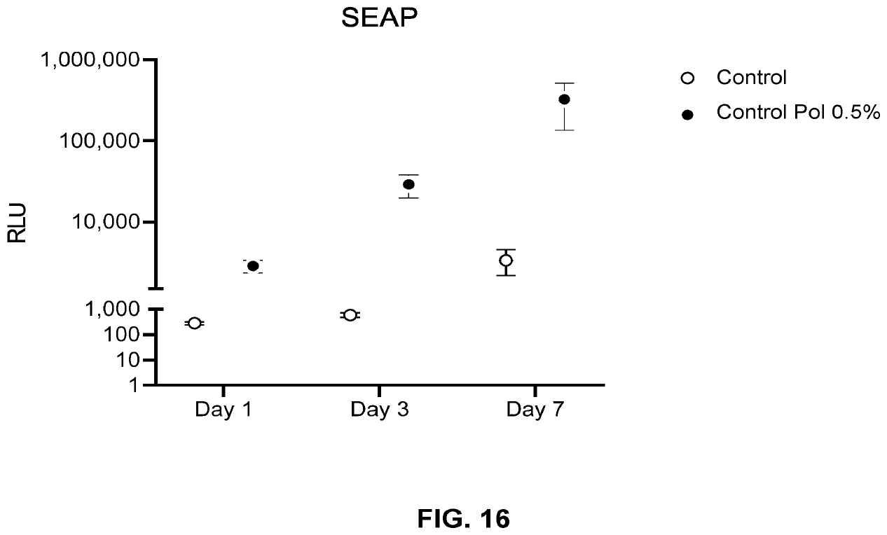

Serum SEAP levels were measured by relative light units (RLU) of mice, 5 animals per group, after intramuscular vaccinations on day 0. Serum was collected for SEAP analysis on days 1, 3 and 7 and were measured for enzyme activity after intramuscular vaccination with and without CP delivery. These results show that plasmid DNA expressing SEAP (pDNA SEAP) delivery improved with formulation of the plasmid with CP compard to control delivery (FIG. 16). Data are represented as arithmetic mean titers of 5 individual mice per group. All constructs produced measurable levels of SEAP in the serum of the vaccinated mice with pDNA SEAP formulated with crown poloxamer (CP) increased the levels of expression, particularly at the day 7 time point.

### 6.9 Vaccine Formulations

### PPC/pVaccine Formulation

Vaccine plasmid DNA was prepared initially at 0.1 mg/mL with PEG-PEI-cholesterol (PPC; MW 4.2 kD), by mixing plasmid DNA with the polymer at (11:1) and (0.5:1) (N:P) ratios in 5% dextrose solution, and the mixture was incubated at room temperature for 10 minutes to allow the formation of nanocomplexes. Then, pDNA/PPC nanocomplexes were concentrated to 1-5 mg/mL using Amicon Ultra Centrifugal Filters (Ultracel-3K MWCO).

The electrophoretic mobility of the PPC/DNA complexes was determined by agarose gel electrophoresis at 70 V for one hour. DNA integrity in the complexes was determined by incubation of the complexes with 50 μg of dextran sulfate or triton-X for 10 minutes at room temperature followed by gel electrophoresis at 100 Volts for one hour. The particle size the nanocomplexes in Milli-Q water was determined at 657 nm at a constant angle of 90° by dynamic light scattering using a Malvern particle size analyzer. Osmolality of the formulation was determined using Fiske210 micro-sample osmometer, DNA quantification was performed using spectrophotometry, formulation pH was measured using Accumet research AR15 pH meter.

### BD15-12/pVaccine Formulation

Vaccine plasmid DNA was prepared initially at 0.1 mg/mL with PEI-base copolymer, BD15-12 (15kD linear PEI; MW 26.5 kD), by mixing plasmid DNA with the polymer at 10:1 and 0.5:1 N:P ratios in 5% dextrose solution, and the mixture was incubated at room temperature for 10 minutes to allow the formation of nanocomplexes. Then, BD15-12/pDNA nanocomplexes were concentrated to 2.5-5 mg/mL using Amicon Ultra Centrifugal Filters (Ultracel-3K MWCO)

The electrophoretic mobility of the BD15-12/DNA complexes was determined by agarose gel electrophoresis at 70 V for one hour. The particle size of the nanocomplexes in Milli-Q water was determined at 657 nm at a constant angle of 90° by dynamic light scattering using a Malvern particle size analyzer. The complexation of plasmid DNA with BD15-12 resulted in DNA condensation into nanoparticles and prevented DNA migration on agarose gel (data not shown). Osmolality of the formulation was determined using Fiske 210 micro-sample osmometer, DNA quantification was performed using spectrophotometry, and formulation pH was measured using Accumet research AR15 pH meter (Table 1).

TABLE 1
DNA concentration, pH, osmolality, and particle
size results of formulated vaccine plasmids

| | DNA | | | | | | |
|---|---|---|---|---|---|---|---|
| | Conc. | | | | | | |
| | mg/mL | mOsm¹ | Zave² | PdI³ | di(10)⁴ | di(50)⁵ | di(90)⁶ |
| BD15-12/pUNO (10:1)(N:P) | 2.25 | 447 | 145.5 | 0.27 | 81.4 | 158 | 396 |
| 2.5 mg/mL | | | | | | | |
| Star:star-mPEG11(10:1)/ | 3.87 | 464 | 135.5 | 0.252 | 85.3 | 155 | 289 |
| pUNO(5:1)(N:P) 2.5 mg/mL | | | | | | | |
| Omnifect/pUNO(10:1)(N:P) | 2.09 | 451 | 84.21 | 0.295 | 42.4 | 91.2 | 291 |
| 2.5 mg/mL | | | | | | | |
| 0.5% Crown-Poloxamer/ | 2.90 | 350 | N/A | | | | |
| pUNO(5:1)(N:P) 2.5 mg/mL | | | | | | | |

¹Milliosmolality (mOSM);
²ZAverage (Zave): mean of measured particles using light scattering;
³Polydispersity index (PdI);
⁴di(10): size (nm) below which 10% of particles are found;
⁵di(50): size (nm) below which 50% of particles are found;
⁶di(90): size (nm) below which 90% of particles are found.

### Staramine:Star-mPEG/pVaccine Formulation:

Staramine: Poly(ethylene glycol) methyl ether (mPEG)/pVaccine formulation is composed of Staramine (0.635 kD) liposomes and plasmid DNA. Staramine alone or 10:1 mixtures of Staramine (Star) and Star-PEG515 (1.2 kD) were rotary-evaporated to a film. The flask of liposome film was held under high vacuum overnight. Water for injection was added to the film to give the desired Staramine concentration and bath sonicated for −30 minutes using Branson Water Bath Sonicator model 2510 followed by a probe sonication for 5 minutes (using a continuous pulse sonication with an output wattage of 5-10 watts (rms) (Model 100; Fisher Scientific Sonic Desmembrator, Pittsburgh, PA). The liposome solution was filtered through a 0.2-am filter, diluted with 5% dextrose, and mixed with the desired amount of plasmid. Particle size of the complexes was measured with Malvern particle size analyzer sizer. The complexation efficiency was determined by gel retardation assay (Life Technologies, Carlsbad, CA). The gel retardation assay was performed by loading the Staramine/plasmid DNA on 1% agarose gel and electrophoresed at 100 V for 1 hour. To release the plasmid DNA from the Staramine nanoparticles, 10% TritonX-100 was added to the complex and the solution was loaded on the agarose gel. The complexation of plasmid DNA with Staramine:Staramine-PEG resulted in DNA condensation into nanoparticles and prevented DNA migration on agarose gel. (Data not shown). The formulation procedure did not result in aggregation of DNA and staramine complexes. The addition of dextran sulfate and triton decomplexed the DNA from staramine causing a free mobility of DNA on agarose gel. The intact appearance of the DNA bands on the agarose gel for the decomplexed DNA formulation suggests that the DNA has not been degraded during formulation process. Osmolality of the formulation was determined using Fiske210 micro-sample osmometer, DNA quantification was performed using spectrophotometry, and formulation pH was measured using Accumet research AR15 pH meter (Table 1).

### Omnifect/pVaccine Formulation

Vaccine plasmid DNA was prepared initially at 0.1 mg/mL with Omnifect (MW 7.3 kD), by mixing plasmid DNA with the polymer at 10:1 and 0.5:1 N:P ratios in 5% dextrose solution, and the mixture was incubated at room temperature for 10 minutes to allow the formation of nanocomplexes. Then, Omnifect/pDNA nanocomplexes were concentrated to 2.5-5 mg/mL using Amicon Ultra Centrifugal Filters (Ultracel-3K MWCO)

The electrophoretic mobility of the Omnifect/DNA complexes was determined by agarose gel electrophoresis at 70 V for one hour. The particle size the nanocomplexes in Milli-Q water was determined at 657 nm at a constant angle of 90° by dynamic light scattering using a Malvern particle size analyzer. DNA integrity in the complexes was determined by incubation of the complexes with 50 μg of dextran sulfate or triton-X for 10 minutes at room temperature followed by gel electrophoresis at 100 Volts for one hour. The complexation of plasmid DNA with Omnifect resulted in DNA condensation into nanoparticles and prevented DNA migration on agarose gel. The formulation procedure did not result in aggregation of DNA and staramine complexes. Further, the addition of dextran sulfate and triton decomplexed the DNA from Omnifect causing a free mobility of DNA on agarose gel. The intact appearance of the DNA bands on the agarose gel for the decomplexed DNA formulation suggests that the DNA had not been degraded during formulation process. (Data not shown). Osmolality of the formulation was determined using Fiske210 micro-sample osmometer, DNA quantification was performed using spectrophotometry, and formulation pH was measured using Accumet research AR15 pH meter (Table 1).

### Crown Poloxamer/pVaccine Formulation

The required concentration of plasmid DNA to produce a final concentration of 1-5 mg/ml was made in PBS or NaCl, the mixture was mixed by low speed vortexing and the required amount of crown poloxamer (MW 2.2 Kd) was add to the DNA solution to produce a final concentration of 0.1-5%.

To store the formulation at −20° C. or for lyophilization purposes, the formulation was made in 20 mM Tris-8% sucrose instead of PBS or NaCl. Osmolality of the formulation was determined using Fiske210 micro-sample osmometer, DNA quantification was performed using spectrophotometry, and formulation pH was measured using Accumet research AR15 pH meter (Table 1).

### Crown Poloxamer/Cationic Carrier Adjuvant/pVaccine Formulation

The required concentration of plasmid DNA to produce a final concentration of 0.1-0.5 mg/ml was made in 5% dextrose or PBS, the mixture was mixed by low speed vortexing and the required amount of cationic adjuvant carrier (Staramine, BD15-12, PPC, Omnifect, Benzalkonium chloride (BAK)) was add to plasmid DNA. pDNA/PPC nanocomplexes were concentrated to 1-5 mg/mL using Amicon Ultra Centrifugal Filters (Ultracel-3K MWCO). Then crown poloxamer (i.e., aza-crown-linked poloxamer) was add to the DNA solution to produce a final concentration of 0.1-5%. Particle size of the CP-cationic carrier-pDNA complexes was measured with Malvern particle size analyzer sizer. Mixing crown poloxamer with plasmid DNA did not form any detectable nanoparticles by particle size measurements or gel electrophoresis. However, when cationic delivery systems were added to crown poloxamer/DNA formulation measurable nanoparticles were observed in the formulation. The complexation efficiency was determined by gel retardation assay (Life Technologies, Carlsbad, CA). The gel retardation assay was performed by loading the formulated plasmid DNA on 1% agarose gel and electrophoresed at 100 V for 1 hour. The intact appearance of the DNA bands on the agarose gel for crown poloxamer/DNA formulation suggested that crown poloxamer did not bind or degrade DNA during formulation process. However, a shift in DNA bands was observed when cationic delivery systems added to crown poloxamer/DNA formulation, suggesting an interaction between DNA and cationic delivery systems.

Osmolality of the formulation was determined using Fiske210 micro-sample osmometer, DNA quantification was performed using spectrophotometry, and formulation pH was measured using Accumet research AR15 pH meter (Table 2).

TABLE 2
DNA concentration, pH, osmolality, and particle
size results of formulated vaccine plasmids

| | | | | | | | | Signs of |
|---|---|---|---|---|---|---|---|---|
| | pH | mOsm¹ | Zave² | PDI³ | di(10)⁴ | di(50)⁵ | di(90)⁶ | Toxicity |
| Star:starPEG/CP/pSEAP | 4.42 | 539 | 109 | 0.377 | 45.1 | 140 | 270 | None |
| PPC/CP/pSEAP | 4.57 | 550 | 137.6 | 0.725 | 35.3 | 312 | 619 | None |
| BAK/CP/pSEAP | 6.94 | 371 | 155.1 | 0.441 | 59.5 | 204 | 373 | None |
| Omnifect/CP/pSEAP | 4.56 | 490 | 178.4 | 1 | 103 | 311 | 1230 | None |
| BD15-12/CP/pSEAP | 4.63 | 509 | 184.9 | 1 | 95 | 562 | 1560 | None |
| CP/pSEAP | 7.05 | 362 | ND⁷ | ND | ND | ND | ND | ND |

¹Milliosmolality (mOSM);
²ZAverage (Zave): mean of measured particles using light scattering;
³Polydispersity index (PdI);
⁴di(10): size (nm) below which 10% of particles are found;
⁵di(50): size (nm) below which 50% of particles are found;
⁶di(90): size (nm) below which 90% of particles are found;
⁷Not Determined (ND)

### Lyophilization Cycle

1 ml of each formulation was aliquoted into 2 ml glass vials and placed into freeze-dryer (FREEZONE Triad freeze dry System from LABCONCO Corp. Kansas City, MO.). Vials were cooled to −45° C. for 16 hours, and then temperature was raised to −15° C. before the start of the primary drying. After 24 hrs, the shelf temperature was raised to 0° C. and kept under vacuum for another 24 hours. Finally, shelf temperature was raised to 25° C. for 72 hours and vials were capped under vacuum at the end of secondary drying segment.

### 6.10 Immune Response to Vaccine Formulations

### Animal Immunization Procedure

The immunogenicity study for vaccine plasmid was carried out in bred BALB/C mouse. All animal testing and research complied with all relevant ethical regulations and studies received ethical approval by the Institute IACUC committee. Female BALB/c mice (-5-6 weeks old) were used in the study (Envigo, Indianapolis). On Day 0, 2 50ug of formulated DNA plasmid with 0.5% crown poloxamer was injected intramuscularly (Quadriceps). Two weeks after first immunization, animals were given first booster dose. Blood was collected from animals on day 14 (2wp1) and 35 (3wp2). Further, IFN response from spleenocytes were assessed on day 35.

For delivering plasmid without formulation injections were made in tibialis followed by electroporation. A Pulse number of 2(1/second), a pulse duration 25 ms, and a pulse strength of 375/cm was used. For DNA plasmid without formulation a 50ug dose was used for IM injections.

A study was also done comparing efficacy of two immunizations to three immunizations. In that study, 5-6 week old mice were immunized intramuscularly with 2 50ug of formulated DNA plasmid with 0.5% crown poloxamer at 2 week intervals. Blood and spleen analyzed were analyzed at 3 weeks post-final injections.

### IgG Antigen S-specific ELISA

Individual serum samples were assayed for the presence of S-specific IgG by enzyme-linked immunosorbent assay (ELISA). ELISA plates (96-well, Nunc) were coated overnight at 4° C. with 1 μg/ml purified S1 antigen in PBS. After washing (PBS with 0.05% Tween-20), plates were blocked with ChonBlock ELISA Buffer (Chondrex, Inc) for 2 h at room temperature. After blocking, the plates were washed and serial dilutions of serum in ChonBlock ELISA Buffer were performed in a separate plate. The serially diluted sera were added to the assay plate and incubated for 2 h at room temperature. After washing, plates were incubated with anti-Mouse IgG, peroxidase-linked species-specific whole antibody (from sheep) Secondary Antibody (Cytiva) diluted 1:500 in PBS for 1h at room temperature. Finally, plates were washed and TMB peroxidase substrate solution (ThermoFisher) was added to the wells. Reactions were stopped by addition of 1M H₃PO₄ (Fisher Scientific) and absorbance was read at 450 nm on the EL808 ULTRA Microplate Reader luminometer (Winooski, VT) with Gen 5 ELISA software using a 1 second integration per well. For each serum sample, a plot of optical density (OD) versus logarithm of the reciprocal serum dilution was generated by nonlinear regression (GraphPad Prism). Titers were defined as the reciprocal serum dilution at an OD of approximately 0.5 (normalized to a standard mouse anti-S monoclonal antibody that is included on every plate).

### Mouse T-Cell Functional Assay

Mouse T cell functional assay was done by using IFN_{γ} ELISPOT assay. Spleens from immunized mice were collected in sterile tubes containing CTL-test media (Immunospot Cat #CTLT-005) media supplemented with 1× Glutamine and Penstrep. Cell suspensions were prepared by dissociating spleens in gentle Macs Octo Dissociator (Miltenyi Biotec, Auburn CA). The cell suspension was then filtered through a 70 μm cell strainer. The cells were pelleted by centrifugation at 4C for 10 min at 300 g in a centrifuge. The pellet was resuspended in CTL media supplemented with 1× Glutamine and Penstrep. Cells were counted using the CTL cell counting program. The cell solution was diluted to contain 250,000 cells/100 μl for each well.

The 96 well mouse IFN ELISPOT kit (Mabtech USA) pre-coated plates were used for this assay. Before use, the plates were hydrated with sterile PBS 4 times (200 μl/well). The plate was then conditioned with CTL media (same as used for splenocyte isolation for at least 30 min at RT. 250,000 mouse spleenocytes were plated into each well and stimulated for 20 h with pools of 15mer peptides covering the N terminal S1 domain of the surface glycoprotein. The peptivator SARS-CoV-2 Prot contains the aa sequence 1-692 of the surface glycoprotein (Miltenyi Biotec, Cat #130-127-041). The spots were developed based on manufacturer instructions. CTL media and PMA (Invitrogen, Waltham, MA) were used for negative and positive control respectively. Spots were scanned and quantified by Immunospot CTL reader S6 (CTL, USA). Spot forming unit per 250,000 cells was calculated and averaged from duplicate wells.

### SARS-CoV-2 Pseudovirus Neutralization Assay

HEK293T cells stably expressing hACE2 and TMPRSS2 (Invivogen, San Diego) were seeded on a 96 well plate for 16 hours. Mouse sera from vaccinated and naïve groups were serially diluted threefold in cell media starting at a 1:10 dilution in a separate dilution plate. Diluted sera were incubated at 37C for 1 hour with a fixed concentration of SARS-CoV-2 S eGFP reporter (D614G) pseudotyped lentivirus obtained from BPS Bioscience (San Diego, CA). The mouse sera and SARS-CoV-2 S pseudotyped virus mixture was then transferred to the 96 well plate containing cells and allowed to incubate in a standard incubator (37% humidity, 5% C₀₂) for 48 hours. GFP positive cells were imaged post-infection with the Immunospot S6 Analyzer (Cellular Technologies Limited), and brightfield images were used to confirm the presence of cells in each of the wells. The SARS-CoV-2 S pseudovirus was unable to infect normal 293T cells without the hACE2 and TRMPSS2 proteins. Neutralization titers were calculated as the serum dilution at which the number of GFP positive cells were reduced by 50% compared with virus control wells.

Netrualization and Immunogenicity with and without Crown Poloxamer (pVac1)

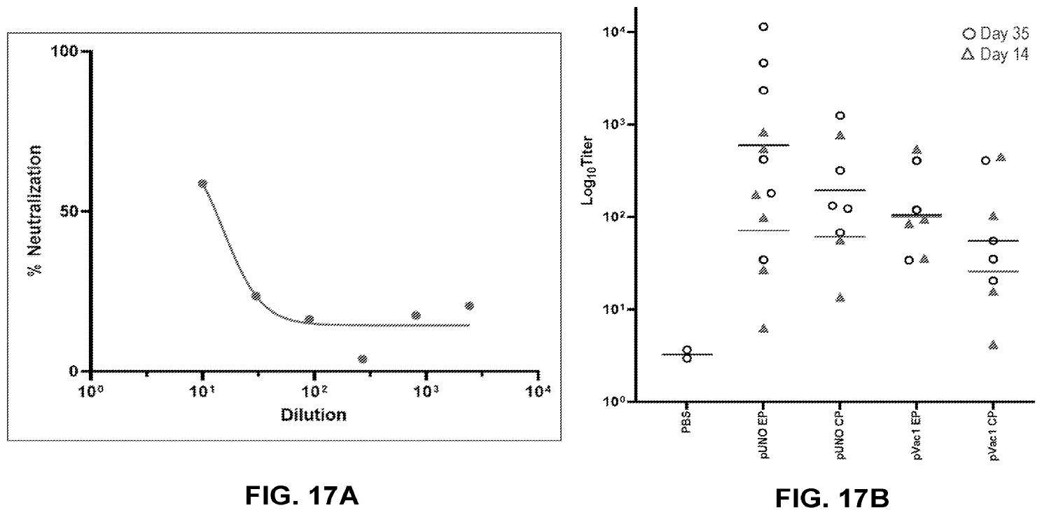

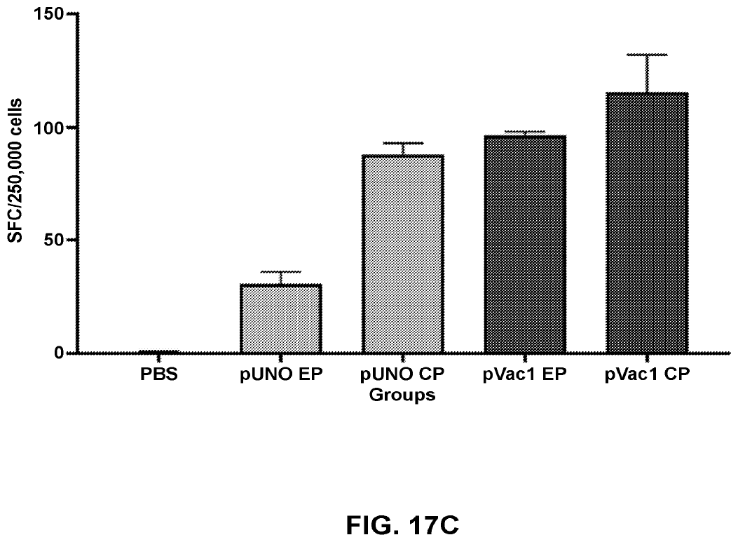

FIG. 17A illustrates that sera from mice immunized with pVac1 delivered by electroporation (EP) is able to partially neutralize the SARS-CoV-2 S eGFP reporter (D614G) pseudotyped lentivirus. Additionally, when delivered with crown poloxamer (CP), pVac1 established an antigen-dependent response at both 14 days and 35 days as measured with IgG antigen S-specific ELISA. (FIG. 17B). Using the Mouse T-cell functional assay, T cell response of pUNO and pVac1 was measured with and without electroporation (FIG. 17C). Both pVac1 and pUNO delivered with CP had increased T cell response compared to delivery via electroporation. Each column FIG. 17C represents the average of a duplicate of a pool of four spleen.

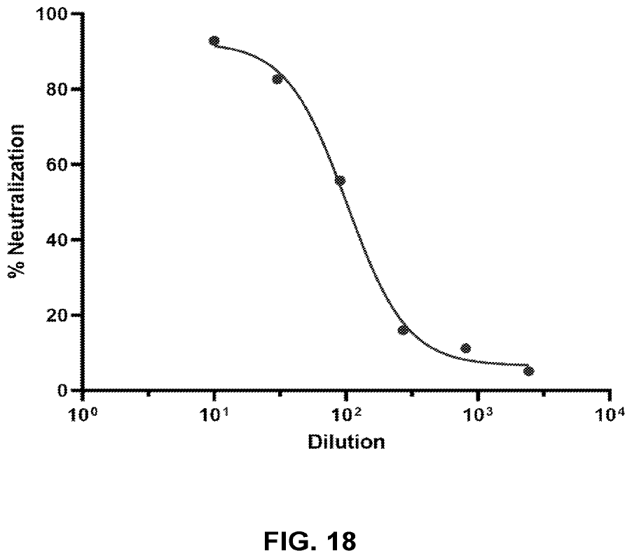

Serum from mice immunized with pUNO S delivered with crown poloxamer (CP) was analysed via neutralization assay (FIG. 18). When delivered with CP, pUNO S can block the Spike-pseudovirus entry indicating viral protection.

Immunogenicity with and without Crown Poloxamer (pVac2 and PVac3)

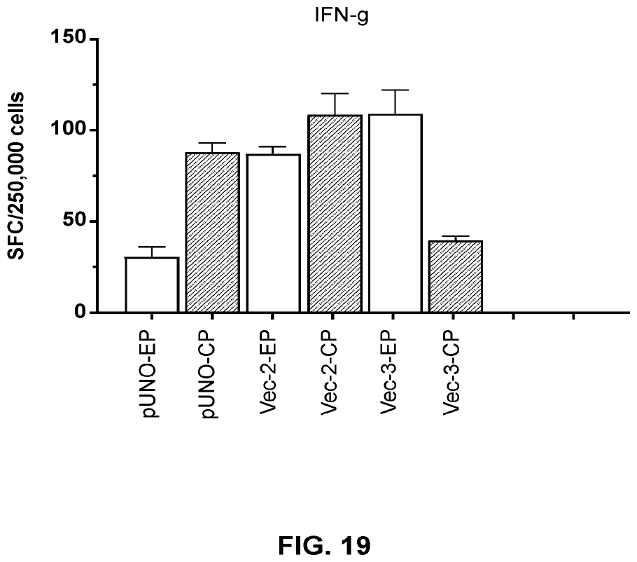

Using the Mouse T-cell functional assay, T cell response of pUNO, pVac2, and pVac3 was measured with and without electroporation (FIG. 19). pUNO, pVac2, and pVac3 delivered either by electroporation (EP) or crown poloxamer (CP) elicited T cell responses (i.e., interferon gamma (IFNγ) production) in the presence of 15mer S overlapping peptides library. Each column in FIG. 19 represents the average of a duplicate of a pool of four spleens.

### Immune Response to Vaccine Formulation and Dosing Regimens

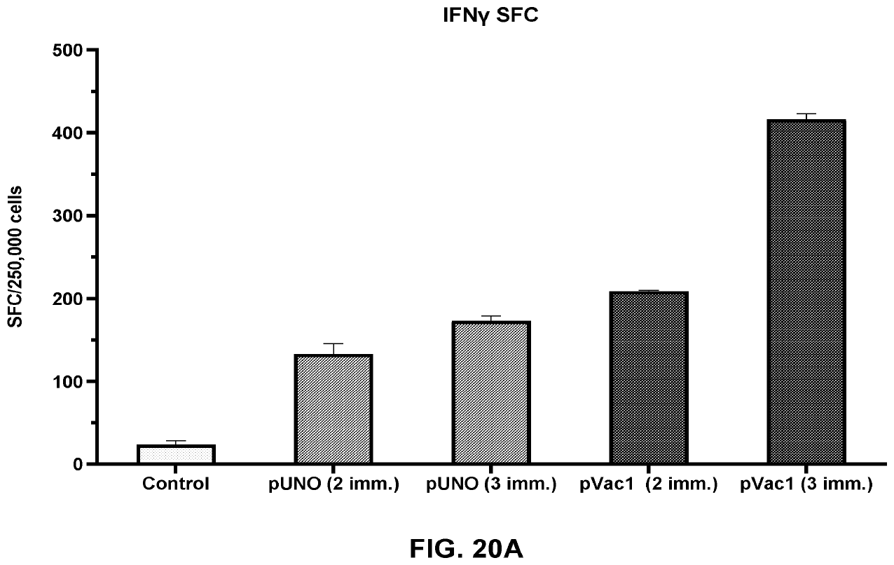

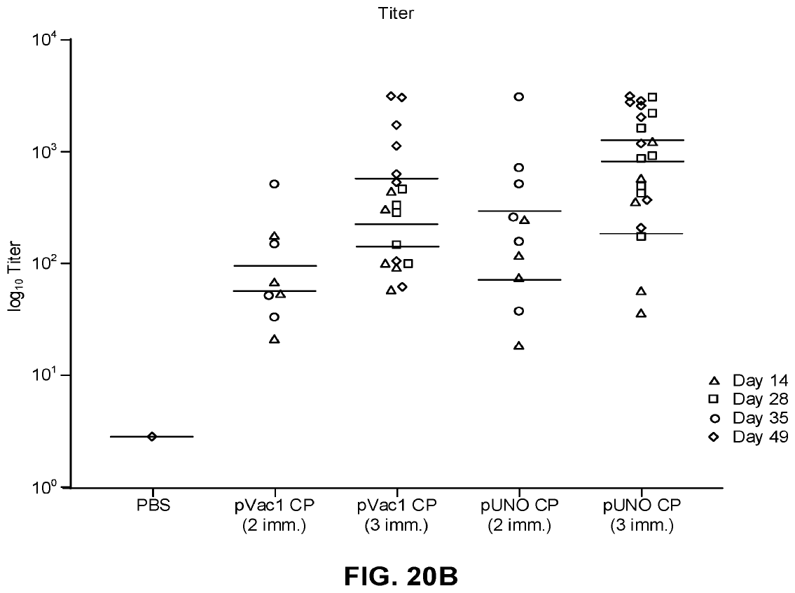

Using the Mouse T-cell functional assay, T cell response of pUNO and pVac1 with 2 and 3 immunization injections was measured (FIG. 20A). Three immunizations with pVac1 elicited a higher T-cell response than both two and three immunizations with pUNO. All immunization schedules elicited a T cell response as compared to control. Each column represents the average of a duplicate of a pool of four spleen. The error bar represents the standard deviation.

B cell response to DNA vaccines (pVac1 and pUNO 2 50ug, IM) following two immunizations or three immunizations was measured (FIG. 20B). The titer is higher in the groups immunized three times compared to the groups immunized twice. Unexpectedly, an improved cell response was observed in mice with three immunizations of pVac1 compared to three immunizations of pUNO. Each column represents the average of a duplicate point of a pool of four spleen. The error bars represent the standard deviation.

### 6.11 Future Pre Clinical Immunization Studies

In order to evaluate the ideal boost interval time, the boost IM injections with 2 50ug of formulated DNA plasmid with 0.5% crown poloxamer are administered at two, three, and four week intervals. Blood and spleen are isolated to evaluate the IgG response and T cell response three weeks post injection, followed by SARS-CoV2 pseudovirus neutralization assay.

In order to evaluate the efficacy of formulated DNA plasmid vaccine, subcutaneous and intradermal route are used for assessment of the DNA plasmid. IgG response and T cell response are evaluated for all of these studies followed by SARS-CoV2 pseudovirus neutralization assay.

TABLE 3
Sequences

| | |
|---|---|
| SEQ ID NO: 1 | ATGTTTGTTTTTCTTGTTTTATTGCCACTAGTCTCTAGTCAGT |
| SARS-COV-2 S | GTGTTAATCTTACAACCAGAACTCAATTACCCCCTGCATACA |
| Protein (codon | CTAATTCTTTCACACGTGGTGTTTATTACCCTGACAAAGTTTT |
| optimized | CAGATCCTCAGTTTTACATTCAACTCAGGACTTGTTCTTACCT |
| nucleic acid | TTCTTTTCCAATGTTACTTGGTTCCATGCTATACATGTCTCTG |
| sequence) | GGACCAATGGTACTAAGAGGTTTGATAACCCTGTCCTACCAT |
| | TTAATGATGGTGTTTATTTTGCTTCCACTGAGAAGTCTAACA |
| | TAATAAGAGGCTGGATTTTTGGTACTACTTTAGATTCGAAGA |
| | CCCAGTCCCTACTTATTGTTAATAACGCTACTAATGTTGTTAT |
| | TAAAGTCTGTGAATTTCAATTTTGTAATGATCCATTTTTGGGT |
| | GTTTATTACCACAAAAACAACAAAAGTTGGATGGAAAGTGA |
| | GTTCAGAGTTTATTCTAGTGCGAATAATTGCACTTTTGAATA |
| | TGTCTCTCAGCCTTTTCTTATGGACCTTGAAGGAAAACAGGG |
| | TAATTTCAAAAATCTTAGGGAATTTGTGTTTAAGAATATTGA |
| | TGGTTATTTTAAAATATATTCTAAGCACACGCCTATTAATTT |
| | AGTGCGTGATCTCCCTCAGGGTTTTTCGGCTTTAGAACCATT |
| | GGTAGATTTGCCAATAGGTATTAACATCACTAGGTTTCAAAC |
| | TTTACTTGCTTTACATAGAAGTTATTTGACTCCTGGTGATTCT |
| | TCTTCAGGTTGGACAGCTGGTGCTGCAGCTTATTATGTGGGT |
| | TATCTTCAACCTAGGACTTTTCTATTAAAATATAATGAAAAT |
| | GGAACCATTACAGATGCTGTAGACTGTGCACTTGACCCTCTC |
| | TCAGAAACAAAGTGTACGTTGAAATCCTTCACTGTAGAAAA |
| | AGGAATCTATCAAACTTCTAACTTTAGAGTCCAACCAACAGA |
| | ATCTATTGTTAGATTTCCTAATATTACAAACTTGTGCCCTTTT |
| | GGTGAAGTTTTTAACGCCACCAGATTTGCATCTGTTTATGCT |
| | TGGAACAGGAAGAGAATCAGCAACTGTGTTGCTGATTATTCT |
| | GTCCTATATAATTCCGCATCATTTTCCACTTTTAAGTGTTATG |
| | GAGTGTCTCCTACTAAATTAAATGATCTCTGCTTTACTAATG |
| | TCTATGCAGATTCATTTGTAATTAGAGGTGATGAAGTCAGAC |
| | AAATCGCTCCAGGGCAAACTGGAAAGATTGCTGATTATAAT |
| | TATAAATTACCAGATGATTTTACAGGCTGCGTTATAGCTTGG |
| | AATTCTAACAATCTTGATTCTAAGGTTGGTGGTAATTATAAT |
| | TACCTGTATAGATTGTTTAGGAAGTCTAATCTCAAACCTTTT |
| | GAGAGAGATATTTCAACTGAAATCTATCAGGCCGGTAGCAC |
| | ACCTTGTAATGGTGTTGAAGGTTTTAATTGTTACTTTCCTTTA |
| | CAATCATATGGTTTCCAACCCACTAATGGTGTTGGTTACCAA |
| | CCATACAGAGTAGTAGTACTTTCTTTTGAACTTCTACATGCA |
| | CCAGCAACTGTTTGTGGACCTAAAAAGTCTACTAATTTGGTT |
| | AAAAACAAATGTGTCAATTTCAACTTCAATGGTTTAACAGGC |
| | ACAGGTGTTCTTACTGAGTCTAACAAAAAGTTTCTGCCTTTC |
| | CAACAATTTGGCAGAGACATTGCTGACACTACTGATGCTGTC |
| | CGTGATCCACAGACACTTGAGATTCTTGACATTACACCATGT |
| | TCTTTTGGTGGTGTCAGTGTTATAACACCAGGAACAAATACT |
| | TCTAACCAGGTTGCTGTTCTTTATCAGGATGTTAACTGCACA |
| | GAAGTCCCTGTTGCTATTCATGCAGATCAACTTACTCCTACT |
| | TGGCGTGTTTATTCTACAGGTTCTAATGTTTTTCAAACACGTG |
| | CAGGCTGTTTAATAGGGGCTGAACATGTCAACAACTCATATG |
| | AGTGTGACATACCCATTGGTGCAGGTATATGCGCTAGTTATC |
| | AGACTCAGACTAATTCTCCTCGGCGGGCACGTAGTGTAGCTA |
| | GTCAATCCATCATTGCCTACACTATGTCACTTGGTGCAGAAA |
| | ATTCAGTTGCTTACTCTAATAACTCTATTGCCATACCCACAA |
| | ATTTTACTATTAGTGTTACCACAGAAATTCTACCAGTGTCTA |
| | TGACCAAGACATCAGTAGATTGTACAATGTACATTTGTGGTG |
| | ATTCAACTGAATGCAGCAATCTTTTGTTGCAATATGGCAGTT |
| | TTTGTACACAATTAAACCGTGCTTTAACTGGAATAGCTGTTG |
| | AACAAGACAAAAACACCCAAGAAGTTTTTGCACAAGTCAAA |
| | CAAATTTACAAAACACCACCAATTAAAGATTTTGGTGGTTTT |
| | AATTTTTCACAAATATTACCAGATCCATCAAAACCAAGCAAG |
| | AGGTCATTTATTGAAGATCTACTTTTCAACAAAGTGACACTT |
| | GCAGATGCTGGCTTCATCAAACAATATGGTGATTGCCTTGGT |
| | GATATTGCTGCTAGAGACCTCATTTGTGCACAAAAGTTTAAC |
| | GGCCTTACTGTTTTGCCACCTTTGCTCACAGATGAAATGATT |
| | GCTCAATACACTTCTGCACTGTTAGCGGGTACAATCACTTCT |
| | GGTTGGACCTTTGGTGCAGGTGCTGCATTACAAATACCATTT |
| | GCTATGCAAATGGCTTATAGGTTTAATGGTATTGGAGTTACA |
| | CAGAATGTTCTCTATGAGAACCAAAAATTGATTGCCAACCA |
| | ATTTAATAGTGCTATTGGCAAAATTCAAGACTCACTTTCTTC |
| | CACAGCAAGTGCACTTGGAAAACTTCAAGATGTGGTCAACC |
| | AAAATGCACAAGCTTTAAACACGCTTGTTAAACAACTTAGCT |
| | CCAATTTTGGTGCAATTTCAAGTGTTTTAAATGATATCCTTTC |
| | ACGTCTTGACAAAGTTGAGGCTGAAGTGCAAATTGATAGGT |
| | TGATCACAGGCAGACTTCAAAGTTTGCAGACATATGTGACTC |
| | AACAATTAATTAGAGCTGCAGAAATCAGAGCTTCTGCTAATC |
| | TTGCTGCTACTAAAATGTCAGAGTGTGTACTTGGACAATCAA |
| | AAAGAGTTGATTTTTGTGGAAAGGGCTATCATCTTATGTCCT |
| | TCCCTCAGTCAGCACCTCATGGTGTAGTCTTCTTGCATGTGA |
| | CTTATGTCCCTGCACAAGAAAAGAACTTCACAACTGCTCCTG |
| | CCATTTGTCATGATGGAAAAGCACACTTTCCTCGTGAAGGTG |
| | TCTTTGTTTCAAATGGCACACACTGGTTTGTAACACAAAGGA |
| | ATTTTTATGAACCACAAATCATTACTACAGACAACACATTTG |
| | TGTCTGGTAACTGTGATGTTGTAATAGGAATTGTCAACAACA |
| | CAGTTTATGATCCTTTGCAACCTGAATTAGACTCATTCAAGG |
| | AGGAGTTAGATAAATATTTTAAGAATCATACATCACCAGAT |
| | GTTGATTTAGGTGACATCTCTGGCATTAATGCTTCAGTTGTA |
| | AACATTCAAAAAGAAATTGACCGCCTCAATGAGGTTGCCAA |
| | GAATTTAAATGAATCTCTCATCGATCTCCAAGAACTTGGAAA |
| | GTATGAGCAGTATATAAAATGGCCATGGTACATTTGGCTAG |
| | GTTTTATAGCTGGCTTGATTGCCATAGTAATGGTGACAATTA |
| | TGCTTTGCTGTATGACCAGTTGCTGTAGTTGTCTCAAGGGCT |
| | GTTGTTCTTGTGGATCCTGCTGCAAATTTGATGAAGACGACT |
| | CTGAGCCAGTGCTCAAAGGAGTCAAATTACATTACACATAA |
| SEQ ID NO: 2 | MFVFLVLLPLVSSQCVNLTTRTQLPPAYTNSFTRGVYYPDKVFR |
| SARS-COV-2 S | SSVLHSTQDLFLPFFSNVTWFHAIHVSGTNGTKRFDNPVLPFND |
| Protein (amino | GVYFASTEKSNIIRGWIFGTTLDSKTQSLLIVNNATNVVIKVCEF |
| acid sequence) | QFCNDPFLGVYYHKNNKSWMESEFRVYSSANNCTFEYVSQPFL |
| | MDLEGKQGNFKNLREFVFKNIDGYFKIYSKHTPINLVRDLPQGF |
| | SALEPLVDLPIGINITRFQTLLALHRSYLTPGDSSSGWTAGAAAY |
| | YVGYLQPRTFLLKYNENGTITDAVDCALDPLSETKCTLKSFTVE |
| | KGIYQTSNFRVQPTESIVRFPNITNLCPFGEVENATRFASVYAW |
| | NRKRISNCVADYSVLYNSASFSTFKCYGVSPTKLNDLCFTNVY |
| | ADSFVIRGDEVRQIAPGQTGKIADYNYKLPDDFTGCVIAWNSN |
| | NLDSKVGGNYNYLYRLFRKSNLKPFERDISTEIYQAGSTPCNGV |
| | EGFNCYFPLQSYGFQPTNGVGYQPYRVVVLSFELLHAPATVCG |
| | PKKSTNLVKNKCVNFNFNGLTGTGVLTESNKKFLPFQQFGRDI |
| | ADTTDAVRDPQTLEILDITPCSFGGVSVITPGTNTSNQVAVLYQ |
| | DVNCTEVPVAIHADQLTPTWRVYSTGSNVFQTRAGCLIGAEHV |
| | NNSYECDIPIGAGICASYQTQTNSPRRARSVASQSIIAYTMSLGA |
| | ENSVAYSNNSIAIPTNFTISVTTEILPVSMTKTSVDCTMYICGDST |
| | ECSNLLLQYGSFCTQLNRALTGIAVEQDKNTQEVFAQVKQIYK |
| | TPPIKDFGGFNFSQILPDPSKPSKRSFIEDLLFNKVTLADAGFIKQ |
| | YGDCLGDIAARDLICAQKFNGLTVLPPLLTDEMIAQYTSALLAG |
| | TITSGWTFGAGAALQIPFAMQMAYRFNGIGVTQNVLYENQKLI |
| | ANQFNSAIGKIQDSLSSTASALGKLQDVVNQNAQALNTLVKQL |
| | SSNFGAISSVLNDILSRLDKVEAEVQIDRLITGRLQSLQTYVTQQ |
| | LIRAAEIRASANLAATKMSECVLGQSKRVDFCGKGYHLMSFPQ |
| | SAPHGVVFLHVTYVPAQEKNFTTAPAICHDGKAHFPREGVFVS |
| | NGTHWFVTQRNFYEPQIITTDNTFVSGNCDVVIGIVNNTVYDPL |
| | QPELDSFKEELDKYFKNHTSPDVDLGDISGINASVVNIQKEIDRL |
| | NEVAKNLNESLIDLQELGKYEQYIKWPWYIWLGFIAGLIAIVMV |
| | TIMLCCMTSCCSCLKGCCSCGSCCKFDEDDSEPVLKGVKLHYT |
| SEQ ID NO: 3 | ATGTTTGTTTTTCTTGTTTTATTGCCACTAGTCTCTAGTCAGT |
| SARS-COV-2 | GTGTTAATCTTACAACCAGAACTCAATTACCCCCTGCATACA |
| D614G S Protein | CTAATTCTTTCACACGTGGTGTTTATTACCCTGACAAAGTTTT |
| (codon | CAGATCCTCAGTTTTACATTCAACTCAGGACTTGTTCTTACCT |
| optimized | TTCTTTTCCAATGTTACTTGGTTCCATGCTATACATGTCTCTG |
| nucleic acid | GGACCAATGGTACTAAGAGGTTTGATAACCCTGTCCTACCAT |
| sequence) | TTAATGATGGTGTTTATTTTGCTTCCACTGAGAAGTCTAACA |
| | TAATAAGAGGCTGGATTTTTGGTACTACTTTAGATTCGAAGA |
| | CCCAGTCCCTACTTATTGTTAATAACGCTACTAATGTTGTTAT |
| | TAAAGTCTGTGAATTTCAATTTTGTAATGATCCATTTTTGGGT |
| | GTTTATTACCACAAAAACAACAAAAGTTGGATGGAAAGTGA |
| | GTTCAGAGTTTATTCTAGTGCGAATAATTGCACTTTTGAATA |
| | TGTCTCTCAGCCTTTTCTTATGGACCTTGAAGGAAAACAGGG |
| | TAATTTCAAAAATCTTAGGGAATTTGTGTTTAAGAATATTGA |
| | TGGTTATTTTAAAATATATTCTAAGCACACGCCTATTAATTT |
| | AGTGCGTGATCTCCCTCAGGGTTTTTCGGCTTTAGAACCATT |
| | GGTAGATTTGCCAATAGGTATTAACATCACTAGGTTTCAAAC |
| | TTTACTTGCTTTACATAGAAGTTATTTGACTCCTGGTGATTCT |
| | TCTTCAGGTTGGACAGCTGGTGCTGCAGCTTATTATGTGGGT |
| | TATCTTCAACCTAGGACTTTTCTATTAAAATATAATGAAAAT |
| | GGAACCATTACAGATGCTGTAGACTGTGCACTTGACCCTCTC |
| | TCAGAAACAAAGTGTACGTTGAAATCCTTCACTGTAGAAAA |
| | AGGAATCTATCAAACTTCTAACTTTAGAGTCCAACCAACAGA |
| | ATCTATTGTTAGATTTCCTAATATTACAAACTTGTGCCCTTTT |
| | GGTGAAGTTTTTAACGCCACCAGATTTGCATCTGTTTATGCT |
| | TGGAACAGGAAGAGAATCAGCAACTGTGTTGCTGATTATTCT |
| | GTCCTATATAATTCCGCATCATTTTCCACTTTTAAGTGTTATG |
| | GAGTGTCTCCTACTAAATTAAATGATCTCTGCTTTACTAATG |
| | TCTATGCAGATTCATTTGTAATTAGAGGTGATGAAGTCAGAC |
| | AAATCGCTCCAGGGCAAACTGGAAAGATTGCTGATTATAAT |
| | TATAAATTACCAGATGATTTTACAGGCTGCGTTATAGCTTGG |
| | AATTCTAACAATCTTGATTCTAAGGTTGGTGGTAATTATAAT |
| | TACCTGTATAGATTGTTTAGGAAGTCTAATCTCAAACCTTTT |
| | GAGAGAGATATTTCAACTGAAATCTATCAGGCCGGTAGCAC |
| | ACCTTGTAATGGTGTTGAAGGTTTTAATTGTTACTTTCCTTTA |
| | CAATCATATGGTTTCCAACCCACTAATGGTGTTGGTTACCAA |
| | CCATACAGAGTAGTAGTACTTTCTTTTGAACTTCTACATGCA |
| | CCAGCAACTGTTTGTGGACCTAAAAAGTCTACTAATTTGGTT |
| | AAAAACAAATGTGTCAATTTCAACTTCAATGGTTTAACAGGC |
| | ACAGGTGTTCTTACTGAGTCTAACAAAAAGTTTCTGCCTTTC |
| | CAACAATTTGGCAGAGACATTGCTGACACTACTGATGCTGTC |
| | CGTGATCCACAGACACTTGAGATTCTTGACATTACACCATGT |
| | TCTTTTGGTGGTGTCAGTGTTATAACACCAGGAACAAATACT |
| | TCTAACCAGGTTGCTGTTCTTTATCAGGGTGTTAACTGCACA |
| | GAAGTCCCTGTTGCTATTCATGCAGATCAACTTACTCCTACT |
| | TGGCGTGTTTATTCTACAGGTTCTAATGTTTTTCAAACACGTG |
| | CAGGCTGTTTAATAGGGGCTGAACATGTCAACAACTCATATG |
| | AGTGTGACATACCCATTGGTGCAGGTATATGCGCTAGTTATC |
| | AGACTCAGACTAATTCTCCTCGGCGGGCACGTAGTGTAGCTA |
| | GTCAATCCATCATTGCCTACACTATGTCACTTGGTGCAGAAA |
| | ATTCAGTTGCTTACTCTAATAACTCTATTGCCATACCCACAA |
| | ATTTTACTATTAGTGTTACCACAGAAATTCTACCAGTGTCTA |
| | TGACCAAGACATCAGTAGATTGTACAATGTACATTTGTGGTG |
| | ATTCAACTGAATGCAGCAATCTTTTGTTGCAATATGGCAGTT |
| | TTTGTACACAATTAAACCGTGCTTTAACTGGAATAGCTGTTG |
| | AACAAGACAAAAACACCCAAGAAGTTTTTGCACAAGTCAAA |
| | CAAATTTACAAAACACCACCAATTAAAGATTTTGGTGGTTTT |
| | AATTTTTCACAAATATTACCAGATCCATCAAAACCAAGCAAG |
| | AGGTCATTTATTGAAGATCTACTTTTCAACAAAGTGACACTT |
| | GCAGATGCTGGCTTCATCAAACAATATGGTGATTGCCTTGGT |
| | GATATTGCTGCTAGAGACCTCATTTGTGCACAAAAGTTTAAC |
| | GGCCTTACTGTTTTGCCACCTTTGCTCACAGATGAAATGATT |
| | GCTCAATACACTTCTGCACTGTTAGCGGGTACAATCACTTCT |
| | GGTTGGACCTTTGGTGCAGGTGCTGCATTACAAATACCATTT |
| | GCTATGCAAATGGCTTATAGGTTTAATGGTATTGGAGTTACA |
| | CAGAATGTTCTCTATGAGAACCAAAAATTGATTGCCAACCA |
| | ATTTAATAGTGCTATTGGCAAAATTCAAGACTCACTTTCTTC |
| | CACAGCAAGTGCACTTGGAAAACTTCAAGATGTGGTCAACC |
| | AAAATGCACAAGCTTTAAACACGCTTGTTAAACAACTTAGCT |
| | CCAATTTTGGTGCAATTTCAAGTGTTTTAAATGATATCCTTTC |
| | ACGTCTTGACAAAGTTGAGGCTGAAGTGCAAATTGATAGGT |
| | TGATCACAGGCAGACTTCAAAGTTTGCAGACATATGTGACTC |
| | AACAATTAATTAGAGCTGCAGAAATCAGAGCTTCTGCTAATC |
| | TTGCTGCTACTAAAATGTCAGAGTGTGTACTTGGACAATCAA |
| | AAAGAGTTGATTTTTGTGGAAAGGGCTATCATCTTATGTCCT |
| | TCCCTCAGTCAGCACCTCATGGTGTAGTCTTCTTGCATGTGA |
| | CTTATGTCCCTGCACAAGAAAAGAACTTCACAACTGCTCCTG |
| | CCATTTGTCATGATGGAAAAGCACACTTTCCTCGTGAAGGTG |
| | TCTTTGTTTCAAATGGCACACACTGGTTTGTAACACAAAGGA |
| | ATTTTTATGAACCACAAATCATTACTACAGACAACACATTTG |
| | TGTCTGGTAACTGTGATGTTGTAATAGGAATTGTCAACAACA |
| | CAGTTTATGATCCTTTGCAACCTGAATTAGACTCATTCAAGG |
| | AGGAGTTAGATAAATATTTTAAGAATCATACATCACCAGAT |
| | GTTGATTTAGGTGACATCTCTGGCATTAATGCTTCAGTTGTA |
| | AACATTCAAAAAGAAATTGACCGCCTCAATGAGGTTGCCAA |
| | GAATTTAAATGAATCTCTCATCGATCTCCAAGAACTTGGAAA |
| | GTATGAGCAGTATATAAAATGGCCATGGTACATTTGGCTAG |
| | GTTTTATAGCTGGCTTGATTGCCATAGTAATGGTGACAATTA |
| | TGCTTTGCTGTATGACCAGTTGCTGTAGTTGTCTCAAGGGCT |
| | GTTGTTCTTGTGGATCCTGCTGCAAATTTGATGAAGACGACT |
| | CTGAGCCAGTGCTCAAAGGAGTCAAATTACATTACACATAA |
| SEQ ID NO: 4 | MFVFLVLLPLVSSQCVNLTTRTQLPPAYTNSFTRGVYYPDKVFR |
| SARS-COV-2 | SSVLHSTQDLFLPFFSNVTWFHAIHVSGTNGTKRFDNPVLPEND |
| D614G S Protein | GVYFASTEKSNIIRGWIFGTTLDSKTQSLLIVNNATNVVIKVCEF |
| (amino acid | QFCNDPFLGVYYHKNNKSWMESEFRVYSSANNCTFEYVSQPFL |
| sequence) | MDLEGKQGNFKNLREFVFKNIDGYFKIYSKHTPINLVRDLPQGF |
| | SALEPLVDLPIGINITRFQTLLALHRSYLTPGDSSSGWTAGAAAY |
| | YVGYLQPRTFLLKYNENGTITDAVDCALDPLSETKCTLKSFTVE |
| | KGIYQTSNFRVQPTESIVRFPNITNLCPFGEVENATRFASVYAW |
| | NRKRISNCVADYSVLYNSASFSTFKCYGVSPTKLNDLCFTNVY |
| | ADSFVIRGDEVRQIAPGQTGKIADYNYKLPDDFTGCVIAWNSN |
| | NLDSKVGGNYNYLYRLFRKSNLKPFERDISTEIYQAGSTPCNGV |
| | EGFNCYFPLQSYGFQPTNGVGYQPYRVVVLSFELLHAPATVCG |
| | PKKSTNLVKNKCVNFNFNGLTGTGVLTESNKKFLPFQQFGRDI |
| | ADTTDAVRDPQTLEILDITPCSFGGVSVITPGTNTSNQVAVLYQ |
| | GVNCTEVPVAIHADQLTPTWRVYSTGSNVFQTRAGCLIGAEHV |
| | NNSYECDIPIGAGICASYQTQTNSPRRARSVASQSIIAYTMSLGA |
| | ENSVAYSNNSIAIPTNFTISVTTEILPVSMTKTSVDCTMYICGDST |
| | ECSNLLLQYGSFCTQLNRALTGIAVEQDKNTQEVFAQVKQIYK |
| | TPPIKDFGGFNFSQILPDPSKPSKRSFIEDLLFNKVTLADAGFIKQ |
| | YGDCLGDIAARDLICAQKFNGLTVLPPLLTDEMIAQYTSALLAG |
| | TITSGWTFGAGAALQIPFAMQMAYRENGIGVTQNVLYENQKLI |
| | ANQFNSAIGKIQDSLSSTASALGKLQDVVNQNAQALNTLVKQL |
| | SSNFGAISSVLNDILSRLDKVEAEVQIDRLITGRLQSLQTYVTQQ |
| | LIRAAEIRASANLAATKMSECVLGQSKRVDFCGKGYHLMSFPQ |
| | SAPHGVVFLHVTYVPAQEKNFTTAPAICHDGKAHFPREGVFVS |
| | NGTHWFVTQRNFYEPQIITTDNTFVSGNCDVVIGIVNNTVYDPL |
| | QPELDSFKEELDKYFKNHTSPDVDLGDISGINASVVNIQKEIDRL |
| | NEVAKNLNESLIDLQELGKYEQYIKWPWYIWLGFIAGLIAIVMV |
| | TIMLCCMTSCCSCLKGCCSCGSCCKFDEDDSEPVLKGVKLHYT |
| SEQ ID NO: 5 | CGGGTCCAACCCACCGAAAGCATTGTGCGGTTCCCAAATATC |
| SARS-COV-2 | ACCAATCTGTGTCCCTTTGGCGAAGTGTTCAATGCTACAAGG |
| RBD S Protein | TTTGCTTCTGTGTACGCATGGAATAGGAAACGCATCTCCAAT |
| (nucleic acid | TGTGTCGCTGATTACTCCGTGCTGTACAATTCCGCCTCTTTCT |
| sequence) | CAACCTTCAAGTGTTATGGCGTTTCACCTACCAAACTTAACG |
| | ACCTGTGCTTCACTAATGTGTATGCCGACTCTTTTGTGATAC |
| | GAGGCGATGAAGTGAGACAGATTGCACCAGGGCAGACCGGC |
| | AAAATTGCCGACTACAACTACAAGCTTCCAGATGACTTTACC |
| | GGATGTGTTATTGCATGGAACTCAAACAATCTGGATTCCAAG |
| | GTGGGTGGCAACTATAACTACCTGTATAGACTGTTCAGGAA |
| | ATCCAACCTGAAACCATTCGAGCGAGATATAAGCACAGAAA |
| | TCTACCAGGCTGGAAGTACGCCCTGCAACGGCGTGGAAGGG |
| | TTCAACTGCTACTTCCCATTGCAGAGTTACGGATTCCAGCCT |
| | ACAAACGGGGTGGGTTACCAACCCTATCGTGTCGTAGTCCTG |
| | AGTTTTGAGCTCCTCCATGCCCCAGCCACAGTCTGTGGCCCC |
| | AAGAAAAGCACCAATCTGGTGAAGAACAAATGCGTGAACTT |
| | TTAG |
| SEQ ID NO: 6 | RVQPTESIVRFPNITNLCPFGEVENATRFASVYAWNRKRISNCV |
| SARS-COV-2 | ADYSVLYNSASFSTFKCYGVSPTKLNDLCFTNVYADSFVIRGDE |
| RBD S Protein | VRQIAPGQTGKIADYNYKLPDDFTGCVIAWNSNNLDSKVGGN |
| (amino acid | YNYLYRLFRKSNLKPFERDISTEIYQAGSTPCNGVEGENCYFPL |
| sequence) | QSYGFQPTNGVGYQPYRVVVLSFELLHAPATVCGPKKSTNLVK |
| | NKCVNF |
| SEQ ID NO: 7 | ATGGCAGATTCCAACGGTACTATTACCGTTGAGGAACTTAAA |
| SARS-COV-2 M | AAGCTCCTTGAACAATGGAACCTAGTAATAGGTTTCCTATTC |
| Protein (nucleic | CTTACATGGATTTGTCTTCTACAATTTGCCTATGCCAACAGG |
| acid sequence) | AATAGGTTTTTGTATATAATTAAGTTAATTTTCCTCTGGCTGT |
| | TATGGCCAGTAACTTTAGCTTGTTTTGTGCTTGCTGCTGTTTA |
| | CAGAATAAATTGGATCACCGGTGGAATTGCTATCGCAATGG |
| | CTTGTCTTGTAGGCTTGATGTGGCTCAGCTACTTCATTGCTTC |
| | TTTCAGACTGTTTGCGCGTACGCGTTCCATGTGGTCATTCAA |
| | TCCAGAAACTAACATTCTTCTCAACGTGCCACTCCATGGCAC |
| | TATTCTGACCAGACCGCTTCTAGAAAGTGAACTCGTAATCGG |
| | AGCTGTGATCCTTCGTGGACATCTTCGTATTGCTGGACACCA |
| | TCTAGGACGCTGTGACATCAAGGACCTGCCTAAAGAAATCA |
| | CTGTTGCTACATCACGAACGCTTTCTTATTACAAATTGGGAG |
| | CTTCGCAGCGTGTAGCAGGTGACTCAGGTTTTGCTGCATACA |
| | GTCGCTACAGGATTGGCAACTATAAATTAAACACAGACCAT |
| | TCCAGTAGCAGTGACAATATTGCTTTGCTTGTACAGTAA |
| SEQ ID NO: 8 | MADSNGTITVEELKKLLEQWNLVIGFLFLTWICLLQFAYANRN |
| SARS-COV-2 M | RFLYIIKLIFLWLLWPVTLACFVLAAVYRINWITGGIAIAMACLV |
| Protein (amino | GLMWLSYFIASFRLFARTRSMWSFNPETNILLNVPLHGTILTRPL |
| acid sequence) | LESELVIGAVILRGHLRIAGHHLGRCDIKDLPKEITVATSRTLSY |
| | YKLGASQRVAGDSGFAAYSRYRIGNYKLNTDHSSSSDNIALLVQ |
| SEQ ID NO: 9 | ATGGCAGATTCCAACGGTACTATTACCGTTGAAGAGCTTAAA |
| WA9-UW6 | AAGCTCCTTGAACAATGGAACCTAGTAATAGGTTTCCTATTC |
| Variant M | CTTACATGGATTTGTCTTCTACAATTTGCCTATGCCAACAGG |
| Protein (nucleic | AATAGGTTTTTGTATATAATTAAGTTAATTTTCCTCTGGCTGT |
| acid sequence) | TATGGCCAGTAACTTTAGCTTGTTTTGTGCTTGCTGCTATTTA |
| | CAGAATAAATTGGATCACCGGTGGAATTGCTATCGCAATGG |
| | CTTGTCTTGTAGGCTTGATGTGGCTCAGCTACTTCATTGCTTC |
| | TTTCAGACTGTTTGCGCGTACGCGTTCCATGTGGTCATTCAA |
| | TCCAGAAACTAACATTCTTCTCAACGTGCCACTCCATGGCAC |
| | TATTCTGACCAGACCGCTTCTAGAAAGTGAACTCGTAATCGG |
| | AGCTGTGATCCTTCGTGGACATCTTCGTATTGCTGGACACCA |
| | TCTAGGACGCTGTGACATCAAGGACCTGCCTAAAGAAATCA |
| | CTGTTGCTACATCACGAACGCTTTCTTATTACAAATTGGGAG |
| | CTTCGCAGCGTGTAGCAGGTGACTCAGGTTTTGCTGCATACA |
| | GTCGCTACAGGATTGGCAACTATAAATTAAACACAGACCAT |
| | TCCAGTAGCAGTGACAATATTGCTTTGCTTGTACAGTAA |
| SEQ ID NO: 10 | MADSNGTITVEELKKLLEQWNLVIGFLFLTWICLLQFAYANRN |
| WA9-UW6 | RFLYIIKLIFLWLLWPVTLACFVLAAIYRINWITGGIAIAMACLV |
| Variant M | GLMWLSYFIASFRLFARTRSMWSFNPETNILLNVPLHGTILTRPL |
| Protein (amino | LESELVIGAVILRGHLRIAGHHLGRCDIKDLPKEITVATSRTLSY |
| acid sequence) | YKLGASQRVAGDSGFAAYSRYRIGNYKLNTDHSSSSDNIALLVQ |
| SEQ ID NO: 11 | ATGGCAGATTCCAACGGTACTATTACCGTTGAAGAGCTTAAA |
| NIHE Variant M | AAGCTCCTTGAACAATGGAACCTAGTAATAGGTTTCCTATTC |
| Protein (nucleic | CTTACATGGATTTGTCTTCTACAATTTGCCTATGCCAACAGG |
| acid sequence) | AATAGGTTTTTGTATATAATTAAGTTAATTTTCCTCTGGCTGG |
| | TATGGCCAGTAACTTTAGCTTGTTTTGTGCTTGCTGCCGTTTA |
| | CAGAATAAATTGGATCACCGGTGGAATTGCTATCGCAATGG |
| | CTTGTCTTGTACGCTTGATGTGGCTCAGCTACTTCATTGCTTC |
| | TTTCAGACTGTTTGCGCGTACGCGTTCCATGTGGTCATTCAA |
| | TCCAGAAACTAACATTCTTCTCAACGTGCCACTCCATGGCAC |
| | TATTCTGACCAGACCGCTTCTAGAAAGTGAACTCGTAATCGG |
| | AGCTGTGATCCTTCGTGGACATCTTCGTATTGCTGGACACCA |
| | TCTAGGACGCTGTGACATCAAGGACCTGCCTAAAGAAATCA |
| | CTGTTGCTACATCACGAACGCTTTCTTATTACAAATTGGGAG |
| | CTTCGCAGCGTGTAGCAGGTGACTCAGGTTTTGCTGCATACA |
| | GTCGCTACAGGATTGGCAACTATAAATTAAACACAGACCAT |
| | TCCAGTAGCAGTGACAATATTGCTTTGCTTGTACAGTAA |
| SEQ ID NO: 12 | MADSNGTITVEELKKLLEQWNLVIGFLFLTWICLLQFAYANRN |
| NIHE Variant M | RFLYIIKLIFLWLVWPVTLACFVLAAVYRINWITGGIAIAMACL |
| Protein (amino | VRLMWLSYFIASFRLFARTRSMWSFNPETNILLNVPLHGTILTRP |
| acid sequence) | LLESELVIGAVILRGHLRIAGHHLGRCDIKDLPKEITVATSRTLS |
| | YYKLGASQRVAGDSGFAAYSRYRIGNYKLNTDHSSSSDNIALLVQ |
| SEQ ID NO: 13 | ATGTCAGATTCCAACGGTACTATTACCGTTGAAGAGCTTAAA |
| Wuhan_IME_BJ | AAGCTCCTTGAACAATGGAACCTAGTAATAGGTTTCCTATTC |
| O7 Variant M | CTTACATGGATTTGTCTTCTACAATTTGCCTATGCCAACAGG |
| Protein (nucleic | AATAGGTTTTTGTATATAATTAAGTTAATTTTCCTCTGGCTGT |
| acid sequence) | TATGGCCAGTAACTTTAGCTTGTTTTGTGCTTGCTGCTGTTTA |
| | CAGAATAAATTGGATCACCGGTGGAATTGCTATCGCAATGG |
| | CTTGTCTTGTAGGCTTGATGTGGCTCAGCTACTTCATTGCTTC |
| | TTTCAGACTGTTTGCGCGTACGCGTTCCATGTGGTCATTCAA |
| | TCCAGAAACTAACATTCTTCTCAACGTGCCACTCCATGGCAC |
| | TATTCTGACCAGACCGCTTCTAGAAAGTGAACTCGTAATCGG |
| | AGCTGTGATCCTTCGTGGACATCTTCGTATTGCTGGACACCA |
| | TCTAGGACGCTGTGACATCAAGGACCTGCCTAAAGAAATCA |
| | CTGTTGCTACATCACGAACGCTTTCTTATTACAAATTGGGAG |
| | CTTCGCAGCGTGTAGCAGGTGACTCAGGTTTTGCTGCATACA |
| | GTCGCTACAGGATTGGCAACTATAAATTAAACACAGACCAT |
| | TCCAGTAGCAGTGACAATATTGCTTTGCTTGTACAGTAA |
| SEQ ID NO: 14 | MSDSNGTITVEELKKLLEQWNLVIGFLFLTWICLLQFAYANRNR |
| Wuhan_IME_BJ | FLYIIKLIFLWLLWPVTLACFVLAAVYRINWITGGIAIAMACLVG |
| O7 Variant M | LMWLSYFIASFRLFARTRSMWSFNPETNILLNVPLHGTILTRPLL |
| Protein (amino | ESELVIGAVILRGHLRIAGHHLGRCDIKDLPKEITVATSRTLSYY |
| acid sequence) | KLGASQRVAGDSGFAAYSRYRIGNYKLNTDHSSSSDNIALLVQ |
| SEQ ID NO: 15 | ATGGCAGATTCCAACGGTACTATTACCGTTGAAGAGCTTAAA |
| WA-UW-1753 | AAGCTCCTTGAACAATGGAACCTAGTAATAGGTTTCCTATTC |
| Variant M | CTTACATGGATTTGTCTTCTACAATTTGCCTATGCCAACAGG |
| Protein (nucleic | AATAGGTTTTTGTATATAATTAAGTTAATTTTCCTCTGGCTGT |
| acid sequence) | TATGGCCAGTAACTTTAGCTTGTTTTGTGCTTGCTGCTGTTTA |
| | CAGAATAAATTGGATCACCGGTGGAATTGCTATCGCAATGTC |
| | TTGTCTTGTAGGCTTGATGTGGCTCAGCTACTTCATTGCTTCT |
| | TTCAGACTGTTTGCGCGTACGCGTTCCATGTGGTCATTCAAT |
| | CCAGAAACTAACATTCTTCTCAACGTGCCACTCCATGGCACT |
| | ATTCTGACCAGACCGCTTCTAGAAAGTGAACTCGTAATCGGA |
| | GCTGTGATCCTTCGTGGACATCTTCGTATTGCTGGACACCAT |
| | CTAGGACGCTGTGACATCAAGGACCTGCCTAAAGAAATCAC |
| | TGTTGCTACATCACGAACGCTTTCTTATTACAAATTGGGAGC |
| | TTCGCAGCGTGTAGCAGGTGACTCAGGTTTTGCTGCATACAG |
| | TCGCTACAGGATTGGCAACTATAAATTAAACACAGACCATTC |
| | CAGTAGCAGTGACAATATTGCTTTGCTTGTACAGTAA |
| SEQ ID NO: 16 | MADSNGTITVEELKKLLEQWNLVIGFLFLTWICLLQFAYANRN |
| WA-UW-1753 | RFLYIIKLIFLWLLWPVTLACFVLAAVYRINWITGGIAIAMSCLV |
| Variant M | GLMWLSYFIASFRLFARTRSMWSFNPETNILLNVPLHGTILTRPL |
| Protein (amino | LESELVIGAVILRGHLRIAGHHLGRCDIKDLPKEITVATSRTLSY |
| acid sequence) | YKLGASQRVAGDSGFAAYSRYRIGNYKLNTDHSSSSDNIALLVQ |
| SEQ ID NO: 17 | ATGGCAGATTCCAACGGTACTATTACCGTTGAAGAGCTTAAA |
| WA UW-1755 | AAGCTCCTTGAACAATGGAACCTAGTAATAGGTTTCCTATTC |
| Variant M | CTTACATGGATTTGTCTTCTACAATTTGCCTATGCCAACAGG |
| Protein (nucleic | AATAGGTTTTTGTATATAATTAAGTTAATTTTCCTCTGGCTGT |
| acid sequence) | TATGGCCAGTAACTTTAGCTTGTTTTGTGCTTGCTGCTGTTTA |
| | CAGAATAAATTGGATCACCGGTGGAATTGCTATCGCAATGG |
| | CTTGTCTTGTAGGCTTGATGTGGCTCAGCTACTTCATTGCTTC |
| | TTTCAGACTGTTTGCGCGTACGCGTTCCATGTGGTCATTCAA |
| | TCCAGAAACTAACATTCTTCTCAACGTGCCACTCCATGGCAC |
| | TATTCTGACCAGACCGCTTCTAGAAAGTGAACTCGTAATCGG |
| | AGCTGTGATCCTTCGTGGACATCTTCGTATTGCTGGACACCA |
| | TCTAGGACGCTGTGACATCAAGGACCTGCCTAAAGAAATCA |
| | CTGTTGCTACATCACGAATGCTTTCTTATTACAAATTGGGAG |
| | CTTCGCAGCGTGTAGCAGGTGACTCAGGTTTTGCTGCATACA |
| | GTCGCTACAGGATTGGCAACTATAAATTAAACACAGACCAT |
| | TCCAGTAGCAGTGACAATATTGCTTTGCTTGTACAGTAA |
| SEQ ID NO: 18 | MADSNGTITVEELKKLLEQWNLVIGFLFLTWICLLQFAYANRN |
| WA UW-1755 | RFLYIIKLIFLWLLWPVTLACFVLAAVYRINWITGGIAIAMACLV |
| Variant M | GLMWLSYFIASFRLFARTRSMWSFNPETNILLNVPLHGTILTRPL |
| Protein (amino | LESELVIGAVILRGHLRIAGHHLGRCDIKDLPKEITVATSRMLSY |
| acid sequence) | YKLGASQRVAGDSGFAAYSRYRIGNYKLNTDHSSSSDNIALLVQ |
| SEQ ID NO: 19 | ATGGCAGGTTCCAACGGTACTATTACCGTTGAAGAGCTTAAA |
| USA_SC_3572 | AAGCTCCTTGAACAATGGAACCTAGTAATAGGTTTCCTATTC |
| Variant M | CTTACATGGATTTGTCTTCTACAATTTGCCTATGCCAACAGG |
| Protein (nucleic | AATAGGTTTTTGTATATAATTAAGTTAATTTTCCTCTGGCTGT |
| acid sequence) | TATGGCCAGTAACTTTAGCTTGTTTTGTGCTTGCTGCTGTTTA |
| | CAGAATAAATTGGATCACCGGTGGAATTGCTATCGCAATGG |
| | CTTGTCTTGTAGGCTTGATGTGGCTCAGCTACTTCATTGCTTC |
| | TTTCAGACTGTTTGCGCGTACGCGTTCCATGTGGTCATTCAA |
| | TCCAGAAACTAACATTCTTCTCAACGTGCCACTCCATGGCAC |
| | TATTCTGACCAGACCGCTTCTAGAAAGTGAACTCGTAATCGG |
| | AGCTGTGATCCTTCGTGGACATCTTCGTATTGCTGGACACCA |
| | TCTAGGACGCTGTGACATCAAGGACCTGCCTAAAGAAATCA |
| | CTGTTGCTACATCACGAACGCTTTCTTATTACAAATTGGGAG |
| | CTTCGCAGCGTGTAGCAGGTGACTCAGGTTTTGCTGCATACA |
| | GTCGCTACAGGATTGGCAACTATAAATTAAACACAGACCAT |
| | TCCAGTAGCAGTGACAATATTGCTTTGCTTGTACAGTAA |
| SEQ ID NO: 20 | MAGSNGTITVEELKKLLEQWNLVIGFLFLTWICLLQFAYANRN |
| USA_SC_3572 | RFLYIIKLIFLWLLWPVTLACFVLAAVYRINWITGGIAIAMACLV |
| Variant M | GLMWLSYFIASFRLFARTRSMWSFNPETNILLNVPLHGTILTRPL |
| Protein (amino | LESELVIGAVILRGHLRIAGHHLGRCDIKDLPKEITVATSRTLSY |
| acid sequence) | YKLGASQRVAGDSGFAAYSRYRIGNYKLNTDHSSSSDNIALLVQ |
| SEQ ID NO: 21 | ATGTACTCATTCGTTTCGGAAGAGACAGGTACGTTAATAGTT |
| SARS-COV-2 E | AATAGCGTACTTCTTTTTCTTGCTTTCGTGGTATTCTTGCTAG |
| Protein (nucleic | TTACACTAGCCATCCTTACTGCGCTTCGATTGTGTGCGTACT |
| acid sequence) | GCTGCAATATTGTTAACGTGAGTCTTGTAAAACCTTCTTTTTA |
| | CGTTTACTCTCGTGTTAAAAATCTGAATTCTTCTAGAGTTCCT |
| | GATCTTCTGGTCTAA |
| SEQ ID NO: 22 | MYSFVSEETGTLIVNSVLLFLAFVVFLLVTLAILTALRLCAYCC |
| SARS-COV-2 E | NIVNVSLVKPSFYVYSRVKNLNSSRVPDLLV |
| Protein (amino | |
| acid sequence) | |
| SEQ ID NO: 23 | ATGTACTCATTCGTTTCGGAAGAGACAGGTACGTTAATAGTT |
| USA_WA-UW- | AATAGCGTACTTCTTTTTCTTGCTTTCGTGGTATTCTTGCTAG |
| 1588 Variant E | TTACACTAGCCATCCTTACTGCGCTTCGATTGTGTGCGTACT |
| Protein (nucleic | GCTGCAATATTGTTAACGTGAGTCTTGTAAAACCTTCTTTTTA |
| acid sequence) | CGTTTACTCTCGTGTTAAAAATCTGAATTCTTCTAGAGTTCTT |
| | GATCTTCTGGTCTAA |
| SEQ ID NO: 24 | MYSFVSEETGTLIVNSVLLFLAFVVFLLVTLAILTALRLCAYCC |
| USA_WA-UW- | NIVNVSLVKPSFYVYSRVKNLNSSRVLDLLV |
| 1588 Variant E | |
| Protein (amino | |
| acid sequence) | |
| SEQ ID NO: 25 | ATGTACTCATTCGTTTCGGAAGAGACAGGTACGTTAATAGTT |
| SNU01 Variant | AATAGCGTACTTCTTTTTCTTGCTTTCGTGGTATTCTTGCTAG |
| E Protein | TTACACTAGCCATCCTTACTGCGCATCGATTGTGTGCGTACT |
| (nucleic acid | GCTGCAATATTGTTAACGTGAGTCTTGTAAAACCTTCTTTTTA |
| sequence) | CGTTTACTCTCGTGTTAAAAATCTGAATTCTTCTAGAGTTCCT |
| | GATCTTCTGGTCTAA |
| SEQ ID NO: 26 | MYSFVSEETGTLIVNSVLLFLAFVVFLLVTLAILTAHRLCAYCC |
| SNU01 Variant | NIVNVSLVKPSFYVYSRVKNLNSSRVPDLLV |
| E Protein (amino | |
| acid sequence) | |
| SEQ ID NO: 27 | ATGTCTGATAATGGACCCCAAAATCAGCGAAATGCACCCCG |
| SARS-COV-2 N | CATTACGTTTGGTGGACCCTCAGATTCAACTGGCAGTAACCA |
| Protein (nucleic | GAATGGAGAACGCAGTGGGGCGCGATCAAAACAACGTCGGC |
| acid sequence) | CCCAAGGTTTACCCAATAATACTGCGTCTTGGTTCACCGCTC |
| | TCACTCAACATGGCAAGGAAGACCTTAAATTCCCTCGAGGA |
| | CAAGGCGTTCCAATTAACACCAATAGCAGTCCAGATGACCA |
| | AATTGGCTACTACCGAAGAGCTACCAGACGAATTCGTGGTG |
| | GTGACGGTAAAATGAAAGATCTCAGTCCAAGATGGTATTTCT |
| | ACTACCTAGGAACTGGGCCAGAAGCTGGACTTCCCTATGGT |
| | GCTAACAAAGACGGCATCATATGGGTTGCAACTGAGGGAGC |
| | CTTGAATACACCAAAAGATCACATTGGCACCCGCAATCCTGC |
| | TAACAATGCTGCAATCGTGCTACAACTTCCTCAAGGAACAAC |
| | ATTGCCAAAAGGCTTCTACGCAGAAGGGAGCAGAGGCGGCA |
| | GTCAAGCCTCTTCTCGTTCCTCATCACGTAGTCGCAACAGTT |
| | CAAGAAATTCAACTCCAGGCAGCAGTAGGGGAACTTCTCCT |
| | GCTAGAATGGCTGGCAATGGCGGTGATGCTGCTCTTGCTTTG |
| | CTGCTGCTTGACAGATTGAACCAGCTTGAGAGCAAAATGTCT |
| | GGTAAAGGCCAACAACAACAAGGCCAAACTGTCACTAAGAA |
| | ATCTGCTGCTGAGGCTTCTAAGAAGCCTCGGCAAAAACGTA |
| | CTGCCACTAAAGCATACAATGTAACACAAGCTTTCGGCAGA |
| | CGTGGTCCAGAACAAACCCAAGGAAATTTTGGGGACCAGGA |
| | ACTAATCAGACAAGGAACTGATTACAAACATTGGCCGCAAA |
| | TTGCACAATTTGCCCCCAGCGCTTCAGCGTTCTTCGGAATGT |
| | CGCGCATTGGCATGGAAGTCACACCTTCGGGAACGTGGTTG |
| | ACCTACACAGGTGCCATCAAATTGGATGACAAAGATCCAAA |
| | TTTCAAAGATCAAGTCATTTTGCTGAATAAGCATATTGACGC |
| | ATACAAAACATTCCCACCAACAGAGCCTAAAAAGGACAAAA |
| | AGAAGAAGGCTGATGAAACTCAAGCCTTACCGCAGAGACAG |
| | AAGAAACAGCAAACTGTGACTCTTCTTCCTGCTGCAGATTTG |
| | GATGATTTCTCCAAACAATTGCAACAATCCATGAGCAGTGCT |
| | GACTCAACTCAGGCCTAA |
| SEQ ID NO: 28 | MSDNGPQNQRNAPRITFGGPSDSTGSNQNGERSGARSKQRRPQ |
| SARS-COV-2 N | GLPNNTASWFTALTQHGKEDLKFPRGQGVPINTNSSPDDQIGY |
| Protein (amino | YRRATRRIRGGDGKMKDLSPRWYFYYLGTGPEAGLPYGANKD |
| acid sequence) | GIIWVATEGALNTPKDHIGTRNPANNAAIVLQLPQGTTLPKGFY |
| | AEGSRGGSQASSRSSSRSRNSSRNSTPGSSRGTSPARMAGNGGD |
| | AALALLLLDRLNQLESKMSGKGQQQQGQTVTKKSAAEASKKP |
| | RQKRTATKAYNVTQAFGRRGPEQTQGNFGDQELIRQGTDYKH |
| | WPQIAQFAPSASAFFGMSRIGMEVTPSGTWLTYTGAIKLDDKD |
| | PNFKDQVILLNKHIDAYKTFPPTEPKKDKKKKADETQALPQRQ |
| | KKQQTVTLLPAADLDDFSKQLQQSMSSADSTQA |
| SEQ ID NO: 29 | CTAGCTGGCCAGACATGATAAGATACATTGATGAGTTTGGA |
| SV40 p(A) tail | CAAACCACAACTAGAATGCAGTGAAAAAAATGCTTTATTTG |
| (nucleic acid | TGAAATTTGTGATGCTATTGCTTTATTTGTAACCATTATAAGC |
| sequence) | TGCAATAAACAAGTTAACAACAACAATTGCATTCATTTTATG |
| | TTTCAGGTTCAGGGGGAGGTGTGGGAGGTTTTTTAAAGCAA |
| | GTAAAACCTCTACAAATGTGGTATGG |
| SEQ ID NO: 30 | CTGTGCCTTCTAGTTGCCAGCCATCTGTTGTTTGCCCCTCCCC |
| bGH p(A) tail | CGTGCCTTCCTTGACCCTGGAAGGTGCCACTCCCACTGTCCT |
| (nucleic acid | TTCCTAATAAAATGAGGAAATTGCATCGCATTGTCTGAGTAG |
| sequence) | GTGTCATTCTATTCTGGGGGGTGGGGTGGGGCAGGACAGCA |
| | AGGGGGAGGATTGGGAAGACAATAGCAGGCATGCTGGGGA |
| | TGCGGTGGGCTCTATGG |
| SEQ ID NO: 31 | TCAATATTGGCCATTAGCCATATTATTCATTGGTTATATAGC |
| Human CMV | ATAAATCAATATTGGCTATTGGCCATTGCATACGTTGTATCT |
| promoter | ATATCATAATATGTACATTTATATTGGCTCATGTCCAATATG |
| (nucleic acid | ACCGCCATGTTGGCATTGATTATTGACTAGTTATTAATAGTA |
| sequence) | ATCAATTACGGGGTCATTAGTTCATAGCCCATATATGGAGTT |
| | CCGCGTTACATAACTTACGGTAAATGGCCCGCCTGGCTGACC |
| | GCCCAACGACCCCCGCCCATTGACGTCAATAATGACGTATGT |
| | TCCCATAGTAACGCCAATAGGGACTTTCCATTGACGTCAATG |
| | GGTGGAGTATTTACGGTAAACTGCCCACTTGGCAGTACATCA |
| | AGTGTATCATATGCCAAGTCCGCCCCCTATTGACGTCAATGA |
| | CGGTAAATGGCCCGCCTGGCATTATGCCCAGTACATGACCTT |
| | ACGGGACTTTCCTACTTGGCAGTACATCTACGTATTAGTCAT |
| | CGCTATTACCATGGTGATGCGGTTTTGGCAGTACACCAATGG |
| | GCGTGGATAGCGGTTTGACTCACGGGGATTTCCAAGTCTCCA |
| | CCCCATTGACGTCAATGGGAGTTTGTTTTGGCACCAAAATCA |
| | ACGGGACTTTCCAAAATGTCGTAATAACCCCGCCCCGTTGAC |
| | GCAAATGGGCGGTAGGCGTGTACGGTGGGAGGTCTATATAA |
| | GCAGAGCTCGTTTAGTGAACCGTCAGATC |
| SEQ ID NO: 32 | GCGCAGCACCATGGCCTGAAATAACCTCTGAAAGAGGAACT |
| SV40 promoter | TGGTTAGGTACCTTCTGAGGCGGAAAGAACCAGCTGTGGAA |
| (nucleic acid | TGTGTGTCAGTTAGGGTGTGGAAAGTCCCCAGGCTCCCCAGC |
| sequence) | AGGCAGAAGTATGCAAAGCATGCATCTCAATTAGTCAGCAA |
| | CCAGGTGTGGAAAGTCCCCAGGCTCCCCAGCAGGCAGAAGT |
| | ATGCAAAGCATGCATCTCAATTAGTCAGCAACCATAGTCCCG |
| | CCCCTAACTCCGCCCATCCCGCCCCTAACTCCGCCCAGTTCC |
| | GCCCATTCTCCGCCCCATGGCTGACTAATTTTTTTTATTTATG |
| | CAGAGGCCGAGGCCGCCTCGGCCTCTGAGCTATTCCAGAAG |
| | TAGTGAGGAGGCTTTTTTGGAGGCCTAGGCTTTTGCAAAAAG |
| | CTT |
| SEQ ID NO: 33 | CGTTACATAACTTACGGTAAATGGCCCGCCTGGCTGACCGCC |
| CAG promoter | CAACGACCCCCGCCCATTGACGTCAATAATGACGTATGTTCC |
| (nucleic acid | CATAGTAACGCCAATAGGGACTTTCCATTGACGTCAATGGGT |
| sequence) | GGAGTATTTACGGTAAACTGCCCACTTGGCAGTACATCAAGT |
| | GTATCATATGCCAAGTACGCCCCCTATTGACGTCAATGACGG |
| | TAAATGGCCCGCCTGGCATTATGCCCAGTACATGACCTTATG |
| | GGACTTTCCTACTTGGCAGTACATCTACGTATTAGTCATCGC |
| | TATTACCATGGTGATGCGGTTTTGGCAGTACATCAATGGGCG |
| | TGGATAGCGGTTTGACTCACGGGGATTTCCAAGTCTCCACCC |
| | CATTGACGTCAATGGGAGTTTGTTTTGGCACCAAAATCAACG |
| | GGACTTTCCAAAATGTCGTAACAACTCCGCCCCATTGACGCA |
| | AATGGGCGGTAGGCGTGTACGGTGGGAGGTCTATATAAGCA |
| | GAGCT |
| SEQ ID NO: 34 | PPFGAGRPGQFGDI |
| Chikungunya | |
| virus antigen | |
| (amino acid | |
| sequence) | |
| SEQ ID NO: 35 | TAECKDKNL |
| Chikungunya | |
| virus antigen | |
| (amino acid | |
| sequence) | |
| SEQ ID NO: 36 | VRYKCNCGG |
| Chikungunya | |
| virus antigen | |
| (amino acid | |
| sequence) | |
| SEQ ID NO: 37 | GGACCTGCAGGGCCTGAAATAACCTCTGAAAGAGGAACTTG |
| SV40 Enhancer | GTTAGGTACCTTCTGAGGCGGAAAGAACCAGCTGTGGAATG |
| (nucleic acid | TGTGTCAGTTAGGGTGTGGAAAGTCCCCAGGCTCCCCAGCA |
| sequence) | GGCAGAAGTATGCAAAGCATGCATCTCAATTAGTCAGCAAC |
| | CAGGTGTGGAAAGTCCCCAGGCTCCCCAGCAGGCAGAAGTA |
| | TGCAAAGCATGCATCTCAATTAGTCAGCAACCATAGTCCCAC |
| | TAGT |
| SEQ ID NO: 38 | GCTCCGGTGCCCGTCAGTGGGCAGAGCGCACATCGCCCACA |
| hEF1-HTLV | GTCCCCGAGAAGTTGGGGGGAGGGGTCGGCAATTGAACGGG |
| Promoter | TGCCTAGAGAAGGTGGCGCGGGGTAAACTGGGAAAGTGATG |
| (nucleic acid | TCGTGTACTGGCTCCGCCTTTTTCCCGAGGGTGGGGGAGAAC |
| sequence) | CGTATATAAGTGCAGTAGTCGCCGTGAACGTTCTTTTTCGCA |
| | ACGGGTTTGCCGCCAGAACACAGCTGAAGCTTCGAGGGGCT |
| | CGCATCTCTCCTTCACGCGCCCGCCGCCCTACCTGAGGCCGC |
| | CATCCACGCCGGTTGAGTCGCGTTCTGCCGCCTCCCGCCTGT |
| | GGTGCCTCCTGAACTGCGTCCGCCGTCTAGGTAAGTTTAAAG |
| | CTCAGGTCGAGACCGGGCCTTTGTCCGGCGCTCCCTTGGAGC |
| | CTACCTAGACTCAGCCGGCTCTCCACGCTTTGCCTGACCCTG |
| | CTTGCTCAACTCTACGTCTTTGTTTCGTTTTCTGTTCTGCGCA |
| | GTTACAGATCCAAGCTGTGACCGGCGCCTACCTGAGATCACC |
| | GGTCACCATGGAGATCAAGGTGCTGTTTGCCCTCATCTGTAT |
| | TGCTGTTGCTGAGGCA |
| SEQ ID NO: 39 | GTTAATCTCACCACCCGAACTCAACTCCCACCCGCATATACA |
| SARS-COV-2 | AATTCCTTCACCAGAGGAGTGTACTATCCTGACAAAGTGTTT |
| D614G Spike | CGGTCAAGTGTCCTCCACTCTACTCAGGACCTCTTTCTGCCTT |
| Truncated S1 | TCTTTTCTAACGTTACATGGTTTCATGCAATCCATGTGTCTGG |
| (nucleic acid | GACAAACGGCACCAAACGCTTCGACAACCCTGTATTGCCATT |
| sequence) | CAATGATGGGGTGTACTTTGCCTCCACAGAGAAATCCAACAT |
| | CATTCGAGGATGGATTTTCGGGACTACTCTGGACTCAAAGAC |
| | ACAGAGCCTGCTGATCGTTAACAACGCCACAAACGTTGTCAT |
| | CAAAGTGTGCGAATTCCAGTTTTGCAATGATCCCTTCCTGGG |
| | AGTGTACTATCACAAGAATAACAAGTCCTGGATGGAGAGCG |
| | AATTTCGGGTCTACAGCAGCGCAAACAACTGCACCTTCGAGT |
| | ACGTGAGTCAACCCTTTCTGATGGACCTGGAAGGGAAACAG |
| | GGAAACTTCAAGAACCTGAGAGAGTTTGTCTTTAAGAACAT |
| | CGACGGCTATTTTAAGATCTATAGTAAGCATACGCCTATCAA |
| | CCTGGTAAGGGATCTTCCCCAGGGCTTTTCAGCCCTGGAACC |
| | TTTGGTTGACTTGCCTATTGGTATCAATATCACCAGATTTCA |
| | GACCCTTCTGGCATTGCATCGGTCTTATCTTACTCCAGGTGA |
| | TTCCTCCTCCGGGTGGACTGCCGGCGCCGCTGCCTACTATGT |
| | CGGCTATCTGCAACCAAGAACGTTCCTGCTCAAGTACAACG |
| | AAAACGGCACTATTACGGATGCTGTTGATTGTGCCCTGGACC |
| | CTCTGTCTGAGACTAAATGCACCCTCAAGAGCTTTACCGTTG |
| | AGAAGGGGATTTACCAAACCAGTAATTTCCGGGTCCAACCC |
| | ACCGAAAGCATTGTGCGGTTCCCAAATATCACCAATCTGTGT |
| | CCCTTTGGCGAAGTGTTCAATGCTACAAGGTTTGCTTCTGTG |
| | TACGCATGGAATAGGAAACGCATCTCCAATTGTGTCGCTGAT |
| | TACTCCGTGCTGTACAATTCCGCCTCTTTCTCAACCTTCAAGT |
| | GTTATGGCGTTTCACCTACCAAACTTAACGACCTGTGCTTCA |
| | CTAATGTGTATGCCGACTCTTTTGTGATACGAGGCGATGAAG |
| | TGAGACAGATTGCACCAGGGCAGACCGGCAAAATTGCCGAC |
| | TACAACTACAAGCTTCCAGATGACTTTACCGGATGTGTTATT |
| | GCATGGAACTCAAACAATCTGGATTCCAAGGTGGGTGGCAA |
| | CTATAACTACCTGTATAGACTGTTCAGGAAATCCAACCTGAA |
| | ACCATTCGAGCGAGATATAAGCACAGAAATCTACCAGGCTG |
| | GAAGTACGCCCTGCAACGGCGTGGAAGGGTTCAACTGCTAC |
| | TTCCCATTGCAGAGTTACGGATTCCAGCCTACAAACGGGGTG |
| | GGTTACCAACCCTATCGTGTCGTAGTCCTGAGTTTTGAGCTC |
| | CTCCATGCCCCAGCCACAGTCTGTGGCCCCAAGAAAAGCAC |
| | CAATCTGGTGAAGAACAAATGCGTGAACTTTAACTTTAACG |
| | GACTCACAGGAACCGGCGTATTGACGGAGAGTAACAAGAAG |
| | TTCCTGCCATTCCAGCAGTTCGGTCGCGATATTGCCGACACT |
| | ACCGACGCTGTCCGAGATCCCCAGACATTGGAGATTCTTGAT |
| | ATCACACCCTGTAGTTTCGGCGGAGTGAGCGTGATTACGCCC |
| | GGAACCAATACCAGCAATCAGGTTGCCGTCCTGTATCAGGA |
| | CGTGAATTGCACCGAGGTACCTGTCGCCATCCACGCTGACCA |
| | ACTTACACCCACATGGCGAGTATATTCCACCGGCTCCAACGT |
| | CTTTCAGACACGTGCTGGATGTCTGATCGGTGCAGAACACGT |
| | TAATAATAGCTACGAGTGTGATATCCCCATCGGTGCTGGAAT |
| | ATGCGCCTCTTATCAAACTCAAACCAACTCTCCTAGGCGGGC |
| | ACGT |
| SEQ ID NO: 40 | VNLTTRTQLPPAYTNSFTRGVYYPDKVFRSSVLHSTQDLFLPFF |
| SARS-COV-2 | SNVTWFHAIHVSGTNGTKRFDNPVLPFNDGVYFASTEKSNIIRG |
| D614G Spike | WIFGTTLDSKTQSLLIVNNATNVVIKVCEFQFCNDPFLGVYYHK |
| Truncated S1 | NNKSWMESEFRVYSSANNCTFEYVSQPFLMDLEGKQGNFKNL |
| (amino acid | REFVFKNIDGYFKIYSKHTPINLVRDLPQGFSALEPLVDLPIGINI |
| sequence) | TRFQTLLALHRSYLTPGDSSSGWTAGAAAYYVGYLQPRTFLLK |
| | YNENGTITDAVDCALDPLSETKCTLKSFTVEKGIYQTSNFRVQP |
| | TESIVRFPNITNLCPFGEVENATRFASVYAWNRKRISNCVADYS |
| | VLYNSASFSTFKCYGVSPTKLNDLCFTNVYADSFVIRGDEVRQI |
| | APGQTGKIADYNYKLPDDFTGCVIAWNSNNLDSKVGGNYNYL |
| | YRLFRKSNLKPFERDISTEIYQAGSTPCNGVEGENCYFPLQSYGF |
| | QPTNGVGYQPYRVVVLSFELLHAPATVCGPKKSTNLVKNKCV |
| | NFNFNGLTGTGVLTESNKKFLPFQQFGRDIADTTDAVRDPQTLE |
| | ILDITPCSFGGVSVITPGTNTSNQVAVLYQDVNCTEVPVAIHAD |
| | QLTPTWRVYSTGSNVFQTRAGCLIGAEHVNNSYECDIPIGAGIC |
| | ASYQTQTNSPRRAR |
| SEQ ID NO: 41 | CCCCTCTCCCTCCCCCCCCCCTAACGTTACTGGCCGAAGCCG |
| IRES Bridge | CTTGGAATAAGGCCGGTGTGCGTTTGTCTATATGTTATTTTCC |
| (nucleic acid | ACCATATTGCCGTCTTTTGGCAATGTGAGGGCCCGGAAACCT |
| sequence) | GGCCCTGTCTTCTTGACGAGCATTCCTAGGGGTCTTTCCCCTC |
| | TCGCCAAAGGAATGCAAGGTCTGTTGAATGTCGTGAAGGAA |
| | GCAGTTCCTCTGGAAGCTTCTTGAAGACAAACAACGTCTGTA |
| | GCGACCCTTTGCAGGCAGCGGAACCCCCCACCTGGCGACAG |
| | GTGCCTCTGCGGCCAAAAGCCACGTGTATAAGATACACCTG |
| | CAAAGGCGGCACAACCCCAGTGCCACGTTGTGAGTTGGATA |
| | GTTGTGGAAAGAGTCAAATGGCTCTCCTCAAGCGTATTCAAC |
| | AAGGGGCTGAAGGATGCCCAGAAGGTACCCCATTGTATGGG |
| | ATCTGATCTGGGGCCTCGGTGCACATGCTTTACATGTGTTTA |
| | GTCGAGGTTAAAAAAACGTCTAGGCCCCCCGAACCACGGGG |
| | ACGTGGTTTTCCTTTGAAAAACACGA |
| SEQ ID NO: 42 | ATGTGTCAATCACGCTACCTCCTCTTTTTGGCCACCCTTGCCC |
| Mouse IL-12 | TCCTAAACCACCTCAGTTTGGCCAGGGTCATTCCAGTCTCTG |
| p35 (nucleic acid | GACCTGCCAGGTGTCTTAGCCAGTCCCGAAACCTGCTGAAG |
| sequence) | ACCACAGATGACATGGTGAAGACGGCCAGAGAAAAACTGA |
| | AACATTATTCCTGCACTGCTGAAGACATCGATCATGAAGACA |
| | TCACACGGGACCAAACCAGCACATTGAAGACCTGTTTACCA |
| | CTGGAACTACACAAGAACGAGAGTTGCCTGGCTACTAGAGA |
| | GACTTCTTCCACAACAAGAGGGAGCTGCCTGCCCCCACAGA |
| | AGACGTCTTTGATGATGACCCTGTGCCTTGGTAGCATCTATG |
| | AGGACTTGAAGATGTACCAGACAGAGTTCCAGGCCATCAAC |
| | GCAGCACTTCAGAATCACAACCATCAGCAGATCATTCTAGA |
| | CAAGGGCATGCTGGTGGCCATCGATGAGCTGATGCAGTCTCT |
| | GAATCATAATGGCGAGACTCTGCGCCAGAAACCTCCTGTGG |
| | GAGAAGCAGACCCTTACAGAGTGAAAATGAAGCTCTGCATC |
| | CTGCTTCACGCCTTCAGCACCCGCGTCGTGACCATCAACAGG |
| | GTGATGGGCTATCTGAGCTCCGCCTGA |
| SEQ ID NO: 43 | MCQSRYLLFLATLALLNHLSLARVIPVSGPARCLSQSRNLLKTT |
| Mouse IL-12 | DDMVKTAREKLKHYSCTAEDIDHEDITRDQTSTLKTCLPLELH |
| p35 (amino acid | KNESCLATRETSSTTRGSCLPPQKTSLMMTLCLGSIYEDLKMYQ |
| sequence) | TEFQAINAALQNHNHQQIILDKGMLVAIDELMQSLNHNGETLR |
| | QKPPVGEADPYRVKMKLCILLHAFSTRVVTINRVMGYLSSA |
| SEQ ID NO: 44 | ATGTGTCCTCAGAAGCTAACCATCTCCTGGTTTGCCATCGTT |
| Mouse IL-12 | TTGCTGGTGTCTCCACTCATGGCCATGTGGGAGCTGGAGAAA |
| p40 (nucleic acid | GACGTTTATGTTGTAGAGGTGGACTGGACTCCCGATGCCCCT |
| sequence) | GGAGAAACAGTGAACCTCACCTGTGACACGCCTGAAGAAGA |
| | TGACATCACCTGGACCTCAGACCAGAGACATGGAGTCATAG |
| | GCTCTGGAAAGACCCTGACCATCACTGTCAAAGAGTTTCTAG |
| | ATGCTGGCCAGTACACCTGCCACAAAGGAGGCGAGACTCTG |
| | AGCCACTCACATCTGCTGCTCCACAAGAAGGAAAATGGAAT |
| | TTGGTCCACTGAAATTTTAAAAAATTTCAAAAACAAGACTTT |
| | CCTGAAGTGTGAAGCACCAAATTACTCCGGACGGTTCACGT |
| | GCTCATGGCTGGTGCAAAGAAACATAGACTTGAAGTTCAAC |
| | ATCAAGAGCAGTAGCAGTTCCCCTGACTCTCGGGCAGTGAC |
| | ATGTGGAATGGCGTCTCTGTCTGCAGAGAAGGTCACACTGG |
| | ACCAAAGGGACTATGAGAAGTATTCAGTGTCCTGCCAGGAG |
| | GATGTCACCTGCCCAACTGCCGAGGAGACCCTGCCCATTGA |
| | ACTGGCGTTGGAAGCACGGCAGCAGAATAAATATGAGAACT |
| | ACAGCACCAGCTTCTTCATCAGGGACATCATCAAACCAGAC |
| | CCGCCCAAGAACTTGCAGATGAAGCCTTTGAAGAACTCACA |
| | GGTGGAGGTCAGCTGGGAGTACCCTGACTCCTGGAGCACTC |
| | CCCATTCCTACTTCTCCCTCAAGTTCTTTGTTCGAATCCAGCG |
| | CAAGAAAGAAAAGATGAAGGAGACAGAGGAGGGGTGTAAC |
| | CAGAAAGGTGCGTTCCTCGTAGAGAAGACATCTACCGAAGT |
| | CCAATGCAAAGGCGGGAATGTCTGCGTGCAAGCTCAGGATC |
| | GCTATTACAATTCCTCATGCAGCAAGTGGGCATGTGTTCCCT |
| | GCAGGGTCCGATCCTAG |
| SEQ ID NO: 45 | MCPQKLTISWFAIVLLVSPLMAMWELEKDVYVVEVDWTPDAP |
| Mouse IL-12 | GETVNLTCDTPEEDDITWTSDQRHGVIGSGKTLTITVKEFLDAG |
| p40 (amino acid | QYTCHKGGETLSHSHLLLHKKENGIWSTEILKNFKNKTFLKCE |
| sequence) | APNYSGRFTCSWLVQRNIDLKFNIKSSSSSPDSRAVTCGMASLS |
| | AEKVTLDQRDYEKYSVSCQEDVTCPTAEETLPIELALEARQQN |
| | KYENYSTSFFIRDIIKPDPPKNLQMKPLKNSQVEVSWEYPDSWS |
| | TPHSYFSLKFFVRIQRKKEKMKETEEGCNQKGAFLVEKTSTEV |
| | QCKGGNVCVQAQDRYYNSSCSKWACVPCRVRS |
| SEQ ID NO: 46 | ATGGGTCCAGCGCGCAGCCTCCTCCTTGTGGCTACCCTGGTC |
| Human IL-12 | CTCCTGGACCACCTCAGTTTGGCCAGAAACCTCCCCGTGGCC |
| p35 (nucleic acid | ACTCCAGACCCAGGAATGTTCCCATGCCTTCACCACTCCCAA |
| sequence) | AACCTGCTGAGGGCCGTCAGCAACATGCTCCAGAAGGCCAG |
| | ACAAACTCTAGAATTTTACCCTTGCACTTCTGAAGAGATTGA |
| | TCATGAAGATATCACAAAAGATAAAACCAGCACAGTGGAGG |
| | CCTGTTTACCATTGGAATTAACCAAGAATGAGAGTTGCCTAA |
| | ATTCCAGAGAGACCTCTTTCATAACTAATGGGAGTTGCCTGG |
| | CCTCCAGAAAGACCTCTTTTATGATGGCCCTGTGCCTTAGTA |
| | GTATTTATGAAGACTTGAAGATGTACCAGGTGGAGTTCAAG |
| | ACCATGAATGCAAAGCTTCTGATGGATCCTAAGAGGCAGAT |
| | CTTTCTAGATCAAAACATGCTGGCAGTTATTGATGAGCTGAT |
| | GCAGGCCCTGAATTTCAACAGTGAGACTGTGCCACAAAAAT |
| | CCTCCCTTGAAGAACCGGATTTTTATAAAACTAAAATCAAGC |
| | TCTGCATACTTCTTCATGCTTTCAGAATTCGGGCAGTGACTA |
| | TTGATAGAGTGATGAGCTATCTGAATGCTTCCTAA |
| SEQ ID NO: 47 | MGPARSLLLVATLVLLDHLSLARNLPVATPDPGMFPCLHHSQN |
| Human IL-12 | LLRAVSNMLQKARQTLEFYPCTSEEIDHEDITKDKTSTVEACLP |
| p35 (amino acid | LELTKNESCLNSRETSFITNGSCLASRKTSFMMALCLSSIYEDLK |
| sequence) | MYQVEFKTMNAKLLMDPKRQIFLDQNMLAVIDELMQALNENS |
| | ETVPQKSSLEEPDFYKTKIKLCILLHAFRIRAVTIDRVMSYLNAS |
| SEQ ID NO: 48 | ATGGGTCACCAGCAGTTGGTCATCTCTTGGTTTTCCCTGGTTT |
| Human IL-12 | TTCTGGCATCTCCCCTCGTGGCCATATGGGAACTGAAGAAAG |
| p40 (nucleic acid | ATGTTTATGTCGTAGAATTGGATTGGTATCCGGATGCCCCTG |
| sequence) | GAGAAATGGTGGTCCTCACCTGTGACACCCCTGAAGAAGAT |
| | GGTATCACCTGGACCTTGGACCAGAGCAGTGAGGTCTTAGG |
| | CTCTGGCAAAACCCTGACCATCCAAGTCAAAGAGTTTGGAG |
| | ATGCTGGCCAGTACACCTGTCACAAAGGAGGCGAGGTTCTA |
| | AGCCATTCGCTCCTGCTGCTTCACAAAAAGGAAGATGGAATT |
| | TGGTCCACTGATATTTTAAAGGACCAGAAAGAACCCAAAAA |
| | TAAGACCTTTCTAAGATGCGAGGCCAAGAATTATTCTGGACG |
| | TTTCACCTGCTGGTGGCTGACGACAATCAGTACTGATTTGAC |
| | ATTCAGTGTCAAAAGCAGCAGAGGCTCTTCTGACCCCCAAG |
| | GGGTGACGTGCGGAGCTGCTACACTCTCTGCAGAGAGAGTC |
| | AGAGGGGACAACAAGGAGTATGAGTACTCAGTGGAGTGCCA |
| | GGAGGACAGTGCCTGCCCAGCTGCTGAGGAGAGTCTGCCCA |
| | TTGAGGTCATGGTGGATGCCGTTCACAAGCTCAAGTATGAA |
| | AACTACACCAGCAGCTTCTTCATCAGGGACATCATCAAACCT |
| | GACCCACCCAAGAACTTGCAGCTGAAGCCATTAAAGAATTC |
| | TCGGCAGGTGGAGGTCAGCTGGGAGTACCCTGACACCTGGA |
| | GTACTCCACATTCCTACTTCTCCCTGACATTCTGCGTTCAGGT |
| | CCAGGGCAAGAGCAAGAGAGAAAAGAAAGATAGAGTCTTC |
| | ACGGACAAGACCTCAGCCACGGTCATCTGCCGCAAAAATGC |
| | CAGCATTAGCGTGCGGGCCCAGGACCGCTACTATAGCTCATC |
| | TTGGAGCGAATGGGCATCTGTGCCCTGCAGTTAGAC |
| SEQ ID NO: 49 | MGHQQLVISWFSLVFLASPLVAIWELKKDVYVVELDWYPDAP |
| Human IL-12 | GEMVVLTCDTPEEDGITWTLDQSSEVLGSGKTLTIQVKEFGDA |
| p40 (amino acid | GQYTCHKGGEVLSHSLLLLHKKEDGIWSTDILKDQKEPKNKTF |
| sequence) | LRCEAKNYSGRFTCWWLTTISTDLTFSVKSSRGSSDPQGVTCG |
| | AATLSAERVRGDNKEYEYSVECQEDSACPAAEESLPIEVMVDA |
| | VHKLKYENYTSSFFIRDIIKPDPPKNLQLKPLKNSRQVEVSWEY |
| | PDTWSTPHSYFSLTFCVQVQGKSKREKKDRVFTDKTSATVICR |
| | KNASISVRAQDRYYSSSWSEWASVPCS |
| SEQ ID NO: 51 | ACCGGCGCCTACCTGAGATCACCGGTAACGTAGATAAGCGC |
| AgeIMhis-F | TTGCCACAACCCGGGATC |
| SEQ ID NO: 52 | TATTTGAATGTATTTAGAAAAATAAACAAATAGGGAAATGA |
| pAcelsion-R | AGAGCCCATACCACATTTGTAGAGGTTTTAC |
| SEQ ID NO: 53 | ACCGGCGCCTACCTGAGATCACCGGTGCCGCCACCATGGAG |
| CelsionAgeIsp1-F | ATCAAGGTGCTGTTTG |
| SEQ ID NO: 54 | TGAGGGAAGCGGCCGCCCGGGTCGACTCTAGAGGATCCCG |
| IRESSalI-R | GGTTGTGGCAAGCTTATCATCGTGTTTTTCAAAGGAAAAC |
| SEQ ID NO: 56 | CGGCTAAGCGGCCGCAGCCTCGAGCAGCTAGCTGGCCAGAC |
| PspXI-SV40pA-R | |
| SEQ ID NO: 57 | TATTTGAATGTATTTAGAAAAATAAACAAATAGG |
| Cls-SapI-R | |
| SEQ ID NO: 58 | ATTGGACATGAGCCAATATAAATGTACGAAGATCTGTCGAG |
| Cls-BglII-R-2 | CCATGTG |
| SEQ ID NO: 59 | AGAACTTTAAAAGTGCTCATCATTG |
| Cls-XmnI-R | |
| SEQ. ID NO. 60 | ATACGTAAGCGGCCGCAGCCTCG |
| CLS-PspXI-IRES-F | |
| SEQ. ID NO. 61 | TCGTGTTTTTCAAAGGAAAACCAC |
| IREScelsion-R | |
| SEQ. ID NO. 62 | ACGTGGTTTTCCTTTGAAAAACACGATGATAAGCTTGCCACA |
| Mhiscelsion-F | ACCCG |
| SEQ. ID NO. 63 | ATCATGTCTGGCCAGCTAGC |
| CLS-M-R | |
| SEQ. ID NO. 64 | CTAGACTCGAGACAGTCCACCACAATCTAGAGCCACCATGG |
| CLS-XbaI-M-F | CTGACTCT |
| SEQ. ID NO. 65 | ACTCATCAATGTATCTTATCATGTCTGCTCGAacgcgtttattgtac |
| CLS-MluI-M-R | |

1-133. (canceled)

134. A composition, pharmaceutical composition, or vaccine comprising:
(a) a polynucleotide or a vector comprising the polynucleotide, and (b) a delivery component,
wherein the polynucleotide comprises a first antigen nucleic acid which encodes a first pathogen protein or an antigenic fragment thereof,
wherein the first antigen nucleic acid is operably linked to a first promoter, and
optionally wherein the delivery component is a poly-inosinic-polycytidylic acid, or a poloxamer, or derivative thereof.

135. The composition, pharmaceutical composition, or vaccine of claim 134, wherein the first antigen nucleic acid which encodes a first pathogen protein is selected from the group consisting of a viral protein, a bacterial protein, a parasite protein, and any antigenic fragment thereof.

136. The composition, pharmaceutical composition, or vaccine of claim 134, wherein the polynucleotide further comprises a second antigen nucleic acid which encodes a second pathogen protein or an antigenic fragment thereof.

137. The composition, pharmaceutical composition, or vaccine of claim 136, wherein the second antigen nucleic acid which encodes a second pathogen protein is selected from the group consisting of a viral protein, a bacterial protein, a parasite protein, and any antigenic fragment thereof.

138. The composition, pharmaceutical composition, or vaccine of claim 134, wherein the first pathogen protein and/or the second pathogen protein is/are selected from the group consisting of a Yersinia pestis antigen, a Mycobacterium tuberculosis antigen, an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, a Toxoplasma gondii antigen, a Plasmodium falciparum antigen, an influenza virus antigen, antigenic fragments thereof, and any combinations thereof.

139. The composition, pharmaceutical composition, or vaccine of claim 138, wherein the first pathogen protein and/or the second pathogen protein is/are selected from the group consisting of: a Yersiniapestis F1-Ag, a Yersiniapestis V-Ag, a Mycobacterium tuberculosis Apa antigen, a Mycobacterium tuberculosis HP65 antigen, a Mycobacterium tuberculosis rAg85A antigen, an E71 VP1 antigen, a GST-tagged E71-VP1 antigen, a Cox protein antigen, a GST-tagged Cox protein antigen, an HSV-1 envelope antigen, an HSV-2 envelope antigen, an HSV-2 gB2 antigen, an HSV-2 gC2 antigen, an HSV-2 gD2 antigen, an HSV-2 gE2 antigen, an HIV Env antigen, an HIV Gag antigen, an HIV Nef antigen, an HIV Pol antigen, an HPV minor capsid protein L2 antigen, an HCV NS3 antigen, a RSV F antigen, a RSV G antigen, a Dengue virus E protein antigen, a Dengue virus EDIII antigen, a Dengue virus NS1 antigen, a Dengue virus DEN-80E antigen, an Ebola virus GB antigen, an Ebola virus VP24 antigen, an Ebola virus VP40 antigen, an Ebola virus NP antigen, an Ebola virus VP30 antigen, an Ebola virus VP35 antigen, a Zika virus envelope domain III antigen, a Zika virus CKD antigen, a Chikungunya virus E1 glycoprotein subunit antigen, the MHC class I epitope PPFGAGRPGQFGDI (SEQ ID NO: 34), the MHC class I epitope TAECKDKNL (SEQ ID NO: 35), the MHC class II epitope VRYKCNCGG (SEQ ID NO: 36), a measles virus hemagglutinin protein MV-H antigen, a measles virus fusion protein MV-F antigen, a MERS-CoV S protein antigen, an antigen from the receptor-binding domain of the MERS-CoV S protein, an antigen from the membrane fusion domain of the MERS-CoV S protein, a SARS-CoV S protein antigen, an antigen from the receptor binding domain of the SARS-CoV S protein, an antigen from the membrane fusion domain of the SARS-CoV S protein, a SARS-CoV E protein antigen, a SARS-CoV M protein antigen, a Toxoplasma gondii MIC8 antigen, a Plasmodium falciparum: SERA5 polypeptide antigen, a Plasmodium falciparum: circumsporozite protein antigen, an influenza virus hemagglutinin (HA) antigen, an influenza virus neuraminidase (NA) antigen, an influenza virus matrix-2 (M2) protein antigen, antigenic fragments thereof, and any combination thereof.

140. (canceled)

141. The composition, pharmaceutical composition, or vaccine claim 136, wherein the second pathogen protein or antigenic fragment thereof is selected from the group consisting of: a SARS-CoV-2 membrane (M) protein or an antigenic fragment thereof, a SARS-CoV-2 envelope (E) protein or an antigenic fragment thereof, a SARS-CoV-2 nucleocapsid (N) protein or an antigenic fragment thereof, and any combination thereof.

142. The composition, pharmaceutical composition, or vaccine of claim 136, wherein the first pathogen protein is a SARS-CoV-2 protein or an antigenic fragment thereof selected from the group consisting of: a SARS CoV-2 S protein, a SARS-CoV-2 M protein, a SARS-CoV-2 E protein, a SARS-CoV-2 N protein, or an antigenic fragment thereof, and wherein the second pathogen protein is a SARS-CoV-2 protein or an antigenic fragment thereof selected from the group consisting of: a SARS CoV-2 S protein, a SARS-CoV-2 M protein, a SARS-CoV-2 E protein, a SARS-CoV-2 N protein, or an antigenic fragment thereof.

143-193. (canceled)

194. The composition, pharmaceutical composition, or vaccine of claim 134, wherein the vector is a DNA plasmid vector.

195. The composition, pharmaceutical composition, or vaccine of claim 194, wherein the DNA plasmid vector is selected from the group consisting of: pVac 1, pVac 4, and pVac 7.

196-253. (canceled)

254. The composition, pharmaceutical composition, or vaccine of claim 134, wherein the delivery component comprises a poloxamer with the following formula: 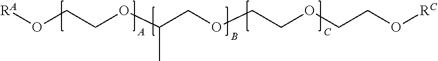
or a pharmaceutically acceptable salt thereof, wherein:
A represents an integer from 2 to 141;
B represents an integer from 16 to 67;
C represents an integer from 2 to 141;
R^{A} and R^{C} are the same or different, and are R′-L- or H, wherein at least one of R^{A} and R^{C} is R′-L-;
L is a bond, —CO—, —CH₂—O—, or —O—CO; and
R′ is a metal chelator.

255. The composition, pharmaceutical composition, or vaccine, of claim 254, wherein the metal chelator is R^{N}NH, R^{N2}N, or (R″—(N(R″)—CH₂CH₂)ₓ)₂ N—CH₂CO, wherein each x is independently 0-2, and wherein R″ is HO₂C—CH₂.

256. The composition, pharmaceutical composition, or vaccine of claim 254, wherein the metal chelator is a crown ether, a substituted-crown ether, a cryptand, or a substituted-cryptand.

257. The composition, pharmaceutical composition, or vaccine of claim 254, wherein the poloxamer is present in a solution with the polynucleotide or DNA plasmid vector from about 0.5%—about 5%.

258-261. (canceled)

262. The composition, pharmaceutical composition, or vaccine of claim 254, wherein the delivery component comprises Crown Poloxamer.

263-281. (canceled)

282. A host cell comprising the composition, pharmaceutical composition, or vaccine of claim 134.

283-284. (canceled)

285. A kit comprising the composition, pharmaceutical composition, or vaccine of claim 134.

286-290. (canceled)

291. A method of inducing an immune response in a subject, the method comprising administering an effective amount of the composition, pharmaceutical composition, or vaccine of claim 134.

292-293. (canceled)

294. A method of preventing, reducing the incidence of, attenuating or treating an infection in a subject, the method comprising administering an effective amount of the composition, pharmaceutical composition, or vaccine of claim 134 to the subject.

295-297. (canceled)

298. A method of making the composition, pharmaceutical composition, or vaccine of claim 134, the method comprising the steps of: (a) combining the delivery component with the polynucleotide in solution or, (b) lyophilizing the combined delivery component and polynucleotide to a powder, and (c) reconstituting the powder with a diluent to form a solution of nucleic acid complexed with the delivery component.
